(19) United States
(12) Patent Application Publication
(10) Pub. No.: US 2007/0149560 A1
(43) Pub. Date: Jun. 28, 2007

(54) Novel fused triazolones and the uses thereof

(75) Inventors: Susan Ashwell, Waltham, MA (US); Jayachandran Ezhuthachan, Waltham, MA (US); Paul Dermot Lyne, Waltham, MA (US); Nicholas John Newcombe, Cheshire (GB); Martin Pass, Cheshire (GB); Vibha Oza, Waltham, MA (US); Mei Su, Waltham, MA (US); Dorin Toader, Waltham, MA (US); Dingwei Yu, Waltham, MA (US); Yan Yu, Waltham, MA (US)

Correspondence Address: ASTRAZENECA R&D BOSTON, 35 GATEHOUSE DRIVE, WALTHAM, MA, 02451-1215, US

(73) Assignee: AstraZeneca AB, Sodertalje (SE)

(21) Appl. No.: 10/549,053

(22) PCT Filed: Mar. 10, 2004
(86) PCT No.: PCT/SE04/00351

§ 371 Date: Jan. 12, 2007

Related U.S. Application Data
(60) Provisional application No. 60/455,073, filed on Mar. 14, 2003.
(60) Provisional application No. 60/467,690, filed on May 2, 2003.
(60) Provisional application No. 60/536,343, filed on Jan. 14, 2004.

(51) Int. Cl. A61K 31/4745; C07D 487/02

(52) U.S. Cl. 514/292; 546/82

(57) ABSTRACT

This invention relates to novel compounds having the structural diagram (I) and to their pharmaceutical compositions and to their methods of use. These novel compounds provide a treatment or prophylaxis of cancer.

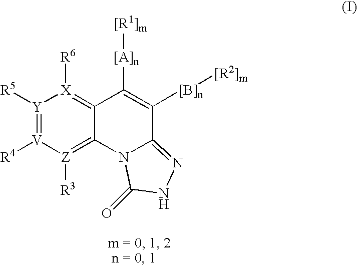

### FIELD OF THE INVENTION

The present invention relates to novel fused trizolones, their pharmaceutical compositions and methods of use. In addition, the present invention relates to therapeutic methods for the treatment and prevention of cancers.

### BACKGROUND OF THE INVENTION

Chemotherapy and radiation exposure are currently the major options for the treatment of cancer, but the utility of both these approaches is severely limited by drastic adverse effects on normal tissue, and the frequent development of tumor cell resistance. It is therefore highly desirable to improve the efficacy of such treatments in a way that does not increase the toxicity associated with them. One way to achieve this is by the use of specific sensitizing agents such as those described herein.

An individual cell replicates by making an exact copy of its chromosomes, and then segregating these into separate cells. This cycle of DNA replication, chromosome separation and division is regulated by mechanisms within the cell that maintain the order of the steps and ensure that each step is precisely carried out. Key to these processes are the cell cycle checkpoints (Hartwell et al., Science, Nov. 3, 1989, 246 (4930):629-34) where cells may arrest to ensure DNA repair mechanisms have time to operate prior to continuing through the cycle into mitosis. There are two such checkpoints in the cell cycle—the G1/S checkpoint that is regulated by p53 and the G2/M checkpoint that is monitored by the Ser/Thr kinase checkpoint kinase 1 (CHK1).

As the cell cycle arrest induced by these checkpoints is a crucial mechanism by which cells can overcome the damage resulting from radio- or chemotherapy, their abrogation by novel agents should increase the sensitivity of tumor cells to DNA damaging therapies. Additionally, the tumor specific abrogation of the G1/S checkpoint by p53 mutations in the majority of tumors can be exploited to provide tumor selective agents. One approach to the design of chemosensitizers that abrogate the G2/M checkpoint is to develop inhibitors of the key G2/M regulatory kinase CHK1, and this approach has been shown to work in a number of proof of concept studies. (Koniaras et al., Oncogene, 2001, 20:7453; Luo et al., Neoplasia, 2001, 3:411; Busby et al., Cancer Res., 2000, 60:2108; Jackson et al., Cancer Res., 2000, 60:566).

### SUMMARY OF THE INVENTION

In accordance with the present invention, the applicants have hereby discovered novel compounds that are potent inhibitors of the kinase CHK1 and therefore possess the ability to prevent cell cycle arrest at the G2/M checkpoint in response to DNA damage. These compounds are accordingly useful for their anti-cell-proliferation (such as anti-cancer) activity and are therefore useful in methods of treatment of the human or animal body. The invention also relates to processes for the manufacture of said fused compounds, to pharmaceutical compositions containing them and to their use in the manufacture of medicaments of use with the production of anti-cell proliferation effect in warm-blooded animals such as man.

The present invention includes pharmaceutically acceptable salts or prodrugs of such compounds. Also in accordance with the present invention applicants provide pharmaceutical compositions and a method to use such compounds in the treatment of cancer.

Such properties are expected to be of value in the treatment of disease states associated with cell cycle and cell proliferation such as cancers (solid tumors and leukemias), fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation.

Definitions

The definitions set forth in this section are intended to clarify terms used throughout this application. The term “herein” means the entire application.

As used in this application, the term “optionally substituted,” as used herein, means that substitution is optional and therefore it is possible for the designated atom to be unsubstituted. In the event a substitution is desired then such substitution means that any number of hydrogens on the designated atom is replaced with a selection from the indicated group, provided that the normal valency of the designated atom is not exceeded, and that the substitution results in a stable compound. For example when a substituent is keto (i.e., ═O), then 2 hydrogens on the atom are replaced. If no selection is provided then the substituent shall be selected from: halogen, nitro, amino, cyano, trifluoromethyl, alkyl, alkenyl, alkynyl, haloalkyl, alkoxy, hydroxy, alkylhydroxy, carbonyl, —CH(OH)CH₃, —CH₂NH-alkyl-OH, alkyl-(OH)CH₃, —Oalkyl, —OCOalkyl, —NHCHO, —N-(alkyl)—CHO, —NH—CO-amino, —N—(alkyl)—CO-amino, —NH—COalkyl, —N—(alkyl)—COalkyl, -carboxy, -amidino, —CO-amino, —CO-alkyl, —CO₂alkyl, mercapto, —S-alkyl, —SO(alkyl), —SO₂(alkyl), —SO₂-amino, -alkylsulfonylamino, phenyl, cycloalkyl, heterocyclic and heteroaryl, -alkly-NH-cycloalkyl, -alkyl-NH-optionally substituted heterocycle, -alkyl-NH-alkyl-OH, —C(═O)OC(CH₃)₃, —N(CH₃)₂, -alkyl-NH-alkyl-optionally substituted heterocycle, alkyl-aryl, alkyl-polycyclyl, alkyl-amino, alkyl-hydroxy, —CH₂NH-alkyl-heterocycle, —CH₂NHCH2CH(CH₃)₂, -CH₂NHCH2CH(CH₃)₂, —C(═O)OC(CH₃)₃, —C₁₋₃alkyl, —OC₁₋₃alkyl, —N(CH₃)₂, —NCH₂NCH₃, —CH₂NCH₃, —CH₂-piperazine, or —CH₂-methylpiperazine.

If the selection is attached to a ring the substituents could also be selected from: vicinal —O(alkyl)O—, vicinal —O(Chaloalkyl)O—, vicinal —CH₂O(alkyl)O—, vicinal —S(alkyl)S— and —O(alkyl)S—.

When any variable (e.g., R¹, R⁴, R^{a}, R^{e} etc.) occurs more than one time in any constituent or formula for a compound, its definition at each occurrence is independent of its definition at every other occurrence. Thus, for example, if a group is shown to be substituted with 0-3 R¹, then said group may optionally be substituted with 0,1, 2 or 3 R¹ groups and R^{a} at each occurrence is selected independently from the definition of R^{e}. Also, combinations of substituents and/or variables are permissible only if such combinations result in stable compounds.

A variety of compounds in the present invention may exist in particular geometric or stereoisomeric forms. The present invention takes into account all such compounds, including cis- and trans isomers, R- and S-enantiomers, diastereomers, (D)-isomers, (L)-isomers, the racemic mixtures thereof, and other mixtures thereof, as being covered within the scope of this invention. Additional asymmetric carbon atoms may be present in a substituent such as an alkyl group. All such isomers, as well as mixtures thereof, are intended to be included in this invention. The compounds herein described may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. When required, separation of the racemic material can be achieved by methods known in the art. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic forms and all geometric isomeric forms of a structure are intended, unless the specific stereochemistry or isomeric form is specifically indicated.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents and/or variables are permissible only if such combinations result in stable compounds.

As used herein, “alkyl” or “alkylene” used alone or as a suffix or prefix, is intended to include both branched and straight-chain saturated aliphatic hydrocarbon groups having from 1 to 12 carbon atoms or if a specified number of carbon atoms is provided then that specific number would be intended. For example “C₁₋₆ alkyl” denotes alkyl having 1, 2, 3, 4, 5 or 6 carbon atoms. Examples of alkyl include, but are not limited to, methyl, ethyl, n-propyl, i-propyl, n-butyl, i-butyl, sec-butyl, t-butyl, pentyl, and hexyl. As used herein, “C₁₋₃ alkyl”, whether a terminal substituent or an alkylene group linking two substituents, is understood to specifically include both branched and straight-chain methyl, ethyl, and propyl.

As used herein “alkylhydroxy” represents an alkyl group straight chain or branched as defined above with the indicated number of carbon atoms with one or more hydroxy groups attached. One such example of alkylhdroxy would be —CH₂OH.

As used herein, the term “cycloalkyl” is intended to include saturated ring groups, having the specified number of carbon atoms. These may include fused or bridged polycyclic systems. Preferred cycloalkyls have from 3 to 10 carbon atoms in their ring structure, and more preferably have 3, 4, 5, and 6 carbons in the ring structure. For example, “C₃₋₆ cycloalkyl” denotes such groups as cyclopropyl, cyclobutyl, cyclopentyl, or cyclohexyl.

As used herein, “alkenyl” or “alkenylene” is intended to include from 2 to 12 hydrocarbon atoms of either a straight or branched configuration with one or more carbon-carbon double bonds that may occur at any stable point along the chain. Examples of “C₃₋₆alkenyl” include, but are not limited to, 1-propenyl, 2-propenyl, 1-butenyl, 2-butenyl, 3-butenyl, 3-methyl-2-butenyl, 2-pentenyl, 3-pentenyl, hexenyl.

As used herein, “alkynyl” or “alkynylene” is intended to include from 2 to 12 hydrocarbon chains of either a straight or branched configuration with one or more carbon-carbon triple bonds that may occur at any stable point along the chain. Examples of alkynyl include but are not limited to ethynyl, 1-propynyl, 2-propynyl, 1-butynyl, 2-butynyl, 3-butynyl.

As used herein, the term “alkylcycloalkyl” is intended to mean an alkyl attached to the formula atom modified with a cycloalkyl. Examples of alkylcycloalkyl include, but are not limited to cyclopropylmethyl, cyclopentylmethyl, cyclohexylmethyl, cycloheptylmethyl, cyclopropylethyl, cyclopentylethyl, cyclohexylethyl, cycloheptylethyl, cyclopropylpropyl, cyclopentylpropyl, cyclohexylpropyl, cycloheptylpropyl.

As used herein, “cycloalkenyl” refers to ring-containing hydrocarbyl groups having at least one carbon-carbon double bond in the ring, and having from 3 to 12 carbons atoms.

As used herein, “cycloalkynyl” refers to ring-containing hydrocarbyl groups having at least one carbon-carbon triple bond in the ring, and having from 7 to 12 carbons atoms.

As used herein, the term “aralkyl” refers to an alkyl group substituted with an aryl group (an aromatic or heteroaromatic group).

As used herein, “aromatic” refers to hydrocarbyl groups having one or more polyunsaturated carbon rings having aromatic character, (e.g., 4n+2 delocalized electrons) and comprising up to about 14 carbon atoms.

The term “aryl” as used herein includes 5-, 6- and 7-membered single-ring aromatic groups that may include from zero to four heteroatoms, for example, benzene, furan, imidazole, isoxazole, nicotinic, isonictinic, oxazole, phenyl, pyrazole, pyrazine, pyridazine, pyridine, pyrimidine, thiazole, thiophene, triazole and the like. Those aryl groups having heteroatoms in the ring structure may also be referred to as “heteroaryl” or “heteroaromatics.” The aromatic ring can be substituted at one or more ring positions with such substituents as described above. The term “aryl” also includes polycyclic ring systems having two or more cyclic rings in which two or more carbons are common to two adjoining rings (the rings are “fused rings”) wherein at least one of the rings is aromatic, for example, the other cyclic rings can be cycloalkyls, cycloalkenyls, cycloalkynyls, aryls and/or heterocyclyls. The terms ortho, meta and para apply to 1,2-, 1,3- and 1,4-disubstituted benzenes, respectively. For example, the names 1,2-dimethylbenzene and ortho-dimethylbenzene are synonymous.

As used herein, the term “heterocycle” or “heterocyclic” or “heterocyclyl” refers to a ring-containing monovalent and divalent structures having one or more heteroatoms, independently selected from N, O and S, as part of the ring structure and comprising from 3 to 20 atoms in the rings, more preferably 3- to 7-membered rings. Heterocyclic groups may be saturated or unsaturated, containing one or more double bonds, and heterocyclic groups may contain more than one ring as in the case of polycyclic systems. The heterocyclic rings described herein may be substituted on carbon or on a heteroatom atom if the resulting compound is stable. If specifically noted, nitrogen in the heterocycle may optionally be quaternized. It is understood that when the total number of S and O atoms in the heterocycle exceeds 1, then these heteroatoms are not adjacent to one another.

Examples of heterocycles include, but are not limited to, 1H-indazole, 2-pyrrolidonyl, 2H, 6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azetidine, aziridine, azocinyl, benzimidazolyl, benzofuranyl, benzothiofuranyl, benzothiophenyl, benzoxazolyl, benzthiazolyl, benzotriazolyl, benzotetrazolyl, benzisoxazolyl, benzisothiazolyl, benzimidazalonyl, carbazolyl, 4aH-carbazolyl, b-carbolinyl, chromanyl, chromenyl, cinnolinyl, decahydroquinolinyl, 2H,6H-1,5,2-dithiazinyl, dioxolane, furyl, 2,3-dihydrofuran, 2,5-dihydrofuran, dihydrofuro[2,3-b]tetrahydrofuran, furanyl, furazanyl, homopiperidinyl, imidazolidine, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxirane, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, purinyl, pyranyl, pyrrolidine, pyrroline, pyrrolidine, pyrazinyl, pyrazolidinyl, pyrazolinyl, pyrazolyl, pyridazinyl, pyridooxazole, pyridoimidazole, pyridothiazole, pyridinyl, N-oxide-pyridinyl, pyridyl, pyrimidinyl, pyrrolidinyl, pyrrolinyl, pyrrolyl, pyridine, quinazolinyl, quinolinyl, 4H-quinolizinyl, quinoxalinyl, quinuclidinyl, carbolinyl, tetrahydrofuranyl, tetrahydroisoquinolinyl, thiophane, thiotetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, thiirane, triazinyl, 1,2,3-triazolyl, 1,2,4-triazolyl, 1,2,5-triazolyl, 1,3,4-triazolyl, xanthenyl.

The terms “polycyclyl” or “polycyclic group” refer to two or more rings (for example, cycloalkyls, cycloalkenyls, cycloalkynyls, aryls and/or heterocyclyls) in which two or more carbons are common to two adjoining rings, for example, the rings are “fused rings.” Rings that are joined through non-adjacent atoms are termed “bridged” rings. Each of the rings of the polycycle can be substituted with such substituents as described above, as for example, halogen, alkyl, aralkyl, alkenyl, alkynyl, cycloalkyl, hydroxyl, amino, nitro, sulfhydryl, imino, amido, carbonyl, carboxyl, ether, alkylthio, sulfonyl, ketone, aldehyde, ester, a heterocyclyl, an aromatic or heteroaromatic moiety, —CF₃, —CN, or the like. Examples of such bridged heterocycles include quinuclidine, diazabicyclo[2.2.1]heptane and 7-oxabicyclo[2.2.1]heptane, substituted piperazine.

As used herein, the term “amine” or “amino” refers to groups of the general formula —NRR′, wherein R and R′ are each independently represented by but not limited to hydrogen, alkyl, cycloalkyl, alkenyl, aryl, heteroaryl, aralkyl, or heteroaralkyl. Example of the amino group include, but are not limited to NH₂, methylamine, ethylamine, dimethylamine, diethylamine, propylamine, benzylamine and the like.

As used herein, the term “acylamino” is art-recognized and refers to a moiety that can be represented by the general formula: 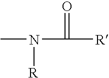
wherein R and R′ are each independently represented by but not limited to hydrogen, alkyl, cycloalkyl, alkenyl, aryl, heteroaryl, heterocyclyl, aralkyl, or heteroaralkyl.

As used herein, the term “amido” is art-recognized as an amino-substituted carbonyl and includes a moiety that can be represented by the general formula: 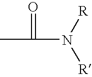
wherein R and R′ are each independently represented by but not limited to hydrogen, alkyl, cycloalkyl, alkenyl, aryl, heteroaryl, heterocyclyl, aralkyl, or heteroaralkyl, or R and R′ may form a ring.

As used herein, “alkoxy” or “alkyloxy” represents an alkyl group as defined above with the indicated number of carbon atoms attached through an oxygen bridge. Examples of alkoxy include, but are not limited to, methoxy, ethoxy, n-propoxy, isopropoxy, n-butoxy, isobutoxy, t-butoxy, n-pentoxy, isopentoxy, cyclopropylmethoxy, allyloxy and propargyloxy. Similarly, “alkylthio” or “thioalkoxy” represent an alkyl group as defined above with the indicated number of carbon atoms attached through a sulphur bridge.

As used herein, the term “acyl” refers to groups of the of the general formula —C(═O)—R, wherein R is hydrogen, hydrocarbyl radical. Examples of acyl groups include, but are not limited to acetyl, propionyl, benzoyl, phenyl acetyl.

As used herein, the term “carbonyl” is art recognized and includes such moieties as can be represented by the general formula: 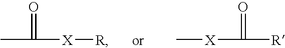
wherein X is a bond or represents an oxygen or sulfur, and R represents a hydrogen, an alkyl, an alkenyl, —(CH₂)ₘ—R″ or a pharmaceutically acceptable salt, R′ represents a hydrogen, an alkyl, an alkenyl or —(CH₂)ₘ—R″, where m is an integer less than or equal to ten, and R″ is alkyl, cycloalkyl, alkenyl, aryl, or heteroaryl. Where X is an oxygen and R and R′ is not hydrogen, the formula represents an “ester”. Where X is an oxygen, and R is as defined above, the moiety is referred to herein as a carboxyl group, and particularly when R′ is a hydrogen, the formula represents a “carboxylic acid.” Where X is oxygen, and R′ is a hydrogen, the formula represents a “formate.” In general, where the oxygen atom of the above formula is replaced by sulfur, the formula represents a “thiolcarbonyl” group. Where X is a sulfur and R and R′ is not hydrogen, the formula represents a “thiolester.” Where X is sulfur and R is hydrogen, the formula represents a “thiolcarboxylic acid.” Where X is sulfur and R′ is hydrogen, the formula represents a “thiolformate.” On the other hand, where X is a bond, and R is not a hydrogen, the above formula represents a “ketone” group. Where X is a bond, and R is hydrogen, the above formula is represents an “aldehyde” group.

As used herein, the term “sulfonylamino” is art-recognized and refers to a moiety that can be represented by the general formula: 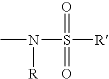
wherein R and R′ are each independently represented by but not limited to hydrogen, alkyl, cycloalkyl, alkenyl, aryl, heteroaryl, heterocyclyl, aralkyl, or heteroaralkyl.

As used herein, the term “sulfamoyl” is art-recognized and refers to a moiety that can be represented by the general formula: 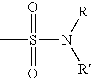
wherein R and R′ are each independently represented by but not limited to hydrogen, alkyl, cycloalkyl, alkenyl, aryl, heteroaryl, heterocyclyl, aralkyl, or heteroaralkyl, or R and R′ may form a ring.

As used herein, the term “sulfonyl” is art-recognized and refers to a moiety that can be represented by the general formula: 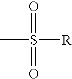
wherein R is represented by but not limited to hydrogen, alkyl, cycloalkyl, alkenyl, aryl, heteroaryl, aralkyl, or heteroaralkyl.

As used herein, the term “sulfoxido” is art-recognized and refers to a moiety that can be represented by the general formula: 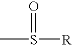
wherein R is represented by but not limited to hydrogen, alkyl, cycloalkyl, alkenyl, aryl, heteroaryl, aralkyl, or heteroaralkyl.

As used herein, “halo” or “halogen” refers to fluoro, chloro, bromo, and iodo. “Counterion” is used to represent a small, negatively charged species such as chloride, bromide, hydroxide, acetate, sulfate, tosylate, benezensulfonate, and the like.

As used herein, “haloalkyl” is intended to include both branched and straight-chain saturated aliphatic hydrocarbon groups having the specified number of carbon atoms, substituted with 1 or more halogen (for example ——CᵥF_{w} where v=1 to 3 and w=1 to (2v+1)). Examples of haloalkyl include, but are not limited to, trifluoromethyl, trichloromethyl, pentafluoroethyl, pentachloroethyl, 2,2,2-trifluoroethyl, 2,2-difluoroethyl, heptafluoropropyl, and heptachloropropyl. “Haloalkoxy” is intended to mean a haloalkyl group as defined above with the indicated number of carbon atoms attached through an oxygen bridge; for example trifluoromethoxy, pentafluoroethoxy, 2,2,2-trifluoroethoxy, and the like. “Haloalkylthio” is intended to mean a haloalkyl group as defined above with the indicated number of carbon atoms attached through a sulphur bridge.

As used herein, “moieties” means alkyl; cycloalkyl; alkenyl; alkynyl; alkylcycloalkyl; cycloalkenyl; cycloalkynyl; aralkyl; aryl; heterocycle; polycyclyl; amine;acylamino; amido; alkoxy; acyl; carbonyl; sulfonylamino; sulfamoyl; sulfonyl; sulfoxido; halo; haloalkyl; haloalkoxy as these terms are defined herein.

As used herein, the phrase “protecting group” means temporary substituents which protect a potentially reactive functional group from undesired chemical transformations. Examples of such protecting groups include esters of carboxylic acids, silyl ethers of alcohols, and acetals and ketals of aldehydes and ketones respectively. The field of protecting group chemistry has been reviewed (Greene, T. W.; Wuts, P. G. M. Protective Groups in Organic Synthesis, 3^{rd} ed.; Wiley: New York, 1999).

As used herein, “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, maleic, tartaric, citric, ascorbic, palmitic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, the disclosure of which is hereby incorporated by reference. “Prodrugs” are intended to include any covalently bonded carriers that release the active parent drug according to formula (I) in vivo when such prodrug is administered to a mammalian subject. Prodrugs of a compound of formula (I) are prepared by modifying functional groups present in the compound in such a way that the modifications are cleaved, either in routine manipulation or in vivo, to the parent compound. Prodrugs include compounds of formula (I) wherein a hydroxy, amino, or sulfhydryl group is bonded to any group that, when the prodrug or compound of formula (I) is administered to a mammalian subject, cleaves to form a free hydroxyl, free amino, or free sulfhydryl group, respectively. Examples of prodrugs include, but are not limited to, acetate, formate and benzoate derivatives of alcohol and amine functional groups in the compounds of formula (I), and the like.

“Stable compound” and “stable structure” are meant to indicate a compound that is sufficiently robust to survive isolation to a useful degree of purity from a reaction mixture, and formulation into an efficacious therapeutic agent.

### DETAILED DESCRIPTION OF THE INVENTION

In a first embodiment, the present invention provides novel compounds having formula (I): 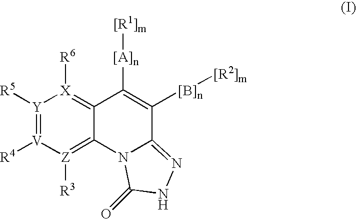
wherein:

m is independently selected at each occurrence from 0,1 or 2;

n is independently selected at each occurrence from 0 or 1;

A is optionally substituted phenyl, optionally substituted phenol, optionally substituted heterocyclic;

B is optionally substituted phenyl, optionally substituted phenol, optionally substituted heterocyclic;

R¹ is H, OH, F, Cl, Br, I, NH₂, —C(═O)R^{c}—C(═O)NHR^{c}, C(═O)CH₂R^{c}—C(═O)(CH₂)₂R^{c}, C(═O)(CH₂)₃R^{c}, —C(═O)NH(CH₂)NH₂, —C(═O)NH(CH₂)₂NH₂, —C(═O)NH(CH₂)₃NH₂, —C(═O)NH(CH₂)N(CH₃)₂, —C(═O)NH(CH₂)₂N(CH₃)₂, —C(═O)NH(CH₂)₃N(CH₃)₂, —C(═O)NH(CH₂)₂NHCH₃, —C(═O)NH(CH₂)₃OH, —C(═O)NHNH₂, —C(═O)NHCH(CH₃)CH₂N(CH₃)₂, —C(═O)NH(CH₂)₂NHC(CH₃)₂, (CH₂)₁₋₃OH, —C(═O)OR^{a}, —C(═O)NHNH₂, —NH(CH₂)₁₋₃R^{a}, —CH₂NH(CH₂)₁₋₃R^{a}, —NHC(═O)OR, —(C₆H₄)NH-cycloalkyl, —(C₆H₄)NH-optionally substituted heterocycle, —(C₆H₄)CH₂NH-alkyl-OH, —(C₆H₄)N(CH₃)₂, —O-alkyl-NH₂, optionally substituted alkyl, optionally substituted N-alkyl, optionally substituted alkenyl, optionally substituted alkynyl, optionally substituted cycloalkyl, optionally substituted cycloalkenyl, optionally substituted cycloalkynyl, optionally substituted aryl, optionally substituted alkoxy, optionally substituted heterocycle, or optionally substituted fused heterocycle;

R² is H, OH, F, Cl, Br, I, NH₂, (CH₂)₁₋₃OH, —C(═O)OR^{a}, —C(═O)NHNH₂, —-NH(CH₂)₁₋₃R^{a}, —CH₂NH(CH₂)₁₋₃R^{a}, —NHC(═O)OR, optionally substituted alkyl, optionally substituted N-alkyl, optionally substituted alkenyl, optionally substituted alkynyl, optionally substituted cycloalkyl, optionally substituted cycloalkenyl, optionally substituted cycloalkynyl, optionally substituted aryl, optionally substituted alkoxy, optionally substituted heterocycle, or optionally substituted fused heterocycle.

R³ is is H, OH, F, Cl, Br, I, NH₂, CH₃;

R⁴ is H, OH, F, Cl, Br, I, NH₂, R^{a}, OCH3, —C(═O)OR^{a}, —C(═O)NHNH₂, —NH(CH₂)₁₋₃R^{a}, —CH₂NH(CH₂)₁₋₃R^{a}, —NHC(═O)OR^{a}, —(C₆H₄)CH₂NH(CH₂)₁₋₃R^{a}, —(C₆H₄)CH₂N(CH₃)(CH₂)₁₋₃R^{a}, —(C₆H₄)(CH₂)₀₋₃R^{a}, —(C₆H₄)(R^{b})CH₂R^{a}, —(C₆H₄)CH₂NHR^{a}, —(C₆H₄)C(═O)R^{a}—(C₆H₄)NHC(═O)R^{a}, —(C₆H₄)CH₂NH(CH₂)₁₋₃R^{a}R^{b}, —(C₆H₄)NHSO₂CH₃, optionally substituted alkyl, optionally substituted N-alkyl, optionally substituted alkenyl, optionally substituted alkynyl, optionally substituted cycloalkyl, optionally substituted cycloalkenyl, optionally substituted cycloalkynyl, optionally substituted aryl, optionally substituted alkoxy, optionally substituted heterocycle, or optionally substituted fused heterocycle;

R⁵ is H, OH, F, Cl, Br, I, NH₂, OCH₃, —C(═O)OR^{a}, —C(═O)NHNH₂, —NH(CH₂)₁₋₃R^{a}, —CH₂NH(CH₂)₁₋₃R^{a}, —NHC(═O)OR^{a}, optionally substituted alkyl, optionally substituted N-alkyl, optionally substituted alkenyl, optionally substituted alkynyl, optionally substituted cycloalkyl, optionally substituted cycloalkenyl, optionally substituted cycloalkynyl, optionally substituted aryl, optionally substituted alkoxy, optionally substituted heterocycle, or optionally substituted fused heterocycle;

R⁶ is H, OH, F, Cl, Br, I, NH₂, NHC₁₋₆alkyl, N(C₁₋₆alkyl)₂, —(C₆H₄)CH₂R^{a}, —(C₆H₄)CH₂NR^{a}R^{b}, optionally substituted aryl;

R^{a} is H. OH, OCH₃, C₁₋₆alkyl, C₁₋₆alkoxy, NH₂, NHCH₃, N(CH₃)₂, CH₂C(CH₃)₂, optionally substituted phenyl, optionally substituted cycloalkyl, optionally substituted 5 or 6 or 7 membered heterocycle having 1 or 2 oxygen or 1 or 2 nitrogen or 1 nitrogen and 1 oxygen or 1 nitrogen and 1 sulfur or 1 oxygen and 1 sulfur ring atoms;

R^{b} is H, OH, OCH₃, C₁₋₆-alkyl, C₁₋₆alkoxy;

R^{c} is optionally substituted C₄₋₇ heterocycle;

X is CH, substituted C, N, O, or any combination thereof;

Y is CH, substituted C, N, O, or any combination thereof;

Z is CH, substituted C, N, O, or any combination thereof;

V is CH, substituted C, N, O, or any combination thereof;

or a pharmaceutically aceptable salt thereof.

In a particular embodiment the present invention provides a compound having formula (I) as recited above wherein m is 0.

In a particular embodiment the present invention provides a compound having formula (I) as recited above wherein n is 0.

In a particular embodiment the present invention provides a compound having formula (I) as recited above wherein R¹ is —C(═O)R^{c}—C(═O)NHR^{c}, C(═O)CH₂R^{c}—C(═O)(CH₂)₂R^{c}, C(═O)(CH₂)₃R^{c}, —C(═O)NH(CH₂)NH₂, —C(═O)NH(CH₂)₂NH₂, —C(═O)NH(CH₂)₃NH₂, —C(═O)NH(CH₂)N(CH₃)₂, —C(═O)NH(CH₂)₂N(CH₃)₂, —C(═O)NH(CH₂)₃N(CH₃)₂, —C(═O)NH(CH₂)₂NHCH₃, —C(═O)NH(CH₂)₃OH, —C(═O)NHNH₂, —C(═O)NHCH(CH₃)CH₂N(CH₃)₂, —C(═O)NH(CH₂)₂NHC(CH₃)₂.

In a particular embodiment the present invention provides a compound having formula (I) as recited above wherein R¹ is NH₂, CH₃, or (CH₂)₁₋₃OH, —(C₆H₄)NHcycloalkyl, O(CH₂)₁₋₃NH₂, —(C₆H₄)NH-cycloalkyl, —(C₆H₄)NH-optionally substituted heterocycle, —(C₆H₄)CH₂NH-alkyl-OH, —(C₆H₄)N(CH₃)₂, —O-alkyl-NH₂.

In a particular embodiment the present invention provides a compound having formula (I) as recited above wherein R² is H or (CH₂)₁₋₃OH.

In a particular embodiment the present invention provides a compound having formula (I) as recited above wherein R³ is H.

In a particular embodiment the present invention provides a compound having formula (I) as recited above wherein R⁴ is H, OCH₃, —(C₆H₄)CH₂NH(CH₂)₁₋₃R^{a}, —(C₆H₄)CH₂N(CH₃)(CH₂)₁₋₃R^{a}, —(C₆H₄)CH₂R^{a}, —(C₆H₄)(R^{b})CH₂R^{a}, —(C₆H₄)CH₂ NHR^{a}—(C₆H₄)C(═O)R^{a}—(C₆H₄)NHC(═O)R^{a}, —(C₆H₄)CH₂NH(CH₂)₁₋₃R^{a}R^{b}, —(C₆H₄)NHSO₂CH₃, optionally substituted aryl, or optionally substituted heterocycle.

In a particular embodiment the present invention provides a compound having formula (I) as recited above wherein R⁴ is halogen, or an optionally substituted 5-membered heterocycle wherein said substitution is selected from —N(CH₃)₂, —NCH₂NCH₃, —CH₂NCH₃, CH₂-piperazine, or CH₂-methylpiperazine.

In a particular embodiment the present invention provides a compound having formula (I) as recited above wherein R⁴ is halogen or an optionally substituted furan, optionally substituted pyridine, or optionally substituted thiophene.

In a particular embodiment the present invention provides a compound having formula (I) as recited above wherein R⁴ is optionally substituted furan, optionally substituted pyridine, or optionally substituted thiophene wherein said substitution is selected from —N(CH₃)₂, —NCH₂NCH₃, —CH₂NCH₃, CH₂-piperazine, CH₂-methylpiperazine.

In a particular embodiment the present invention provides a compound having formula (I) as recited above wherein R⁵ is H, OH, or OCH₃.

In a particular embodiment the present invention provides a compound having formula (I) as recited above wherein R₆ is H, —(C₆H₄)CH₂R^{a}, —(C₆H₄)CH₂NR^{a}R^{b}.

In a particular embodiment the present invention provides a compound having formula (I) as recited above wherein X is CH or N.

In a particular embodiment the present invention provides a compound having formula (I) as recited above wherein Y is CH or N.

In a particular embodiment the present invention provides a compound having formula (I) as recited above wherein Z is CH or N.

In a particular embodiment the present invention provides a compound having formula (I) as recited above wherein V is an optionally substituted carbon.

In a particular embodiment the present invention provides a compound having formula (I) as recited above wherein:

m is 0 or 1;

n is 0;

R¹ is NH₂, CH₃, or (CH₂)₁₋₃OH; —(C₆H₄)NHcycloalkyl, O(CH₂)₁₋₃NH₂, —(C₆H₄)NH-cycloalkyl, —(C6H₄)NH-optionally substituted heterocycle, —(C₆H₄)CH₂NH-alkyl-OH, —(C₆H₄)N(CH₃)₂, —O-alkyl-NH₂;

R² is H or (CH₂)₁₋₃OH;

R³ is H;

R⁴ is OCH₃, —(C₆H₄)CH₂NH(CH₂)₁₋₃R^{a}, —(C₆H₄)CH₂N(CH₃)(CH₂)₁₋₃R^{a}, —(C₆H₄)CH₂R^{a}, —(C₆H₄)(R^{b})CH₂R^{a}, —(C₆H₄)CH₂ NHR^{a}—(C₆H₄)C(═O)R^{a}—(C₆H₄)NHC(═O)R^{a}, —(C₆H₄)CH₂NH(CH₂)₁₋₃R^{a}R^{b}, —(C₆H₄)NHSO₂CH₃, optionally substituted aryl, or optionally substituted heterocycle;

R⁵ is H, OH, or OCH₃;

R⁶ is H; —(C₆H₄)CH₂R^{a}, —(C₆H₄)CH₂NR^{a}R^{b};

R^{a} is OH, OCH₃, C₁₋₆alkyl, NH₂, NHCH₃, N(CH₃)₂, CH₂C(CH₃)₂, optionally substituted cycloalkyl, optionally substituted 5 or 6 or 7 membered heterocycle having 1 or 2 oxygen, or 1 or 2 nitrogen, or 1 nitrogen and 1 oxygen, or 1 nitrogen and 1 sulfur, or 1 oxygen and 1 sulfur ring atoms;

R^{b} is OH, OCH₃, C₁₋₆allyl;

X, Y, Z and V are CH.

In a particular embodiment the present invention provides a compound having formula (I) as recited above wherein:

m is 1;

n is 0;

R¹ is —C(═O)R^{c}—C(═O)NHR^{c}, C(═O)CH₂R^{c}—C(═O)(CH₂)₂R^{c}, C(═O)(CH₂)₃R^{c}, —C(═O)NH(CH₂)NH₂, —C(═O)NH(CH₂)₂NH₂, —C(═O)NH(CH₂)₃NH₂, —C(═O)NH(CH₂)N(CH₃)₂, —C(═O)NH(CH₂)₂N(CH₃)₂, —C(═O)NH(CH₂)₃N(CH₃)₂, —C(═O)NH(CH₂)₂NHCH₃, —C(═O)NH(CH₂)₃OH, —C(═O)NHNH₂, —C(═O)NHCH(CH₃)CH₂N(CH₃)₂, —C(═O)NH(CH₂)₂NHC(CH₃)₂;

R² is H;

R³ is H;

R⁴ is halogen, or an optionally substituted 5-membered heterocycle;

R⁵ is H;

R⁶ is H;

X, Y, Z and V are CH.

In a particular embodiment the present invention provides a compound having formula (I) as recited above wherein:

m is 1;

n is 0;

R¹ is —C(═O)R^{c}—C(═O)NHR^{c}, C(═O)CH₂R^{c}—C(═O)(CH₂)₂R^{c}, C(═O)(CH₂)₃R^{c}, —C(═O)NH(CH₂)NH₂, —C(═O)NH(CH₂)₂NH₂, —C(═O)NH(CH₂)₃NH₂, —C(═O)NH(CH₂)N(CH₃)₂, —C(═O)NH(CH₂)₂N(CH₃)₂, —C(═O)NH(CH₂)₃N(CH₃)₂, —C(═O)NH(CH₂)₂NHCH₃, —C(═O)NH(CH₂)₃OH, —C(═O)NHNH₂, —C(═O)NHCH(CH₃)CH₂N(CH₃)₂, —C(═O)NH(CH₂)₂NHC(CH₃)₂;

R² is H;

R³ is H;

R⁴ is halogen, or an optionally substituted 5-membered heterocycle wherein said substitution is selected from —N(CH₃)₂, —NCH₂NCH₃, —CH₂NCH₃, —CH₂-piperazine or —CH₂-methylpiperazine.

R⁵ is H;

R⁶ is H;

X, Y, Z and V are CH.

In a particular embodiment the present invention provides a compound having formula (I) as recited above selected from:
5-methyl[1,2,4]triazolo[4,3-a]quinolin-1(2H)-one;
5,9-dimethyl[1,2,4]triazolo[4,3-a]quinolin-1(2H)-one;
8-methoxy-5-methyl[1,2,4]triazolo[4,3-a]quinolin-1(2H)-one;
8-fluoro-5-methyl[1,2,4]triazolo[4,3-a]quinolin-1(2H)-one;
5-bromo-5-(hydroxymethyl)[1,2,4]triazolo[4,3-a]quinolin-1(2H)-one [1,2,4]triazolo[4,3-a]quinolin-1(2H)-one;
ethyl-7-bromo-1-oxo-1,2-dihydro[1,2,4]triazolo[4,3-a]quinoline-5-carboxylate;
Ethyl-7-methyl-1-oxo-1,2-dihydro[1,2,4]triazolo[4,3-a]quinoline-5-carboxylate;
7-methyl-1-oxo-1,2-dihydro[1,2,4]triazolo[4,3-a]quinoline-5-carbohydrazide;
5-amino[1,2,4]triazolo[4,3-a]quinolin-1(2H)-one;
5-amino-7-bromo[1,2,4]triazolo[4,3-a]quinolin-1(2H)-one;
7-methoxy-5-methyl[1,2,4]triazolo[4,3-a]quinolin-1(2H)-one;
7-hydroxy-5-methyl[1,2,4]triazolo[4,3-a]quinolin-1(2H)-one;
8-hydroxy-5-methyl[1,2,4]triazolo[4,3-a]quinolin-1(2H)-one;
5,7-dimethyl[1,2,4]triazolo[4,3-a]quinolin-1(2H)-one;
5,8-dimethyl[1,2,4]triazolo[4,3-a]quinolin-1(2H)-one;
9-hydroxy[1,2,4]triazolo[4,3-a]quinolin-1(2H)-one;
T-butyl-7-bromo-1-oxo-1,2-dihydro[1,2,4]triazolo[4,3-a]quinolin-5-ylcarbamate;
7,8-dihydroxy-5-methyl[1,2,4]triazolo[4,3-a]quinolin-1(2H)-one;
7,8-methoxy-5-methyl[1,2,4]triazolo[4,3-a]quinolin-1(2H)-one;
7,8-methoxy[1,2,4]triazolo[4,3-a]quinolin-1(2H)-one;
7,8-dihydroxy[1,2,4]triazolo[4,3-a]quinolin-1(2H)-one;
5-chloro[1,2,4]triazolo[4,3-a]quinolin-1(2H)-one;
1-oxo-1,2-dihydro[1,2,4]triazolo[4,3-a]quinoline-5-carbohydrazide;
7-bromo-5-methyl[1,2,4]triazolo[4,3-a]quinolin-1(2H)-one;
7-iodo-5-methyl[1,2,4]triazolo[4,3-a]quinolin-1(2H)-one;
7-(3-aminophenyl)-5-methyl[1,2,4]triazolo[4,3-a]quinolin-1(2H)-one;
7-(3-hydroxyphenyl)-5-methyl[1,2,4]triazolo[4,3-a]quinolin-1(2H)-one;
8-(3-hydroxyphenyl)-5-methyl[1,2,4]triazolo[4,3-a]quinolin-1(2H)-one;
8-[3-(hydroxymethyl)phenyl-5-methyl[1,2,4]triazolo[4,3-a]quinolin-1(2H)-one;
8-[4-(hydroxymethyl)phenyl-5-methyl[1,2,4]triazolo[4,3-a]quinolin-1(2H)-one;
8-(3-aminophenyl)-5-methyl[1,2,4]triazolo[4,3-a]quinolin-1(2H)-one;
5-(3-aminophenyl)[1,2,4]triazolo[4,3-a]quinolin-1(2H)-one;
5-[4-(hydroxymethyl)phenyl[1,2,4]triazolo[4,3-a]quinolin-1(2H)-one;
Ethyl 7-methyl-1-oxo-1,2-dihydro[1,2,4]triazolo[4,3-a]quinoline-5-carboxylate;
5-amino-7-(3-aminophenyl)[1,2,4]triazolo[4,3-a]quinolin-1(2H)-one;
7-(2-hydroxyphenyl)-5-methyl[1,2,4]triazolo[4,3-a]quinolin-1(2H)-one;
4-amino[1,2,4]triazolo[4,3-a]quinolin-1(2H)-one;
5-amino-7-(3-hydroxyphenyl)[1,2,4]triazolo[4,3-a]quinolin-1(2H)-one;
5-amino-7-[3-(hydroxymethyl)phenyl][1,2,4]triazolo[4,3-a]quinolin-1(2H)-one;
5-(1-benzothien-2-yl)[1,2,4]triazolo[4,3-a]quinolin-1(2H)-one;
5-[3-(hydroxymethyl)phenyl][1,2,4]triazolo[4,3-a]quinolin-1(2H)-one;
5-[(E)-2-(4-chlorophenyl)vinyl][1,2,4]triazolo[4,3-a]quinolin-1(2H)one;
5-(2,4-dihydroxyphenyl)[1,2,4]triazolo[4,3-a]quinolin-1(2H)-one;
7-(2-hydroxyphenyl)[1,2,4]triazolo[4,3-a]quinolin-1(2H)-one;
5-(2-furyl)[1,2,4]triazolo[4,3-a]quinolin-1(2H)-one;
7-(2,4-dihydroxyphenyl)-5-methyl[1,2,4]triazolo[4,3-a]quinolin-1(2H)-one;
5-phenyl[1,2,4]triazolo[4,3-a]quinolin-1(2H)-one;
5-{[2-(3,4-dimethoxyphenyl)ethyl]amino}[1,2,4]triazolo[4,3-a]quinolin-1(2H)-one;
5-[2,6-difluorobenzyl)amino][1,2,4]triazolo[4,3-a]quinolin-1(2H)-one;
Ethyl 1-oxo-1,2-dihydro[1,2,4]triazolo[4,3-a]quinoline-5-carboxylate;
5-{4-{[(2-pyridin-2-ylethyl)amino]methyl}phenyl}[1,2,4]triazolo[4,3-a]quinolin-1(2H)-one;
5-{4-{[(2-hydroxyethyl)amino]methyl}phenyl}[1,2,4]triazolo[4,3-a]quinolin-1(2H)-one;
8-bromo-1-oxo-1,2-dihydro[1,2,4]triazolo[4,3-a]quinoline-5-carboxylic acid;
7-[(4-hydroxymethyl)phenyl]-5-methyl[1,2,4]triazolo[4,3-a]quinolin-1(2H)-one;
5-{4-[(4-methylpiperazin-1-yl)methyl]phenyl}[1,2,4]triazolo[4,3-a]quinolin-1(2H)-one;
5-(benzylamino)-7-bromo[1,2,4]triazolo[4,3-a]quinolin-1(2H)-one;
Ethyl 7-methoxy-1-oxo-1,2-dihydro[1,2,4]triazolo[4,3-a]quinoline-5-carboxylate;
5-[4-{{[3-(dimethylamino)propyl]amino}methyl}phenyl][1,2,4]triazolo[4,3-a]quinolin-1(2H)-one;
5-{4-{[(3-morpholin-4-ylpropyl)amino]methyl}phenyl}[1,2,4]triazolo[4,3-a]quinolin-1(2H)-one;
5-amino-7-methyl[1,2,4]triazolo[4,3-a]quinolin-1(2H)-one;
T-butyl 7-methoxy-1-oxo-1,2-dihydro[1,2,4]triazolo[4,3-a]quinolin-5-yl carbamate;
5-amino-7-methoxy[1,2,4]triazolo[4,3-a]quinolin-1(2H)-one;
8-dimethylamino-5-methyl[1,2,4]triazolo[4,3-a]quinolin-1(2H)-one;
5-amino-8-[4-(hydroxymethyl)phenyl][1,2,4]triazolo[4,3-a]quinolin-1(2H)-one;
5-(4-{[(2S)-2-(hydroxymethyl)pyrrolidin-1-yl]methyl}phenyl)[1,2,4]triazolo[4,3-a]quinolin-1(2H)-one;
5-[4-({[1-(hydroxymethyl)-2-methylpropyl]amino}methyl)phenyl][1,2,4]triazolo[4,3-a]quinolin-1(2H)-one;
5-(4-{[4-(3-methylphenyl)piperazin-1-yl]methyl}phenyl)[1,2,4]triazolo[4,3-a]quinolin-1(2H)-one;
7-[4-({[3-(dimethylamino)propyl]amino}methyl)phenyl]-5-methyl[1,2,4]triazolo[4,3-a]quinolin-1(2H)-one;
5-methyl-7-(4-{[(3-morpholin-4-ylpropyl)amino]methyl }phenyl)[1,2,4]triazolo[4,3-a]quinolin-1(2H)-one;
5-methyl-7-(4-{[(2-pyridin-2-ylethyl)amino]methyl}phenyl)[1,2,4]triazolo[4,3-a]quinolin-1(2H)-one;
7-(4-{[(2-hydroxyethyl)amino]methyl}phenyl)-5-methyl[1,2,4]triazolo[4,3-a]quinolin-1(2H)-one;
5-methyl-7-{4-[(4-methylpiperazin-1-yl)methyl]phenyl}[1,2,4]triazolo[4,3-a]quinolin-1(2H)-one;
7-(4-{[(2S)-2-(hydroxymethyl)pyrrolidin-1-yl]methyl}phenyl)-5-methyl[1,2,4]triazolo[4,3-a]quinolin-1(2H)-one;
7-[4-({[1-(hydroxymethyl)-2-methylpropyl]amino}methyl)phenyl]-5-methyl[1,2,4]triazolo[4,3-a]quinolin-1(2H)-one;
7-(4-{[ethyl(pyridin-4-ylmethyl)amino]methyl}phenyl)-5-methyl[1,2,4]triazolo[4,3-a]quinolin-1(2H)-one;
5-methyl-7-[4-({[3-(2-oxopyrrolidin-1-yl)propyl]amino}methyl)phenyl][1,2,4]triazolo[4,3-a]quinolin-1(2H)-one;
5-methyl-7-[4-({4-[3-(trifluoromethyl)phenyl]piperazin-1-yl}methyl)phenyl][1,2,4]triazolo[4,3-a]quinolin-1(2H)-one;
7-{4-[(isobutylamino)methyl]phenyl}-5-methyl[1,2,4]triazolo[4,3-a]quinolin-1(2H)-one;
5-[3-({[3-(dimethylamino)propyl]amino}methyl)-4-methoxyphenyl][1,2,4]triazolo[4,3-a]quinolin-1(2H)-one;
5-amino-8-[3-(hydroxymethyl)phenyl][1,2,4]triazolo[4,3-a]quinolin-1(2H)-one;
5-{3-[(dimethylamino)methyl]phenyl}[1,2,4]triazolo[4,3-a]quinolin-1(2H)-one;
5-{4-[(dimethylamino)methyl]phenyl}[1,2,4]triazolo[4,3-a]quinolin-1(2H)-one;
8-[3-(dimethylamino)phenyl]-5-methyl[1,2,4]triazolo[4,3-a]quinolin-1(2H)-one;
5-methyl-7-[4-({[2-(1H-pyrrol-1-yl)phenyl]amino}methyl)phenyl][1,2,4]triazolo[4,3-a]quinolin-1(2H)-one;
3-hydroxy-2-(1-oxo-1,2-dihydro[1,2,4]triazolo[4,3-a]quinolin-5-yl)benzaldehyde;
7-[4-({[3-(1H-imidazol-1-yl)propyl]amino}methyl)phenyl]-5-methyl[1,2,4]triazolo[4,3-a]quinolin-1(2H)-one;
5-(4-methoxy-3-{[(2-pyridin-2-ylethyl)amino]methyl}phenyl)[1,2,4]triazolo[4,3-a]quinolin-1(2H)-one;
5-[3-({[1-(hydroxymethyl)-2-methylpropyl]amino}methyl)-4-methoxyphenyl][1,2,4]triazolo[4,3-a]quinolin-1(2H)-one;
5-{4-methoxy-3-[(4-methylpiperazin-1-yl)methyl]phenyl}[1,2,4]triazolo[4,3-a]quinolin-1(2H)-one;
5-(3-{[(2R)-2-(hydroxymethyl)pyrrolidin-1-yl]methyl}-4-methoxyphenyl)[1,2,4]triazolo[4,3-a]quinolin-1(2H)-one;
5-[2-({[3-(dimethylamino)propyl]amino}methyl)-6-methoxyphenyl][1,2,4]triazolo[4,3-a]quinolin-1(2H)-one;
5-(2-{[(2S)-2-(hydroxymethyl)pyrrolidin-1-yl]methyl}-6-methoxyphenyl)[1,2,4]triazolo[4,3-a]quinolin-1(2H)-one;
5-(2-methoxy-6-{[(2-pyridin-2-ylethyl)amino]methyl}phenyl)[1,2,4]triazolo[4,3-a]quinolin-1(2H)-one;
5-{2-methoxy-6-[(4-methylpiperazin-1-yl)methyl]phenyl}[1,2,4]triazolo[4,3-a]quinolin-1(2H)-one;
5-[4-({[3-(1 H-imidazol-1-yl)propyl]amino }methyl)phenyl][1,2,4]triazolo[4,3-a]quinolin-1(2H)-one;
5-(4-{[ethyl(pyridin4-ylmethyl)amino]methyl}phenyl)[1,2,4]triazolo[4,3-a]quinolin-1(2H)-one;
5-[4-({[3-(2-oxopyrrolidin-1-yl)propyl]amino}methyl)phenyl][1,2,4]triazolo[4,3-a]quinolin-1(2H)-one;
5-[4-({[2-(1H-pyrrol-1-yl)phenyl]amino}methyl)phenyl][1,2,4]triazolo[4,3-a]quinolin-1(2H)-one;
5-(4-hydroxy-3-{[(2R)-2-(hydroxymethyl)pyrrolidin-1-yl]methyl}phenyl)[1,2,4]triazolo[4,3-a]quinolin-1(2H)-one;
5-[4-hydroxy-3-({[1-(hydroxymethyl)-2-methylpropyl]amino}methyl)phenyl][1,2,4]triazolo[4,3-a]quinolin-1(2H)-one;
5-(2-hydroxy-6-{[(2S)-2-hydroxymethyl)pyrrolidin-1-yl]methyl}phenyl)[1,2,4]triazolo[4,3-a]quinolin-1(2H)-one;
5-{2-hydroxy-6-[(4-methylpiperazin-1-yl)methyl]phenyl}[1,2,4]triazolo[4,3-a]quinolin-1(2H)-one;
5-[4-({[4-(4-methylpiperazin-1-yl)phenyl]amino}methyl)phenyl][1,2,4]triazolo[4,3-a]quinolin-1(2H)-one;
5-(4-{[methyl(2-pyridin-2-ylethyl)amino]methyl}phenyl)[1,2,4]triazolo[4,3-a]quinolin-1(2H)-one;
5-(4-{[(2-furylmethyl)amino]methyl}phenyl)[1,2,4]triazolo[4,3-a]quinolin-1(2H)-one;
5-(4-{[(3-furylmethyl)amino]methyl}phenyl)[1,2,4]triazolo[4,3-a]quinolin-1(2H)-one;
5-{4-[({2-[({5-[(dimethylamino)methyl]-2-furyl}methyl)thio]ethyl}amino)methyl]phenyl}[1,2,4]triazolo[4,3-a]quinolin-1(2H)-one;
5-(4-{[(2,3-dihydro-1-benzofuran-3-ylmethyl)amino]methyl}phenyl)[1,2,4]triazolo[4,3-a]quinolin-1(2H)-one;
5-[4-({[(1-methyl-1H-pyrrol-2-yl)methyl]amino }methyl)phenyl][1,2,4]triazolo[4,3-a]quinolin-1(2H)-one;
5-[4-({[2-(4-benzylpiperazin-1-yl)ethyl]amino}methyl)phenyl][1,2,4]triazolo[4,3-a]quinolin-1(2H)-one;
5-(4-{[(pyridin-4-ylmethyl)amino]methyl}phenyl)[1,2,4]triazolo[4,3-a]quinolin-1(2H)-one;
5-(4-{[(4-morpholin-4-ylphenyl)amino]methyl}phenyl)[1,2,4]triazolo[4,3-a]quinolin-1(2H)-one;
5-amino-8-bromo[1,2,4]triazolo[4,3-a]quinolin-1(2H)-one;
7-(4-{[4-(hydroxymethyl)piperidin-1-yl]methyl}phenyl)-5-methyl[1,2,4]triazolo[4,3-a]quinolin-1(2H)-one;
5-(4-{[4-(hydroxymethyl)piperidin-1-yl]methyl}phenyl)[1,2,4]triazolo[4,3-a]quinolin-1(2H)-one;
7-(4-{[(2-furylmethyl)amino]methyl}phenyl)-5-methyl[1,2,4]triazolo[4,3-a]quinolin-1(2H)-one;
5-(4-{[4-(2-hydroxyethyl)piperidin-1-yl]methyl}phenyl)[1,2,4]triazolo[4,3-a]quinolin-1(2H)-one;
5-{4-[(4-pyridin-2-ylpiperazin-1-yl)methyl]phenyl}[1,2,4]triazolo[4,3-a]quinolin-1(2H)-one;
5-[4-({4-[4-(trifluoromethyl)pyrimidin-2-yl]-1,4-diazepan-1-yl}methyl)phenyl][1,2,4]triazolo[4,3-a]quinolin-1(2H)-one;
4-[4-(1-oxo-1,2-dihydro[1,2,4]triazolo[4,3-a]quinolin-5-yl)benzyl]piperazine-1-carbaldehyde;
4-[4-(1-oxo-1,2-dihydro[1,2,4]triazolo[4,3-a]quinolin-5-yl)benzyl]piperazine-1-carboxamide;
5-(4-{[(piperidin-4-ylmethyl)amino]methyl}phenyl)[1,2,4]triazolo[4,3-a]quinolin-1(2H)-one;
5-(4-{[4-(2-hydroxyethyl)-1,4-diazepan-1-yl]methyl }phenyl)[1,2,4]triazolo[4,3-a]quinolin-1(2H)-one;
5-[⁴-({4-[3-chloro-5-(trifluoromethyl)pyridin-2-yl]-1,4-diazepan-1-yl}methyl)phenyl][1,2,4]triazolo[4,3-a]quinolin-1(2H)-one;
5-(4-{[4-(3-nitropyridin-2-yl)-1,4-diazepan-1-yl]methyl}phenyl)[1,2,4]triazolo[4,3-a]quinolin-1(2H)-one;
7-(4-methoxy-3-{[(3-morpholin-4-ylpropyl)amino]methyl}phenyl)-5-methyl[1,2,4]triazolo[4,3-a]quinolin-1(2H)-one;
7-(3-{[(2-hydroxyethyl)amino]methyl}-4-methoxyphenyl)-5-methyl[1,2,4]triazolo[4,3-a]quinolin-1(2H)-one;
7-(4-methoxy-3-{[(2-pyridin-2-ylethyl)amino]methyl}phenyl)-5-methyl[1,2,4]triazolo[4,3-a]quinolin-1(2H)-one;
7-(3-{[4-(2-hydroxyethyl)piperidin-1-yl]methyl}-4-methoxyphenyl)-5-methyl[1,2,4]triazolo[4,3-a]quinolin-1(2H)-one;
7-{3-[({2-[({5-[(dimethylamino)methyl]-2-furyl}methyl)thio]ethyl}amino)methyl]-4-methoxyphenyl}-5-methyl[1,2,4]triazolo[4,3-a]quinolin-1(2H)-one;
5-{2-[({2-[({5-[(dimethylamino)methyl]-2-furyl}methyl)thio]ethyl}amino)methyl]-6-methoxyphenyl}[1,2,4]triazolo[4,3-a]quinolin-1(2H)-one;
5-{2-methoxy-6-[(4-pyridin-2-ylpiperazin-1-yl)methyl]phenyl}[1,2,4]triazolo[4,3-a]quinolin-1(2H)-one;
5-(3-{[(3-furylmethyl)amino]methyl}-4-methoxyphenyl)[1,2,4]triazolo[4,3-a]quinolin-1(2H)-one;
5-{3-[({2-[({5-[(dimethylamino)methyl]-2-furyl}methyl)thio]ethyl}amino)methyl]-4-methoxyphenyl}[1,2,4]triazolo[4,3-a]quinolin-1(2H)-one;
5-[3-({[2-(4-benzylpiperazin-1-yl)ethyl]amino}methyl)4-methoxyphenyl][1,2,4]triazolo[4,3-a]quinolin-1(2H)-one;
5-{4-methoxy-3-[(4-pyridin-2-ylpiperazin-1-yl)methyl]phenyl}[1,2,4]triazolo[4,3-a]quinolin-1(2H)-one;
8-chloro-7-methoxy-5-methyl[1,2,4]triazolo[4,3-a]quinolin-1(2H)-one;
7-methoxy-5-methyl-8-pyridin-4-yl[1,2,4]triazolo[4,3-a]quinolin-1(2H)-one;
8-[3-(benzyloxy)phenyl]-7-methoxy-5-methyl[1,2,4]triazolo[4,3-a]quinolin-1(2H)-one;
7-methoxy-8-[4-(methoxymethyl)phenyl]-5-methyl[1,2,4]triazolo[4,3-a]quinolin-1(2H)-one;
Tert-butyl 3-(7-methoxy-5-methyl-1-oxo-1,2-dihydro[1,2,4]triazolo[4,3-a]quinolin-8-yl)benzylcarbamate;
8-[4-(aminomethyl)phenyl]-7-methoxy-5-methyl[1,2,4]triazolo[4,3-a]quinolin-1(2H)-one;
4-methoxy-3-(7-methoxy-5-methyl-1-oxo-1,2-dihydro[1,2,4]triazolo[4,3-a]quinolin-8-yl)benzaldehyde;
8-(3,4-dimethoxyphenyl)-7-methoxy-5-methyl[1,2,4]triazolo[4,3-a]quinolin-1(2H)-one;
8-(3-chloro-4-fluorophenyl)-7-methoxy-5-methyl[1,2,4]triazolo[4,3-a]quinolin-1(2H)-one;
8-[4-(dimethylamino)phenyl]-7-methoxy-5-methyl[1,2,4]triazolo[4,3-a]quinolin-1(2H)-one;
8-(3-aminophenyl)-7-methoxy-5-methyl[1,2,4]triazolo[4,3-a]quinolin-1(2H)-one;
8-(2,6-dimethoxyphenyl)-7-methoxy-5-methyl[1,2,4]triazolo[4,3-a]quinolin-1(2H)-one;
7-methoxy-8-(3-methoxyphenyl)-5-methyl[1,2,4]triazolo[4,3-a]quinolin-1(2H)-one;
8-(4-chlorophenyl)-7-methoxy-5-methyl[1,2,4]triazolo[4,3-a]quinolin-1(2H)-one;
7-methoxy-8-[3-(methoxymethyl)phenyl]-5-methyl[1,2,4]triazolo[4,3-a]quinolin-1(2H)-one;
8-[3-(hydroxymethyl)phenyl]-7-methoxy-5-methyl[1,2,4]triazolo[4,3-a]quinolin-1(2H)-one;
8-[4-(hydroxymethyl)phenyl]-7-methoxy-5-methyl[1,2,4]triazolo[4,3-a]quinolin-1(2H)-one;
8-[3-(aminomethyl)phenyl]-7-methoxy-5-methyl[1,2,4]triazolo[4,3-a]quinolin-1(2H)-one;
8-(3-{[(2-hydroxyethyl)amino]methyl}phenyl)-7-methoxy-5-methyl[1,2,4]triazolo[4,3-a]quinolin-1(2H)-one;
7-methoxy-5-methyl-8-[3-({[(1-methyl-i H-pyrrol-2-yl)methyl]amino}methyl)phenyl][1,2,4]triazolo[4,3-a]quinolin-1(2H)-one;
8-(3-{[4-(2-hydroxyethyl)piperidin-1-yl]methyl}phenyl)-7-methoxy-5-methyl[1,2,4]triazolo[4,3-a]quinolin-1(2H)-one;
7-methoxy-5-methyl-8-(3-{[(pyridin-4-ylmethyl)amino]methyl}phenyl)[1,2,4]triazolo[4,3-a]quinolin-1(2H)-one;
8-{3-[(isobutylamino)methyl]phenyl}-7-methoxy-5-methyl[1,2,4]triazolo[4,3-a]quinolin-1(2H)-one;
7-methoxy-5-methyl-8-(3-{[(3-morpholin-4-ylpropyl)amino]methyl}phenyl)[1,2,4]triazolo[4,3-a]quinolin-1(2H)-one;
8-(3-{[4-(hydroxymethyl)piperidin-1-yl]methyl}phenyl)-7-methoxy-5-methyl[1,2,4]triazolo[4,3-a]quinolin-1(2H)-one;
8-[3-({[3-(1H-imidazol-1-yl)propyl]amino}methyl)phenyl]-7-methoxy-5-methyl[1,2,4]triazolo[4,3-a]quinolin-1(2H)-one;
8-(3-{[(3-chlorobenzyl)amino]methyl}phenyl)-7-methoxy-5-methyl[1,2,4]triazolo[4,3-a]quinolin-1(2H)-one;
7-methoxy-5-methyl-8-[3-({methyl[2-(methylamino)ethyl]amino}methyl)phenyl][1,2,4]triazolo[4,3-a]quinolin-1(2H)-one;
8-(4-methoxyphenyl)-5-methyl[1,2,4]triazolo[4,3-a]quinolin-1(2H)-one;
5-hydroxy-8-[4-(hydroxymethyl)phenyl][1,2,4]triazolo[4,3-a]quinolin-1(2H)-one;
5-methyl-8-pyridin-4-yl[1,2,4]triazolo[4,3-a]quinolin-1(2H)-one;
7-hydroxy-8-[2-(hydroxymethyl)phenyl]-5-methyl[1,2,4]triazolo[4,3-a]quinolin-1(2H)-one;
8-[3-(aminomethyl)phenyl]-7-hydroxy-5-methyl[1,2,4]triazolo[4,3-a]quinolin-1(2H)-one;
8-[3-({[3-(dimethylamino)propyl]amino}methyl)phenyl]-7-methoxy-5-methyl[1,2,4]triazolo[4,3-a]quinolin-1(2H)-one;;
8-[3-({[3-(dimethylamino)propyl]amino}methyl)-4-methoxyphenyl]-7-methoxy-5-methyl[1,2,4]triazolo[4,3-a]quinolin-1(2H)-one;
7-methoxy-8-{4-methoxy-3-[(4-methylpiperazin-1-yl)methyl]phenyl}-5-methyl[1,2,4]triazolo[4,3-a]quinolin-1(2H)-one;
8-(3-{[(2-hydroxyethyl)amino]methyl}-4-methoxyphenyl)-7-methoxy-5-methyl[1,2,4]triazolo[4,3-a]quinolin-1(2H)-one;
7-methoxy-5-methyl-8-(1H-pyrrol-2-yl)[1,2,4]triazolo[4,3-a]quinolin-1(2H)-one;
N-[4-(5-methyl-1-oxo-1,2-dihydro[1,2,4]triazolo[4,3-a]quinolin-8-yl)phenyl]acetamide;
5-methyl-8-thien-2-yl[1,2,4]triazolo[4,3-a]quinolin-1(2H)-one;
5-amino-8-thien-2-yl[1,2,4]triazolo[4,3-a]quinolin-1(2H)-one;
5-amino-8-(2-furyl)[1,2,4]triazolo[4,3-a]quinolin-1(2H)-one;
5-methyl-8-(1H-pyrrol-2-yl)[1,2,4]triazolo[4,3-a]quinolin-1(2H)-one;
8-{2-furyl)-5-methyl[1,2,4]triazolo[4,3-a]quinolin-1(2H)-one;
5-amino-8-thien-3-yl[1,2,4]triazolo[4,3-a]quinolin-1(2H)-one;
5-methyl-8-thien-3-yl[1,2,4]triazolo[4,3-a]quinolin-1(2H)-one;
N,N-dimethyl-3-(5-methyl-1-oxo-1,2-dihydro[1,2,4]triazolo[4,3-a]quinolin-8-yl)benzamide;
5-{4-[(cyclopentylamino)methyl]phenyl}[1,2,4]triazolo[4,3-a]quinolin-1(2H)-one;
5-(4-{[(tetrahydrofuran-2-ylmethyl)amino]methyl}phenyl)([1,2,4]triazolo[4,3-a]quinolin-1(2H)-one;
5-(4-{[(2-hydroxypropyl)amino]methyl }phenyl)[1,2,4]triazolo[4,3-a]quinolin-1(2H)-one T-butyl 4-{[4-(1-oxo-1,2-dihydro[1,2,4]triazolo[4,3-a]quinolin-5-yl)benzyl]amino}piperidine-1-carboxylate;
7-hydroxy-8-(2-{[(2-hydroxypropyl)amino]methyl}phenyl)-5-methyl[1,2,4]triazolo[4,3-a]quinolin-1(2H)-one;
7-hydroxy-5-methyl-8-phenyl[1,2,4]triazolo[4,3-a]quinolin-1(2H)-one;
8-(4-chlorophenyl)-7-hydroxy-5-methyl[1,2,4]triazolo[4,3-a]quinolin-1(2H)-one;
8-(3-chloro-4-fluorophenyl)-7-hydroxy-5-methyl[1,2,4]triazolo[4,3-a]quinolin-1(2H)-one;
8-[4-(dimethylamino)phenyl]-7-hydroxy-5-methyl[1,2,4]triazolo[4,3-a]quinolin-1(2H)-one;
8-(3-aminophenyl)-7-hydroxy-5-methyl[1,2,4]triazolo[4,3-a]quinolin-1(2H)-one;
7-hydroxy-8-(2-methoxypyridin-4-yl)-5-methyl[1,2,4]triazolo[4,3-a]quinolin-1(2H)-one;
5-(3-aminopropoxy)[1,2,4]triazolo[4,3-a]quinolin-1(2H)-one;
8-(2-aminophenyl)-7-hydroxy-5-methyl[1,2,4]triazolo[4,3-a]quinolin-1(2H)-one;
8-{2-[(cyclopentylamino)methyl]phenyl}-7-hydroxy-5-methyl[1,2,4]triazolo[4,3-a]quinolin-1(2H)-one;
7-hydroxy-8-(3-hydroxyphenyl)-5-methyl[1,2,4]triazolo[4,3-a]quinolin-1(2H)-one;
7-hydroxy-5-methyl-8-[3-({methyl[2-(methylamino)ethyl]amino}methyl)phenyl][1,2,4]triazolo[4,3-a]quinolin-1(2H)-one;
7-hydroxy-8-[3-({[3-(1H-imidazol-1-yl)propyl]amino}methyl)phenyl]-5-methyl[1,2,4]triazolo[4,3-a]quinolin-1(2H)-one;
8-[3-({[3-(dimethylamino)propyl]amino}methyl)phenyl]-7-hydroxy-5-methyl[1,2,4]triazolo[4,3-a]quinolin-1(2H)-one;
7-hydroxy-5-methyl-8-(3-{[(pyridin-4-ylmethyl)amino]methyl}phenyl)[1,2,4]triazolo[4,3-a]quinolin-1(2H)-one;
4-amino-8-[4-(hydroxymethyl)phenyl][1,2,4]triazolo[4,3-a]quinolin-1(2H)-one;
7-hydroxy-5-methyl-8-(3-{[(3-morpholin-4-ylpropyl)amino]methyl}phenyl)[1,2,4]triazolo[4,3-a]quinolin-1(2H)-one;
8-(3-{[(3-chlorobenzyl)amino]methyl}phenyl)-7-hydroxy-5-methyl[1,2,4]triazolo[4,3-a]quinolin-1(2H)-one;
5-[3-({[2-(4-benzylpiperazin-1-yl)ethyl]amino}methyl)-4-hydroxyphenyl][1,2,4]triazolo[4,3-a]quinolin-1(2H)-one;
5-{2-hydroxy-6-[(4-pyridin-2-ylpiperazin-1-yl)methyl]phenyl}[1,2,4]triazolo[4,3-a]quinolin-1(2H)-one;
5-[2-({[2-(4-benzylpiperazin-1-yl)ethyl]amino}methyl)-6-hydroxyphenyl][1,2,4]triazolo[4,3-a]quinolin-1(2H)-one;
4-amino-8-thien-2-yl[1,2,4]triazolo[4,3-a]quinolin-1(2H)-one;
4-amino-8-[3-hydroxymethyl)phenyl][1,2,4]triazolo[4,3-a]quinolin-1(2H)-one;
6-[3-(aminomethyl)phenyl][1,2,4]triazolo[4,3-a]quinolin-1(2H)-one;
5-(hydroxymethyl)-8-[3-(hydroxymethyl)phenyl]-7-methoxy[1,2,4]triazolo[4,3-a]quinolin-1(2H)-one;
8-(3-aminophenyl)-5-(hydroxymethyl)-7-methoxy 1,2,4]triazolo[4,3-a]quinolin-1(2H)-one;
7-hydroxy-5-(hydroxymethyl)-8-[2-(hydroxymethyl)phenyl][1,2,4]triazolo [4,3-a]quinolin-1(2H)-one;
7-hydroxy-5(hydroxymethyl)-8-[3-(hydroxymethyl)phenyl][1,2,4]triazolo[4,3-a]quinolin-1(2H)-one;
8-(3-aminophenyl)-7-hydroxy-5-(hydroxymethyl)[1,2,4]triazolo[4,3-a]quinolin-1(2H)-one;
6-{3-[(cyclohexylamino)methyl]phenyl}[1,2,4]triazolo[4,3-a]quinolin-1(2H)-one;
6-{3-[(cyclopentylamino)methyl]phenyl}[1,2,4]triazolo[4,3-a]quinolin-1(2H)-one;
6-(3-{[(tetrahydrofuran-2-ylmethyl)amino]methyl}phenyl)[1,2,4]triazolo[4,3-a]quinolin-1(2H)-one;
6-(3-{[4-(hydroxymethyl)piperidin-1-yl]methyl}phenyl)[1,2,4]triazolo[4,3-a]quinolin-1(2H)-one;
4-(hydroxymethyl)-8-pyridin-4-yl[1,2,4]triazolo[4,3-a]quinolin-1(2H)-one;
8-(3-furyl)-5-methyl[1,2,4]triazolo[4,3-a]quinolin-1(2H)-one;
6-{3-[(isobutylamino)methyl]phenyl}[1,2,4]triazolo[4,3-a]quinolin-1(2H)-one;
6-(3-{[4-(2-hydroxyethyl)-1,4-diazepan-1-yl]methyl}phenyl)[1,2,4]triazolo [4,3-a]quinolin-1(2H)-one;
8-(2-aminophenyl)-7-hydroxy-5-(hydroxymethyl)[1,2,4]triazolo[4,3-a]quinolin-1(2H)-one;
6-(4-hydroxy-3-{[(tetrahydro furan-2-ylmethyl)amino]methyl}phenyl) [1,2,4]triazolo[4,3-a]quinolin-1(2H)-one;
6-{4-hydroxy-3-[(isobutylamino)methyl]phenyl}[1,2,4]triazolo[4,3-a]quinolin-1(2H)-one;
4-(hydroxymethyl)-8-thien-2-yl[1,2,4]triazolo[4,3-a]quinolin-1(2H)-one;
5-(hydroxymethyl)-8-thien-2-yl[1,2,4]triazolo[4,3-a]quinolin-1(2H)-one;
5-(hydroxymethyl)-8-thien-3-yl[1,2,4]triazolo[4,3-a]quinolin-1(2H)-one;
7-methoxy-5-methyl-8-thien-3-yl[1,2,4]triazolo[4,3-a]quinolin-1(2H)-one;
7-hydroxy-5-methyl-8-thien-3-yl[1,2,4]triazolo[4,3-a]quinolin-1(2H)-one;
5-amino-7-hydroxy-8-thien-3-yl[1,2,4]triazolo[4,3-a]quinolin-1(2H)-one;
N-[3-(5-methyl-1-oxo-1,2-dihydro[1,2,4]triazolo[4,3-a]quinolin-8-yl)phenyl]methanesulfonamide;
5-amino-8-(1H-pyrrol-2-yl)[1,2,4]triazolo[4,3-a]quinolin-1(2H)-one;
5-(hydroxymethyl)-8-(1H-pyrrol-2-yl)[1,2,4]triazolo[4,3-a]quinolin-1(2H)-one;
5-methyl-8-(1H-pyrazol-4-yl)[1,2,4]triazolo[4,3-a]quinolin-1(2H)-one;
5-amino-8-(1H-pyrazol-4-yl)[1,2,4]triazolo[4,3-a]quinolin-1(2H)-one;
5-amino-8-(3-furyl)[1,2,4]triazolo[4,3-a]quinolin-1(2H)-one;
5-methyl-8-(4-methylthien-2-yl)[1,2,4]triazolo[4,3-a]quinolin-1(2H)-one;
8-[5-(hydroxymethyl)thien-2-yl]-5-methyl[1,2,4]triazolo[4,3-a]quinolin-1(2H)-one;
8-[5-(1-hydroxyethyl)thien-2-yl]-5-methyl[1,2,4]triazolo[4,3-a]quinolin-1(2H)-one;
Tert-butyl 4-{[(1-oxo-8-thien-3-yl-1,2-dihydro[1,2,4]triazolo[4,3-a]quinolin-5-yl)amino]methyl}piperidine-1-carboxylate;
8-bromo-5-(hydroxymethyl)[1,2,4]triazolo[4,3-a]quinolin-1(2H)-one;
7-methoxy-8-[4-methoxy-3-({[(1-methyl-1H-pyrrol-2-yl)methyl]amino}methyl)phenyl]-5-methyl[1,2,4]triazolo[4,3-a]quinolin-1(2H)-one;
7-methoxy-8-(4-methoxy-3-{[(pyridin4-ylmethyl)amino]methyl}phenyl)-5-methyl[1,2,4]triazolo[4,3-a]quinolin-1(2H)-one;
8-(3-{[4-(2-hydroxyethyl)piperidin-1-yl]methyl }-4-methoxyphenyl)-7-methoxy-5-methyl[1,2,4]triazolo[4,3-a]quinolin-1(2H)-one;
7-methoxy-8-(4-methoxy-3-{[(2-pyridin-2-ylethyl)amino]methyl}phenyl)-5-methyl[1,2,4]triazolo[4,3-a]quinolin-1(2H)-one;
8-(3-{[4-(hydroxymethyl)piperidin-1-yl]methyl}-4-methoxyphenyl)-7-methoxy-5-methyl[1,2,4]triazolo[4,3-a]quinolin-1(2H)-one;
7-methoxy-8-(4-methoxy-3-{[(2-methoxyethyl)amino]methyl}phenyl)-5-methyl[1,2,4]triazolo[4,3-a]quinolin-1(2H)-one;
7-methoxy-8-(3-{[(2-methoxyethyl)amino]methyl}phenyl)-5-methyl[1,2,4]triazolo[4,3-a]quinolin-1(2H)-one;
8-{3-[(cyclopentylamino)methyl]-4-methoxyphenyl}-7-methoxy-5-methyl[1,2,4]triazolo[4,3-a]quinolin-1(2H)-one;
8-(3-{[(4-fluorobenzyl)amino]methyl }-4-methoxyphenyl)-7-methoxy-5-methyl[1,2,4]triazolo[4,3-a]quinolin-1(2H)-one;
8-{3-[(cyclobutylamino)methyl]-4-methoxyphenyl}-7-methoxy-5-methyl[1,2,4]triazolo[4,3-a]quinolin-1(2H)-one;
8-{3-[(cyclohexylamino)methyl]-4-methoxyphenyl}-7-methoxy-5-methyl[1,2,4]triazolo[4,3-a]quinolin-1(2H)-one;
8-{3-[(cyclopentylamino)methyl]phenyl}-7-methoxy-5-methyl[1,2,4]triazolo[4,3-a]quinolin-1(2H)-one;
8-{3-[(cyclobutylamino)methyl]phenyl}-7-methoxy-5-methyl[1,2,4]triazolo[4,3-a]quinolin-1(2H)-one;
8-{3-[(cyclohexylamino)methyl]phenyl}-7-methoxy-5-methyl[1,2,4]triazolo[4,3-a]quinolin-1(2H)-one;
8-(3-{[(2-hydroxypropyl)amino]methyl}phenyl)-7-methoxy-5-methyl[1,2,4]triazolo[4,3-a]quinolin-1(2H)-one;
8-(3-{[4-(2-hydroxyethyl)-1,4-diazepan-1-yl]methyl}phenyl)-7-methoxy-5-methyl[1,2,4]triazolo[4,3-a]quinolin-1(2H)-one;
8-{3-[(cyclopropylamino)methyl]phenyl}-7-methoxy-5-methyl[1,2,4]triazolo[4,3-a]quinolin-1(2H)-one;
7-methoxy-5-methyl-8-(3-{[(tetrahydrofuran-2-ylmethyl)amino]methyl}phenyl)[1,2,4]triazolo[4,3-a]quinolin-1(2H)-one;
7-methoxy-5-methyl-8-(3-{[(2-phenoxyethyl)amino]methyl}phenyl)[1,2,4]triazolo[4,3-a]quinolin-1(2H)-one;
7-methoxy-5-methyl-8-[3-({[2-(2-thienyl)ethyl]amino}methyl)phenyl][1,2,4]triazolo[4,3-a]quinolin-1(2H)-one;
8-{3-[(cyclopropylamino)methyl]-4-methoxyphenyl}-7-methoxy-5-methyl[1,2,4]triazolo[4,3-a]quinolin-1(2H)-one;
5-methyl-8-pyridin-3-yl[1,2,4]triazolo[4,3-a]quinolin-1(2H)-one;
7-hydroxy-8-{3-[(isobutylamino)methyl]phenyl}-5-methyl[1,2,4]triazolo[4,3-a]quinolin-1(2H)-one;
7-hydroxy-8-(3-{[4-(2-hydroxyethyl)piperidin-1-yl]methyl}phenyl)-5-methyl[1,2,4]triazolo[4,3-a]quinolin-1(2H)-one;
7-hydroxy-5-methyl-8-{3-[(4-methylpiperazin-1-yl)methyl]phenyl}[1,2,4]triazolo[4,3-a]quinolin-1(2H)-one;
7-hydroxy-8-(4-hydroxy-3-{[(pyridin-4-ylmethyl)amino]methyl}phenyl)-5-methyl[1,2,4]triazolo[4,3-a]quinolin-1(2H)-one;
8-{3-[(cyclopentylamino)methyl]phenyl}-7-hydroxy-5-methyl[1,2,4]triazolo[4,3-a]quinolin-1(2H)-one;
8-(3-{[(4-fluorobenzyl)amino]methyl}phenyl)-7-hydroxy-5-methyl[1,2,4]triazolo[4,3-a]quinolin-1(2H)-one;
8-[2-(hydroxymethyl)phenyl]-7-methoxy-5-methyl[1,2,4]triazolo[4,3-a]quinolin-1(2H)-one;
7-methoxy-5-methyl-8-[4-(morpholin-4-ylcarbonyl)phenyl][1,2,4]triazolo[4,3-a]quinolin-1(2H)-one;
7-methoxy-5-methyl-8-[4-(pyrrolidin-1-ylcarbonyl)phenyl][1,2,4]triazolo[4,3-a]quinolin-1(2H)-one;
7-methoxy-5-methyl-8-[4-(piperidin-1-ylcarbonyl)phenyl][1,2,4]triazolo[4,3-a]quinolin-1(2H)-one;
8-chloro-7-hydroxy-5-methyl[1,2,4]triazolo[4,3-a]quinolin-1(2H)-one;
8-{3-[(cyclobutylamino)methyl]phenyl}-7-hydroxy-5-methyl[1,2,4]triazolo[4,3-a]quinolin-1(2H)-one;
7-hydroxy-5-methyl-8-(3-{[(tetrahydrofuran-2-ylmethyl)amino]methyl}phenyl)[1,2,4]triazolo[4,3-a]quinolin-1(2H)-one;
7-hydroxy-8-(3-{[4-(2-hydroxyethyl)-1,4-diazepan-1-yl]methyl}phenyl)-5-methyl[1,2,4]triazolo[4,3-a]quinolin-1(2H)-one;
8-{3-[(cyclopropylamino)methyl]phenyl}-7-hydroxy-5-methyl[1,2,4]triazolo[4,3-a]quinolin-1(2H)-one;
8-{3-[(cyclopropylamino)methyl]phenyl}-7-hydroxy-5-methyl[1,2,4]triazolo[4,3-a]quinolin-1(2H)-one;
8-{3-[(cyclohexylamino)methyl]phenyl}-7-hydroxy-5-methyl[1,2,4]triazolo[4,3-a]quinolin-1(2H)-one;
N-cyclohexyl-4-(7-methoxy-5-methyl-1-oxo-1,2-dihydro[1,2,4]triazolo[4,3-a]quinolin-8-yl)benzamide;
8-(2-{[(4-fluorobenzyl)amino]methyl}phenyl)-7-methoxy-5-methyl[1,2,4]triazolo[4,3-a]quinolin-1(2H)-one;
8-(2-{[(2-hydroxyethyl)amino]methyl}phenyl)-7-methoxy-5-methyl[1,2,4]triazolo[4,3-a]quinolin-1(2H)-one;
8-(2-{[4-(hydroxymethyl)piperidin-1-yl]methyl}phenyl)-7-methoxy-5-methyl[1,2,4]triazolo[4,3-a]quinolin-1(2H)-one;
8-(2-{[4-(hydroxymethyl)piperidin-1-yl]methyl}phenyl)-7-methoxy-5-methyl[1,2,4]triazolo[4,3-a]quinolin-1(2H)-one;
7-bromo-N-(4-methoxybenzyl)-1-oxo-1,2-dihydro[1,2,4]triazolo[4,3-a]quinoline-5-carboxamide;
8-(benzylamino)-5-methyl[1,2,4]triazolo[4,3-a]quinolin-1(2H)-one;
N,N-dimethyl-4-(5-methyl-1-oxo-1,2-dihydro[1,2,4]triazolo[4,3-a]quinolin-8-yl)benzamide;
5-methyl-8-[4-(pyrrolidin-1-ylcarbonyl)phenyl][1,2,4]triazolo[4,3-a]quinolin-1(2H)-one;
5-methyl-8-[4-(piperidin-1-ylcarbonyl)phenyl][1,2,4]triazolo[4,3-a]quinolin-1(2H)-one;
[4-(5-methyl-1-oxo-1,2-dihydro[1,2,4]triazolo[4,3-a]quinolin-8-yl)phenyl]acetonitrile;
8-[3-(hydroxymethyl)phenyl]-1-oxo-1,2-dihydro[1,2,4]triazolo[4,3-a]quinoline-5-carboxylic acid;
3-(5-methyl-1-oxo-1,2-dihydro[1,2,4]triazolo[4,3-a]quinolin-8-yl)benzonitrile;
5-methyl-8-[4-(morpholin-4-ylcarbonyl)phenyl][1,2,4]triazolo[4,3-a]quinolin-1(2H)-one;
8-[2-(hydroxymethyl)phenyl]-1-oxo-N-piperidin-4-yl-1,2-dihydro[1,2,4]triazolo[4,3-a]quinoline-5-carboxamide;
7-[3-(hydroxymethyl)phenyl]-1-oxo-N-piperidin-4-yl-1,2-dihydro[1,2,4]triazolo[4,3-a]quinoline-5-carboxamide;
[3-(7-methoxy-5-methyl-1-oxo-1,2-dihydro[1,2,4]triazolo[4,3-a]quinolin-8-yl)phenyl]acetonitrile;
N-(2-cyanoethyl)-3-(7-methoxy-5-methyl-1-oxo-1,2-dihydro[1,2,4]triazolo[4,3-a]quinolin-8-yl)benzamide;
6-chloro[1,2,4]triazolo[4,3-a]quinolin-1(2H)-one;
7-hydroxy-5-methyl-8-[4-(piperidin-1-ylcarbonyl)phenyl][1,2,4]triazolo[4,3-a]quinolin-1(2H)-one;
N-cyclohexyl-4-(7-hydroxy-5-methyl-1-oxo-1,2-dihydro[1,2,4]triazolo[4,3-a]quinolin-8-yl)benzamide;
6-[4-(hydroxymethyl)phenyl][1,2,4]triazolo[4,3-a]quinolin-1(2H)-one;
6-[3-(hydroxymethyl)phenyl][1,2,4]triazolo[4,3-a]quinolin-1(2H)-one;
6-(3-aminophenyl)[1,2,4]triazolo[4,3-a]quinolin-1(2H)-one;
7-methoxy-4-([(pyridin-4-ylmethyl)amino]methyl}[1,2,4]triazolo[4,3-a]quinolin-1(2H)-one;
5-amino-8-pyridin-4-yl[1,2,4]triazolo[4,3-a]quinolin-1(2H)-one;
3-(5-methyl-1-oxo-1,2-dihydro[1,2,4]triazolo[4,3-a]quinolin-8-yl)benzamide;
2-(5-methyl-1-oxo-1,2-dihydro[1,2,4]triazolo[4,3-a]quinolin-8-yl)benzamide;
8-chloro-7-(3-chloropropoxy)-5-methyl[1,2,4]triazolo[4,3-a]quinolin-1(2H)-one;
8-chloro-7-(2-methoxyethoxy)-5-methyl[1,2,4]triazolo[4,3-a]quinolin-1(2H)-one;
8-[3-(hydroxymethyl)phenyl]-7-(2-methoxyethoxy)-5-methyl[1,2,4]triazolo[4,3-a]quinolin-1(2H)-one;
7-(3-aminopropoxy)-8-[3-(hydroxymethyl)phenyl]-5-methyl [1,2,4]triazolo [4,3-a]quinolin-1(2H)-one;
7-(3-aminopropoxy)-8-[2-(hydroxymethyl)phenyl]-5-methyl[1,2,4]triazolo[4,3-a]quinolin-1(2H)-one;
7-hydroxy-8-(2-hydroxyphenyl)-5-methyl[1,2,4]triazolo[4,3-a]quinolin-1(2H)-one;
8-bromo-4-(hydroxymethyl)[1,2,4]triazolo[4,3-a]quinolin-1(2H)-one;
5-methyl-8-(2-thienyl)[1,2,4]triazolo[4,3-a]quinolin-1(2H)-one;
N,N-dimethyl-3-(5-methyl-1-oxo-1,2-dihydro[1,2,4]triazolo[4,3-a]quinolin-8-yl)benzamide;
5-{4-[(cyclopentylamino)methyl]phenyl}[1,2,4]triazolo[4,3-a]quinolin-1(2H)-one;
5-(4-{[(tetrahydrofuran-2-ylmethyl)amino]methyl}phenyl)[1,2,4]triazolo[4,3-a]quinolin-1(2H)-one;
5-(4-{[(2-hydroxypropyl)amino]methyl}phenyl)[1,2,4]triazolo[4,3-a]quinolin-1(2H)-one;
tert-butyl 4-{[4-(1-oxo-1,2-dihydro[1,2,4]triazolo[4,3-a]quinolin-5-yl)benzyl]amino}piperidine-1-carboxylate;
7-hydroxy-8-(2-{[(2-hydroxypropyl)amino]methyl}phenyl)-5-methyl[1,2,4]triazolo[4,3-a]quinolin-1(2H)-one;
8-(2-{[(4-fluorobenzyl)amino]methyl}phenyl)-7-hydroxy-5-methyl[1,2,4]triazolo[4,3-a]quinolin-1(2H)-one;
7-hydroxy-8-(2-{[4-(hydroxymethyl)piperidin-1-yl]methyl}phenyl)-5-methyl[1,2,4]triazolo[4,3-a]quinolin-1(2H)-one;
4-(hydroxymethyl)-8-methoxy[1,2,4]triazolo[4,3-a]quinolin-1(2H)-one;
8-{2-[(cyclopentylamino)methyl]phenyl}-7-methoxy-5-methyl[1,2,4]triazolo[4,3-a]quinolin-1(2H)-one;
4-[(cyclobutylamino)methyl]-7-hydroxy[1,2,4]triazolo[4,3-a]quinolin-1(2H)-one;
7-hydroxy-5-methyl-8-phenyl[1,2,4]triazolo[4,3-a]quinolin-1(2H)-one;
8-(4-chlorophenyl)-7-hydroxy-5-methyl[1,2,4]triazolo[4,3-a]quinolin-1(2H)-one;
4-{[(4-fluorobenzyl)amino]methyl}-7-hydroxy[1,2,4]triazolo[4,3-a]quinolin-1(2H)-one;
8-[4-(dimethylamino)phenyl]-7-hydroxy-5-methyl[1,2,4]triazolo[4,3-a]quinolin-1(2H)-one;
8-(3-aminophenyl)-7-hydroxy-5-methyl[1,2,4]triazolo[4,3-a]quinolin-1(2H)-one;
8-(2-aminophenyl)-7-methoxy-5-methyl[1,2,4]triazolo[4,3-a]quinolin-1(2H)-one;
7-hydroxy-8-(2-methoxypyridin-4-yl)-5-methyl[1,2,4]triazolo[4,3-a]quinolin-1(2H)-one;
5-(3-aminopropoxy)[1,2,4]triazolo[4,3-a]quinolin-1(2H)-one;
8-[2-(hydroxymethyl)-4-methoxyphenyl]-S-methyl[1,2,4]triazolo[4,3-a]quinolin-1(2H)-one;
8-(2-aminophenyl)-7-hydroxy-5-methyl[1,2,4]triazolo[4,3-a]quinolin-1(2H)-one;
8-{2-[(cyclopentylamino)methyl]phenyl}-7-hydroxy-5-methyl[1,2,4]triazolo[4,3-a]quinolin-1(2H)-one;
7-hydroxy-8-(3-hydroxyphenyl)-5-methyl[1,2,4]triazolo[4,3-a]quinolin-1(2H)-one;
7-hydroxy-5-methyl-8-[3-({methyl[2-(methylamino)ethyl]amino}methyl)phenyl][1,2,4]triazolo[4,3-a]quinolin-1(2H)-one;
7-hydroxy-8-[3-({[3-(1H-imidazol-1-yl)propyl]amino}methyl)phenyl]-5-methyl[1,2,4]triazolo[4,3-a]quinolin-1(2H)-one;
7-hydroxy-5-methyl-8-(3-{[(pyridin-4-ylmethyl)amino]methyl}phenyl)[1,2,4]triazolo[4,3-a]quinolin-1(2H)-one;
4-amino-8-[4-(hydroxymethyl)phenyl][1,2,4]triazolo[4,3-a]quinolin-1(2H)-one;
8-hydroxy-4-(hydroxymethyl)[1,2,4]triazolo[4,3-a]quinolin-1(2H)-one;
2-(5-amino-1-oxo-1,2-dihydro[1,2,4]triazolo[4,3-a]quinolin-8-yl)benzamide;
7-hydroxy-5-methyl-8-(3-{[(3-morpholin-4-ylpropyl)amino]methyl}phenyl)[1,2,4]triazolo[4,3-a]quinolin-1(2H)-one;
8-(3-{[(3-chlorobenzyl)amino]methyl}phenyl)-7-hydroxy-5-methyl[1,2,4]triazolo[4,3-a]quinolin-1(2H)-one;
5-[3-({[2-(4-benzylpiperazin-1-yl)ethyl]amino}methyl)-4-hydroxyphenyl][1,2,4]triazolo[4,3-a]quinolin-1(2H)-one;
5-{2-hydroxy-6-[(4-pyridin-2-ylpiperazin-1-yl)methyl]phenyl}[1,2,4]triazolo[4,3-a]quinolin-1(2H)-one;
ethyl 8-chloro-7-methoxy-1-oxo-1,2-dihydro[1,2,4]triazolo[4,3-a]quinoline-5-carboxylate;
2-(5-hydroxy-1-oxo-1,2-dihydro[1,2,4]triazolo[4,3-a]quinolin-8-yl)benzamide;
5-[2-({[2-(4-benzylpiperazin-1-yl)ethyl]amino}methyl)-6-hydroxyphenyl][1,2,4]triazolo[4,3-a]quinolin-1(2H)-one;
4-amino-8-(2-thienyl)[1,2,4]triazolo[4,3-a]quinolin-1(2H)-one;
8-chloro-7-hydroxy-5-hydroxymethyl)[1,2,4]triazolo[4,3-a]quinolin-1(2H)-one;
5-(hydroxymethyl)-8-[2-(hydroxymethyl)phenyl]-7-methoxy[1,2,4]triazolo[4,3-a]quinolin-1(2H)-one;
5-amino-8-(4-methoxyphenyl)[1,2,4]triazolo[4,3-a]quinolin-1(2H)-one;
5-methyl-8-(1H-pyrrol-2-yl)[1,2,4]triazolo[4,3-a]quinolin-1(2H)-one;
6-[4-(aminomethyl)phenyl][1,2,4]triazolo[4,3-a]quinolin-1(2H)-one;
N-[2-(1-oxo-1,2-dihydro[1,2,4]triazolo[4,3-a]quinolin-6-yl)phenyl]acetamide;
6-[2-(hydroxymethyl)phenyl][1,2,4]triazolo[4,3-a]quinolin-1(2H)-one;
4-amino-8-[3-(hydroxymethyl)phenyl][1,2,4]triazolo[4,3-a]quinolin-1(2H)-one;
4-amino-8-(2-furyl)[1,2,4]triazolo[4,3-a]quinolin-1(2H)-one;
6-[3-(aminomethyl)phenyl][1,2,4]triazolo[4,3-a]quinolin-1(2H)-one;
5-(hydroxymethyl)-8-[3-(hydroxymethyl)phenyl]-7-methoxy[1,2,4]triazolo[4,3-a]quinolin-1(2H)-one;
8-(3-aminophenyl)-5-(hydroxymethyl)-7-methoxy[1,2,4]triazolo[4,3-a]quinolin-1(2H)-one;
7-hydroxy-5-(hydroxymethyl)-8-[2-(hydroxymethyl)phenyl][1,2,4]triazolo[4,3-a]quinolin-1(2H)-one;
7-hydroxy-5-(hydroxymethyl)-8-[3-(hydroxymethyl)phenyl][1,2,4]triazolo[4,3-a]quinolin-1(2H)-one;
8-(3-aminophenyl)-7-hydroxy-5-(hydroxymethyl)[1,2,4]triazolo[4,3-a]quinolin-1(2H)-one;
6-{3-[(cyclohexylamino)methyl]-4-methoxyphenyl}[1,2,4]triazolo[4,3-a]quinolin-1(2H)-one;
6-(4-methoxy-3-{[(tetrahydrofuran-2-ylmethyl)amino]methyl}phenyl)[1,2,4]triazolo[4,3-a]quinolin-1(2H)-one;
6-{3-[(cyclohexylamino)methyl]phenyl}[1,2,4]triazolo[4,3-a]quinolin-1(2H)-one;
6-{3-[(cyclopentylamino)methyl]phenyl}[1,2,4]triazolo[4,3-a]quinolin-1(2H)-one;
6-(3-{[(tetrahydrofuran-2-ylmethyl)amino]methyl}phenyl)[1,2,4]triazolo[4,3-a]quinolin-1(2H)-one;
6-(3-{[4-(hydroxymethyl)piperidin-1-yl]methyl}-4-methoxyphenyl)[1,2,4]triazolo[4,3-a]quinolin-1(2H)-one;
6-(3-{[4-(hydroxymethyl)piperidin-1-yl]methyl}phenyl)[1,2,4]triazolo[4,3-a]quinolin-1(2H)-one;
6-(3-hydroxyphenyl)[1,2,4]triazolo[4,3-a]quinolin-1(2H)-one;
4-(hydroxymethyl)-8-pyridin-4-yl[1,2,4]triazolo[4,3-a]quinolin-1(2H)-one;
4-methyl-N-[1-oxo-8-(2-thienyl)-1,2-dihydro[1,2,4]triazolo[4,3-a]quinolin-4-yl]benzenesulfonamide;
6-{3-[(isobutylamino)methyl]-4-methoxyphenyl}[1,2,4]triazolo[4,3-a]quinolin-1(2H)-one;
6-(3-{[4-(2-hydroxyethyl)-1,4-diazepan-1-yl]methyl}-4-methoxyphenyl)[1,2,4]triazolo[4,3-a]quinolin-1(2H)-one;
6-{3-[(isobutylamino)methyl]phenyl}[1,2,4]triazolo[4,3-a]quinolin-1(2H)-one;
6-(3-{[4-(2-hydroxyethyl)-1,4-diazepan-1-yl]methyl}phenyl)[1,2,4]triazolo[4,3-a]quinolin-1(2H)-one;
8-(2-aminophenyl)-7-hydroxy-5-(hydroxymethyl)[1,2,4]triazolo[4,3-a]quinolin-1(2H)-one;
6-(4-hydroxy-3-{[(tetrahydrofuran-2-ylmethyl)amino]methyl}phenyl)[1,2,4]triazolo[4,3-a]quinolin-1(2H)-one;
6-{4-hydroxy-3-[(isobutylamino)methyl]phenyl}[1,2,4]triazolo[4,3-a]quinolin-1(2H)-one;
6-(4-hydroxy-3-{[4-(hydroxymethyl)piperidin-1-yl]methyl}phenyl)[1,2,4]triazolo[4,3-a]quinolin-1(2H)-one;
4-(hydroxymethyl)-8-(2-thienyl)[1,2,4]triazolo[4,3-a]quinolin-1(2H)-one;
6-(4-hydroxy-3-{[4-(2-hydroxyethyl)-1,4-diazepan-1-yl]methyl}phenyl)[1,2,4]triazolo[4,3-a]quinolin-1(2H)-one;
5-amino-8-(3-thienyl)[1,2,4]triazolo[4,3-a]quinolin-1(2H)-one;
5-amino-8-chloro-7-hydroxy[1,2,4]triazolo[4,3-a]quinolin-1(2H)-one;
7-methoxy-5-methyl-8-(3-thienyl) [1,2,4]triazolo [4,3-a]quinolin-1(2H)-one;
7-hydroxy-5-methyl-8-(3-thienyl)[1,2,4]triazolo[4,3-a]quinolin-1(2H)-one;
5-amino-7-hydroxy-8-(3-thienyl)[1,2,4]triazolo[4,3-a]quinolin-1(2H)-one;
N-[2-(5-methyl-1-oxo-1,2-dihydro[1,2,4]triazolo[4,3-a]quinolin-8-yl)phenyl]methanesulfonamide;
8-(1H-indol-3-yl)-5-methyl[1,2,4]triazolo[4,3-a]quinolin-1(2H)-one;
N-[3-(5-methyl-1-oxo-1,2-dihydro[1,2,4]triazolo[4,3-a]quinolin-8-yl)phenyl]methanesulfonamide;
5-amino-8-(1H-pyrrol-2-yl)[1,2,4]triazolo[4,3-a]quinolin-1(2H)-one;
5-(hydroxymethyl)-8-(1H-pyrrol-2-yl)[1,2,4]triazolo[4,3-a]quinolin-1(2H)-one;
5-methyl-8-(1H-pyrazol-4-yl)[1,2,4]triazolo[4,3-a]quinolin-1(2H)-one;
5-amino-8-(1H-pyrazol-4-yl)[1,2,4]triazolo[4,3-a]quinolin-1(2H)-one;
5-amino-8-(3-furyl)[1,2,4]triazolo[4,3-a]quinolin-1(2H)-one;
5-methyl-8-(4-methyl-2-thienyl)[1,2,4]triazolo[4,3-a]quinolin-1(2H)-one;
8-(3-furyl)-5-(hydroxymethyl)[1,2,4]triazolo[4,3-a]quinolin-1(2H)-one;
8-[5-(hydroxymethyl)-2-thienyl]-5-methyl[1,2,4]triazolo[4,3-a]quinolin-1(2H)-one;
8-[5-(1-hydroxyethyl)-2-thienyl]-5-methyl[1,2,4]triazolo[4,3-a]quinolin-1(2H)-one;
5-(5-methyl-1-oxo-1,2-dihydro 1,2,4]triazolo[4,3-a]quinolin-8-yl)thiophene-2-carboxylic acid;
tert-butyl 4-({[1-oxo-8-(3-thienyl)-1,2-dihydro[1,2,4]triazolo[4,3-a]quinolin-5-yl]amino}methyl)piperidine-1-carboxylate;
5-amino-8-[5-(1-hydroxyethyl)-2-thienyl][1,2,4]triazolo[4,3-a]quinolin-1(2H)-one;
8-(1H-imidazol-4-yl)-5-methyl[1,2,4]triazolo[4,3-a]quinolin-1(2H)-one;
5-(hydroxymethyl)-8-(1H-pyrazol-4-yl)[1,2,4]triazolo[4,3-a]quinolin-1(2H)-one;
8-bromo-5-[(dimethylamino)methyl][1,2,4]triazolo[4,3-a]quinolin-1(2H)-one;
8-(2-furyl)-5-(hydroxymethyl)[1,2,4]triazolo[4,3-a]quinolin-1(2E)-one;
5-methyl-8-(1,3-thiazol-2-yl)[1,2,4]triazolo[4,3-a]quinolin-1(2H)-one;
5-[(dimethylamino)methyl]-8-(1H-pyrrol-2-yl)[1,2,4]triazolo[4,3-a]quinolin-1(2H)-one;
5-methyl-8-pyrazin-2-yl[1,2,4]triazolo[4,3-a]quinolin-1(2H)-one;
5-(hydroxymethyl)-8-pyridin-4-yl[1,2,4]triazolo[4,3-a]quinolin-1(2H)-one;
(5-methyl-1-oxo-1,2-dihydro[1,2,4]triazolo[4,3-a]quinolin-8-yl)boronic acid;
8-(2-furyl)-5-phenyl[1,2,4]triazolo[4,3-a]quinolin-1(2H)-one;
5-phenyl-8-(1 H-pyrrol-2-yl)[1,2,4]triazolo[4,3-a]quinolin-1(2H)-one;
8-(3-furyl)-5-(morpholin-4-ylmethyl)[1,2,4]triazolo[4,3-a]quinolin-1(2H)-one;
tert-butyl [2-({[1-oxo-8-(1H-pyrrol-2-yl)-1,2-dihydro[1,2,4]triazolo[4,3-a]quinolin-5-yl]methyl}amino)ethyl]carbamate;
5-{[(2-aminoethyl)amino]methyl}-8-(1H-pyrrol-2-yl)[1,2,4]triazolo[4,3-a]quinolin-1(2H)-one;
N-(2-aminoethyl)-8-bromo-1-oxo-1,2-dihydro[1,2,4]triazolo[4,3-a]quinoline-5-carboxamide;
8-(3-furyl)-1-oxo-1,2-dihydro[1,2,4]triazolo[4,3-a]quinoline-5-carboxylic acid;
8-[3-(aminomethyl)phenyl]-5-phenyl[1,2,4]triazolo[4,3-a]quinolin-1(2H)-one;
N-[2-(dimethylamino)ethyl]-8-(3-furyl)-1-oxo-1,2-dihydro[1,2,4]triazolo[4,3-a]quinoline-5-carboxamide;
5-methyl-8-[4-(2-morpholin-4-ylethoxy)phenyl][1,2,4]triazolo[4,3-a]quinolin-1(2H)-one;
8-{4-[2-(diethylamino)ethoxy]phenyl}-5-methyl[1,2,4]triazolo[4,3-a]quinolin-1(2H)-one;
8-[3-(dimethylamino)prop-1-yn-1-yl]-5-methyl[1,2,4]triazolo[4,3-a]quinolin-1(2H)-one;
7-piperazin-1-yl-5-(2-thienyl)[1,2,4]triazolo[4,3-a]quinolin-1(2H)-one;
5-methyl-8-[3-(methylamino)prop-1-yn-1-yl][1,2,4]triazolo[4,3-a]quinolin-1(2H)-one;
5-methyl-8-[4-(morpholin-4-ylmethyl)phenyl][1,2,4]triazolo[4,3-a]quinolin-1(2H)-one;
N-[2-(dimethylamino)ethyl]-1-oxo-8-(1H-pyrrol-2-yl)-1,2-dihydro[1,2,4]triazolo[4,3-a]quinoline-5-carboxamide;
N-[2-(dimethylamino)ethyl]-1-oxo-8-(3-thienyl)-1,2-dihydro[1,2,4]triazolo[4,3-a]quinoline-5-carboxamide;
5-{[(3R)-piperidin-3-ylamino]methyl}-8-(1H-pyrrol-2-yl)[1,2,4]triazolo[4,3-a]quinolin-1(2H)-one;
5-methyl-8-{4-[(4-methylpiperazin-1-yl)methyl]phenyl}[1,2,4]triazolo[4,3-a]quinolin-1(2H)-one;
tert-butyl (3S)-3-({[1-oxo-8-(3-thienyl)-1,2-dihydro[1,2,4]triazolo[4,3-a]quinolin-5-yl]carbonyl}amino)piperidine-1-carboxylate;
5-methyl-8-{4-[(methylamino)methyl]phenyl}[1,2,4]triazolo[4,3-a]quinolin-1(2H)-one;
8-(3,3-diethoxyprop-1-yn-1-yl)-5-methyl[1,2,4]triazolo[4,3-a]quinolin-1(2H)-one;
5-methyl-8-[5-(morpholin-4-ylmethyl)-3-thienyl][1,2,4]triazolo[4,3-a]quinolin-1(2H)-one;
tert-butyl 4-[4-(5-methyl-1-oxo-1,2-dihydro[1,2,4]triazolo[4,3-a]quinolin-8-yl)benzyl]piperazine-1-carboxylate;
5-methyl-8-{5-[(methylamino)methyl]-3-thienyl}[1,2,4]triazolo[4,3-a]quinolin-1(2H)-one;
5-methyl-8-{5-[(4-methylpiperazin-1-yl)methyl]-3-thienyl}[1,2,4]triazolo[4,3-a]quinolin-1(2H)-one;
tert-butyl 4-{[4-(5-methyl-1-oxo-1,2-dihydro[1,2,4]triazolo[4,3-a]quinolin-8-yl)-2-thienyl]methyl}piperazine-1-carboxylate;
5-methyl-8-[4-(piperazin-1-ylmethyl)phenyl][1,2,4]triazolo[4,3-a]quinolin-1(2H)-one;
1-oxo-N-[(3S)-piperidin-3-yl]-8-(3-thienyl)-1,2-dihydro[1,2,4]triazolo[4,3-a]quinoline-5-carboxamide;
tert-butyl (3S)-3-({[1-oxo-8-(1H-pyrrol-2-yl)-1,2-dihydro[1,2,4]triazolo[4,3-a]quinolin-5-yl]carbonyl}amino)piperidine-1-carboxylate;
5-methyl-8-[5-(piperazin-1-ylmethyl)-3-thienyl][1,2,4]triazolo[4,3-a]quinolin-1(2H)-one;
N-(2-aminoethyl)-1-oxo-8-(3-thienyl)-1,2-dihydro[1,2,4]triazolo[4,3-a]quinoline-5-carboxamide;
1-oxo-N-[(3S)-piperidin-3-yl]-8-(1H-pyrrol-2-yl)-1,2-dihydro[1,2,4]triazolo[4,3-a]quinoline-5-carboxamide;
5-methyl-8-(1H-pyrrol-3-yl)[1,2,4]triazolo[4,3-a]quinolin-1(2H)-one;
5-methyl-8-(3-thienylethynyl)[1,2,4]triazolo[4,3-a]quinolin-1(2H)-one;
8-[5-({[3-(dimethylamino)propyl]amino}methyl)-3-thienyl]-5-methyl[1,2,4]triazolo[4,3-a]quinolin-1(2H)-one;
5-methyl-8-{5-[(methylamino)methyl]-2-thienyl}[1,2,4]triazolo[4,3-a]quinolin-1(2H)-one;
5-(hydroxymethyl)-8-[5-(morpholin-4-ylmethyl)-3-thienyl][1,2,4]triazolo[4,3-a]quinolin-1(2H)-one;
5-(hydroxymethyl)-8-{5-[(methylamino)methyl]-3-thienyl}[1,2,4]triazolo[4,3-a]quinolin-1(2H)-one;
5-(hydroxymethyl)-8-{5-[(4-methylpiperazin-1-yl)methyl]-3-thienyl}[1,2,4]triazolo[4,3-a]quinolin-1(2H)-one;
8-{5-[(dimethylamino)methyl]-3-thienyl}-5-(hydroxymethyl)[1,2,4]triazolo[4,3-a]quinolin-1(2H)-one;
5-(hydroxymethyl)-8-[5-(piperazin-1-ylmethyl)-3-thienyl][1,2,4]triazolo[4,3-a]quinolin-1(2H)-one;
N-[2-(methylamino)ethyl]-1-oxo-8-(3-thienyl)-1,2-dihydro[1,2,4]triazolo[4,3-a]quinoline-5-carboxamide;
N-[2-(methylamino)ethyl]-1-oxo-8-(1H-pyrrol-2-yl)-1,2-dihydro[1,2,4]triazolo[4,3-a]quinoline-5-carboxamide;
8-bromo-5-{[(2-methoxyethyl)(methyl)amino]methyl}[1,2,4]triazolo[4,3-a]quinolin-1(2H)-one;
N-[2-(dimethylamino)ethyl]-8-{5-[(4-methylpiperazin-1-yl)methyl]-3-thienyl}-1-oxo-1,2-dihydro[1,2,4]triazolo[4,3-a]quinoline-5-carboxamide;
8-bromo-5-{[2-(dimethylamino)ethoxy]methyl}[1,2,4]triazolo[4,3-a]quinolin-1(2H)-one;
N-(2-morpholin-4-ylethyl)-1-oxo-8-(3-thienyl)-1,2-dihydro[1,2,4]triazolo[4,3-a]quinoline-5-carboxamide;
5-[(4-methylpiperazin-1-yl)methyl]-8-(3-thienyl)[1,2,4]triazolo[4,3-a]quinolin-1(2H)-one;
8-[5-({[3-(dimethylamino)propyl]amino}methyl)-3-thienyl]-5-(hydroxymethyl)[1,2,4]triazolo[4,3-a]quinolin-1(2H)-one;
5-amino-8-{5-[(methylamino)methyl]-3-thienyl}[1,2,4]triazolo[4,3-a]quinolin-1(2H)-one;
5-(hydroxymethyl)-8-[5-(morpholin-4-ylmethyl)-2-thienyl][1,2,4]triazolo[4,3-a]quinolin-1(2H)-one;
5-(hydroxymethyl)-8-{5-[(methylamino)methyl]-2-thienyl}[1,2,4]triazolo[4,3-a]quinolin-1(2H)-one;
5-(hydroxymethyl)-8-{5-[(4-methylpiperazin-1-yl)methyl]-2-thienyl}[1,2,4]triazolo[4,3-a]quinolin-1(21H)-one;
8-{5-[(dimethylamino)methyl]-2-thienyl}-5-(hydroxymethyl)[1,2,4]triazolo[4,3-a]quinolin-1(2H)-one;
5-[(3-hydroxypyrrolidin-1-yl)methyl]-8-(3-thienyl)[1,2,4]triazolo[4,3-a]quinolin-1(2H)-one;
5-amino-8-{5-[(4-methylpiperazin-1-yl)methyl]-3-thienyl}[1,2,4]triazolo[4,3-a]quinolin-1(2H)-one;
5-(hydroxymethyl)-8-[5-(piperazin-1-ylmethyl)-2-thienyl][1,2,4]triazolo[4,3-a]quinolin-1(2H)-one;
5-(morpholin-4-ylmethyl)-8-(3-thienyl)[1,2,4]triazolo[4,3-a]quinolin-1(2H)-one;
5-{[(2-methoxyethyl)(methyl)amino]methyl}-8-(3-thienyl)[1,2,4]triazolo[4,3-a]quinolin-1(2H)-one;
1-oxo-N-(2-piperidin-1-ylethyl)-8-(3-thienyl)-1,2-dihydro[1,2,4]triazolo[4,3-a]quinoline-5-carboxamide;
5-{[(2-morpholin-4-ylethyl)amino]methyl}-8-(3-thienyl)[1,2,4]triazolo[4,3-a]quinolin-1(2H)-one;
5-[(dimethylamino)methyl]-8-(3-thienyl)[1,2,4]triazolo[4,3-a]quinolin-1(2H)-one;
N-[2-(dimethylamino)ethyl]-8-{4-[(4-methylpiperazin-1-yl)methyl]phenyl}-1-oxo-1,2-dihydro[1,2,4]triazolo[4,3-a]quinoline-5-carboxamide;
[1-oxo-8-(3-thienyl)-1,2-dihydro[1,2,4]triazolo[4,3-a]quinolin-5-yl]methyl glycinate;
5-{[2-(hydroxymethyl)morpholin-4-yl]methyl}-8-(1H-pyrrol-2-yl)[1,2,4]triazolo[4,3-a]quinolin-1(2H)-one;
5-[(4-methylpiperazin-1-yl)methyl]-8-(1H-pyrrol-2-yl)[1,2,4]triazolo[4,3-a]quinolin-1(2H)-one;
5-{[2-hydroxymethyl)morpholin-4-yl]methyl}-8-(3-thienyl)[1,2,4]triazolo[4,3-a]quinolin-1(2H)-one;
5-{[4-(2-hydroxyethyl)piperazin-1-yl]methyl}-8-(3-thienyl)[1,2,4]triazolo[4,3-a]quinolin-1(2H)-one;
N³,N³-dimethyl-N¹-[1-oxo-8-(3-thienyl)-1,2-dihydro[1,2,4]triazolo[4,3-a]quinolin-5-yl]-beta-alaninamide;
5-{[(3R)-3-hydroxypyrrolidin-1-yl]methyl}-8-(3-thienyl)[1,2,4]triazolo[4,3-a]quinolin-1(2H)-one;
5-{[(3R)-3-hydroxypyrrolidin-1-yl]methyl}-8-(3-thienyl)[1,2,4]triazolo[4,3-a]quinolin-1(2H)-one;
5-{[(2,3-dihydroxypropyl)(methyl)amino]methyl}-8-(3-thienyl)[1,2,4]triazolo[4,3-a]quinolin-1(2H)-one;
5-({methyl[2-(methylamino)ethyl]amino}methyl)-8-(3-thienyl)[1,2,4]triazolo[4,3-a]quinolin-1(2H)-one;
5-{[(3-methoxypropyl)amino]methyl}-8-(3-thienyl)[1,2,4]triazolo[4,3-a]quinolin-1(2H)-one;
5-{[(2-hydroxyethyl)(methyl)amino]methyl}-8-(3-thienyl)[1,2,4]triazolo[4,3-a]quinolin-1(2H)-one;
5-{[(3-hydroxypropyl)amino]methyl}-8-(3-thienyl)[1,2,4]triazolo[4,3-a]quinolin-1(2H)-one;
N-[2-(dimethylamino)ethyl]-8-{5-[(methylamino)methyl]-3-thienyl}-1-oxo-1,2-dihydro[1,2,4]triazolo[4,3-a]quinoline-5-carboxamide;
N-[2-(dimethylamino)ethyl]-8-[3-(dimethylamino)prop-1-yn-1-yl]-1-oxo-1,2-dihydro[1,2,4]triazolo[4,3-a]quinoline-5-carboxamide;
5-{[[3-(dimethylamino)propyl](methyl)amino]methyl}-8-(3-thienyl)[1,2,4]triazolo[4,3-a]quinolin-1(2H)-one;
5-({[3-(dimethylamino)propyl]amino}methyl)-8-(3-thienyl)[1,2,4]triazolo[4,3-a]quinolin-1(2H)-one;
N-[2-(dimethylamino)ethyl]-1-oxo-8-[4-(piperazin-1-ylmethyl)phenyl]-1,2-dihydro[1,2,4]triazolo[4,3-a]quinoline-5-carboxamide;
5-(aminomethyl)-8-(1H-pyrrol-2-yl)[1,2,4]triazolo[4,3-a]quinolin-1(2H)-one;
[1-oxo-8-(3-thienyl)-1,2-dihydro[1,2,4]triazolo[4,3-a]quinolin-5-yl]methyl N-methylglycinate;
5-{[(3-methoxypropyl)amino]methyl}-8-{5-[(methylamino)methyl]-3-thienyl}[1,2,4]triazolo[4,3-a]quinolin-1(2H)-one;
5-{[(2-hydroxyethyl)(methyl)amino]methyl}-8-{5-[(methylamino)methyl]-3-thienyl}[1,2,4]triazolo[4,3-a]quinolin-1(2H)-one;
5-{[4-(2-hydroxyethyl)piperazin-1-yl]methyl}-8-(1H-pyrrol-2-yl)[1,2,4]triazolo[4,3-a]quinolin-1(2H)-one;
5-{[(2,3-dihydroxypropyl)(methyl)amino]methyl}-8-{5-[(methylamino)methyl]-3-thienyl}[1,2,4]triazolo[4,3-a]quinolin-1(2H)-one;
5-{[(3R)-3-hydroxypyrrolidin-1-yl]carbonyl}-8-(3-thienyl)[1,2,4]triazolo[4,3-a]quinolin-1(2H)-one;
1-oxo-8-(3-thienyl)-1,2-dihydro[1,2,4]triazolo[4,3-a]quinoline-5-carboxylic acid;
8-{5-[(methylamino)methyl]-3-thienyl}-5-({methyl[2-(methylamino)ethyl]amino}methyl)[1,2,4]triazolo[4,3-a]quinolin-1(2H)-one;
5-{[[3-(dimethylamino)propyl](methyl)amino]methyl}-8-{5-[(methylamino)methyl]-3-thienyl}[1,2,4]triazolo[4,3-a]quinolin-1(2H)-one;
5-{[(3-hydroxypropyl)amino]methyl}-8-{5-[(methylamino)methyl]-3-thienyl}[1,2,4]triazolo[4,3-a]quinolin-1(2H)-one;
5-({[3-(dimethylamino)propyl]amino}methyl)-8-{5-[(methylamino)methyl]-3-thienyl}[1,2,4]triazolo[4,3-a]quinolin-1(2H)-one;
5-methyl-8-[3-(piperazin-1-ylmethyl)phenyl][1,2,4]triazolo[4,3-a]quinolin-1(2H)-one;
N-[2-(dimethylamino)ethyl]-8-{5-[(4-methylpiperazin-1-yl)methyl]-2-thienyl}-1-oxo-1,2-dihydro[1,2,4]triazolo[4,3-a]quinoline-5-carboxamide;
5-{[(3R)-3-hydroxypyrrolidin-1-yl]carbonyl}-8-{5-[(methylamino)methyl]-3-thienyl}[1,2,4]triazolo[4,3-a]quinolin-1(2H)-one;
5-[(methylamino)methyl]-8-(3-thienyl)[1,2,4]triazolo[4,3-a]quinolin-1(2H)-one;
5-({[2-(methylamino)ethyl]amino}methyl)-8-(3-thienyl)[1,2,4]triazolo[4,3-a]quinolin-1(2H)-one;
5-methyl-8-(5-{[(3S)-pyrrolidin-3-ylamino]methyl}-2-thienyl)[1,2,4]triazolo[4,3-a]quinolin-1(2H)-one;
5-methyl-8-(5-{[(3R)-pyrrolidin-3-ylamino]methyl}-2-thienyl)[1,2,4]triazolo[4,3-a]quinolin-1(2H)-one;
N-azetidin-3-yl-1-oxo-8-(3-thienyl)-1,2-dihydro[1,2,4]triazolo[4,3-a]quinoline-5-carboxamide;
8-{5-[(azetidin-3-ylamino)methyl]-2-thienyl}-5-methyl[1,2,4]triazolo[4,3-a]quinolin-1(2H)-one;
5-{[2-(dimethylamino)ethoxy]methyl}-8-(3-thienyl)[1,2,4]triazolo[4,3-a]quinolin-1(2H)-one;
5-({[(2S)-2,3-dihydroxypropyl]amino}methyl)-8-(3-thienyl)[1,2,4]triazolo[4,3-a]quinolin-1(2H)-one;
5-{[(3S)-3-hydroxypyrrolidin-1-yl]methyl}-8-(3-thienyl)[1,2,4]triazolo[4,3-a]quinolin-1(2H)-one;
5-{[(3-amino-2-hydroxypropyl)amino]methyl}-8-(3-thienyl)[1,2,4]triazolo[4,3-a]quinolin-1(2H)-one;
5-{[(3R)-3-(dimethylamino)pyrrolidin-1-yl]methyl}-8-(3-thienyl)[1,2,4]triazolo[4,3-a]quinolin-1(21H)-one;
5-{[(2-hydroxyethyl)amino]methyl}-8-(3-thienyl)[1,2,4]triazolo[4,3-a]quinolin-1(2H)-one;
5-(aminomethyl)-8-[4-(methoxymethyl)phenyl][1,2,4]triazolo[4,3-a]quinolin-1(2H)-one;
N-(3-hydroxypropyl)-1-oxo-8-(3-thienyl)-1,2-dihydro[1,2,4]triazolo[4,3-a]quinoline-5-carboxamide;
5-{[(3S)-3-hydroxypyrrolidin-1-yl]methyl}-8-(1H-pyrrol-2-yl)[1,2,4]triazolo[4,3-a]quinolin-1(2H)-one;
5-{[(3S)-3-hydroxypyrrolidin-1-yl]carbonyl}-8-(3-thienyl)[1,2,4]triazolo[4,3-a]quinolin-1(2H)-one;
5-({[2-(dimethylamino)ethyl]amino}methyl)-8-(1H-pyrrol-2-yl)[1,2,4]triazolo[4,3-a]quinolin-1(2H)-one;
8-{5-[(ethylamino)methyl]-3-thienyl}-5-methyl[1,2,4]triazolo[4,3-a]quinolin-1(2H)-one;
8-{5-[(isopropylamino)methyl]-3-thienyl}-5-methyl[1,2,4]triazolo[4,3-a]quinolin-1(2H)-one;
N-azetidin-3-yl-8-{5-[(methylamino)methyl]-3-thienyl}-1-oxo-1,2-dihydro[1,2,4]triazolo[4,3-a]quinoline-5-carboxamide;
5-(1H-imidazol-1-ylmethyl)-8-(1H-pyrrol-2-yl)[1,2,4]triazolo[4,3-a]quinolin-1(2H)-one;
5-(1H-imidazol-1-ylmethyl)-8-(3-thienyl)[1,2,4]triazolo[4,3-a]quinolin-1(2H)-one;
5-{[(3-hydroxypropyl)amino]methyl}-8-(1H-pyrrol-2-yl)[1,2,4]triazolo[4,3-a]quinolin-1(2H)-one;
5-[(pyrrolidin-3-ylamino)methyl]-8-(3-thienyl)[1,2,4]triazolo[4,3-a]quinolin-1(2H)-one;
5-{[(3R)-pyrrolidin-3-ylamino]methyl}-8-(3-thienyl)[1,2,4]triazolo[4,3-a]quinolin-1(2H)-one;
5-[(azetidin-3-ylamino)methyl]-8-(3-thienyl)[1,2,4]triazolo[4,3-a]quinolin-1(2H)-one;
5-{[(3S)-3-aminopyrrolidin-1-yl]methyl}-8-(3-thienyl)[1,2,4]triazolo[4,3-a]quinolin-1(2H)-one;
5-{[(3R)-3-aminopyrrolidin-1-yl]methyl}-8-(3-thienyl)[1,2,4]triazolo[4,3-a]quinolin-1(21)-one;
5-{[(3R)-3-(dimethylamino)pyrrolidin-1-yl]methyl}-8-(1H-pyrrol-2-yl)[1,2,4]triazolo[4,3-a]quinolin-1(21H)-one;
5-{[(2-hydroxyethyl)amino]methyl}-8-(1H-pyrrol-2-yl)[1,2,4]triazolo[4,3-a]quinolin-1(2H)-one;
5-[(3-aminoazetidin-1-yl)methyl]-8-(3-thienyl)[1,2,4]triazolo[4,3-a]quinolin-1(2H)-one;
5-({[2-(dimethylamino)ethyl]amino}methyl)-8-(3-thienyl)[1,2,4]triazolo[4,3-a]quinolin-1(2H)-one;
5-({[2-(1H-imidazol-4-yl)ethyl]amino}methyl)-8-(3-thienyl)[1,2,4]triazolo[4,3-a]quinolin-1(2H)-one;
5-({[3-(1H-imidazol-4-yl)propyl]amino}methyl)-8-(3-thienyl)[1,2,4]triazolo[4,3-a]quinolin-1(2H)-one;
5-[(isopropylamino)methyl]-8-(3-thienyl)[1,2,4]triazolo[4,3-a]quinolin-1(2H)-one;
5-[(ethylamino)methyl]-8-(3-thienyl)[1,2,4]triazolo[4,3-a]quinolin-1(2H)-one;
5-[(cyclopropylamino)methyl]-8-(3-thienyl)[1,2,4]triazolo[4,3-a]quinolin-1(2H)-one;
5-{[(cyclopropylmethyl)amino]methyl}-8-(3-thienyl)[1,2,4]triazolo[4,3-a]quinolin-1(2H)-one;
5-({[2-(dimethylamino)-1-methylethyl]amino}methyl)-8-(3-thienyl)[1,2,4]triazolo[4,3-a]quinolin-1(2H)-one;
N-[2-(dimethylamino)-1-methylethyl]-1-oxo-8-(3-thienyl)-1,2-dihydro[1,2,4]triazolo[4,3-a]quinoline-5-carboxamide;
5-{[methyl(2-pyridin4-ylethyl)amino]methyl}-8-(3-thienyl)[1,2,4]triazolo[4,3-a]quinolin-1(2H)-one;
5-[(3-aminoazetidin-1-yl)carbonyl]-8-(3-thienyl)[1,2,4]triazolo[4,3-a]quinolin-1(2H)-one;
N-[2-(1H-imidazol-4-yl)ethyl]-1-oxo-8-(3-thienyl)-1,2-dihydro[1,2,4]triazolo[4,3-a]quinoline-5-carboxamide;
N-[3-(1H-imidazol-4-yl)propyl]-1-oxo-8-(3-thienyl)-1,2-dihydro[1,2,4]triazolo[4,3-a]quinoline-5-carboxamide;
5-({[2-(isopropylamino)ethyl]amino}methyl)-8-(3-thienyl)[1,2,4]triazolo[4,3-a]quinolin-1(2H)-one;
N-[2-(isopropylamino)ethyl]-1-oxo-8-(3-thienyl)-1,2-dihydro[1,2,4]triazolo[4,3-a]quinoline-5-carboxamide;
N-[(1-ethylpyrrolidin-2-yl)methyl]-1-oxo-8-(3-thienyl)-1,2-dihydro[1,2,4]triazolo[4,3-a]quinoline-5-carboxamide;
5-({[3-(dimethylamino)propyl]amino}methyl)-8-(1H-pyrrol-2-yl)[1,2,4]triazolo[4,3-a]quinolin-1(2H)-one;
5-{[4-(hydroxymethyl)-1H-1,2,3-triazol-1-yl]methyl}-8-(3-thienyl)[1,2,4]triazolo[4,3-a]quinolin-1(2H)-one;
5-{[(pyridin-2-ylmethyl)amino]methyl}-8-(3-thienyl)[1,2,4]triazolo[4,3-a]quinolin-1(2H)-one;
5-({[(5-methyl-2-furyl)methyl]amino}methyl)-8-(3-thienyl)[1,2,4]triazolo[4,3-a]quinolin-1(2H)-one;
5-{[(2-pyridin-2-ylethyl)amino]methyl )-8-(3-thienyl)[1,2,4]triazolo[4,3-a]quinolin-1(2H)-one;
5-(methoxymethyl)-8-(3-thienyl)[1,2,4]triazolo[4,3-a]quinolin-1(2H)-one;
5-({[(5-methylpyrazin-2-yl)methyl]amino}methyl)-8-(3-thienyl)[1,2,4]triazolo[4,3-a]quinolin-1(21H)-one;
5-({4-[(methylamino)methyl]-1H-1,2,3-triazol-1-yl}methyl)-8-(3-thienyl)[1,2,4]triazolo[4,3-a]quinolin-1(2H)-one;
5-(aminomethyl)-8-(3-thienyl)[1,2,4]triazolo[4,3-a]quinolin-1(2H)-one;
5-{[(2-aminoethyl)amino]methyl}-8-(3-thienyl)[1,2,4]triazolo[4,3-a]quinolin-1(2H)-one;
5-chloro-2H-[1,2,4]triazolo[4,3-α]quinolin-1-one;
4-(1-oxo-1,2-dihydro-[1,2,4]triazolo[4,3-α]quinolin-5-yl)-benzaldehyde;
3-methoxy-2-(1-oxo-1,2-dihydro[1,2,4]triazolo[4,3-a]quinolin-5-yl)benzaldehyde;
8-bromo-1-oxo-1,2-dihydro -[1,2,4]-triazolo[4,3-a]quinoline-5-caroxylic acid;
8-bromo-1-oxo-1,2-dihydro-[1,2,4]-triazolo[4,3-a]quinoline-5-caroxylic acid ethyl ester;
7-chloro-5-methyl-2H-[1,2,4]triazolo[4,3-α]quinolin-1-one;
4-(5-methyl-1-oxo-1,2-dihydro-[1,2,4]triazolo[4,3-α]quinolin-7-yl)-benzaldehyde;
2-methoxy-5-(5-methyl-1-oxo-1,2-dihydro[1,2,4]triazolo[4,3-a]quinolin-7-yl)benzaldehyde;
3-(7-methoxy-5-methyl-1-oxo-1,2-dihydro-[1,2,4]triazolo[4,3-α]quinolin-8-yl)-benzaldehyde;
2-methoxy-5-(7-methoxy-5-methyl-1-oxo-1,2-dihydro[1,2,4]triazolo[4,3-a]quinolin-8-yl)benzaldehyde;
2-(7-methoxy-5-methyl-1-oxo-1,2-dihydro[1,2,4]triazolo[4,3-a]quinolin-8-yl)benzaldehyde;
8-bromo-5-bromomethyl[1,2[4]triazolo[4,3-a]quinolin-1(2H)-one;
8-bromo-5-hydroxymethyl[1,2[4]triazolo[4,3-a]quinolin-1(2H)-one;
8-bromo-1-oxo-1,2-dihydro[1,2,4]triazolo[4,3-a]quinoline-4-carboxylic acid;
4-amino-8-bromo[1,2,4]triazolo[4,3-a]quinolin-1(2H)-one;
8-bromo-5-phenyl[1,2,4]triazolo[4,3-a]quinolin-1(2H)-one;
8-bromo-5-methoxymethy[1,2,4]triazolo[4,3-a]quinolin-1(2H)-one;
8-Bromo-5-(aminomethyl)[1,2,4]triazolo[4,3-a]quinolin-1(2H)-one;
5-(azidomethyl)-8-bromo[1,2,4]triazolo[4,3-a]quinolin-1(2H)-one;
7-bromo-5-thien-2-yl[1,2,4]triazolo[4,3-a]quinolin-1(2H)-one;
ethyl 8-chloro-7-methoxy-1-oxo-1,2-dihydro[1,2,4]triazolo[4,3-a]quinoline-5-carboxylate;
8-chloro-7-methoxy-1-oxo-1,2-dihydro[1,2,4]triazolo[4,3-a]quinoline-5-carboxylic acid;
5-amino-8-chloro-7-methoxy[1,2,4]triazolo[4,3-a]quinolin-1(2H)-one;
8-chloro-5-(hydroxymethyl)-7-methoxy[1,2,4]triazolo[4,3-a]quinolin-1(2H)-one;
2-{3-[(1-oxo-1,2-dihydro[1,2,4]triazolo[4,3-a]quinolin-5-yl)oxy]propyl}-1H-isoindole-1,3(2H)-dione;
2-{3-[(8-chloro-5-methyl-1-oxo-1,2-dihydro[1,2,4]triazolo(4,3-a]quinolin-7-yl)oxy]propyl}-1H-isoindole-1,3(2H)-dione;
7-(3-aminopropoxy)-8-chloro-5-methyl[1,2,4]triazolo[4,3-a]quinolin-1(2H)-one.

In a particular embodiment the present invention provides a compound as recited in any of the embodiments above, wherein one or more of the atoms is a radioisotope of the same element.

In a particular embodiment the present invention provides a compound as recited in any of the embodiments above for the use in treatment of cancer.

In a particular embodiment the present invention provides a compound as recited in any of the embodiments above for use in treatment of neoplastic disease such as carcinoma of the breast, ovary, lung, colon, prostate or other tissues, as well as leukemias and lymphomas, tumors of the central and peripheral nervous system, and other tumor types such as melanoma, fibrosarcoma and osteosarcoma.

In a particular embodiment the present invention provides a compound as recited in any of the embodiments above for use in treatment of proliferative diseases including autoimmune, inflammatory, neurological, and cardiovascular diseases.

In a particular embodiment the present invention provides a method for treating a human or or animal by limiting cell replication by administering to such human or animal an effective amount of a compound as recited in any of the embodiments above or a pharmaceutically acceptable salt of said compound.

In a particular embodiment the present invention provides a method for treating a human or animal suffering from cancer by administering to such human or animal an effective amount of a compound as recited in any of embodiments above or a pharmaceutically acceptable salt of said compound.

In a particular embodiment the present invention provides a method for treating a human or animal suffering from neoplastic disease such as carcinoma of the breast, ovary, lung, colon, prostate or other tissues, as well as leukemias and lymphomas, tumors of the central and peripheral nervous system, and other tumor types such as melanoma, fibrosarcoma and osteosarcoma by administering to such human or animal an effective amount of a compound as recited in any of embodiments above or a pharmaceutically acceptable salt of said compound.

In a particular embodiment the present invention provides a method for treating a human or animal suffering from proliferative diseases including autoimmune, inflammatory, neurological, and cardiovascular diseases by administering to such human or animal an effective amount of a compound as recited in any of embodiments above or a pharmaceutically acceptable salt of said compound.

In a particular embodiment the present invention provides the use of a compound as recited in any of the embodiments above in the preparation of a medicament for the treatment of cancer. In a particular embodiment the present invention provides the use of a compound as recited in any one of the embodiments above in the preparation of a medicament for the treatment of neoplastic disease such as carcinoma of the breast, ovary, lung, colon, prostate or other tissues, as well as leukemias and lymphomas, tumors of the central and peripheral nervous system, and other tumor types such as melanoma, fibrosarcoma and osteosarcoma. In a particular embodiment the present invention provides the use of a compound as recited in any of the embodiments above in the preparation of a medicament for the treatment of proliferative diseases including autoimmune, inflammatory, neurological, and cardiovascular diseases.

In a particular embodiment the present invention provides a process for preparing a compound of formula (I) as recited in any of the embodiments above or a pharmaceutically acceptable salt or an in vivo hydrolysable ester therof which process comprises:

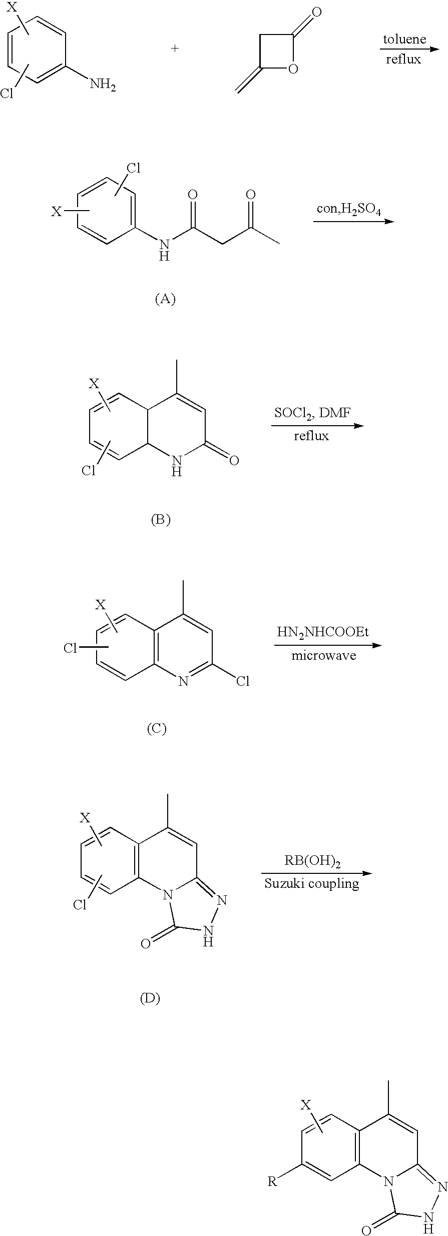

Diketene (32 ml, 32 g, 381 mmol) was added to the suspension of the appropriately substituted chloro aniline (317.25 mmol) in toluene (300 ml). The mixture was refluxed for 6 hr, cooled down and let stand overnight. The precipitated solid was filtered off, washed with ether and dried under vacuum to yield the intermediate (A).

A mixture of the appropriately substituted chloro acetoacetanilide (199.6 mmol) and concentrated sulfuric acid (80 ml) were heated on an oil-bath at 70-80° C. for 0.5 h and for 1.0 h at 100° C. The mixture was cooled down and poured into crushed ice. The precipitated solid was filtered off, and recrystallized from ethanol to obtain intermediate (B).

A mixture of the appropriately substituted 4-methyl-1H-quinolin-2-one (134.2 mmol), DMF (10 ml) and thionyl chloride (300 g) was heated at reflux for 3 hr. The mixture was cooled to room temperature and the resultant solid filtered off, washed with acetone and dried under vacuum to give the intermediate dichloroquinoline (C).

To a suspension of the appropriately substituted dichloro-4-methyl-quinoline (1.5 mmol) and ethyl carbazate (173 mg 1.66 mmol) in 3.7 ml of ethanol was added 6 drops of HCl (4N in dioxane). The reaction mixture was subject to irradiation with microwave at 170° C. for 20 min. After cooling to room temperature the precipitated solid was filtered off, washed with methanol (3×10 ml) and dried under vacuum to give the desired triazolone (D).

To a 5 ml vial, the appropriately substitutes 5-methyl-2H-[1,2,4]triazolo[4,3-a]quinolin-1-one (0.5 mmol), boronic acid (0.6 mmol), cesium carbonate (651 mg, 2.0 mmol), and tetrakis(trisphenylphosphine)palladium (40 mg, 7 mol %) were added in 4 ml of dioxane:water (4:1). The reaction was subject to irradiation with microwave at 165° C. for 20 min. After cooling down, the lower layer was separated and discarded, the upper layer was evaporated, the residue dissolved in the minimum amount of DMSO and filtered. The crude product was purified by HPLC.

Compounds of the present invention may be administered orally, parenteral, buccal, vaginal, rectal, inhalation, insufflation, sublingually, intramuscularly, subcutaneously, topically, intranasally, intraperitoneally, intrathoracially, intravenously, epidurally, intrathecally, intracerebroventricularly and by injection into the joints.

The dosage will depend on the route of administration, the severity of the disease, age and weight of the patient and other factors normally considered by the attending physician, when determining the individual regimen and dosage level as the most appropriate for a particular patient.

An effective amount of a compound of the present invention for use in therapy of infection is an amount sufficient to symptomatically relieve in a warm-blooded animal, particularly a human the symptoms of infection, to slow the progression of infection, or to reduce in patients with symptoms of infection the risk of getting worse.

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or tablet disintegrating agents; it can also be an encapsulating material.

In powders, the carrier is a finely divided solid, which is in a mixture with the finely divided active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized molds and allowed to cool and solidify.

Suitable carriers include magnesium carbonate, magnesium stearate, talc, lactose, sugar, pectin, dextrin, starch, tragacanth, methyl cellulose, sodium carboxymethyl cellulose, a low-melting wax, cocoa butter, and the like.

Some of the compounds of the present invention are capable of forming salts with various inorganic and organic acids and bases and such salts are also within the scope of this invention. Examples of such acid addition salts include acetate, adipate, ascorbate, benzoate, benzenesulfonate, bicarbonate, bisulfate, butyrate, camphorate, camphorsulfonate, choline, citrate, cyclohexyl sulfamate, diethylenediamine, ethanesulfonate, flumarate, glutamate, glycolate, hemisulfate, 2-hydroxyethylsulfonate, heptanoate, hexanoate, hydrochloride, hydrobromide, hydroiodide, hydroxymaleate, lactate, malate, maleate, methanesulfonate, meglumine, 2-naphthalenesulfonate, nitrate, oxalate, pamoate, persulfate, phenylacetate, phosphate, diphosphate, picrate, pivalate, propionate, quinate, salicylate, stearate, succinate, sulfamate, sulfanilate, sulfate, tartrate, tosylate p-toluenesulfonate), trifluoroacetate, and undecanoate. Base salts include ammonium salts, alkali metal salts such as sodium, lithium and potassium salts, alkaline earth metal salts such as aluminum, calcium and magnesium salts, salts with organic bases such as dicyclohexylamine salts, N-methyl-D-glucamine, and salts with amino acids such as arginine, lysine, ornithine, and so forth. Also, basic nitrogen-containing groups may be quaternized with such agents as: lower alkyl halides, such as methyl, ethyl, propyl, and butyl halides; dialkyl sulfates like dimethyl, diethyl, dibutyl; diamyl sulfates; long chain halides such as decyl, lauryl, myristyl and stearyl halides; aralkyl halides like benzyl bromide and others. Non-toxic physiologically-acceptable salts are preferred, although other salts are also useful, such as in isolating or purifying the product.

The salts may be formed by conventional means, such as by reacting the free base form of the product with one or more equivalents of the appropriate acid in a solvent or medium in which the salt is insoluble, or in a solvent such as water, which is removed in vacuo or by freeze drying or by exchanging the anions of an existing salt for another anion on a suitable ion-exchange resin.

In order to use a compound of the formula (I) or a pharmaceutically acceptable salt thereof for the therapeutic treatment (including prophylactic treatment) of mammals including humans, it is normally formulated in accordance with standard pharmaceutical practice as a pharmaceutical composition.

In addition to the compounds of the present invention, the pharmaceutical composition of this invention may also contain, or be co-administered (simultaneously or sequentially) with, one or more pharmacological agents of value in treating one or more disease conditions referred to herein.

The term composition is intended to include the formulation of the active component or a pharmaceutically acceptable salt with a pharmaceutically acceptable carrier. For example this invention may be formulated by means known in the art into the form of, for example, tablets, capsules, aqueous or oily solutions, suspensions, emulsions, creams, ointments, gels, nasal sprays, suppositories, finely divided powders or aerosols or nebulisers for inhalation, and for parenteral use (including intravenous, intramuscular or infusion) sterile aqueous or oily solutions or suspensions or sterile emulsions.

Liquid form compositions include solutions, suspensions, and emulsions. Sterile water or water-propylene glycol solutions of the active compounds may be mentioned as an example of liquid preparations suitable for parenteral administration. Liquid compositions can also be formulated in solution in aqueous polyethylene glycol solution. Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

The pharmaceutical compositions can be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms.

Compounds of the present invention can be labeled with a radioisotope including but not limited to tritium. Such radiolabeled compounds can be useful in the discovery of targets, or novel medicinail compounds which bind to and modulate the activity, by agonism, partial agonism, or antagonism, of CHK1. Such labeled compounds may be used in assays that measure the displacement of such compounds to assess the binding of ligand that bind to CHK1 receptors. Such radiolabeled compounds can be synthesized either by incorporating radiolabeled starting materials or, in the case of tritium, exchange of hydrogen for tritium by known methods. Known methods include (1) electrophilic halogenation, followed by reduction of the halogen in the presence of a tritium source, for example, by hydrogenation with tritium gas in the presence of a palladium catalyst, or (2) exchange of hydrogen for tritium performed in the presence of tritium gas and a suitable organometallic (e.g. palladium) catalyst.

The anti-cancer treatment defined herein may be applied as a sole therapy or may involve, in addition to the compound of the invention, conventional surgery or radiotherapy or chemotherapy. Such chemotherapy may include one or more of the following categories of anti-tumour agents:
(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin);
(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) agents which inhibit cancer cell invasion (for example metalloproteinase inhibitors like marimastat and inhibitors of urokinase plasminogen activator receptor function);
(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine, N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033), for example inhibitors of the platelet-derived growth factor family and for example inhibitors of the hepatocyte growth factor family;
(v) antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

Synthesis

The compounds of the present invention can be prepared in a number of ways well known to one skilled in the art of organic synthesis. The compounds of the present invention can be synthesized using the methods described below, together with synthetic methods known in the art of synthetic organic chemistry, or variations thereon as appreciated by those skilled in the art. Such methods include, but are not limited to, those described below. All references cited herein are hereby incorporated in their entirety by reference.

The novel compounds of this invention may be prepared using the reactions and techniques described herein. The reactions are performed in solvents appropriate to the reagents and materials employed and are suitable for the transformations being effected. Also, in the description of the synthetic methods described below, it is to be understood that all proposed reaction conditions, including choice of solvent, reaction atmosphere, reaction temperature, duration of the experiment and workup procedures, are chosen to be the conditions standard for that reaction, which should be readily recognized by one skilled in the art. It is understood by one skilled in the art of organic synthesis that the functionality present on various portions of the molecule must be compatible with the reagents and reactions proposed. Such restrictions to the substituents, which are not compatible with the reaction conditions, will be readily apparent to one skilled in the art and alternate methods must then be used.

The starting materials for the Examples contained herein are either commercially available or are readily prepared by standard methods from known materials. For example the following reactions are illustrations but not limitations of the preparation of some of the starting materials and examples used herein.

A general process for making the compounds of the invention is as follows:

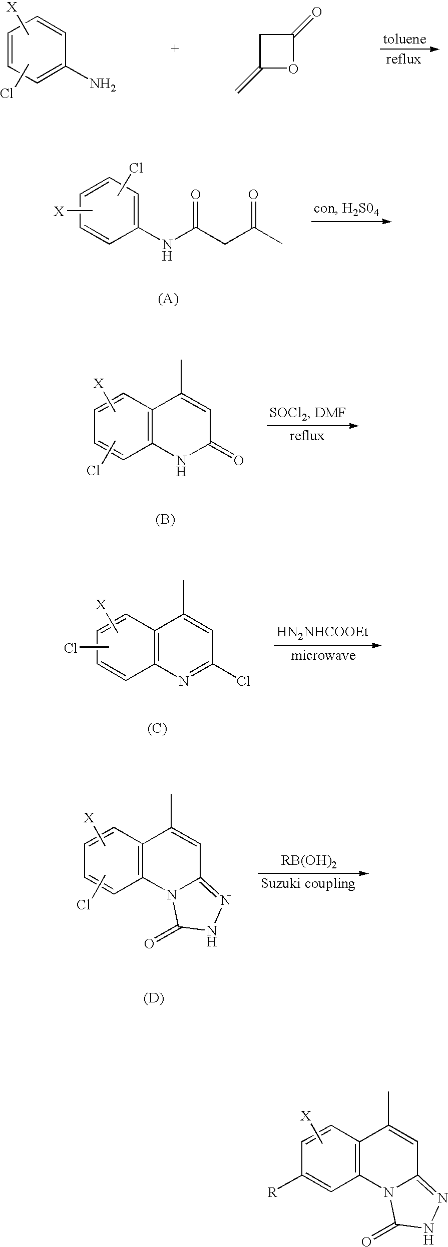

Diketene (32 ml, 32 g, 381 mmol) was added to a suspension of the appropriately substituted chloro aniline (317.25 mmol) in toluene (300 ml). The mixture was refluxed for 6 hr, cooled down and let stand overnight. The precipitated solid was filtered off, washed with ether and dried under vacuum to yield the intermediate (A).

A mixture of the appropriately substituted chloro acetoacetanilide (199.6 mmol) and concentrated sulfuric acid (80 ml) were heated on an oil-bath at 70-80° C. for 0.5 h and for 1.0 h at 100° C. The mixture was cooled down and poured into crushed ice. The precipitated solid was filtered off, and recrystallized from ethanol to obtain intermediate (B).

A mixture of the appropriately substituted 4-methyl-1H-quinolin-2-one (134.2 mmol), DMF (10 ml) and thionyl chloride (300 g) was heated at reflux for 3 hr. The mixture was cooled to room temperature and the resultant solid filtered off, washed with acetone and dried under vacuum to give the intermediate dichloroquinoline (C).

To a suspension of the appropriately substituted dichloro-4-methyl-quinoline (1.5 mmol) and ethyl carbazate (173 mg 1.66 mmol) in 3.7 ml of ethanol was added 6 drops of HCl (4N in dioxane). The reaction mixture was subject to irradiation with microwave at 170° C. for 20 min. After cooling to room temperature the precipitated solid was filtered off, washed with methanol (3×10 ml) and dried under vacuum to give the desired triazolone (D).

To a 5 ml vial, the appropriately substituted 5-methyl-2H-[1,2,4]triazolo[4,3-α]quinolin-1-one (0.5 mmol), boronic acid (0.6 mmol), cesium carbonate (651 mg, 2.0 mmol), and tetrakis(trisphenylphosphine)palladium (40 mg, 7 mol %) were added in 4 ml of dioxane:water (4:1). The reaction was subject to microwave irradiation at 165° C. for 20 min. After cooling to room temperature, the lower layer was separated and discarded, the upper layer was evaporated, the residue dissolved in the minimum amount of DMSO and filtered. The crude product was purified by HPLC.

### EXAMPLES

### Examples 1-30

A general procedure for preparation of 5-substituted-2H-[1,2,4]triazolo[4,3-α]quinolin-1-ones:

To a 5 ml reaction vial, 5-chloro-2H-[1,2,4]triazolo[4,3-α]quinolin-1-one (110 mg, 0.5 mmol), the appropriate boronic acids (of general fomula, RB(OH)₂₎ (0.6 mmol), cesium carbonate (651 mg, 2.0 mmol), and tetrakis(trisphenylphosphine)palladium(0) (40 mg, 7 mol %) were added followed by dioxane:water 4:1(4 ml). The reaction mixture was heated with stirring in a microwave synthesizer for 1200 seconds at 165° C. After cooling to ambient temperature, the lower aqueous layer was removed by pipette and discarded. The upper layer was collected and concentrated. The residual solid was dissolved in the minimum amount of DMSO (2-4 mL) followed by filtration. The crude product was purified by chroamatography to afford the title compounds.

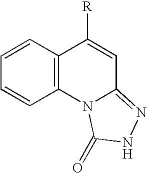

| Ex. | R— | 1H NMR(400 MHz, DMSO-d6) |
|---|---|---|
| 1 | 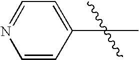 | 7.23-7.73(m, 6H), 8.85/9.15(m, 3H), 12.75(s, 1H) |
| 2 | 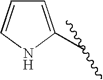 | 6.24/6.55(M, 2H), 7.05(S, 2H), 7.43/7.70(m, 2H), 8.19(d, 1H), 9.08(d, 1H), 11.50(s, 1H), 12.55(s, 1H) |
| 3 | 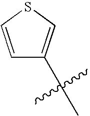 | 7.19-7.98(m, 7H), 9.15(d, 1H), 12.64(s, 1H) |
| 4 | 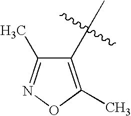 | 1.85(s, 3H), 2.08(s, 3H), 7.46-6.98(m, 4H), 8.8(d, J=8Hz, 1H), 12.42(s, 1H) |
| 5 | 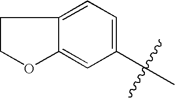 | 4.10(m, 2H), 4.62(t, J=8.2Hz, 2H), 6.91-7.65(m, 7H), 9.05(d, J=7.6Hz, 1H), 12.55(s, 1H) |
| 6 | 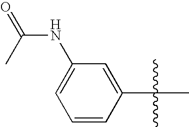 | 2.06(s, 3H), 7.02-7.75(m, 8H), 9.08(d, 1H), 10.14(s, 1H), 12.60(s, 1H) |
| 7 | 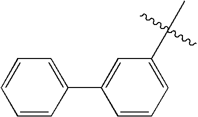 | 7.15-7.85(m, 13H), 9.15(d, 1H), 12.64(s, 1H) |
| 8 | 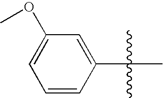 | 3.82(s, 3H), 7.04-7.70(m, 8H), 9.10(d, 1H), 12.60(s, 1H) |
| 9 | 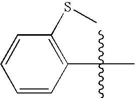 | 2.37(s, 3H), 7.00-7.63(m, 8H), 9.02(d, J=7.6Hz, 1H), 12.65(s, 1H) |
| 10 | 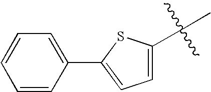 | 7.25-8.05(m, 11H), 9.10(d, 1H), 12.80(s, 1H) |
| 11 | 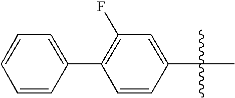 | 7.10-7.85(m, 12H), 9.05(d, 1H), 12.75(s, 1H) |
| 12 | 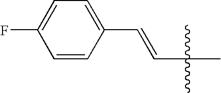 | 7.22-8.27(m, 10H), 9.05(d, 1H), 12.55(s, 1H) |
| 13 | 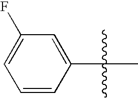 | 7.25-7.95(m, 8H), 9.18(d, 1H), 12.82(s, 1H) |
| 14 | 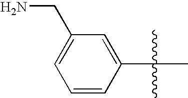 | 4.15(s, 2H), 7.10-7.75(m, 8H), 8.20(s, 2H), 9.10(d, 1H), 12.65(s, 1H) |
| 15 | 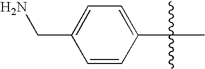 | 4.20(s, 2H), 6.75-7.75(m, 8H), 8.25(s, 2H), 9.10(d, 1H), 12.70(s, 1H) |
| 16 | 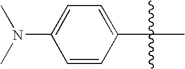 | 2.99(s, 6H), 6.90-7.70(m, 8H), 9.10(d, 1H), 12.60(s, 1H) |
| 17 | 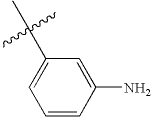 | 6.96-7.68(m, 8H), 9.05(d, J=7.6Hz, 1H), 12.61(s, 1H) |
| 18 | 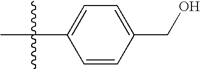 | 4.65(s, 2H), 5.35(t, 1H), 6.90-8.35(m, 8H), 9.05(d, 1H), 12.60(s, 1H) |
| 19 | 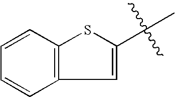 | 7.25-8.00(m, 9H), 9.00(d, 1H), 12.65(s, 1H) |
| 20 | 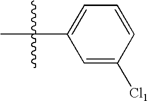 | 6.96-7.43(m, 8H), 8.90(d, 1H), 12.50(s, 1H) |
| 21 | 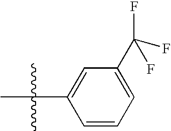 | 7.14-7.81(m, 8H), 9.03(d, J=7.6Hz, 1H), 12.61(s, 1H) |
| 22 | 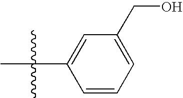 | 4.47(s, 1H), 6.89-7.56(m, 8H), 8.93(d, J=7.6Hz, 1H), 12.46(s, 1H) |
| 23 | 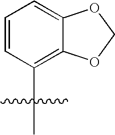 | 6.12(s, 2H), 6.91-7.70(m, 7H), 9.05(d, 1H), 12.59(s, 1H) |
| 24 | 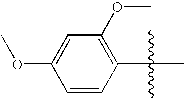 | 3.68(s, 3H), 3.85(s, 3H), 6.70-7.65(m, 7H), 9.00(d, 1H), 12.58(s, 1H) |
| 25 | 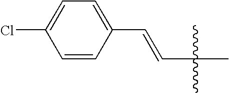 | 7.25-7.65(m, 9H), 8.05(d, J=8.0Hz, 1H), 8.85(d, 1H), 12.35(s, 1H) |
| 26 | 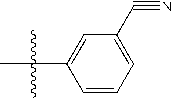 | 7.10-8.05(m, 8H), 9.08(d, 1H), 16.50(s, 1H) |
| 27 | 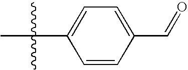 | 7.15-8.10(m, 8H), 9.05(d, 1H), 10.15(s, 1H), 12.70(s, 1H) |
| 28 | 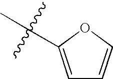 | 6.80-8.20(m, 7H), 9.10(d, 1H), 12.65(s, 1H) |
| 29 | 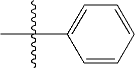 | 7.05-7.75(m, 9H), 9.12(d, 1H), 12.70(s, 1H) |
| 30 | 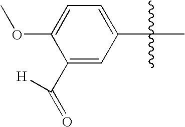 | 4.02(s, 3H), 7.09-7.83(m, 7H), 9.06(d, J=7.6Hz, 1H), 10.43(s, 1H), 12.60(s, 1H) |

### Examples 31-66

A general procedure for preparation of 5-(4′-substituted aminomethylenephenyl)-2H-[1,2,4]tiazolo[4,3-α]quinolin-1-ones:

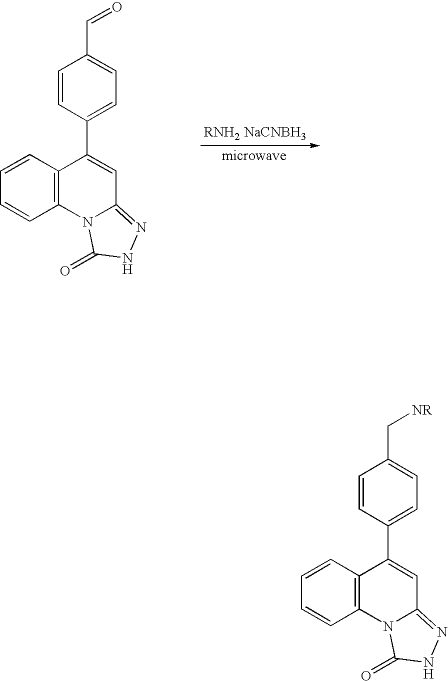

To a suspension of 4-(1-oxo-1,2-dihydro-[1,2,4]triazolo[4,3-α]quinolin-5-yl)-benzaldehyde (0.5 mmol) in DMF (4 ml), the appropriate amine (1 mmol) was added. The mixture was stirred overnight at room temperature. To the reaction mixture was added NaCNBH₃ (63 mg, 1 mmol) and 2 drops of AcOH. The reaction mixture was sealed, stirred and heated under microwave conditions for 5 minutes at 150° C. After cooling to ambient temperature, the reaction mixture was quenched with water (1 mL). The crude product was isolated and purified by chroamatography.

| Ex. | RN— | ¹H NMR(400 MHz, DMSO-D6) |
|---|---|---|
| 31 | 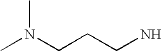 | 2.05(m, 2H), 2.80(s, 6H), 3.05-3.25(m, 4H), 4.32(s, 2H), 7.06-7.75(m, 8H), 9.10(m, 2H), 12.65(s, 1H) |
| 32 | 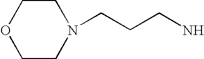 | 2.10(m, 2H), 3.05-3.20(m, 12H), 4.25(s, 2H), 7.05-7.70(m, 8H), 9.05(d, 1H), 9.15(s, br, 1H), 12.70(s, 1H) |
| 33 | 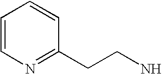 | 3.22-3.26(t, J=15.2Hz, 7.6Hz, 2H), 3.42-3.44(d, J=7.2Hz, 2H), 4.35(s, 2H), 7.06-7.96(m, 11H), 8.61-8.62(d, J=4.4Hz, 1H), 9.06-9.19(s, d, 2H), 12.64(s, 1H) |
| 34 | 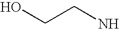 | 3.05(m, 2H), 3.70(m, 2H), 4.30(s, 2H), 5.30(s, br, 1H), 7.10-7.70(m, 8H), 8.90(s, br, 1H), 9.10(d, 1H), 12.60(s, 1H) |
| 35 | 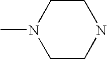 | 2.81(s, 3H), 3.17(m, 8H), 3.83(s, 2H), 7.03-7.78(m, 8H), 9.15(d, 1H), 12.65(s, 1H) |
| 36 | 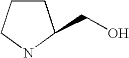 | 1.75-2.30(m, 6H), 3.78(m, 3H), 4.45(m, 1H), 4.70(m, 1H), 5.60(s, 1H), 7.08-7.78(m, 8H), 9.10(d, 1H), 12.65(s, 1H) |
| 37 | 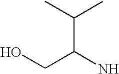 | 0.97(dd, J=6.8Hz, 6H), 2.20(m, 1H), 3.05(m, 1H), 4.20-4.50(m, 4H), 5.45(s, 1H), 7.10-7.80(m, 8H), 8.90(s, br, 1H), 9.15(d, 1H), 12.65(s, 1H) |
| 38 | 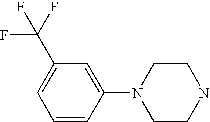 | 3.10(m, 8H), 4.05(s, 2H), 7.10-7.70(m, 12H), 9.10(d, 1H), 12.65(s, 1H) |
| 39 | 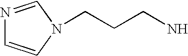 | 1.91(m, 2H), 3.02(m, 2H), 4.26(s, 2H), 4.32(m, 2H), 7.03-7.77(m, 10H), 9.04-9.23(m, 3H), 12.65(s, 1H) |
| 40 | 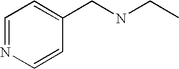 | 1.30(t, 3H), 2.50(s, 2H), 3.02(s, 2H), 4.40(s, 2H), 7.67-8.00(m, 10H), 8.75(m, 2H), 9.10(d, 1H), 12.70(s, 1H) |
| 41 | 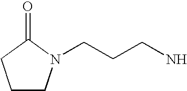 | 1.80-2.20(m, 4H), 2.25(m, 2H), 2.95(m, 2H), 3.30(m, 2H), 3.35(m, 2H), 4.25(s, 2H), 7.05-7.75(m, 8H), 8.85(s, br, 1H), 9.10(d, 1H), 12.65(s, 1H) |
| 42 | 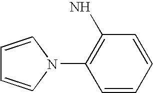 | 4.40(s, 2H), 6.30-7.70(m, 16H), 9.10(d, 1H), 12.65(s, 1H) |
| 43 | 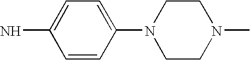 | 2.51(s, 3H), 2.85(m, 4H), 3.14(m, 4H), 4.39(s, 2H), 6.77-7.02(m, 5H), 7.38-7.68(m, 7H), 9.06(d, J=7.6Hz, 1H), 9.67(s, br, 1H), 12.60(s, 1H) |
| 44 | 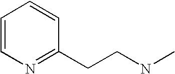 | 2.86(s, 3H), 3.31(m, 4H), 4.52(s, 2H), 7.09-7.86(m, 11H), 8.56(d, J=4.8Hz, 1H), 9.08(d, J=8Hz, 1H), 12.64(s, 1H) |
| 45 | 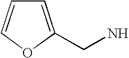 | 4.27(s, 2H), 4.31(s, 2H), 6.65-6.75(m, 2H), 7.05-7.85(m, 9H), 9.05(d, 1H), 9.50(s, br, 1H), 12.65(s, 1H) |
| 46 | 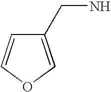 | 4.13(s, 2H), 4.26(s, 2H), 6.67(s, 1H), 7.05(s, 1H), 7.43-7.85(m, 9H), 9.05(d, 1H), 9.30(s, br, 1H), 12.65(s, 1H) |
| 47 | 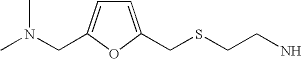 | 2.73(s, 6H), 2.80(m, 2H), 3.20(m, 2H), 3.88(s, 2H), 4.29(s, 2H), 4.35(s, 2H), 6.40(s, 1H), 6.65(s, 1H), 7.05(s, 1H), 7.61-7.67(m, 7H), 9.10-9.25(m, 2H), 12.65(s, 1H) |
| 48 | 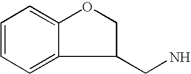 | 3.20(t, J=8.6Hz, 1H), 4.16(s, 2H), 4.26(s, 2H), 4.57(t, J=8.6Hz, 2H), 6.83-7.67(m, 12H), 9.07(d, J=8.4Hz, 1H), 9.30(s, br, 1H), 12.64(s, 1H) |
| 49 | 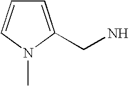 | 3.64(s, 3H), 4.25(s, 2H), 4.31(s, 2H), 6.04(s, 1H), 6.30(s, 1H), 6.84(s, 1H), 7.06(s, 1H), 7.43-7.69(m, 7H), 9.10(d, 1H), 9.30(s, br, 1H), 12.64(s, 1H) |
| 50 | 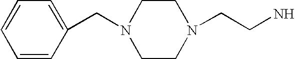 | 2.70-3.30(m, 16H), 4.29(s, 2H), 4.33(s, 2H), 7.06-7.70(m, 13H), 8.90(s, br, 1H), 9.07(d, J=8.8Hz, 1H), 12.66(s, 1H) |
| 51 | 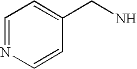 | 4.18-4.40(m, 4H), 7.05-7.75(m, 10H), 8.30(s, 1H), 9.10(d, 1H), 9.55(s, br, 1H), 12.65(s, 1H) |
| 52 | 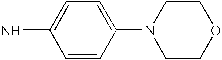 | 3.28(m, 4H), 3.83(m, 4H), 4.45(s, 2H), 6.87-7.68(m, 12H), 9.05(d, J=8Hz, 1H), 12.61(s, 1H) |
| 53 | 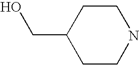 | 1.30-1.90(m, 5H), 3.00-3.29(m, 6H), 4.38(s, 2H), 7.07-7.68(m, 8H), 9.10(d, 1H), 12.66(s, 1H) |
| 54 | 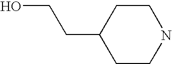 | 1.36-1.91(m, 7H), 2.99-3.35(m, 6H), 4.39(s, 2H), 7.08-7.71(m, 8H), 9.07(d, J=8.4Hz, 1H), 12.66(s, 1H) |
| 55 | 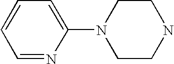 | 2.60-3.35(m, 8H), 4.48(s, 2H), 7.06-8.17(m, 12H), 9.06(d, 1H), 12.65(s, 1H) |
| 56 | 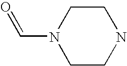 | 2.90-3.35(m, 8H), 4.44(s, 2H), 7.07-7.68(m, 8H), 8.09(s, 1H), 9.07(d, J=8Hz, 1H), 12.64(s, 1H) |
| 57 | 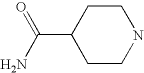 | 1.76(m, 2H), 1.96(m, 2H), 2.38(m, 1H), 2.90-3.35(m, 4H), 4.40(s, 2H), 6.96 & 7.08(s, s, 2H), 7.42-7.69(m, 8H), 9.07(d, J=8.4Hz, 1H), 12.64(s, 1H) |
| 58 | 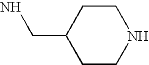 | 1.39(m, 2H), 1.85-1.95(m, 3H), 2.73-3.33(m, 6H), 4.27-4.39(d, J=48.8Hz, 2H), 7.06-7.87(m, 9H), 8.99(s, br, 1H), 9.08(d, J=8.4Hz, 1H), 12.65(s, 1H) |
| 59 | 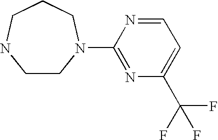 | 1.90(m, 2H), 2.65(m, 2H), 2.78(m, 2H), 3.71(s, 2H), 3.78-3.90(m, 4H), 6.99-7.68(m, 9H), 8.68(s, 1H), 9.06(d, J=7.6Hz, 1H), 12.65(s, 1H) |
| 60 | 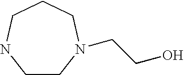 | 2.18(m, 2H), 3.30-3.75(m, 12H), 4.40(s, 2H), 7.06-7.95(m, 8H), 9.07(d, J=8.4Hz, 1H), 12.64(s, 1H) |
| 61 | 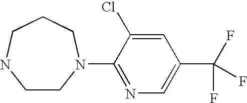 | 2.28(m, 2H), 3.27-4.19(m, 8H), 4.52(s, 2H), 7.08-7.72(m, 8H), 8.17(s, 1H), 8.50(s, 1H), 9.07(d, J=8.0Hz, 1H), 12.65(s, 1H) |
| 62 | 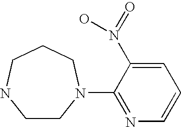 | 1.15-4.47(m, 10H), 4.60(s, 2H), 6.94-8.46(m, 11H), 9.08(d, J=7.6Hz, 1H), 12.64(s, 1H) |
| 63 | 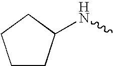 | 1.35-2.05(m, 8H), 3.57(m, 1H), 4.61(s, 2H), 7.10-7.71(m, 8H), 8.94(s, br, 1H), 8.09(d, J=8.4Hz, 1H), 12.63(s, 1H). |
| 64 | 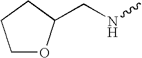 | 1.61-2.08(m, 4H), 2.97(m, 1H), 3.13(m, 1H), 3.72-3.87(m, 2H), 4.15(m, 1H), 4.28(s, 2H), 7.08-7.63(m, 8H), 9.07(m, 2H), 12.62(s, 1H). |
| 65 | 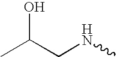 | 1.13(d, J=6.4Hz, 3H), 2.73(m, 2H), 3.98(m, 1H), 4.27(s, 2H), 7.06-7.70(m, 8H), 8.92(s, br, 1H), 9.08(d, J=8.4Hz, 1H), 12.63(s, 1H). |
| 66 | 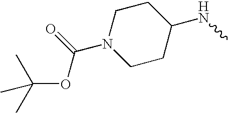 | 1.46(m, 11H), 2.12(m, 2H), 2.81(m, 2H), 3.35(m, 1H), 4.05(m, 2H), 4.30(s, 2H), 7.02-7.96(m, 8H), 8.96(s, br, 1H), 9.08(d, J=8.4Hz, 1H), 12.63(s, 1H). |

### Examples 66-73

The following examples were prepared by the procedure described for examples 31-62 using 3-methoxy-2-(1-oxo-1,2-dihydro[1,2,4]triazolo[4,3-a]quinolin-5-yl)benzaldehyde (prepared as described for example 27) and the appropriate amine.

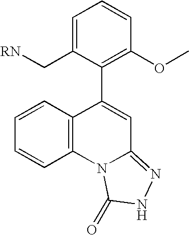

| Ex. | RN— | 1H NMR(400 MHz, DMSO-d6) |
|---|---|---|
| 66 | 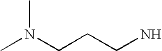 | 2.00(m, 2H), 2.78(s, s, 6H), 3.00(m, 2H), 3.10(m, 2H), 3.73(s, 3H), 4.18(s, 2H), 7.10-7.70(m, 7H), 8.90(s, br, 1H), 9.10(d, 1H), 12.65(s, 1H) |
| 67 | 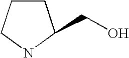 | 1.70-2.20(m, 4H), 3.20(m, 2H), 3.58(m, 3H), 3.73(s, 3H), 4.28(m, 1H), 4.55(m, 1H), 7.05-7.68(m, 7H), 9.02(d, J=8.4Hz, 1H), 12.63(s, 1H) |
| 68 | 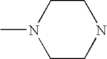 | 2.78(s, 3H), 3.25-3.70(m, 8H), 3.73(s, 3H), 3.91(s, 2H), 7.02-7.63(m, 7H), 9.01(d, J=8.4Hz, 1H), 12.62(s, 1H) |
| 69 | 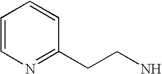 | 3.17(m, 2H), 3.35(m, 2H), 3.72(s, 3H), 4.24(s, 2H), 7.00-7.88(m, 10H), 8.55(d, J=4.8Hz, 1H), 9.02(d, J=8.4Hz, 1H), 12.65(s, 1H) |
| 70 | 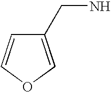 | 3.73(s, 3H), 4.06(s, 2H), 4.17(s, 2H), 6.63-7.82(m, 10H), 9.01(d, J=8.4Hz, 1H), 9.09(s, br, 1H), 12.60(s, 1H) |
| 71 | 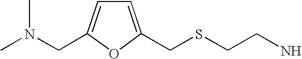 | 2.75(s, 6H), 3.20(m, 4H), 3.72(s, 3H), 3.86(s, 2H), 4.19(s, 2H), 4.33(s, 2H), 6.40-7.64(m, 9H), 9.02(s, d, J=7.6Hz, 2H), 12.63(s, 1H) |
| 72 | 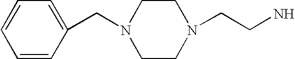 | 2.65(m, 2H), 3.10(m, 2H), 3.75(s, 3H), 4.19(s, 2H), 4.30(s, 2H), 7.01-7.65(m, 12H), 9.02(s, br, 1H), 9.03(d, J=8.4Hz, 1H), 12.63(s, 1H) |
| 73 | 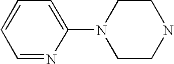 | 3.12(m, 4H), 3.48(m, 4H), 3.70(s, 3H), 4.40(s, 2H), 6.75-8.17(m, 11H), 9.03(d, J=8.0Hz, 1H), 12.61(s, 1H) |

### Examples 74-82

The following examples were prepared by the procedure described for examples 31-62 using the appropriate aldehyde (example 30) and amine.

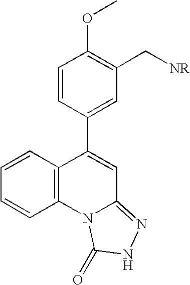

| Ex. | RN— | 1H NMR(400 MHz, DMSO-d6) |
|---|---|---|
| 74 | 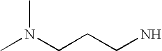 | 2.10(m, 2H), 2.80(s, 6H), 3.05(m, 4H), 3.95(s, 3H), 4.23(s, 2H), 7.03-7.75(m, 7H), 8.85(s, br, 1H), 9.10(d, 1H), 12.62(s, 1H) |
| 75 | 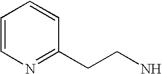 | 3.18(m, 2H), 3.43(m, 2H), 3.96(s, 3H), 4.30(s, 2H), 7.03-7.86(m, 10H), 8.57(s, 1H), 9.00(s, br, 1H), 9.07(d, J=8.0Hz, 1H), 12.62(s, 1H) |
| 76 | 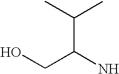 | 1.00(d, 6H), 2.12(m, 1H), 3.00-3.25(m, 3H), 3.94(s, 3H), 4.30(s, 2H), 7.02-7.70(m, 7H), 8.50(s, br, 1H), 9.07(d, J=8.4Hz, 1H), 12.62(s, 1H) |
| 77 | 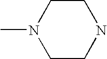 | 2.79(s, 3H), 3.00-3.30(m, 8H), 3.85(s, 2H), 3.89(s, 3H), 7.05-7.75(m, 7H), 9.10(d, 1H), 12.65n(s, 1H) |
| 78 | 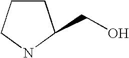 | 1.70-2.20(m, 4H), 3.63-3.74(m, 5H), 3.95(s, 3H), 4.30(m, 1H), 4.61(m, 1H), 7.09-7.70(m, 7H), 9.07(d, J=8.4Hz, 1H), 12.62(s, 1H) |
| 79 | 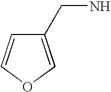 | 3.95(s, 3H), 4.11(d, J=5.2Hz, 2H), 4.15(d, J=.6Hz, 2H), 6.67-7.95(m, 10H), 9.04(s, br, 1H), 9.07(d, J=8.0Hz, 1H), 12.60(s, 1H) |
| 80 | 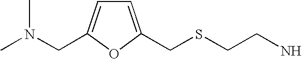 | 2.78(s, 6H), 2.80(m, 2H), 3.20(m, 2H), 3.87(s, 2H), 3.99(s, 3H), 4.24(s, 2H), 4.34(s, 2H), 6.41-7.70(m, 9H), 8.97(s, br, 1H), 9.08(d, J=8.0Hz, 1H), 12.61(s, 1H) |
| 81 | 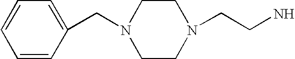 | 2.25-2.65(m, 14H), 3.81(s, 2H), 3.91(s, 3H), 6.96-7.95(m, 12H), 9.05(d, J=8.0Hz, 1H), 12.65(s, 1H) |
| 82 | 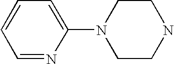 | 3.45(m, 8H), 3.59(s, 2H), 3.85(s, 3H), 6.59-8.08(m, 11H), 9.04(d, J=8.4Hz, 1H), 12.65(s, 1H) |

### Example 83

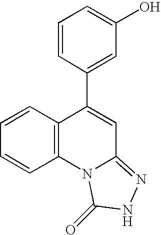

To the methoxy compound prepared in Example 8, (29.1 mg, 0.1 mmol) was added BBr₃ (1M in CH₂Cl₂, 3 ml). The mixture was stirred at room temperature for 3 hours and quenched with crushed ice. The precipitate was collected by filtration to afford the crude product. The crude product was dissolved in the minimum amount of DMSO (2-4 mL) and purified by chromatography to give the desired compound. ¹H NMR (400 MHz, DMSO-d6): 6.85-7.67 (m, 8H), 9.05 (d, J=7.6 Hz, 1H), 9.73 (s, 1H, OH), 12.57 (s, 1H)

### Examples 84-90

The following examples were prepared by demethylation of the corresponding methoxy analogue using the procedure described in Example 80.

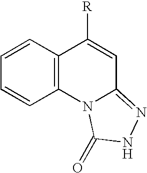

| Ex. | R— | ¹H NMR(400 MHz, DMSO-d6) |
|---|---|---|
| 84 | 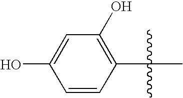 | 6.30-7.75(m, 7H), 9.00(d, 1H), 9.50-9.75(s, s, 2H), 12.50(s, 1H) |
| 85 | 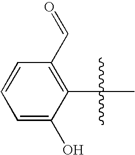 | 7.18-8.22(m, 7H), 9.10(d, 1H), 9.88(s, 1H), 10.75(s, 1H), 12.65(s, 1H) |
| 86 | 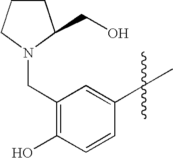 | 1.82-2.15(m, 4H), 3.30-3.90(m, 5H), 4.30(m, 1H), 4.58(m, 1H), 5.50(s, br, 1H), 7.05-7.69(m, 7H), 9.07(d, J=8.0Hz, 1H), 10.47(s, 1H), 12.59(s, 1H) |
| 87 | 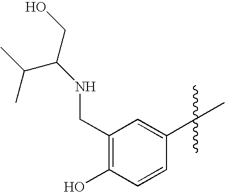 | 0.95(d, J=6.8Hz, 3H), 1.01(d, J=6.8Hz, 3H), 2.16(q, J=6.8Hz, 1H), 3.77-4.12(m, 3H), 4.28(s, 2H), 5.38(s, 1H), 6.99-7.70(m, 7H), 9.07(d, J=8.4Hz, 1H), 10.77(s, 1H), 12.59(s, 1H) |
| 88 | 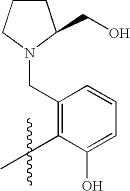 | 1.79-2.13(m, 4H), 3.17-3.34(m, 5H), 4.25(m, 1H), 4.47(m, 1H), 5.49(s, br, 1H), 7.04-7.66(m, 7H), 9.03(d, J=8.4Hz, 1H), 10.11(s, 1H), 12.61(s, 1H) |
| 89 | 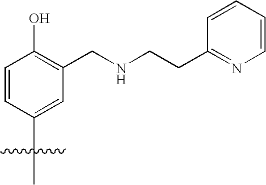 | 3.18(m, 4H), 4.26(s, 2H), 7.00-7.85(m, 10H), 8.53(d, J=4.4Hz, 1H), 8.95(s, br, 1H), 9.07(d, J=8.4Hz, 1H), 10.47(s, 1H), 12.59(s, 1H) |
| 90 | 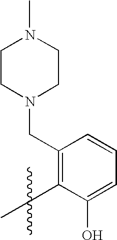 | 2.75(s, 3H), 3.10-3.75(m, 8H), 3.85(s, 2H), 7.00-7.65(m, 7H), 9.00(d, 1H), 9.85(s, 1H), 12.60(s, 1H) |

### Examples 90-101

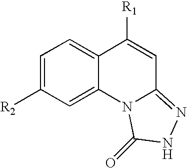 Synthesis of Intermediates:

### 7-Bromo-2-oxo-1,2-dihydroquinoline-4-carboxylic acid

A mixture of 6-bromoisatin (226 mg, 1 mmol), malonic acid, (114 mg, 1.1 mmols) and sodium acetate (103 mg, 1.25 mmols) and acetic acid (2.5 ml) the was stirred under a nitrogen atmosphere for 5 h. Additional sodium acetate (100 mg) was added and the resultant mixture was heated overnight. The reaction mixture was cooled to room temperature, excess acetic acid was removed under reduced pressure and the resultant pinkish brown solid was washed with copious amounts of water and dried under vacuum to obtain the desired product (234 mg, 88%).

¹H NMR(300 MHZ, DMSO-d₆): 6.88 (s, 1H), 7.44 (d, 1H), 7.58 (s, 1H), 8.18 (d, 1H), 12.11 (br s, 1H), m/z 268

### 7-Bromo-2-oxo-1,2-dihydroquinoline-4-carboxylic acid ethyl ester

7-Bromo-2-oxo-1,2-dihydroquinoline-4-carboxylic acid: (1 g, 3.74 mmols), absolute ethanol (4 ml) and conc. sulphuric acid (4 ml) were heated to reflux for 45 mins. The reaction mixture was cooled to room temperature and the ethanol removed under reduced pressure. The resultant dark brown precipitate was washed with and dried under vacuum to yield the desired product (0.95 g, 86%).

¹H NMR(300 MHZ, DMSO-d₆): 1.44 (t, 3H), 4.47 (q, 2H), 7.28 (s, 1H), 7.40 (dd, 1H), 7.58 (d, 1H), 8.28 (d, 1H), 11.80 (br s, 1H), m/z 296

### 7-bromo-2-chloro-quinoline-4-carboxylic acid ethyl ester

7-Bromo-2-oxo-1,2-dihydroquinoline-4-carboxylic acid ethyl ester (0.5 g, 1.69 mmols) phosphorous oxychloride (10 ml) and phosphorous pentoxide (50 mg) were heated at reflux for 1.5 hrs under an inert atmosphere. The reaction mixture was cooled to room temperature, phosphorous oxychloride evaporated under vacuum and dichloromethane (200 ml) added. The resultant organic solution was washed with sat'd NaHCO₃ (50 ml), followed by brine (50 ml). The organic layer was separated, dried over Na₂SO₄ (anhyd.), and solvent removed. The product was purified by silica gel chromatography using an ethyl acetate and hexanes gradient to obtain the title compound (408 mg, 76%).

¹H NMR(300 MHZ, CDCl₃): 1.48 (t, 3H), 4.51(q, 2H), 7.74 (dd, 1H), 7.92 (s, 1H), 8.25 (d, 1H), 8.66 (d, 1H), m/z 314

### 8-bromo-1-oxo-1,2,-dihydro-[1,2,4]-triazolo[4,3-a]quinoline-5-carboxylic acid ethyl ester

7-Bromo-2-chloro-quinoline-4-carboxylic acid ethyl ester (305 mg, 1 mmol) ethyl carbazate (1.2 mmols), 4 M HCl in dioxane (0.2 ml) and abs. ethanol (5 ml) were placed in a pyrex vial and the resultant mixture was heated at 160° C. for 20 minutes in a microwave synthesizer. The mixture was cooled and the precipitated product filtered off, washed with a small amount of methanol followed by hexanes and dried under vacuum to obtain the title compound (193 mg, 57.4%).

¹H NMR(300 MHZ, DMSO-d₆): 1.36 (t, 3H), 4.38 (q, 2H), 7.68 (d, 1H), 7.77 (s, 1H), 8.30 (d, 1H), 9.17 (s, 1H), 13.03 (s, 1H), m/z 308

### 8-bromo-1-oxo-1,2-dihydro-[1,2,4]-triazolo[4,3-a]quinoline-5-caroxylic acid

8-Bromo-1-oxo-1,2-dihydro-[1,2,4]-triazolo[4,3-a]quinoline-5-caroxylic acid ethyl ester (100 mg, 0.3 mmols) and lithium hydroxide.monohydrate (0.9 mmols, 38 mg) in a mixture of THF, methanol, and water (1:1:1,. 2.3 ml) was stirred at room temperature for 2 hours. The solvent was removed to yield a pink solid. Water (5 ml) was added, and the pH of the resultant solution adjusted to 1-2. The resulting precipitate was washed with water then hexanes and dried to yield the desired product (58.7 mg, 63.5%).

¹H NMR(300 MHZ, DMSO-d₆): 7.68 (dd, 1H), 7.73 (s, 1H), 8.46(d, 1H), 12.99 (s, 1H), m/z 278

### 5-amino-8-bromo[1,2[4]triazolo[4,3-a]quinolin-1(2H)-one (example 90)

8-Bromo-1-oxo-1,2-dihydro-[1,2,4-triazolo[4,3-a]quinoline-5-carboxylic acid (0.5 g, 1.62 mmols) was dissolved in t-butanol (8 ml), and diisopropylethylamine (0.31 ml, 1.78 mmols) followed by diphenylphosphorylazide (0.39 ml, 1.78 mmols) added. The reaction mixture was heated at reflux for 5 hrs under anhydrous conditions. The solvent was removed to obtain a slurry of the Boc protected analogue of the title compound. 5% trifluoroacetic acid in dichloromethane was added and the reaction mixture stirred at room temperature for 1 hour. Additional TFA (1 mL) was added and the resultant precipitate filtered off, washed with hexanes and dried under vacuum to obtain the desired product (0.326 g, 72.4%).

### Example 90

### 5-methyl-8-pyridin4-yl[1,2,4]triazolo[4,3-a]quinolin-1(2H)-one

8-bromo-5-methyl-[1,2,4]-triazolo[4,3-a-quinolin-1-one (139 mg, 0.5 mmols), 4-pyridyl-boronic acid (74 mg, 0.6 mmols), Cs₂CO₃ (0.65 g, 2 mmols) and Pd(PPh₃)₄ (35 mg, 7 mol %) were placed in a pyrex microwave tube and dioxane (4 ml) and water (1 ml)added. The resultant heterogeneous mixture was heated at 165° C. for 10 minutes in a microwave Synthesizer. At the end of this time, the top organic layer was separated, the crude product isolated and purified by RP-HPLC to yield the desired product following lyophilization.

Example 91 was prepared by the procedure described above starting with 8-bromo-5-methyl-[1,2,4]-triazolo[4,3-a-quinolin-1-one and coupling with the boronic acid.

Example 92 was synthesized in 6 steps from 6-bromoisatin as outlined above.

Example 93 was prepared via Suzuki coupling of the appropriate boronic acid starting and 5-amino-8-bromo{1,2,4}triazolo[4,3-a]quinolin-1-(2H)-one (example 90).

Example 94 was synthesized from (2-chloro-4-methyl-quinolin-7-yl)-dimethyl-amine (which was generated according to a published procedure) and placed in a pyrex microwave tube with ethyl carbazate (1.2 mmols), 4 M HCl in dioxane (0.2 ml) and abs. ethanol (5 ml). The resultant mixture was heated at 160° C. for 20 minutes in a microwave synthesizer. The precipitated product was filtered, washed with a small amount of methanol followed by heaxnes and dried under vacuum to obtain the title product (193 mg, 57.4%).

### Examples 93-99

Were prepared as described above for either example 89 (5-methyl analogs) or example 91 (5-amino analogs).

| Ex. | R₁ | R₂ | ¹H NMR(DMSO-d₆) | m/z |
|---|---|---|---|---|
| 90 | Me | 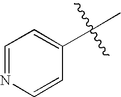 | 2.55(s, 3H), 7.14(d, 1H), 8.00(m, 4H), 8.90(d, 2H), 9.43(s, 1H), 12.56(s, 1H) | 276 |
| 91 | Me | 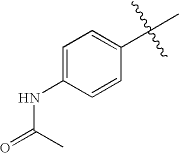 | 2.55(s, 3H), 7.14(d, 1H), 8.00(m, 4H), 8.90(d, 2H), 9.43(s, 1H), 12.56(s, 1H) | 332 |
| 92 | NH₂ | —Br | 5.90(s, 1H), 6.30(br s, 2H), 7.58(dd, 1H), 7.87(d, 1H), 9.09(d, 1H), 11.82(s, 1H) | 279 |
| 93 | NH₂ | 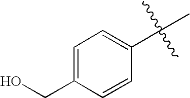 | 4.57(s, 2H), 5.95(s, 1H), 6.37(br s, 2H), 7.47(d, 2H), 7.72(d, 2H), 7.76(dd, 1H), 8.07(d, 1H), 9.33(s, 1H), 11.83(s, 1H) | 306 |
| 94 | Me | 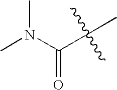 | 2.36(s, 3H), 3.03(s, 6H), 6.63(s, 1H), 6.84(dd, 1H), 7.57(d, 1H), 8.37(d, 1H), 12.20(s, 1H) | 242 |
| 95 | Me | 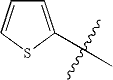 | 2.53(s, 3H), 6.95(s, 1H), 7.20(m, 1H), 7.73(m, 2H), 7.80(m, 2H), 9.27(s, 1H) | 282 |
| 96 | NH₂ | 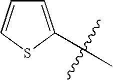 | 5.95(s, 1H), 6.30(s, 2H), 7.18(m, 1H), 7.60(m, 2H), 7.77(d, 1H), 8.02(d, 1H), 9.26(s, 1H), 11.80(s, 1H) | 283 |
| 97 | NH₂ | 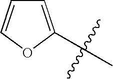 | 5.96(s, 1H), 6.64(d, 1H), 7.20(m, 1H), 7.14(d, 1H), 7.80(d, 1H), 7.86(s, 1H), 8.06(d, 1H), 9.35(s, 1H), 11.83(br s, 1H) | 267 |
| 98 | Me | 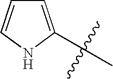 | 2.52(s, 3H), 6.18(m, 1H), 6.67(s, 1H), 6.90(m, 2H), 7.73(m, 2H), 9.17(s, 1H), 11.52(s, 1H) | 265 |
| 99 | Me | 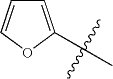 | 2.50(s, 3H), 6.67(m, 1H), 6.98(m, 1H), 7.10(m, 1H), 7.82(m, 3H), 9.30(s, 1H) | 266 |
| 100 | NH₂ | 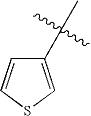 | 5.90(s, 1H), 6.32(s, 2H), 7.57(d, 1H), 7.69(m, 1H), 7.78(d, 1H), 7.98(m, 2H), 9.30(s, 1H), 11.85(s, 1H) | 283 |
| 101 | Me | 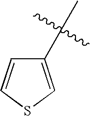 | 2.51(s, 3H), 6.55(s, 1H), 7.01(s, 1H), 7.56(d, 1H), 7.70(m, 1H), 7.80(m, 2H), 7.95(m, 1H), 9.30(s, 1H) | 282 |

### Examples 102-126

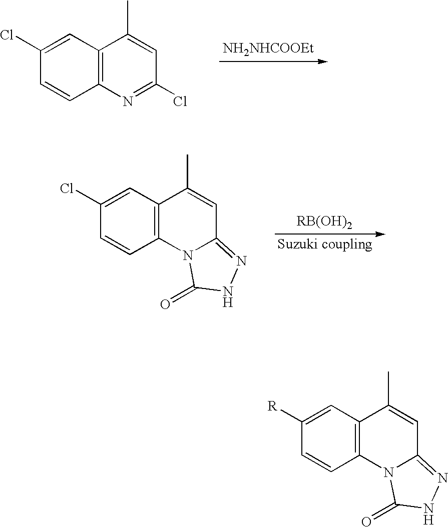

### 7-chloro-5-methyl-2H-[1,2,4]triazolo[4,3-α]quinolin-1-one

To a suspension of 2,6-dichloro4-methylquinoline (212 mg, 1.0 mmol) and ethyl carbazate (125 mg 1.2 mmol) in 4 ml of ethanol was added 4 drops of HCl (4N in dioxane). The reaction mixture was subject to microwave irradiation at 150° C. for 20 min. After cooling to room temperature the precipitated yellow solid was filtered off, washed with methanol (3×10 ml) and dried under vacuum to give the title compound as a yellow solid (76.4 mg, 32.7%).

### 7-Substituted-5-methyl-2H-[1,2,4]triazolo[4,3-α]quinolin-1-ones

To a 5 ml vial, 7-chloro-5-methyl-2H-[1,2,4]triazolo[4,3-α]quinolin-1-one (117 mg, 0.5 mmol), boronic acid (0.6 mmol), cesium carbonate (651 mg, 2.0 mmol), and tetrakis(trisphenylphosphine)palladium (40 mg, 7 mol %) were added in dioxane:water (4:1, 4 ml). The reaction was subject to microwave irradiation at 165° C. for 20 min. After cooling to room temperature, the lower layer was removed and discarded, solvent was removed from the upper layer and the resulting residue dissolved in minimum amount of DMSO. The solution was and purified by HPLC.

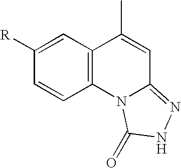

| Ex. | R— | 1H NMR(400 MHz, DMSO-d6) |
|---|---|---|
| 102 | 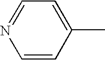 | 2.58(s, 3H), 7.10(s, 1H), 8.20(m, 4H), 8.85(m, 2H), 9.10(d, 1H), 12.53(s, 1H) |
| 103 | 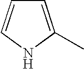 | 2.44(s, 3H), 6.15-8.97(m, 7H), 11.55(s, 1H), 12.50(s, 1H) |
| 104 | 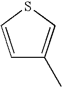 | 2.35(s, 3H), 6.87(s, 1H), 7.51-7.86(m, 5H), 8.78(d, J=8.4Hz, 1H), 12.23(s, 1H) |
| 105 | 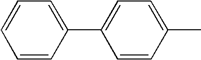 | 2.46(s, 3H), 6.95-8.00(m, 12H), 8.92(d, 1H), 12.30(s, 1H) |
| 106 | 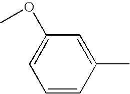 | 2.55(s, 3H), 3.85(s, 3H), 6.95-8.00(m, 7H), 9.01(d, 1H), 12.42(s, 1H) |
| 107 | 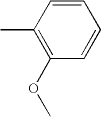 | 2.60(s, 3H), 3.89(s, 3H), 7.15-7.96(m, 7H), 9.07(d, J=8.4Hz, 1H), 12.51(s, 1H) |
| 108 | 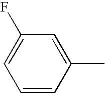 | 2.51(s, 3H), 7.10-8.08(m, 7H), 9.08(d, 1H), 12.42(s, 1H) |
| 109 | 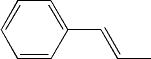 | 2.51(s, 3H), 7.05-7.98(m, 10H), 8.94(d, J=8.8Hz, 1H), 12.42(s, 1H) |
| 110 | 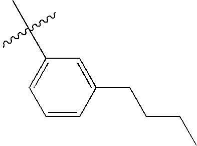 | 0.92(t, 3H), 1.35(m, 2H), 1.58(m, 2H), 2.55(s, 3H), 2.65(t, 2H), 6.99-8.00(m, 7H), 9.03(d, J=8.8Hz, 1H), 12.45(s, 1H) |
| 111 | 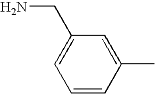 | 2.56(s, 3H), 4.16(d, J=5.6Hz, 2H), 7.12-8.04(m, 7H), 8.22(s, br, 2H), 9.07(d, J=8.4Hz, 1H), 12.48(s, 1H) |
| 112 | 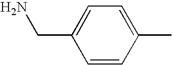 | 2.51(s, 3H), 4.15(d, 2H), 7.10-8.02(m, 7H), 8.19(s, br, 2H), 9.07(d, 1H), 12.50(s, 1H) |
| 113 | 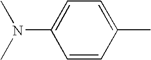 | 2.45(s, 3H), 2.96(s, 6H), 6.84(d, J=8Hz, 2H), 7.04(s, 1H), 7.64(d, J=8.4Hz, 2H), 7.88(m, 2H), 8.96(d, J=8.4Hz, 1H), 12.38(s, 1H) |
| 114 | 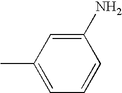 | 2.35(s, 3H), 6.70-7.78(m, 7H), 8.86(d, J=8.8Hz, 1H), 12.35(s, 1H) |
| 115 | 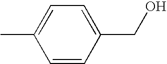 | 2.52(s, 3H), 4.55(d, 2H), 5.30(t, 1H), 7.10-7.98(m, 7H), 9.02(d, 1H), 12.40(s, 1H) |
| 116 | 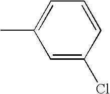 | 2.65(s, 3H), 7.26-8.21(m, 7H), 9.21(d, J=8.4Hz, 1H), 12.67(s, 1H) |
| 117 | 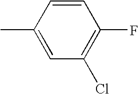 | 2.55(s, 3H), 7.10-8.11(m, 6H), 9.04(d, J=8.4Hz, 1H), 12.48(s, 1H) |
| 118 | 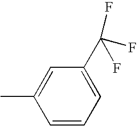 | 2.48(s, 3H), 7.08(d, J=1.2Hz, 1H), 7.76(m, 2H), 8.07-8.12(m, 4H), 9.05(d, J=8.8Hz, 1H), 12.45(s, 1H) |
| 119 | 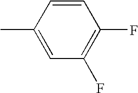 | 2.58(s, 3H), 7.15(s, 1H), 7.330-7.71(m, 3H), 8.09(m, 2H), 9.09(d, J=8.8Hz, 1H), 12.53(s, 1H) |
| 120 | 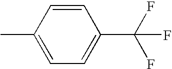 | 2.69(s, 3H), 7.23(d, J=1.2Hz, 1H), 7.99-8.22(m, 6H), 9.20(d, J=8.8Hz, 1H), 12.60(s, 1H) |
| 121 | 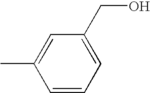 | 2.51(s, 3H), 4.60(s, 2H), 5.30(s, br, 1H), 7.07-7.99(m, 7H), 9.02(d, J=8.4Hz, 1H), 12.40(s, 1H) |
| 122 | 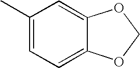 | 2.62(s, 3H), 6.17(s, 2H), 7.01-8.02(m, 6H), 9.06(d, J=8.4Hz, 1H), 12.49(s, 1H) |
| 123 | 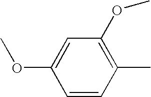 | 2.50(s, 3H), 3.80/3.82(s/s, 6H), 6.60-7.81(m, 6H), 8.92(d, 1H), 12.39(s, 1H) |
| 124 | 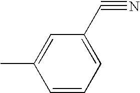 | 2.80(s, 3H), 7.32(s, 1H), 7.90-8.60(m, 6H), 9.30(d, 1H), 12.70(s, 1H) |
| 125 | 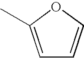 | 2.51(s, 3H), 6.65-8.05(m, 6H), 9.00(d, 1H), 12.45(s, 1H) |
| 126 | 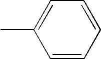 | 2.25(s, 3H), 6.75-7.75(m, 8H), 8.77(d, J=8.4Hz, 1H), 12.18(s, 1H) |

### Examples 127-141

The following examples were prepared by the following procedure using 4-(5-methyl-1-oxo-1,2-dihydro-[1,2,4]triazolo[4,3-α]quinolin-7-yl)-benzaldehyde and the appropriate amine.

### 4-(5-methyl-1-oxo-1,2-dihydro-[1,2,4)triazolo[4,3-α]quinolin-7-yl)-benzaldehyde

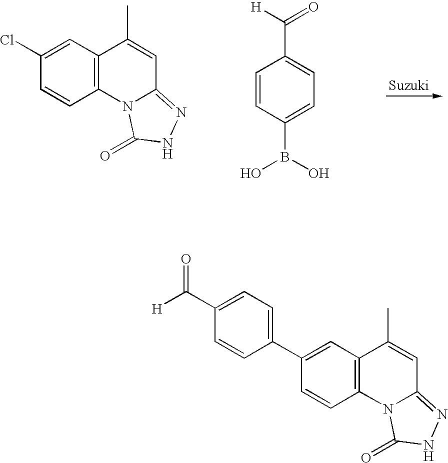

To a 5 ml vial, 7-chloro-5-methyl-2H-[1,2,4]triazolo[4,3-a]quinolin-1-one (117 mg, 0.5 mmol), 4-formylphenylboronic acid (90 mg, 0.6 mmol), cesium carbonate (651 mg, 2.0 mmol), and tetrakis(trisphenylphosphine)palladium (40 mg, 7 mol %) were added in dioxane:water (4:1, 4 ml). The reaction was subject to microwave irradiation at 165° C. for 20 min. After cooling to room temperature, the lower layer was removed and discarded, the solid that precipitated was filtered off, washed with methanol and dried under vacuum to give the desired product which was used without further purification.

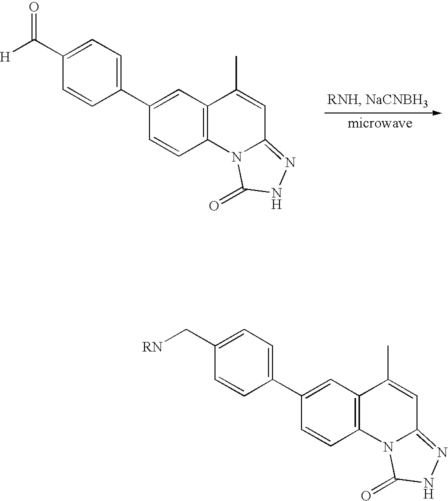

To a suspension of 4-(5-methyl-1-oxo-1,2-dihydro-[1,2,4]triazolo[4,3-α]quinolin-7-yl)-benzaldehyde (0.5 mmol, 64% pure) in DMF (4 ml), the appropriate amine (1 mmol) was added. The mixture was stirred overnight at room temperature and NaCNBH₃ (63 mg, 1 mmol) added, followed by 2 drops of AcOH. The reaction was subjected microwave irradiation at 150° C. for 5 min. Water (1 ml)was added, and the crude product isolated and purified by HPLC.

| Ex. | RN— | ¹H NMR(400 MHz, DMSO-D6) |
|---|---|---|
| 127 | 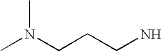 | 2.03(m, 2H), 2.55(s, 3H), 2.79(s, 6H), 3.03(m, 2H), 3.17(m, 2H), 4.25(s, 2H), 7.09-8.03(m, 7H), 9.05/9.11(d/s, J=8.4Hz/br, 2H), 12.47(s, 1H) |
| 128 | 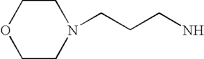 | 2.06(m, 2H), 2.56(s, 3H), 3.04(m, 4H), 3.19(m, 4H), 3.70(m, 2H), 4.00(m, 2H), 4.25(s, 2H), 7.10-8.03(m, 7H), 9.05(d, J=8.4Hz, 1H), 9.20(s, br, 1H), 12.47(s, 1H) |
| 129 | 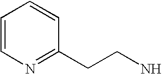 | 2.55(s, 3H), 3.17(m, 2H), 3.38(m, 2H), 4.31(s, 2H), 7.09-8.04(m, 10H), 8.60(s, 1H), 9.10(d, 1H), 12.50(s, 1H) |
| 130 | 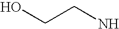 | 2.56(s, 3H), 3.00(m ,2H), 3.68(t, J=5Hz, 2H), 4.24(s, 2H), 5.30(s, br, 1H), 7.10(s, 1H), 7.63-8.04(m, 6H), 8.91(s, br, 1H), 9.05(d, J=8.4Hz, 1H), 12.46(s, 1H) |
| 131 | 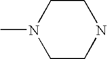 | 2.55(s, 3H), 2.80(s, 3H), 3.00-3.40(m, 8H), 3.88(s, 2H), 7.09-8.01(m, 7H), 9.04(d, J=8.4Hz, 1H), 12.46(s, 1H) |
| 132^{a} | 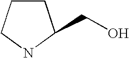 | 2.13-2.34(m, 6H), 2.60(s, 3H), 3.80-3.90(m, 3H), 4.49(t, J=6.4Hz, 1H), 4.95(d, J=12.8Hz, 1H), 6.98(s, 1H), 7.75-8.08(m, 6H), 9.13(d, J=8.4Hz, 1H), 11.42(s, 1H) |
| 133 | 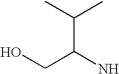 | 0.95(d, d, J=6.8Hz, 6H), 2.10(m, 1H), 2.56(s, 3H), 2.92(m, 1H), 3.73(m, 2H), 4.32(d, J=18.8Hz, 2H), 5.45(s, br, 1H), 7.10(s, 1H), 7.67-8.04(m, 6H), 8.90(s, br, 1H), 9.05(d, J=8.8Hz, 1H), 12.46(s, 1H) |
| 134 | 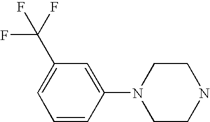 | 2.55(m, 11H), 4.50(s, 2H), 7.10-8.15(m, 10H), 9.10(d, 1H), 10.10(s, 1H), 12.50(s, 1H) |
| 135 | 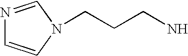 | 2.20(m, 2H), 2.55(s, 3H), 3.00(m, 2H), 4.20-4.30(m, 4H), 7.08(s, 1H), 7.60-8.05(m, 9H), 9.08(m, 2H), 12.50(s, 1H) |
| 136^{a} | 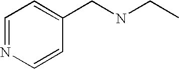 | 1.41(t, d=7.2Hz, 3H), 2.61(s, 3H), 3.16(q, J=7.2Hz, 2H), 4.41/4.50(s/s, 4H), 6.99(s, 1H), 7.72-8.84(m, 10H), 9.14(d, J=8.8Hz, 1H), 11.48(s, 1H) |
| 137 | 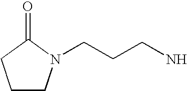 | 1.80-2.00(m, 4H), 2.50(s, 3H), 2.55(m, 6H), 2.90(m, 2H), 4.23(s, 2H), 7.10(s, 1H), 7.61-8.03(m, 6H), 8.85(s, br, 1H), 9.05(d, 1H), 12.45(s, 1H) |
| 138 | 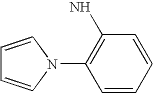 | 2.51(s, 3H), 3.30(s, 2H), 6.25-8.00(m, 15H), 9.00(d, 1H), 12.40(s, 1H) |
| 139 | 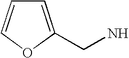 | 2.55(s, 3H), 4.22(s, 2H), 4.28(s, 2H), 6.58-6.68(m, 2H), 7.10(s, 1H), 7.88-8.01(m, 7H), 9.05(d, 1H), 9.42(s, br, 1H), 12.45(s, 1H) |
| 140 | 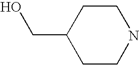 | 1.30-1.88(m, 5H), 2.55(s, 3H), 2.95(m, 2H), 3.20(m, 2H), 3.30(m, 2H), 4.32(s, 2H), 7.10(s, 1H), 7.60-8.00(m, 6H), 9.02(d, 1H), 12.45(s, 1H) |
| 141 | 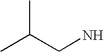 | 0.95(d, J=6.8Hz, 6H), 2.00(m, 1H), 2.56(s, 3H), 2.85(m, 2H), 4.23(s, 2H), 7.10(s, 1H), 7.64-8.04(m, 6H), 8.75(s, br, 1H), 9.05(d, 1H), 12.50(s, 1H) |

^{a}Acetone-d6 as solvent

### Examples 142-146

The following examples were prepared using the same procedure as that described for examples 120-134 using 2-methoxy-5-(5-methyl-1-oxo-1,2-dihydro[1,2,4]triazolo[4,3-a]quinolin-7-yl)benzaldehyde (prepared as described above) and the appropriate amine.

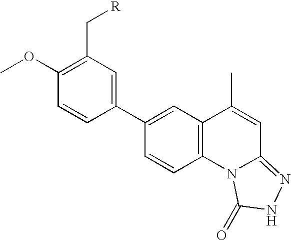

| Ex. | R— | 1H NMR(400 MHz, DMSO-d6) |
|---|---|---|
| 142 | 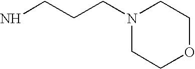 | 2.11(m, 2H), 2.54(s, 3H), 2.14-3.22(m, 10H), 3.84-3.97(m, 5H), 4.24(s, 2H), 7.09(d, J=12Hz, 1H), 7.24(t, J=4.5Hz, 1H), 7.88-7.97(m, 4H), 9.01/9.09(d/s, J=8.4Hz/br, 2H), 12.46(s, 1H) |
| 143 | 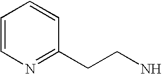 | 2.51(s, 3H), 3.23(m, 2H), 3.42(m, 2H), 3.99(s, 3H), 4.31(s, 2H), 6.97-8.57(m, 10H), 9.00(s, br, 1H), 9.03(d, J=8.8Hz, 1H), 12.45(s, 1H) |
| 144 | 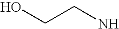 | 2.55(s, 3H), 3.03(m, 2H), 3.70(m, 2H), 3.92(s, 3H), 4.25(s, 2H), 7.10(s, 1H), 7.21(d, 1H), 7.89-7.98(m, 4H), 8.70(s, br, 1H), 9.03(d, J=8.8Hz, 1H), 12.45(s, 1H) |
| 145 | 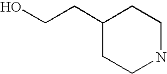 | 1.30-1.85(m, 7H), 2.55(s, 3H), 3.02(m, 2H), 3.45(m, 4H), 3.90(s, 3H), 4.31(s, 2H), 7.08(d, J=0.8Hz, 1H), 7.26(d, J=8.4Hz, 1H), 7.90-7.97(m, 4H), 9.02(d, J=8.8Hz, 1H), 12.45(s, 1H) |
| 146 | 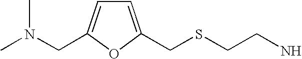 | 2.55(s, 3H), 2.73(s, 6H), 2.80(t, J=8.0Hz, 2H), 3.19(m, 2H), 3.88/3.92(s/s, 5H), 4.25(s, 2H), 4.33(s, 2H), 6.42(d, J=3.2Hz, 1H), 6.62(d, J=3.2Hz, 1H), 7.10(d, J=0.8Hz, 1H), 7.25(d, J=8.8Hz, 1H), 7.88-7.98(m, 4H), 8.95(s, br, 1H), 9.04(d, J=8.8Hz, 1H), 12.46(s, 1H) |

### Example 147

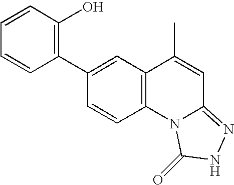

The methoxy analogue (example 105) (30.5 mg, 0.1 mmol) in BBr₃ (1M in CH₂Cl₂, 3 ml) was stirred for 3 hours at room temperature. Crushed ice was added into the mixture and solvent removed under reduced pressure. The residue was dissolved in the minimum amount of DMSO and purified by HPLC. ¹H NMR (400 MHz, DMSO-d6): 2.50 (s, 3H), 6.85-7.95 (m, 7H), 8.90 (d, 1H), 9.85 (s, 1H), 12.40 (s, 1H)

### Examples 148-149

The following examples were prepared using the procedure described for Example 147 using the appropriate methoxy-substituted triazolone.

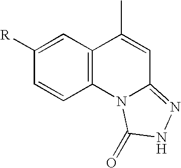

| Ex. | R— | ¹H NMR(400 MHz, DMSO-d6) |
|---|---|---|
| 148 | 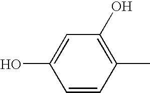 | 2.51(s, 3H), 6.65-7.90(m, 6H), 8.95(d, 1H), 9.82(s, 1H), 10.29(s, 1H), 12.40(s, 1H) |
| 149 | 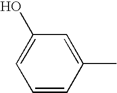 | 2.50(s, 3H), 6.80-7.95(m, 7H), 9.00(d, 1H), 9.73(s, 1H), 12.45(s, 1H) |

### Examples 150-184

The following examples were prepared using the procedure described below:

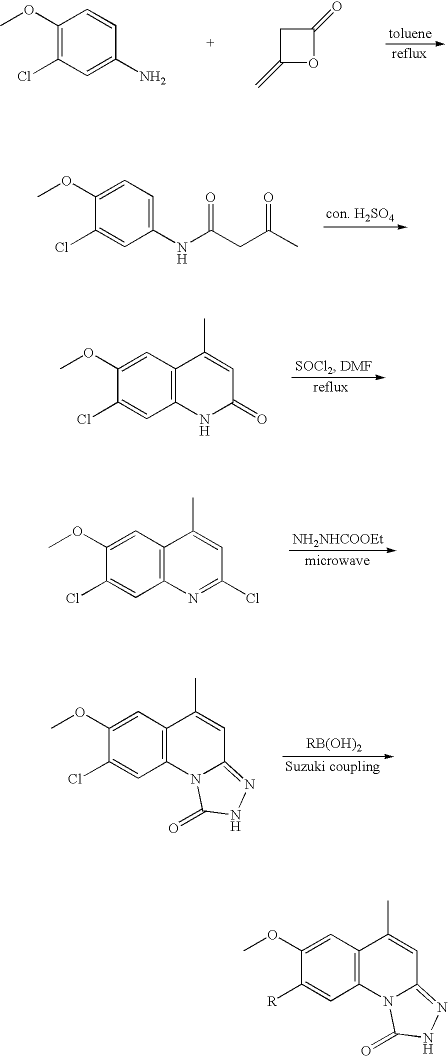

Diketene (32 ml, 32 g, 381 mmol) was added to the suspension of 3-chloro-4-methoxyphenylamine (50 g, 317.25 mmol) in toluene (300 ml). The mixture was refluxed for 6 hrs, cooled to room temperature and allowed to stand overnight. The precipitated solid was filtered off, washed with ether and dried under vacuum, to give the desired product as a light yellow solid (48 g, 62.9%).

A mixture of 3-chloro-4-methoxy acetoacetanilide (48 g, 199.6 mmol) and concentrated sulfuric acid (80 ml) was heated on an oil-bath at 70-80° C. for 0.5 h followed by 1.0 h at 100° C. The mixture was cooled to room temperature and poured onto crushed ice. The precipitated solid-was filtered off and recrystallized from ethanol to give the desired compound as a white solid (30 g, 67.26%).

A mixture of 7-chloro-6-methoxy-4-methyl-1H-quinolin-2-one (30 g, 134.2 mmol), DMF (10 ml) and thionyl chloride (300 g) was refluxed for 3 hr. The mixture was cooled to room temperature and the solid that crystallized out filtered off, washed with acetone and dried under vacuum. The desired product was obtained as a yellow solid (16.4 g, 50.5%).

To a suspension of 2,7-dicloro-6-methoxy-4-methyl-quinoline (363 mg, 1.5 mmol) and ethyl carbazate (173 mg 1.66 mmol) in ethanol (3.7 ml) was added 6 drops of HCl (4N in dioxane). The reaction mixture was subject to microwave irradiation at 170° C. for 20 min. After cooling to room temperature the orange precipitate was removed by filtration, washed with methanol (3×10 ml), and dried under vacuum, to give 8-Choloro-7-methoxy-5-methyl-2H-[1,2,4]triazolo[4,3-α]quinolin-1-one was obtained (225 mg, 57.0%). ¹H NMR (400 MHz, DMSO-d6): 3.37 (s, 3H), 4.04 (s, 3H), 7.07 (s, 1H), 7.35 (s, 1H), 8.98 (s, 1H), 12.46 (s, 1H)

To a 5 ml vial, 8-Choloro-7-methoxy-5-methyl-2H-[1,2,4]triazolo[4,3-α]quinolin-1-one (132 mg, 0.5 mmol), the appropriate boronic acid (0.6 mmol), cesium carbonate (651 mg, 2.0 mmol), and tetrakis(trisphenylphosphine)palladium (40 mg, 7 mol %) were added in dioxane:water (4:1, 4 ml). The reaction was subject to microwave irradiation at 165° C. for 20 min. The mixture was cooled to room temperature and the lower layer removed and discarded. Solvent was removed from the upper layer, and the residue obtained dissolved in minimum amount of DMSO. The DMSO solution was filtered and purified by HPLC.

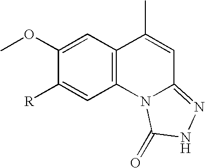

| Ex. | R— | 1H NMR(400 MHz, DMSO-d6) |
|---|---|---|
| 150 | 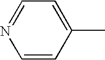 | 2.53(s, 3H), 3.97(s, 3H), 7.16(s, 1H), 7.44(s, 1H), 7.86(m, 2H), 8.80(m, 2H), 9.06(s, 1H), 12.50(s, 1H) |
| 151 | 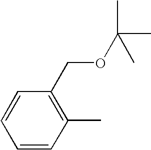 | 0.99(s, 9H), 2.54(s, 3H), 3.85(s, 3H), 4.20(d, 2H), 7.08-7.52(m, 6H), 8.78(s, 1H), 12.37(s, 1H) |
| 152 | 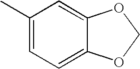 | 2.54(s, 3H), 3.91(s, 3H), 6.08(s, 2H), 7.01-7.33(m, 5H), 8.92(s, 1H), 12.39(s, 1H) |
| 153 | 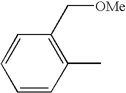 | 2.53(s, 3H), 3.13(s, 3H), 3.85(s, 3H), 4.03(d, 2H), 7.08-7.51(m, 6H), 8.77(s, 1H), 12.39(s, 1H) |
| 154 | 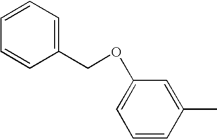 | 2.51(s, 3H), 3.88(s, 3H), 5.16(s, 2H), 7.00-7.50(m, 11H), 8.95(s, 1H), 12.40(s, 1H) |
| 155 | 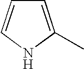 | 2.54(s, 3H), 4.02(s, 3H), 6.15-7.30(m, 5H), 9.20(s, 1H), 11.10(s, 1H), 12.30(s, 1H) |
| 156 | 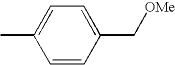 | 2.50(s, 3H), 3.32(s, 3H), 3.90(s, 3H), 4.47(s, 2H), 7.05-7.54(m, 6H), 8.93(s, 1H), 12.40(s, 1H) |
| 157 | 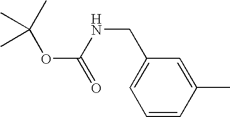 | 1.39(s, 9H), 2.51(s, 3H), 3.89(s, 3H), 4.20(d, 2H), 7.06-7.39(m, 7H), 8.92(s, 1H), 12.40(s, 1H) |
| 158 | 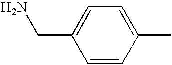 | 2.54(s, 3H), 3.90(s, 3H), 4.10(t, 2H), 7.08-7.58(m, 6H), 8.15(t, br, 2H), 8.94(s, 1H), 12.42(s, 1H) |
| 159 | 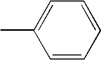 | 2.52(s, 3H), 3.90(s, 3H), 7.05-7.65(m, 7H), 8.92(s, 1H), 12.35(s, 1H) |
| 160 | 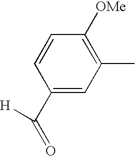 | 2.51(s, 3H), 3.86(s, 6H), 7.10-8.00(m, 5H), 8.80(s, 1H), 9.93(s, 1H), 12.40(s, 1H) |
| 161 | 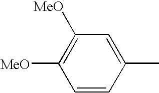 | 2.50(s, 3H), 3.79/3.81(s/s, 6H), 3.90(s, 3H), 7.03-7.32(m, 5H), 8.94(s, 1H), 12.39(s, 1H) |
| 162 | 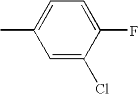 | 2.51(s, 3H), 3.92(s, 3H), 7.10-7.71(m, 5H), 8.92(s, 1H), 12.42(s, 1H) |
| 163 | 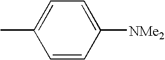 | 2.50(s, 3H), 2.98(s, 6H), 3.89(s, 3H), 6.87-7.46(m, 6H), 8.93(s, 1H), 12.36(s, 1H) |
| 164 | 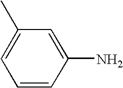 | 2.51(s, 3H), 4.09(s, 3H), 6.94-7.61(m, 6H), 8.99(s, 1H), 12.41(s, 1H) |
| 165 | 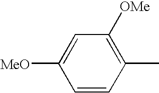 | 2.51(s, 3H), 3.70(s, 3H), 3.82(s, 6H), 6.61-7.29(m, 5H), 8.75(s, 1H), 12.34(s, 1H) |
| 166 | 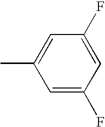 | 2.54(s, 3H), 3.94(s, 3H), 7.13-7.40(m, 5H), 8.98(s, 1H), 12.50(s, 1H) |
| 167 | 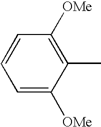 | 2.52(s, 3H), 3.64(s, 6H), 3.79(s, 3H), 6.76-7.34(m, 5H), 8.64(s, 1H), 12.34(s, 1H) |
| 168 | 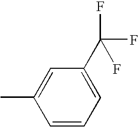 | 2.51(s, 3H), 3.92(s, 3H), 7.09-7.85(m, 6H), 8.96(s, 1H), 12.44(s, 1H) |
| 169 | 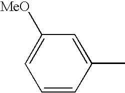 | 2.51(s, 3H), 3.80(s, 3H), 3.91(s, 3H), 6.95-7.42(m, 6H), 8.95(s, 1H), 12.40(s, 1H) |
| 170 | 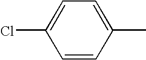 | 2.51(s, 3H), 3.91(s, 3H), 7.06-7.56(m, 6H), 8.94(s, 1H), 12.40(s, 1H) |
| 171 | 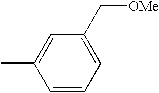 | 2.51(s, 3H), 3.41(s, 3H), 3.90(s, 3H), 4.49(s, 2H), 7.06-7.46(m, 6H), 8.93(s, 1H), 12.40(s, 1H) |
| 172 | 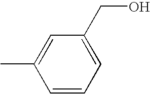 | 2.50(s, 3H), 3.89(s, 3H), 4.57(s, 2H), 5.27(s, br, 1H), 7.04-7.47(m, 6H), 8.92(s, 1H), 12.39(s, 1H) |
| 173 | 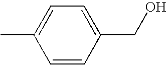 | 2.49(s, 3H), 3.88(s, 3H), 4.55(s, 2H), 7.32-7.48(m, 6H), 8.91(s, 1H), 12.40(s, 1H) |
| 174 | 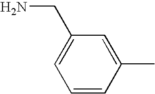 | 2.53(s, 3H), 3.90(s, 3H), 4.13(s, 2H), 7.10-7.62(m, 6H), 8.22(s, br, 2H), 8.95(s, 1H), 12.42(s, 1H) |
| 175 | 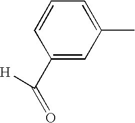 | 2.55(s, 3H), 3.92(s, 3H), 7.10-8.08(m, 6H), 9.00(s, 1H), 10.10(s, 1H), 12.40(s, 1H) |
| 176 | 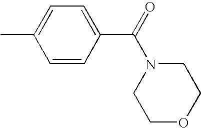 | 2.54(s, 3H), 3.60-3.80(m, 8H), 3.92(s, 3H), 7.09-7.63(m, 6H), 8.96(s, 1H), 12.43(s, 1H) |
| 177 | 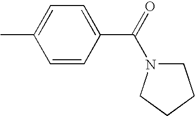 | 1.84-1.91(m, 4H), 2.54(s, 3H), 3.45-3.52(m, 4H), 3.93(s, 3H), 7.09-7.61(m, 6H), 8.97(s, 1H), 12.45(s, 1H) |
| 178 | 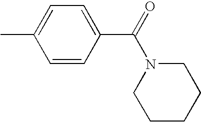 | 1.50-1.65(m, 6H), 2.52(s, 3H), 3.40-3.60(m, 4H), 3.93(s, 3H), 7.09-7.62(m, 6H), 8.97(s, 1H), 12.43(s, 1H) |
| 179 | 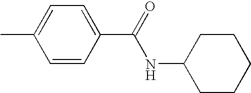 | 1.10-1.90(m, 10H), 2.52(s, 3H), 3.80(m, 1H), 3.92(s, 3H), 7.05-8.22(m, 7H), 8.99(s, 1H), 12.40(s, 1H) |
| 180 | 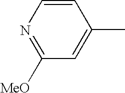 | 2.54(s, 3H), 3.90(s, 6H), 6.92(d, 1H), 7.05(s, 1H), 7.32(s, 1H), 7.89(dd, 1H), 8.32(d, 1H), 8.90(s, 1H), 12.42(s, 1H) |
| 181 | 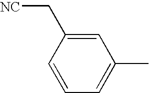 | 2.60(s, 3H), 4.01(s, 2H), 4.24(s, 3H), 7.14-7.71(m, 6H), 9.00(s, 1H), 12.47(s, 1H) |
| 182 | 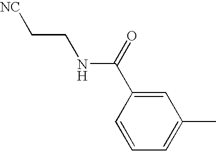 | 2.54(s, 3H), 2.86(t, J=6.4Hz, 2H), 3.59(t, J=6.4Hz, 2H), 3.97(s, 3H), 7.14-8.08(m, 6H), 9.03(m, 2H), 12.49(s, 1H) |
| 183 | 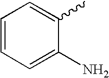 | 2.50(s, 3H), 3.93(s, 3H), 7.05-8.84(m, 6H), 8.88(s, 1H), 12.42(s, 1H). |
| 184 | 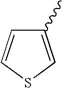 | 2.53(s, 3H), 3.98(s, 3H), 7.03(s, 1H), 7.33(s, 1H), 7.46(d, J=4.8Hz, 1H), 7.65(d, J=2.8Hz, 1H), 7.89(s, 1H), 9.14(s, 1H), 12.40(s, 1H). |

### Examples 185-207

The following examples were prepared using the following procedure.

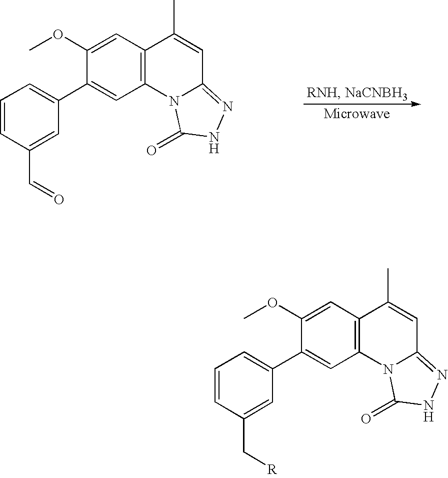

To a suspension of 3-(7-methoxy-5-methyl-1-oxo-1,2-dihydro-[1,2,4]triazolo[4,3-α]quinolin-8-yl)-benzaldehyde (0.5 mmol) in DMF (4 ml), the appropriate amine (1 mmol) was added. The mixture was stirred overnight at room temperature and NaCNBH₃ (63 mg, 1 mmol) added, followed by 2 drops of AcOH. The reaction was subjected to the microwave irradiation at 150° C. for 5 min. Water (1 ml) was added. The crude product was isolated and purified by HPLC.

| Ex. | R— | ¹H NMR(400 MHz, DMSO-D6) |
|---|---|---|
| 185 | 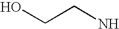 | 2.54(s, 3H), 3.03(m, 2H), 3.68(t, J=5.2Hz, 2H), 3.91(s, 3H), 4.26(s, 2H), 5.25(s, br, 1H), 7.10-7.69(m, 6H), 8.90(s, br, 1H), 8.96(s, 1H), 12.43(s, 1H) |
| 186 | 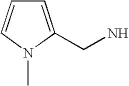 | 2.51(s, 3H), 3.67(s, 3H), 3.90(s, 3H), 4.23(s, 2H), 4.29(s, 2H), 6.03(m, 1H), 6.27(m, 1H), 6.82(t, J=2Hz, 1H), 7.09(d, J=0.8Hz, 1H), 7.38-7.67(m, 5H), 8.96(s, 1H), 9.15(s, br, 1H), 12.43(s, 1H) |
| 187 | 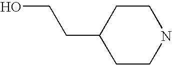 | 1.30-1.90(m, 7H), 2.54(s, 3H), 2.93(m, 2H), 3.40(m, 4H), 3.90(s, 3H), 4.37(s, 2H), 7.09-7.73(m, 6H), 8.97(s, 1H), 12.43(s, 1H) |
| 188 | 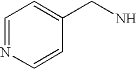 | 2.51(s, 3H), 3.85(s, 3H), 4.34/4.36(s/s, 4H), 7.09-7.68(m, 8H), 8.86(m, 2H), 8.96(s, 1H), 9.59(s, br, 1H), 12.43(s, 1H) |
| 189 | 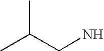 | 0.95(d, J=6.8Hz, 6H), 1.99(m, 1H), 2.51(s, 3H), 2.82(m, 2H), 3.91(s, 3H), 4.25(s, 2H), 7.10-7.69(m, 6H), 8.78(s, br, 1H), 8.96(s, 1H), 12.43(s, 1H) |
| 190 | 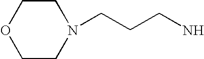 | 2.05(m, 2H), 2.51(s, 3H), 3.05(m, 4H), 3.18(m, 4H), 3.68(m, 2H), 3.91(s, 3H), 3.96(m, 2H), 4.27(s, 2H), 7.10-7.67(m, 6H), 8.96(s, 1H), 9.10(s, br, 1H), 12.44(s, 1H) |
| 191 | 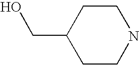 | 1.18(m, 2H), 1.35(m, 1H), 1.63(m, 2H), 1.92(m, 2H), 2.51(s, 3H), 2.84(m, 2H), 3.22(m, 2H), 3.52(s, 2H), 3.95(s, 3H), 4.39(t, J=5.2Hz, 1H), 7.07-7.46(m, 6H), 8.95(s, 1H), 12.38(s, 1H) |
| 192 | 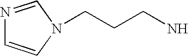 | 2.20(m, 2H), 2.51(s, 3H), 3.17(m, 2H), 3.96(s, 3H), 4.26(s, 2H), 4.31(t, J=6.8Hz, 2H), 7.10-7.95(m, 8H), 8.96(s, 1H), 9.07/9.11(s/s, 2H), 12.44(s, 1H) |
| 193 | 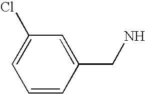 | 2.54(s, 3H), 3.91(s, 3H), 4.30(m, 4H), 7.10-7.67n(m, 10H), 8.96(s, 1H), 9.34(s, br, 1H), 12.43(s, 1H) |
| 194 | 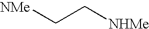 | 2.50(s, 3H), 2.62(s, 6H), 3.17-3.30(m, 4H), 3.92(s, 3h), 4.32(s, 2H), 7.10-7.68(m, 6H), 8.70(s, br, 1H), 8.97(s, 1H), 12.44(s, 1H) |
| 195 | 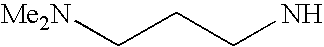 | 2.03(m, 2H), 2.53(s, 3H), 2.73(s, 6H), 3.04(m, 2H), 3.17(m, 2H), 3.91(s, 3H), 4.27(s, 2H), 7.09(s, 1H), 7.37-7.67(m, 5H), 8.95(s, 1H), 9.15(s, br, 1H), 12.45(s, 1H) |
| 196 | 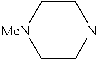 | 2.12(s, 3H), 2.30-2.40(m, 8H), 3.35(s, 3H), 3.50(s, 2H), 3.90(s, 3H), 7.00-7.45(m, 6H), 8.91(s, 1H), 12.30(s, 1H) |
| 197 | 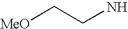 | 2.54(s, 3H), 3.15(m, 2H), 3.36(s, 3H), 3.60(t, J=5.2Hz, 2H), 3.96(s, 3H), 4.26(s, 2H), 7.10-7.69(m, 6H), 8.96(s, 1H), 9.00(s, br, 1H), 12.44(s, 1H) |
| 198 | 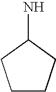 | 1.54-1.71(m, 6H), 2.01(m, 2H), 2.50(s, 3H), 3.54(m, 1H), 3.91(s, 3H), 4.25(m, 2H), 7.10-7.68(m, 6H), 8.89(s, br, 1H), 8.96(s, 1H), 12.43(s, 1H) |
| 199 | 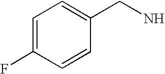 | 2.50(s, 3H), 3.95(s, 3H), 4.24/4.27(s/s, 4H), 7.10-7.66(m, 10H), 8.96(s, 1H), 9.32(s, br, 1H), 12.43(s, 1H) |
| 200 | 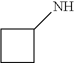 | 1.80(m, 2H), 2.17(m, 4H), 2.51(s, 3H), 3.74(m, 1H), 3.91(s, 3H), 4.13(s, 2H), 7.10-7.65(m, 6H), 8.95(s, 1H), 9.08(s, br, 1H), 12.43(s, 1H) |
| 201 | 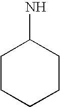 | 1.13-2.14(m, 10H), 2.50(s, 3H), 3.06(m, 1H), 3.90(s, 3H), 4.27(s, 2H), 7.10-7.91(m, 6H), 8.77(s, br, 1H), 8.95(s, 1H), 12.43(s, 1H) |
| 202 | 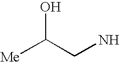 | 1.11(d, J=6.0Hz, 3H), 2.52(s, 3H), 2.73(m, 2H), 2.96(m, 1H), 3.91(s, 3H), 4.24(s, 2H), 5.34(s, br, 1H), 7.09-7.58(m, 6H), 8.91/8.96(s/s, 2H), 12.43(s, 1H) |
| 203 | 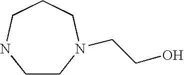 | 2.21(m, 2H), 2.59(s, 3H), 3.23(m, 4H), 3.78(m, 8H), 3.98(s, 3H), 4.46(s, 2H), 7.17-7.77(m, 6H), 9.04(s, 1H), 12.50(s, 1H) |
| 204 | 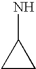 | 0.81(m, 4H), 2.51(s, 3H), 2.76(m, 1H), 3.90(s, 3H), 4.34(s, 2H), 7.10-7.67(m, 6H), 8.95(s, 1H), 9.05(s, br, 1H), 12.43(s, 1H) |
| 205 | 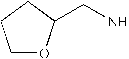 | 1.83-2.05(m, 4H), 2.51(s, 3H), 2.94-3.08(m, 2H), 3.73(m, 2H), 3.95(s, 3H), 4.12(m, 1H), 4.27(s, 2H), 7.09-7.69(m, 6H), 8.96(s, 1H), 9.05(s, br, 1H), 12.43(s, 1H) |
| 206 | 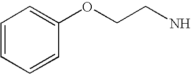 | 2.54(s, 3H), 3.40(m, 2H), 3.98(s, 3H), 4.26(t, J=5.0Hz, 2H), 4.36(s, 2H), 6.96-7.72(m, 11H), 8.97(s, 1H), 9.19(s, br, 1H), 12.43(s, 1H) |
| 207 | 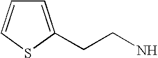 | 2.54(s, 3H), 3.17-3.36(m, 4H), 3.91(s, 3H), 4.30(s, 2H), 6.90-7.68(m, 9H), 8.96(s, 1H), 9.05(s, br, 1H), 12.43(s, 1H) |

### Example 208-221

The following examples were prepared using the same procedure as described for examples 185-207 using 2-methoxy-5-(7-methoxy-5-methyl-1-oxo-1,2-dihydro[2,4]triazolo[4,3-a]quinolin-8-yl)benzaldehyde and the appropriate amine.

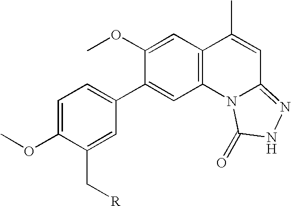

| Ex. | R— | 1H NMR(400 MHz, DMSO-d6) |
|---|---|---|
| 208 | 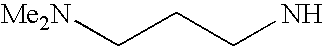 | 2.02(m, 2H), 2.54(s, 3H), 2.78(s, 6H), 3.02(m, 2H), 3.12(m, 2H), 3.91/3.92(s/s, 6H), 4.23(s, 2H), 7.10-7.63(m, 5H), 8.72(s, br, 1H), 8.95(s, 1H), 12.43(s, 1H) |
| 209 | 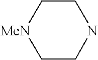 | 2.54(s, 3H), 2.68-3.17(m, 8H), 3.88-4.00(m, 11H), 7.08-7.95(m, 5H), 8.96(s, 1H), 12.42(s, 1H) |
| 210 | 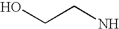 | 2.52(s, 3H), 3.02(m, 2H), 3.68(t, J=5.0Hz, 2H), 3.91(s, s, 6H), 4.23(s, 2H), 5.25(s, br, 1H), 7.08-7.62(m, 5H), 8.65(s, br, 1H), 8.95(s, 1H), 12.42(s, 1H) |
| 211 | 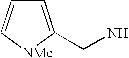 | 2.54(s, 3H), 3.60(s, 3H), 3.95(s, 6H), 4.21(s, s, 4H), 6.03-7.65(m, 8H), 8.90(s, br, 1H), 9.05(s, 1H), 12.42(s, 1H) |
| 212 | 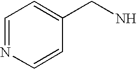 | 2.52(s, 3H), 3.90(s, s, 6H), 4.23(s, 2H), 4.33(s, 2H), 7.08-7.62(m, 7H), 8.70(m, 2H), 8.95(s, 1H), 9.28(s, br, 1H), 12.42(s, 1H) |
| 213 | 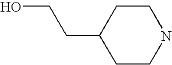 | 1.17-1.97(m, 7H), 2.51(s, 3H), 2.89(m, 2H), 3.42(m, 4H), 3.82(s, 3H), 3.89(s, 3H), 4.32(s, 2H), 7.04-7.54(m, 5H), 8.94(s, 1H), 12.37(s, 1H) |
| 214 | 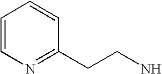 | 2.54(s, 3H), 3.19(t, J=7.2Hz, 2H), 3.41(m, 2H), 3.91/3.92(s/s, 6H), 4.30(s, 2H), 7.08-7.95(m, 8H), 8.56(d, J=4.8Hz, 1H), 8.95(m, 2H), 12.42(s, 1H) |
| 215 | 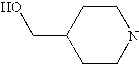 | 1.15(m, 2H), 1.32(m, 1H), 1.62(m, 2H), 2.00(t, J=6.8Hz, 2H), 2.56(s, 3H), 2.95(m, 2H), 3.30(t, J=5.6Hz, 2H), 3.52(s, 2H), 3.89/3.95(s/s, 6H), 4.45(t, J=5.2Hz, 1H), 7.10-8.02(m, 5H), 9.01(s, 1H), 12.44(s, 1H) |
| 216 | 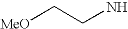 | 2.52(s, 3H), 3.15(m, 2H), 3.32(s, 3H), 3.62(t, J=5.2Hz, 2H), 3.91(s, s, 6H), 4.22(s, 2H), 7.08-7.66(m, 5H), 8.73(s, br, 1H), 8.95(s, 1H), 12.42(s, H) |
| 217 | 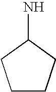 | 1.54-1.71(m, 8H), 2.51(s, 3H), 3.42(m, 1H), 3.90(s, s, 6H), 4.18(s, 2H), 7.07-7.61(m, 5H), 8.69(s, br, 1H), 8.94(s, 1H), 12.41(s, 1H) |
| 218 | 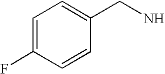 | 2.51(s, 3H), 3.90(s, s, 6H), 4.24/4.26(s/s, 4H), 7.07-7.58(m, 9H), 8.93(s, 1H), 9.11(s, br, 1H), 12.41(s, 1H) |
| 219 | 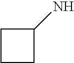 | 1.79(m, 2H), 2.16(m, 4H), 2.51(s, 3H), 3.72(m, 1H), 3.91(s, s, 6H), 4.07(s, 2H), 7.08-7.62(m, 5H), 8.85(s, br, 1H), 8.95(s, 1H), 12.41(s, 1H) |
| 220 | 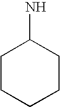 | 1.28-2.12(m, 10H), 2.51(s, 3H), 3.06(m, 1H), 3.91(s, s, 6H), 4.20(s, 2H), 7.08-7.92(m, 5H), 8.50(s, br, 1H), 8.95(s, 1H), 12.41(s, 1H) |
| 221 | 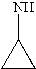 | 0.69(m, 2H), 0.78(m, 2H), 2.50(m, 1H), 2.92(s, 3H), 4.18(s, 2H), 4.27/4.31(s/s, 6H), 7.46-7.91(m, 5H), 9.35(s, 1H), 12.80(s, 1H) |

### Examples 222-225

The following examples were prepared using the same procedure as described for examples 208-221 using 2-(7-methoxy-5-methyl-1-oxo-1,2-dihydro[1,2,4]triazolo[4,3-a]quinolin-8-yl)benzaldehyde and the appropriate amine.

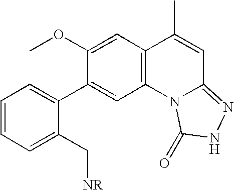

| Ex. | RN— | 1H NMR(400 MHz, DMSO-d6) |
|---|---|---|
| 222 | 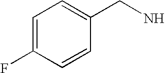 | 2.49(s, 3H), 3.85(s, 3H), 4.00-4.30(m, 4H), 7.00-7.70(m, 10H), 8.74(s, 1H), 9.34(s, br, 1H), 12.43(s, 1H) |
| 223 | 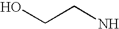 | 2.55(s, 3H), 2.86(m, 2H), 3.53(m, 2H), 3.90(s, 3H), 4.07(s, 2H), 5.12(s, br, 1H), 7.13-7.73(m, 6H), 8.71(s, br, 1H), 8.85(s, 1H), 12.43(s, 1H) |
| 224 | 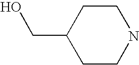 | 1.33-1.72(m, 5H), 2.49(s, 3H), 2.70-3.30(m, 6H), 3.96(s, 3H), 4.28(m, 2H), 5.26(s, br, 1H), 7.13-7.79(m, 6H), 8.77(s, 1H), 12.44(s, 1H) |
| 225 | 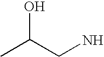 | 1.41(m, 3H), 2.92(s, 3H), 3.17(m, 1H), 4.12-4.37(m, 7H), 7.55-8.15(m, 6H), 9.15(s, br, 1H), 9.24(s, 1H), 12.86(s, 1H) |

### Example 226-272

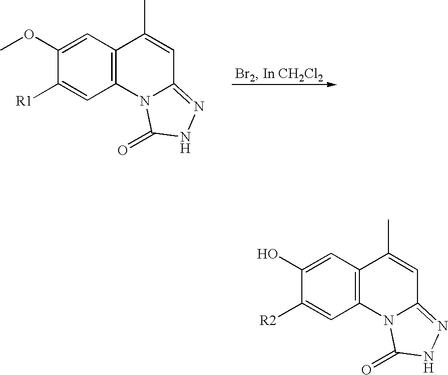

As previously described, the methoxy analog (0.1 mmol) in BBr₃ (1M in CH₂Cl₂, 3 ml) was stirred for 3 hours at room temperature. Crushed ice was added and the solvent removed under reduced pressure. The residue was dissolved in minimum amount of DMSO and purified by HPLC.

| Ex. | R1— | R2— | ¹H NMR(400 MHz, DMSO-d6) |
|---|---|---|---|
| 226 | 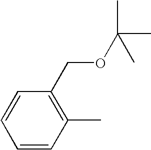 | 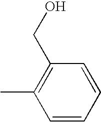 | 2.38(s, 3H), 4.36(s, 2H), 7.03-7.60(m, 6H), 8.68(s, 1H), 9.88(s, 1H), 12.34(, 1H) |
| 227 | 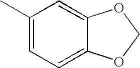 | 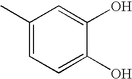 | 2.40(s, 3H), 6.75-7.25(m, 5H), 8.91-9.08(m, 3H), 9.80(s, 1H), 12.35(s, 1H) |
| 228 | 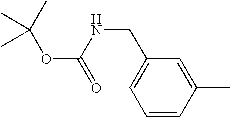 | 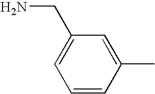 | 2.45(s, 3H), 4.20(s, 2H), 7.10-7.78(m, 6H), 8.25(s, br, 2H), 8.98(s, 1H), 10.10(s, 1H), 12.40(s, 1H) |
| 229 | 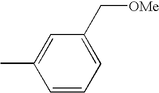 | 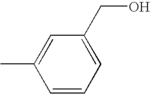 | 2.45(s, 3H), 4.60(s, 2H), 7.10-7.55(m, 6H), 9.00(s, 1H), 10.02(s, 1H), 12.42(s, 1H) |
| 230 | 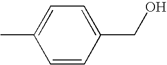 | 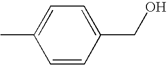 | 2.41(s, 3H), 4.55(s, 2H), 5.30(s, br, 1H), 7.10-7.56(m, 6H), 8.93(s, 1H), 9.97(s, 1H), 12.35(s, 1H) |
| 231 | 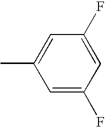 | 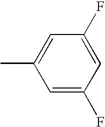 | 2.40(s, 3H), 7.10-7.50(m, 5H), 8.95(s, 1H), 10.35(s, 1H), 12.45(s, 1H) |
| 232 | Cl | Cl | 2.40(s, 3H), 7.04(s, 1H), 7.31(s, 1H), 8.92(s, |
| | | | 1H), 10.57(s, 1H), 12.42(s, 1H) |
| 233 | 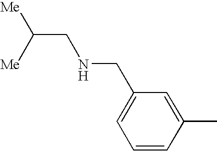 | 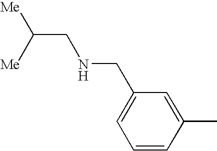 | 0.94(d, J=6.4Hz, 6H), 2.01(m, 1H), 2.42(s, 3H), 2.82(m, 2H), 4.44(s, 2H), 7.05-7.75(m, 6H), 8.70(s, br, 1H), 8.95(s, 1H), 10.15(s, 1H), 12.48(s, 1H) |
| 234 | 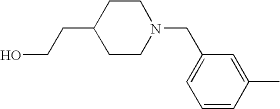 | 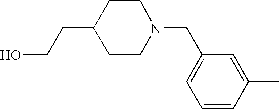 | 1.20-1.90(m, 7H), 2.40(m, 2H), 2.50(s, 3H), 2.77(m, 4H), 4.38(s, 2H), 7.05-7.76(m, 6H), 8.94(s, 1H), 10.18(s, 1H), 12.38(s, 1H) |
| 235 | 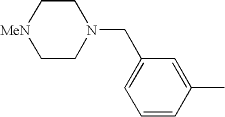 | 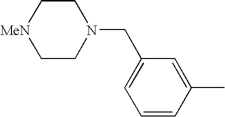 | 2.41(s, 3H), 2.79(s, 3H), 3.20-3.70(m, 10H), 7.04-7.64(m, 6H), 8.92(s, 1H), 10.05(s, 1H), 12.36(s, 1H) |
| 236 | 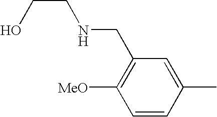 | 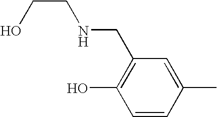 | 2.40(s, 3H), 3.02(m, 2H), 4.20(m, 4H), 7.00-7.63(m, 5H), 8.63(s, br, 1H), 8.89(s, 1H), 9.99(s, 1H), 10.47(s, 1H), 12.34(s, 1H) |
| 237 | 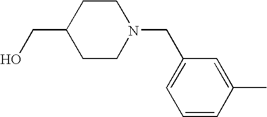 | 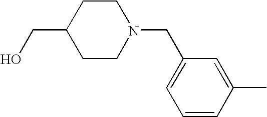 | 1.35-1.85(m, 5H), 2.41(s, 3H), 2.85(m, 2H), 3.30(m, 2H), 3.50(m, 2H), 4.40(s, 2H), 7.04-7.71(m, 6H), 8.94(s, 1H), 10.15(s, 1H), 12.38(s, 1H) |
| 238 | 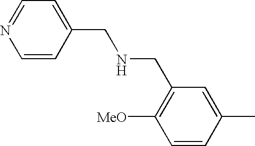 | 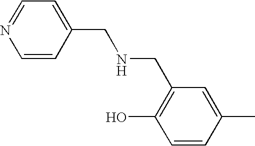 | 2.40(s, 3H), 4.31/4.35(s/s, 4H), 7.01-7.84(m, 7H), 8.91(m, 3H), 9.35(s, 1H), 10.16(m, 2H), 12.38(s, 1H) |
| 239 | 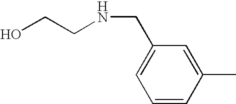 | 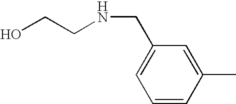 | 2.42(s, 3H), 3.02(m, 2H), 3.67(m, 2H), 4.26(s, 2H), 5.25(s, br, 1H), 7.05-7.52(m, 6H), 8.93(s, s, 2H), 10.05(s, 1H), 12.37(s, 1H) |
| 240 | 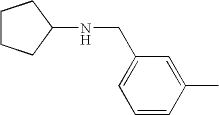 | 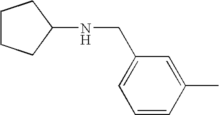 | 1.54-2.03(m, 9H), 2.42(s, 3H), 4.24(t, J=5.8Hz, 2H), 7.05-7.75(m, 6H), 8.88(s, br, 1H), 8.94(s, 1H), 10.11(s, 1H), 12.38(s, 1H) |
| 241 | 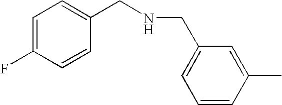 | 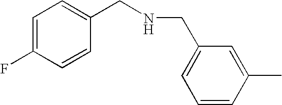 | 2.42(s, 3H), 4.26/4.27(s/s, 4H), 7.05-7.74(m, 10H), 8.94(s, 1H), 9.28(s, br, 1H), 10.11(s, 1H), 12.38(s, 1H) |
| 242 | 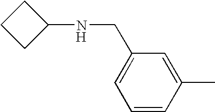 | 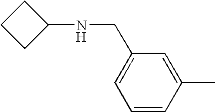 | 1.82(m, 2H), 2.17(m, 4H), 2.42(s, 3H), 3.70(m, 1H), 4.12(s, 2H), 7.05-7.71(m, 6H), 8.93(s, 1H), 9.05(s, br, 1H), 10.15(s, 1H), 12.39(s, 1H) |
| 243 | 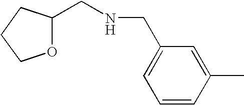 | 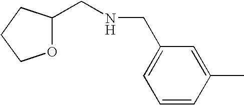 | 1.25-2.20(m, 5H), 2.40(s, 3H), 2.70-3.00(m, 4H), 4.45(m, 2H), 7.00-7.80(m, 6H), 8.90(s, 1H), 9.50(m, 1H), 10.15(s, 1H), 12.40(s, 1H) |
| 244 | 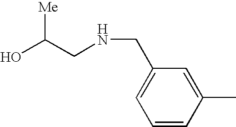 | 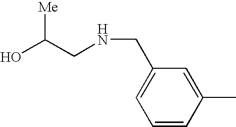 | 1.11(d, J=6.4Hz, 3H), 2.42(s, 3H), 2.75(m, 2H), 2.95(m, 1H), 3.95(m, 1H), 4.26(s, 2H), 7.04-7.53(m, 6H), 8.90/8.93(s/s, 2H), 10.15(s, 1H), 12.40(s, 1H) |
| 245 | 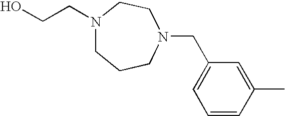 | 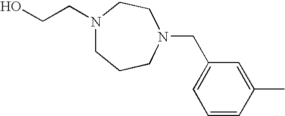 | 2.10(m, 2H), 2.42(s, 3H), 3.23(m, 6H), 3.71(m, 6H), 4.40(s, 2H), 7.05-7.78(m, 6H), 8.95(s, 1H), 10.20(s, 1H), 12.40(s, 1H) |
| 246 | 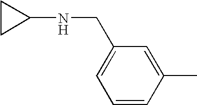 | 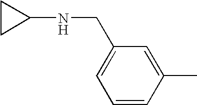 | 0.80(m, 4H), 2.42(s, 3H), 2.75(m, 1H), 4.33(s, 2H), 7.04-7.74(m, 6H), 8.93(s, 1H), 9.10(s, br, 1H), 10.18(s, 1H), 12.40(s, 1H) |
| 247 | 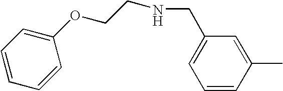 | 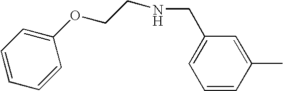 | 2.42(s, 3H), 3.47(m, 2H), 4.25-4.35(m, 4H), 6.90-7.80(m, 11H), 8.96(s, 1H), 9.15(s, br, 1H), 10.15(s, 1H), 12.38(s, 1H) |
| 248 | 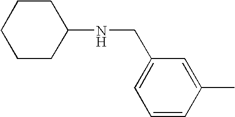 | 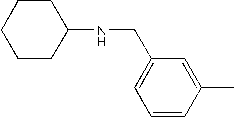 | 1.10-2.12(m, 11H), 2.41(s, 3H), 4.25(s, 2H), 7.03-7.72(m, 6H), 8.74(s, br, 1H), 8.92(s, 1H), 10.15(s, 1H), 12.38(s, 1H) |
| 249 | 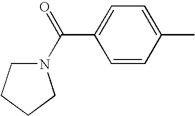 | 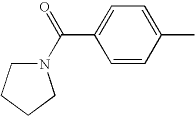 | 1.85(m, 4H), 2.42(s, 3H), 3.48(m, 4H), 7.04-7.69(m, 6H), 8.96(s, 1H), 10.10(s, 1H), 12.37(s, 1H) |
| 250 | 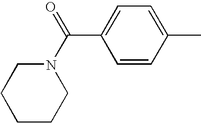 | 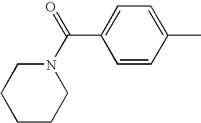 | 1.61-1.70(m, 6H), 2.58(s, 3H), 3.42(m, 2H), 3.67(m, 2H), 7.09-7.75(m, 6H), 9.01(s, 1H), 10.14(s, 1H), 12.42(s, 1H) |
| 251^{a} | 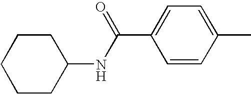 | 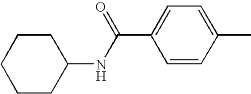 | 1.10-1.90(m, 10H), 2.40(s, 3H), 3.85(m, 1H), 6.83(s, 1H), 7.21(s, 1H), 7.68-7.77(m, 4H), 8.92(s, 1H) |
| 252 | 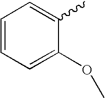 | 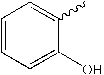 | 2.42(s, 3H), 6.85-7.25(m, 6H), 8.80(s, 1H), 9.36(s, 1H), 9.59(s, 1H), 12.30(s, 1H). |
| 253 | 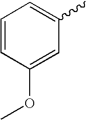 | 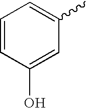 | 2.41(s, 3H), 7.02-7.29(m, 6H), 8.91(s, 1H), 9.48(s, 1H), 9.92(s, 1H), 12.34(s, 1H) |
| 254 | 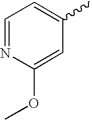 | 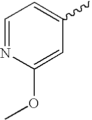 | 2.39(s, 3H), 3.91(s, 3H), 6.93-7.31(m, 3H), 7.95(d, J=8.4Hz, 1H), 8.40(s, 1H), 8.92(s, 1H), 10.12(s, 1H), 12.37(s, 1H). |
| 255 | 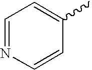 | 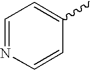 | 2.43(s, 3H), 7.15(s, 1H), 7.38(s, 1H), 7.98(m, 2H), 8.80(m, 2H), 9.10(s, 1H), 10.60(s, 1H), 12.45(s, 1H) |
| 256 | 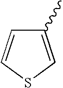 | 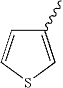 | 2.45(s, 3H), 7.05(s, 1H), 7.64(s, 1H), 7.80(d, J=4.8Hz, 1H), 8.23(d, J=4.8Hz, 1H), 9.47(s, 1H), 9.73(s, br, 1H), 12.45(s, 1H). |
| 257 | 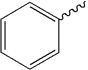 | 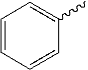 | 2.40(s, 3H), 7.03-7.63(m, 7H), 8.93(s, 1H), 9.98(s, 1H), 12.35(s, 1H). |
| 258 | 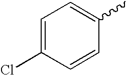 | 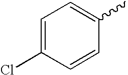 | 2.40(s, 3H), 7.04-7.66(m, 6H), 8.93(s, 1H), 10.11(s, 1H), 12.37(s, 1H). |
| 259 | 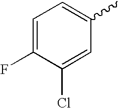 | 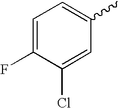 | 2.41(s, 3H), 7.06-7.81(m, 5H), 8.92(s, 1H), 10.20(s, 1H), 12.40(s, 1H). |
| 260 | 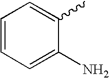 | 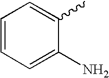 | 2.40(s, 3H), 7.00-7.40(m, 6H), 8.80(s, 1H), 12.40(s, br, 1H). |
| 261 | 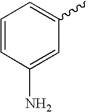 | 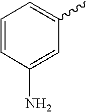 | 2.42(s, 3H), 7.05-7.50(m, 6H), 8.94(s, 1H), 10.13(s, br, 1H), 12.38(s, 1H). |
| 262 | 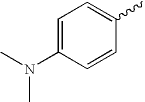 | 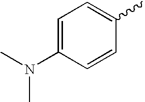 | 2.35(s, 3H), 3.04(s, 6H), 6.90-7.56(m, 6H), 8.92(s, 1H), 9.86(s, br, 1H), 12.31(s, 1H). |
| 263 | 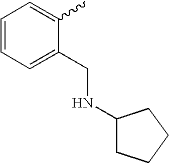 | 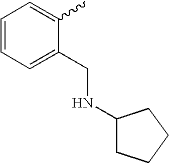 | 1.42-1.77(m, 8H), 2.44(s, 3H), 3.90(m, 1H), 4.12(s, 2H), 7.09-7.70(m, 6H), 8.60(s, br, 1H), 8.81m(s, 1H), 10.27(s, 1H), 12.39(s, 1H). |
| 264 | 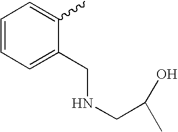 | 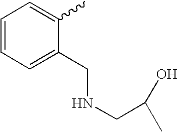 | 1.08(m, 3H), 2.51(s, 3H), 2.80(m ,2H), 3.65-4.20(m, 3H), 7.10-7.72(m, 6H), 8.80(m, 2H), 10.25(s, 1H), 12.40(s, 1H) |
| 265 | 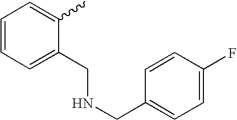 | 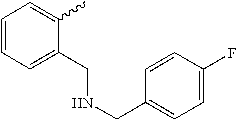 | 2.43(s, 3H), 3.18(s, 2H), 4.09(s, 2H), 7.10-7.70(m, 10H), 9.10(s, br, 1H), 10.10(s, 1H), 12.39(s, 1H) |
| 266 | 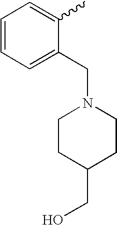 | 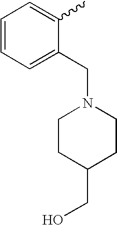 | 1.10-1.80(m, 7H), 2.44(s, 3H), 2.50-2.80(m, 4H), 4.00(m, 1H), 4.35(m, 1H), 7.10-7.75(m, 6H), 8.74(s, 1H), 10.14(s, 1H), 12.40(s, 1H). |
| 267 | 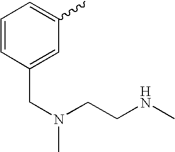 | 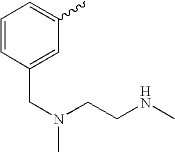 | 2.42(s, 3H), 2.60(s, 6H), 2.85(m, 2H), 3.30(m, 2H), 4.40(s, 2H), 7.06(s, 1H), 7.35(s, 1H), 7.52-7.76(m, 4H), 8.66(s, br, 1H), 8.95(s, 1H), 10.12(s, br, 1H), 12.38(s, 1H) |
| 268 | 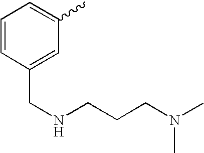 | 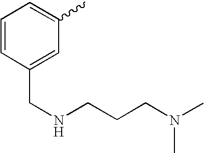 | 2.01(m, 2H), 2.50(s, 3H), 2.78(s, 6H), 3.04(m, 2H), 3.13(m, 2H), 4.27(s, 2H), 7.06-7.74(m, 6H), 8.94(s, s, 2H), 10.11(s, 1H), 12.38(s, 1H) |
| 269 | 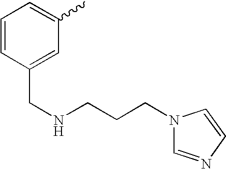 | 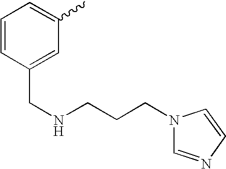 | 2.20(m, J=6.8Hz, 2H), 2.45(s, 3H), 2.99(m, 2H), 4.29(m, 4H), 7.06(s, 1H), 7.49(s, 1H), 7.51-7.77(m, 6H), 9.00(s, 1H), 9.05(s, br, 1H), 9.10(s, 1H), 10.14(s, 1H), 12.38(s, 1H) |
| 270 | 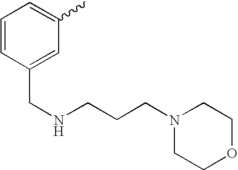 | 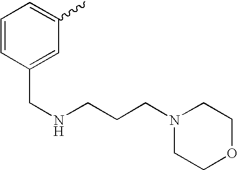 | 2.10(m, 2H), 2.42(s, 3H), 3.04(m, 4H), 3.17(m, 4H), 3.75(m, 4H), 4.05(m, 2H), 4.28(s, 2H), 7.07-7.74(m, 6H), 8.94(s, s, 2H), 10.12(s, 1H), 12.39(s, 1H) |
| 271 | 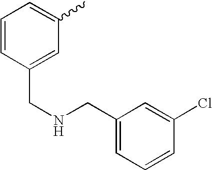 | 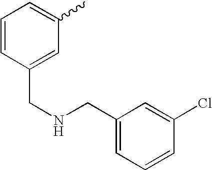 | 2.38(s, 3H), 4.28(s, 4H), 7.05-7.80(m, 10H), 8.95(s, 1H), 9.31(s, br, 1H), 10.10(s, 1H), 12.38(s, 1H). |
| 272 | 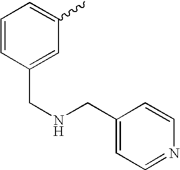 | 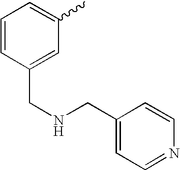 | 2.42(s, 3H), 4.33(s, 4H), 7.06-7.76(m, 8H), 8.70(m, 2H), 8.95(s, 1H), 9.45(s, br, 1H), 10.08(s, 1H), 12.38(s, 1H). |

^{b}MeOD as solvent

### Example 273-289

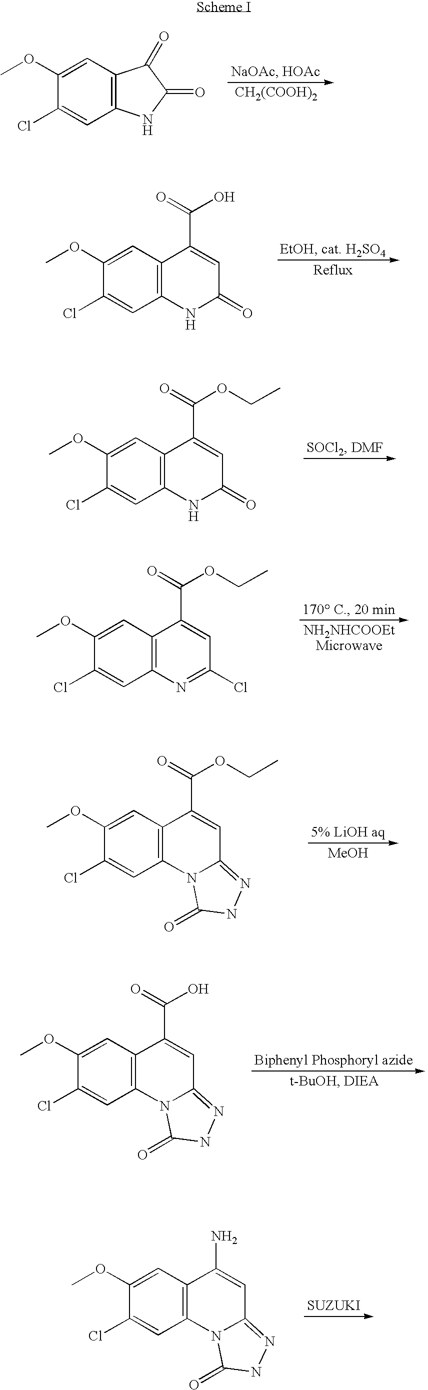

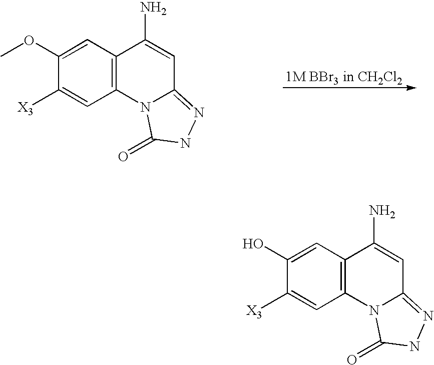

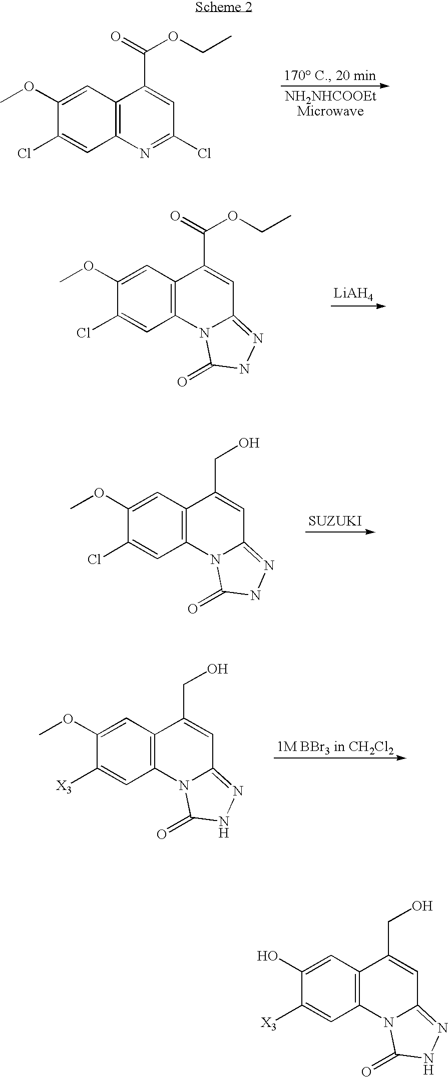

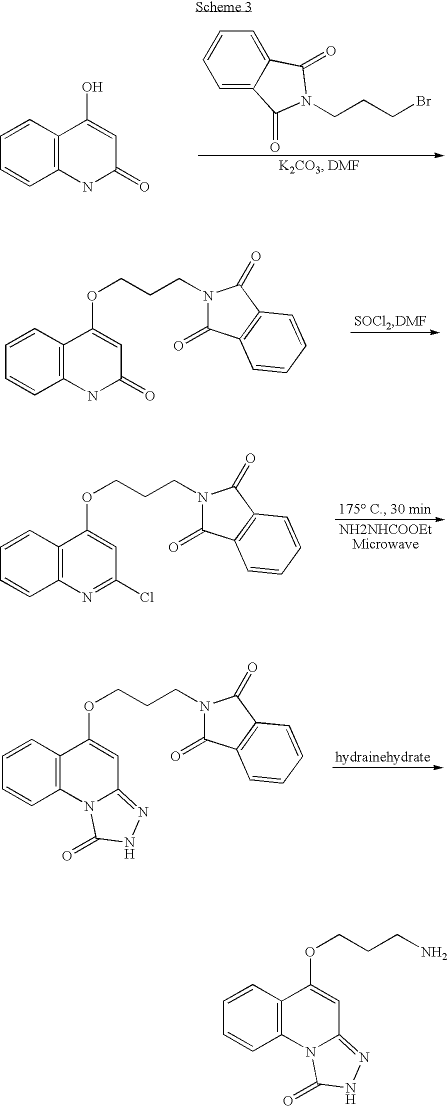

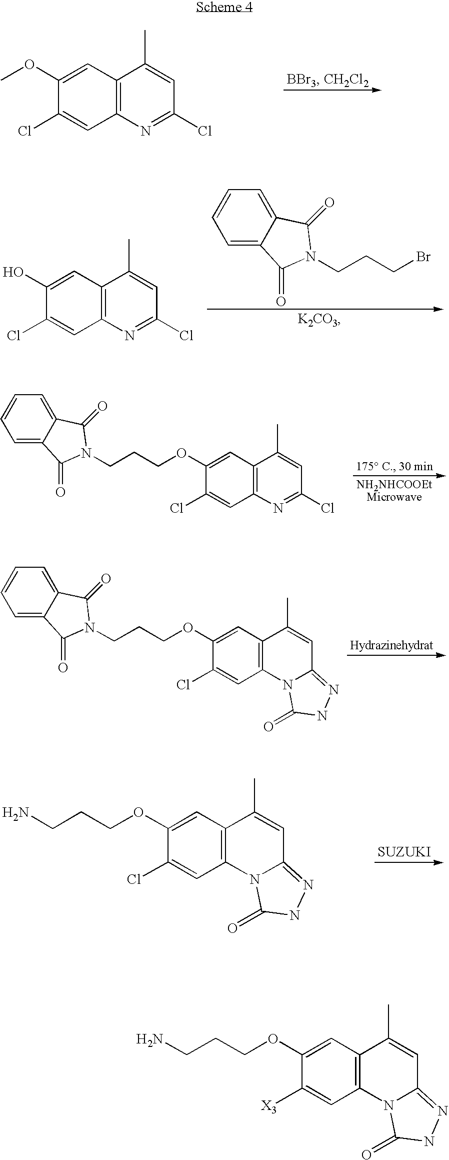

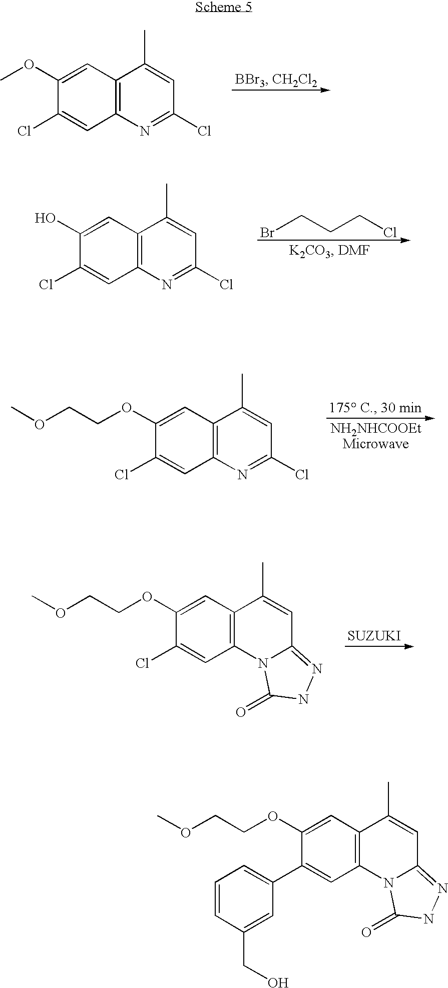

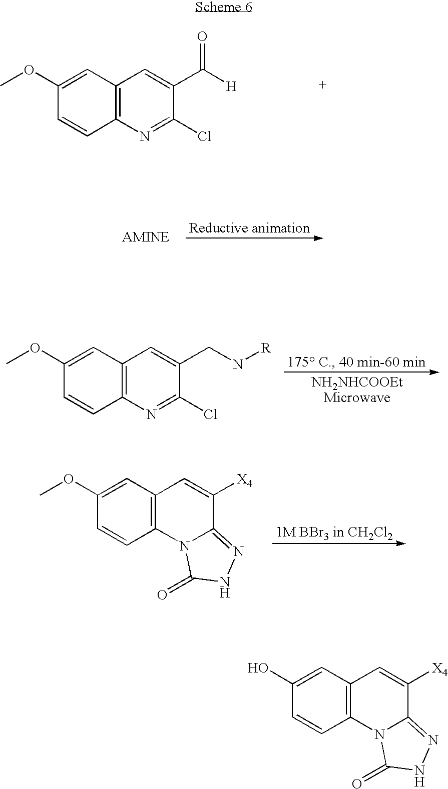

The following examples were prepared as described in Schemes 1-6 above:

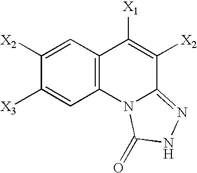

| Ex. | X1 | X2 | X3 | X4 | 1HNMR |
|---|---|---|---|---|---|
| 273 | 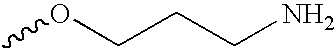 | H | H | H | 1.90(m, 2H), 2.76(m, 2H), 4.23(m, 2H), 6.50-7.66(m, 3H), 7.93(d, J=7.2Hz, 1H), 8.92(d, J=8.0Hz, 1H). |
| 274 | 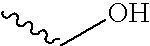 | OMe | Cl | H | 3.97(s, 3H), 4.78(s, 2H), 5.62(s, br, 1H), 7.15(s, 1H), 7.35(s, 1H), 9.01(s, 1H), 12.53(s, 1H) |
| 275 | 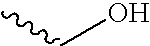 | OH | Cl | H | 4.65(s, 2H), 5.53(s, br, 1H), 7.10(s, 1H), 7.32(s, 1H), 8.94(s, 1H), 10.54(s, 1H), 12.49(s, 1H). |
| 276 | 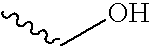 | OMe | 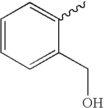 | H | 3.81(s, 3H), 4.30(m, 2H), 4.83(s, 2H), 5.02(s, br, 1H), 5.60(s, br, 1H), 7.16-7.60(m, 6H), 8.76(s, 1H), 12.44(s, 1H). |
| 277 | 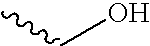 | OMe | 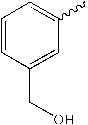 | H | 3.87(s, 3H), 4.60(d, 2H), 4.85(d, 2H), 5.25(t, 1H), 5.63(t, 1H), 7.15-7.48(m, 6H), 8.99(s, 1H), 12.45(s, 1H). |
| 278 | 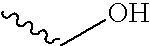 | OMe | 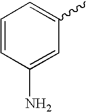 | H | 3.88(s, 3H), 4.82(s, 2H), 7.01-7.38(m, 6H), 8.95(s, 1H), 12.45(s, 1H). |
| 279 | 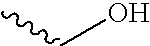 | OH | 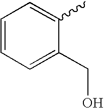 | H | 4.38(s, br, 2H), 4.71(s, 2H), 7.10-7.60(m, 6H), 8.70(s, 1H), 9.85(s, 1H), 12.39(s, 1H). |
| 280 | 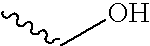 | OH | 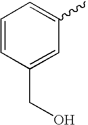 | H | 4.58(s, 2H), 4.70(s, H), 7.08-7.55(m, 6H), 8.92(s, 1H), 9.90(s, 1H), 12.35(s, 1H). |
| 281 | 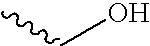 | OH | 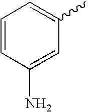 | H | 4.69(s, 2H), 7.05-7.35(m, 6H), 8.94(s, 1H), 10.01(s, 1H), 12.41(s, 1H). |
| 282 | 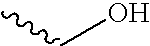 | OMe | 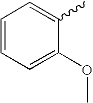 | H | 3.71(s, 3H), 3.81(s, 3H), 4.82(s, 2H), 5.50(s, br, 1H), 7.02-7.39(m, 6H), 8.78(s, 1H), 12.42(s, 1H). |
| 283 | 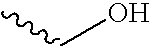 | OH | 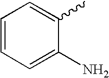 | H | 4.71(s, 2H), 6.90-7.31(m, 6H), 8.82(s, 1H), 12.42(s, 1H) |
| 284 | NH2 | OMe | 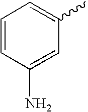 | H | 3.89(s, 3H), 6.00(s, 1H), 7.01-7.64(m, 6H), 8.97(s, 1H), 11.90 9s, 1H). |
| 285 | NH2 | OH | Cl | H | 6.40(s, br, 2H), 7.63(s, 1H), |
| | | | | | 9.96(s, 1H), 10.40(s, 1H), 12.09(s, |
| | | | | | 1H). |
| 286 | NH2 | OH | 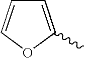 | H | 6.90-7.50(m, 5H), 8.60(s, 1H), 13.18(s, 1H) |
| 287 | NH2 | OH | 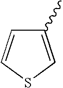 | H | 6.65(s, 1H), 7.82(d, 1H), 8.20(s, 1H), 8.29(d, 1H), 9.55(s, 1H), 9.80(s, br, 1H), 12.15(s, 1H). |
| 288 | H | OH | H | 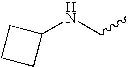 | 1.24(m, 2H), 1.76(m, 4H), 3.82(m, 1H), 4.10(s, 2H), 7.12(d, J=7.2Hz, 2H), 7.63(s, 1H), 8.74(d, J=9.6Hz, 1H), 9.16(s, br, 1H), 9.93(s, br, 1H), 12.71(s, 1H). |
| 289 | H | OH | H | 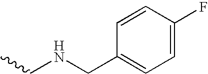 | 4.24, 4.31(s, s, 4H), 7.01-7.61(m, 7H), 8.74(t, J=4.8Hz, 1H), 9.32(s, br, 1H), 9.92(s, 1H), 12.68(s, 1H). |

### Examples 290-291

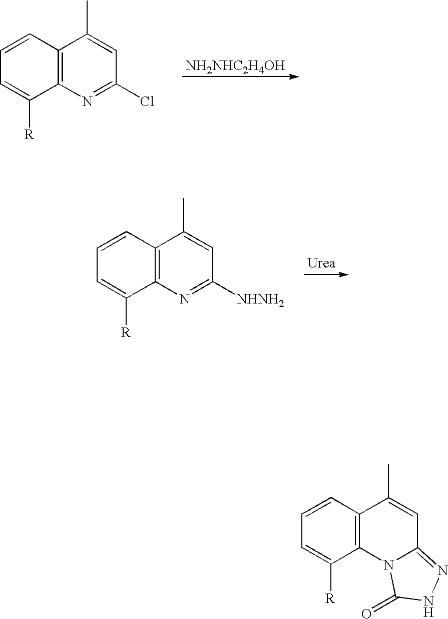

### 5-Methyl[1,2,4]triazolo[4,3a]quinolin(2H)one

### 5,9-Dimethyl[1,2,4]triazolo[4,3a]quinolin(2H)one

A solution of the chloroquinoline (0.02 mol) and NH₂NHC₂H₄OH (0.02 mol) in cellusolve (10 ml) was heated to reflux for four hours. Ether was added and the resultant precipitate removed by filtration. The crude solid was recrystallized from ethanol to yield the intermediate hydrazine.

A solution of the intermediate hydrazine (0.01 mol) and urea (0.01 mol) in DMF (10 ml) was heated to reflux for two hours. The solution was cooled and the resultant solid filtered off and recrystallized from DMF to give the pure triazolone.

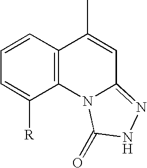

| Ex. | R— | Mpt |
|---|---|---|
| 290 | H | 275° C. |
| 291 | Me | 220° C. |

### Examples 292-293

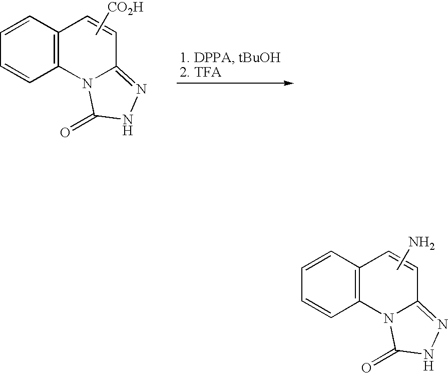

### 4-Amino-2H-[1,2,4]triazolo[4,3-α]quinolin(2H)one

### 5-Amino-2H-[1,2,4]triazolo[4,3-α]quinolin(2H)one

A mixture of the starting carboxylic acid (0.2 mmol), diphenylphosphoryl azide (0.22 mmol), diisopropylethylamine (0.22 mmol) in t-butanol (1 ml) was heated at 80° C. for six hours. Excess t-butanol was removed in vacuo and the residue suspended in CH₂Cl₂/MeOH. The solid was removed by filtration, the filtrate evaporated and purified by silica gel chromatography.

The BOC protected amine (0.1 mmol) was suspended in CH₂Cl₂ (0.5 ml) and trifluoroacetic acid (0.5 ml) added. The resultant mixture was stirred at room temperature for two hours. The reaction mixture was evaporated and the residue triturated to give a solid. This solid was filtered off and dried under high vacuum to give the desired amine.

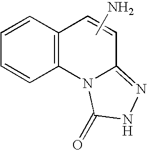

| Ex. | R— | 1H NMR(400 MHz, DMSO-d6) |
|---|---|---|
| 292 | 4-NH₂ | 5.73(s, 2H), 6.40(s, 1H), 7.19-7.31(m, 1H), |
| | | 7.40-7.50(m, 1H), 8.76-8.84(m, 1H), 12.50(s, 1H) |
| 293 | 5-NH₂ | 5.94(s, 1H), 7.41(t, 7H), 7.60(t, 1H), 7.98(d, 1H), |
| | | 8.98(d, 1H) |

### Examples 294-296

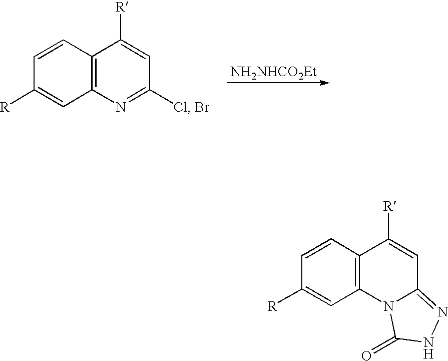

### [1,2,4]triazolo[4,3a]quinolin(2H)one

### 8-Methoxy[1,2,4]triazolo[4,3a]quinolin(2H)one

### 8-Fluoro[1,2,4]triazolo[4,3a]quinolin(2H)one

The starting haloquinoline was dissolved in NMP (1.9 ml) in a 20×125 reaction tube. A catalytic quantity of HC₁ (4M in dioxane) was added and the reactions heated in a block at 135° C. until complete as determined by LC MS.

The mixtures were cooled and the precipitated product removed by filtration. If necessary the product was purified by chromatography.

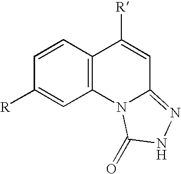

| Ex. | R— | R′— | LC MS |
|---|---|---|---|
| 294 | H | H | 186 (M + H)⁺ |
| 295 | OMe | Me | 230 (M + H)⁺ |
| 296 | F | Me | 218 (M + H)⁺ |

### Examples 297-332

The following examples were prepared via Suzuki (previously described), Sonogashira or Stille coupling as appropriate:

A typical procedure for Sonogashira coupling is as outlined below: 8-bromo-5-methyl{1,2[4}triazolo[4,3-a]quinolin-1(2H)-one, (100 mg, 0.36 mmol), dichloro bistriphenyl phosphine palladium (13 mg, 0.018 mmol), copper iodide (3.5 mg, 0.018 mmol) were dissolved in dry THF (1 mL), triethylamine(0.15 mL, 1.08 mmol), alkyne (0.54 mmol) were added, the solution was degassed for 5 minutes, then heated up at 60° C. under argon for 2.5 hours. The crude product was purified by prep. HPLC to obtain 6% and 35% of the desired product.

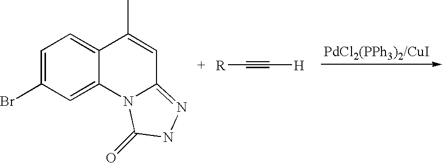

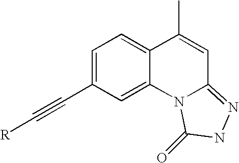

| | | | MS |
|---|---|---|---|
| EX | R | ¹HNMR | (MH+) |
| 297 | 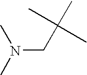 | 2.45(s, 3H), 2.91(s, 6H), 4.39(s, 2H), 7.13(s, 1H), 7.63(d, 1H), 7.86(d, 1H), 9.10(s, 1H) | 281 |
| 298 | 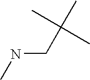 | 2.45(s, 3H), 2.69(bs, 3H), 4.22(bs, 2H), 7.12(s, 1H), 7.57(d, 1H), 7.86(d, 1H), 9.10(s, 1H) | 267 |
| 299 | 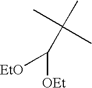 | 0.95(t, 6H), 2.20(s, 3H), 3.39(q, 2H), 3.47(q, 2H), 5.36(s, 1H), 6.86(s, 1H), 7.33(d, 1H), 7.58(d, 1H), 8.78(s, 1H) | 326 |

Examples using the Stille coupling procedure were prepared as follows: The appropriate triazolone (1 eq) was placed in a microwave tube containing a stir bar and the desired stannane (1.5 eq) was added together with palladium tetrakistriphenylphosphine (7 mol %) and dioxane (3 mL). A few grains of NaCl were added and the contents were heated in a Smith Synthesizer (microwave) for 1800 secs at 140° C. followed by 1200 secs at 165° C. The desired product was isolated by HPLC purification (5-20%).

| | | |
|---|---|---|
| 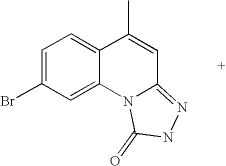 | 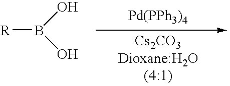 | 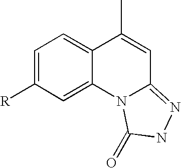 |

| EX | R | ¹HNMR | MS(MH+) |
|---|---|---|---|
| 300 | 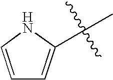 | 2.32(s, 3H), 6.13(m, 1H), 6.51(s, 1H), 6.88(s, 2H), 7.68(m, 2H), 9.12(s, 1H), 11.51(s, 1H) | 265 |
| 301 | 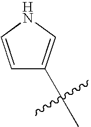 | 2.43(m, 5H), 6.48(t, 1H), 6.90(m, 21H), 7.32(sm, 1H), 7.69(d + d, 2H), 9.13(s, 1H) | 265 |
| 302 | 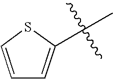 | 2.47(s, 3H), 6.98(s, 1H), 7.20(m, 1H), 7.61(m, 2H), 7.80(s, 2H), 9.28(s, 1H) | 282 |
| 303 | 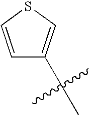 | 2.46(s, 3H), 6.56(br s, 1H), 7.01(s, 1H), 7.60(d, 1H), 7.76(d, 1H), 7.84(s, 2H), 7.99(d, 1H), 9.30(s, 1H) | 282 |
| 304 | 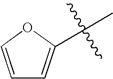 | 2.46(s, 3H), 6.68(m, 1H), 7.02(s, 1H), 7.11(d, 1H), 7.82(m, 3H), 9.30(s, 1H) | 266 |
| 305 | 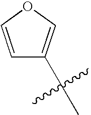 | 2.45(s, 3H), 6.94(s, 1H), 7.00(s, 1H), 7.73(d, 1H), 7.83(m, 2H), 8.29(s, 1H), 9.15(s, 1H) | 266 |
| 306 | 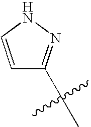 | 2.43(s, 3H), 6.93(s, 1H), 7.74(m, 2H), 8.12(s, 2H), 9.18(s, 1H) | 266 |
| 307 | 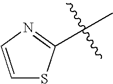 | 2.46(s, 3H), 6.55(br s, 1H), 7.20(s, 1H), 8.0(m, 2H), 8.10(m, 2H), 9.64(s, 1H) | 283 |
| 308 | 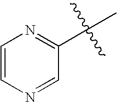 | 2.50(s, 3H), 6.54(s, 1H), 7.12(s, 1H), 7.94(d, 1H), 8.24(m, 1H), 8.70(m, 1H), 8.82(m, 1H), 9.33(s, 1H), 9.78(s, 1H) | 278 |
| 309 | 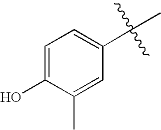 | 2.2(s, 3H), 2.4(s, 3H), 6.8(d, 1H), 7.0(s, 1H), 7.4(d, 1H), 7.5(s, 1H), 7.7(d, 1H), 7.9(d, 1H), 9.25(s, 1H), 9.7(s, 1H) | 306 |
| 310 | 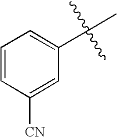 | 2.4(s, 3H), 7.1(s, 1H), 7.8(m, 1H), 7.9(m, 3 H), 8.0 (m, 1H), 8.2(s, 1H), 9.3(s, 1H), 12.5(s, 1H) | 301 |
| 311 | 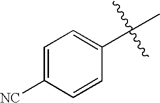 | 2.4(s, 3H), 6.5(s, 1H), 7.2(s, 1H), 7.9(m, 5H), 9.4(s, 1H), 12.6(s, 1H) | 301 |
| 312 | 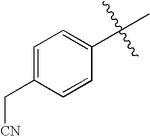 | 2.4(s, 3H), 4.1(s, 3H), 6.6(s, 1H), 7.0(s, 1H), 7.5(d, 2H), 7.8(m, 3H), 7.9(d, 1H), 9.3(s, 1H) | 315 |
| 313 | 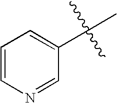 | 2.4(s, 3H), 7.0(s, 1H), 7.7(m, 1H), 7.9(m, 2H), 8.2 (m, 1H), 8.7(d, 1H), 8.9(s, 1H), 9.3(s, 1H) | 277 |
| 314 | 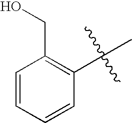 | 2.4(s, 3H), 6.9(1H, s), 7.25(d, 1H), 7.4-7.6(m, 4H), 7.7(d, 1H), 7.85(d, 1H), 8.9(s, 1H) | 306 |
| 315 | 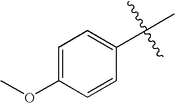 | 2.4(s, 3H), 3.8(s, 3H), 7.0(s, 1H), 7.1(d, 1H), 7.7 (m, 3H), 7.9(d, 1H), 9.4(s, 1H) | 306 |
| 316 | 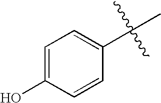 | 2.4(s, 3H), 6.5(bs, 1H0, 6.9(m, 3H), 7.7(d, 2H), 7.8 (d, 1H), 7.9(d, 1H), 9.2(s, 1H), 9.8(bs, 1H) | 292 |
| 317 | 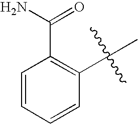 | 2.4(s, 3H), 7.0(s, 1H), 7.3(s, 1H), 7.5(m, 4H), 7.8 (m, 2H), 9.1(s, 1H) | 319 |
| 318 | 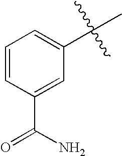 | 2.4(s, 3H), 6.9(s, 1H), 7.5(s, 1H), 7.55-7.9(m, 4H), 8.1(s, 1H), 8.2(s, 1H), 9.5(s, 1H) | 319 |
| 319 | 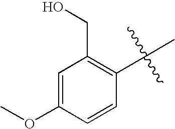 | 2.4(s, 3H), 4.5(s, 3H), 6.8(m, 1H), 7.05(s, 1H), 7.25 (m, 3H), 7.5(d, 1H), 7.8(d, 1H), 8.8(s, 1H) | 336 |
| 320 | 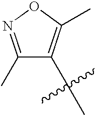 | 2.4(s, 3H), 2.5(s, 6H), 7.05(s, 1H), 7.5(d, 1H) 7.09 (d, 1H), 9(s, 1H) | 295 |
| 321 | 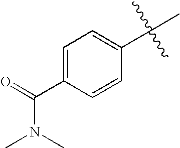 | 2.38(s, 3H), 2.48(s, 3H), 3.00(s, 3H), 6.92(s, 1H), 7.56(d, 2H) 7.72(m, 4H), 9.50(s, 1H) | 347 |
| 322 | 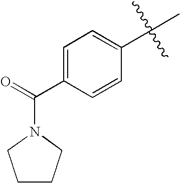 | 1.85(t, 4H), 2.34(s, 3H), 3.42(t, 4H), 6.92(s, 1H), 7.65(m, 6H), 9.56(s, 1H) | 373 |
| 323 | 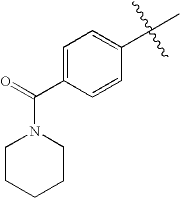 | 1.50 (m, 6H), 2.45 (s, 3H), 3.32(m, 2H), 3.60(m, 2H), 7.02(s, 1H), 7.50 (d, 2H) 7.80(m, 3H), 9.35(s, 1H) | 387 |
| 324 | 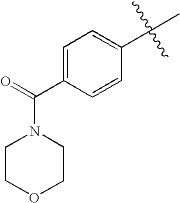 | 2.48(s, 3H), 3.62 (m, 8H), 7.02(s, 1H), 7.56(d, 2H) 7.84(m, 4H), 9.28 (s, 1H) | 389 |
| 325 | 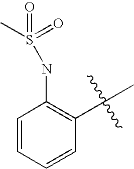 | 2.48(s, 3H), 2.90(s, 3H), 7.05(s, 1H), 7.45(m, 5H) 7.90(d, 1H), 8.98(s, 1H), 9.05(s, 1H) | 369 |
| 326 | 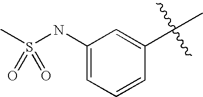 | 2.48(s, 3H), 3.00(s, 3H), 7.05(s, 1H), 7.28(d, 1H) 7.50(m, 3H), 7.71(d, 1H), 7.90(d, 1H), 9.28(s, 1H) | 369 |
| 327 | 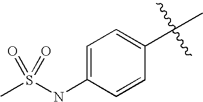 | 2.48(s, 3H), 3.02(s, 3H), 7.02(s, 1H), 7.35(d, 2H) 7.70(m, 3H), 7.88(d, 1H), 9.28(s, 1H) | 369 |
| 328 | 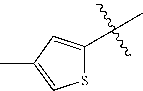 | 2.20(s, 3H), 2.38(s, 3H), 6.90(s, 1H), 7.18(s, 1H), 7.40(s, 1H) 7.68(d, 1H), 7.74(d, 1H), 9.18(s, 1H) | 296 |
| 329 | 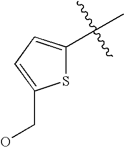 | 2.48(s, 3H), 4.60(d, 2H), 5.52(t, 1H), 6.95(m, 2H), 7.40(S. 1H) 7.70(m, 2H), 9.18(s, 1H) | 312 |
| 330 | 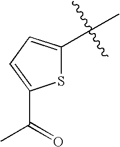 | 2.48(s, 3H), 2.60(s, 3H), 7.05(s, 1H), 7.75(d, 1H) 7.90(m, 2H), 8.00(d, 1H), 9.35(s, 1H) | 324 |
| 331 | 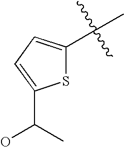 | 1.38(d, 3H), 2.48(s, 3H), 4.90(q, 1H), 6.96(m, 2H), 7.40(s, 1H) 7.65(m, 2H), 9.18(s, 1H) | 326 |
| 332 | 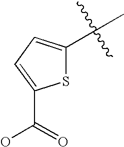 | 2.48(s, 3H), 7.02(s, 1H), 7.60(d, 1H) 7.74(d, 1H), 7.82(m, 1H), 9.28(s, 1H) | 326 |

### Examples 333-339

Fused triazolones with alkoxy substituents were generated from alkylation of the boronate ester of phenol using the appropriate alkyl chloride (1.1 eq) and heating the reactants in DMF in the presence of cesium carbonate (1.1 eq). The alkylated methyleneaminophenyl substituted triazolones were synthesized via amination of bromomethylphenylboronic acid with the appropriate amine (2M in THF) at reflux (2 h to overnight) to yield the corresponding aminated boronic acid.

The alkylated boronic acids prepared as described above were used to synthesize the following examples using the Suzuki coupling conditions previously described.

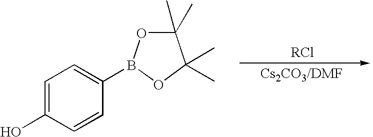

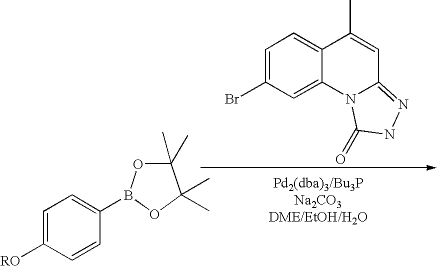

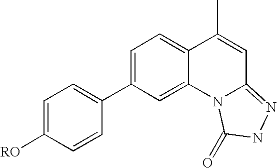

| EX | R | ¹HNMR | MS(MH+) |
|---|---|---|---|
| 333 | 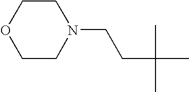 | 4.69(s, 2H), 5.45(bs, 1H), 6.88(s, 1H), 7.01(s, 1H), 7.65(d, 1H), 7.71(d, 1H), 7.77(s, 1H), 8.22(s, 1H), 9.10(s, 1H) | 405 |
| 334 | 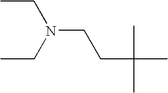 | 1.25(t, 6H), 2.47(s, 3H), 3.26(q, 4H), 3.57(t, 2H), 4.40(t, 2H), 7.03(s, 1H), 7.19(d, 2H), 7.76(m, 3H), 7.87(d, 1H), 9.27(s, 1H) | 391 |

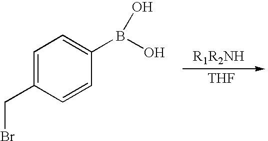

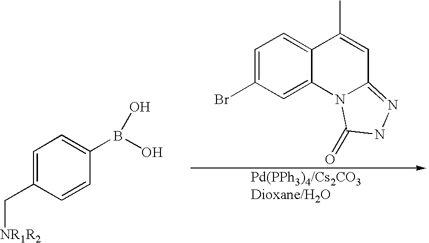

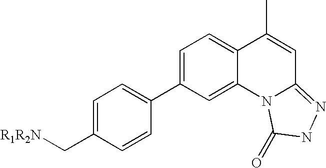

| EX | NR1R2 | ¹HNMR(DMSO-d6) | MS(MH+) |
|---|---|---|---|
| 335 | 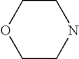 | 2.43(s, 3H), 3.15(t, 3H), 3.24(t, 1H), 3.57(t, 1H), 3.89(t, 1H), 4.37(s, 2H), 7.02(s, 1H), 7.61(d, 2H), 7.79(m, 3H), 7.86(d, 1H), 9.26(s, 1H) | 375 |
| 336 | 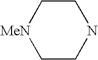 | 2.26(s, 3H), 2.55(bs, 3H), 2.80(m, 2H), 3.14(m, 2H), 3.49(m, 6H), 6.83(s, 1H), 7.28(d, 2H), 7.53(m, 3H), 7.66(d, 1H), 9.07(s, 1H) | 388 |
| 337 | CH₃NH | 2.31(s, 3H), 2.42(t, 3H), 4.02(t, 2H), | 319 |
| | | 6.88(s, 1H), 7.45(d, 2H), 7.63(m, 3H), | |
| | | 7.72(d, 1H), 9.13(s, 1H) | |
| 338 | 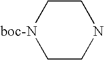 | 1.42(s, 9H), 2.49(s, 3H), 3.10(m, 4H), 3.72(m, 2H), 4.08(m, 2H), 4.82(s, 2H), 7.08(s, 1H), 7.67(d, 2H), 7.84(m, 3H), 7.92(d, 1H), 9.32(s, 1H) | 474 |
| 339 | 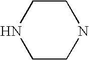 | 2.46(s, 3H), 3.57(m, 8H), 4.45(s, 2H), 7.07(s, 1H), 7.82(m, 4H), 7.90(d, 1H), 9.31(s, 1H) | 374 |

8-bromo-5-bromomethyl[1,2[4]triazolo[4,3-a]quinolin-1(2H)-one was synthesized using the following procedure:

8-bromo-5-hydroxymethyl[1,2[4]triazolo[4,3-a]quinolin-1(2H)-one (5.92 mmols, 1.5 g) was suspended in DMF (30 mL) and CBr4 (7.11 mmols, 1.2 eq, 2.36 g) and Ph₃P (7.11 mmols, 1.2 eq., 1.86 g) added. The resultant mixture was heated with stirring at 80 deg and the reaction progress monitored by LC-MS. After heating for 4 h, a further 0.6 eq. each of CBr4 (0.98 g) and Ph₃P (0.78 g) were added and heating continued until complete disappearance of the starting alcohol was observed. The reaction mixture was cooled to room temperature and the precipitated product was filtered off, washed with methanol followed by DCM followed by hexanes to give the required product as a light gray powder (55-58%).

### Examples 340-364

5-formyl 3-thiophene boronic acid (100 mg, 0.64 mmol) was dissolved in DME (3 ml), and the appropriate amine (3.2 mmol) was added, followed by a drop of HOAc. The resulting solution was stirred for 5 minutes at room temperature. Sodium triacetoxyborohydride (271 mg, 1.28 mmol) was added and the resulting solution was heated at 60° C. for 5 hours. The solvent and excess amine were evaporated under vacuum and the crude product used without further purification to couple with the appropriate 8-bromotriazolone as previously described.

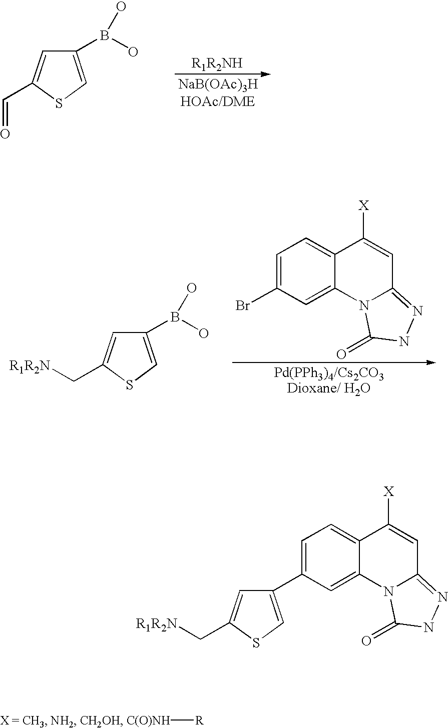

For the following examples X=methyl

| EX | R1R2N | ¹HNMR(DMSO-d6) | MS(MH+) |
|---|---|---|---|
| 340 | 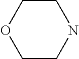 | 2.47(s, 3H), 3.15(bs, 4H), 3.30(bs, 4H), 4.68(s 2H), 7.05(s, 1H), 7.79(s, 1H), 7.86(m, 2H), 8.07(s, 13H), 8.20(s, 1H), 9.30(s, 1H) | 422 |
| 341 | CH₃NH | 2.47(s, 3H), 2.62(bs, 2H), 4.44(bs, 2H), | 325 |
| | | 7.05(s, 1H), 7.757(s, 1H), 7.85(m, 2H), | |
| | | 8.11(s, 1H), 9.29(s, 1H) | |
| 342 | 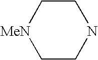 | 2.46(m, 5H), 2.80(s, 34H), 3.05(m, 4H), 3.40(m, 2H), 3.90(s, 2H), 7.03(s, 1H), 7.47(s, 1H), 7.83(m, 2H), 7.95(s, 1H), 9.26(s, 1H) | 394 |
| 343 | 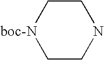 | 1.41(s, 9H), 2.47(s, 3H), 3.05(m, 2H), 3.43(m, 4H), 4.03(m, 2H), 4.65(s, 2H), 7.05(s, 1H), 7.86(m, 3H), 8.17(s, 1H), 9.22(s, 1H) | 480 |
| 344 | 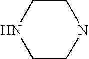 | 2.47(s, 3H), 3.46(m, 8H), 4.72(s, 2H), 7.03(s, 1H), 7.85(m, 3H), 8.16(s, 1H), 9.28(s, 1H) | 380 |
| 345 | 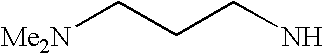 | 1.90(m, 2H), 2.47(s, 3H), 2.78(s, 6H), 3.11(m, 4H), 4.50(bs, 2H), 7.05(s, 1H), 7.77(s, 1H), 7.84(m, 2H), 8.13(s, 1H), 9.30(s, 1H) | 396 |
| 346 | EtNH | 1.15(t, 3H), 2.39(s, 3H), 2.94(m, 2H), | 339 |
| | | 4.37(bs, 2H), 6.99(s, 1H), 7.26(d, 1H), | |
| | | 7.56(d, 1H), 7.79(m, 2H), 9.22(s, 1H) | |
| 347 | 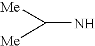 | 1.53(d, 6H), 2.69(s, 3H), 3.59(m, 1H), 4.69(bs, 2H), 7.29(s, 1H), 7.59(d, 1H), 7.86(d, 1H), 8.08(m, 2H), 9.52(s, 1H) | 353 |

For the following compounds X=—CH₂OH:

| EX | R₁R₂N | NMR(DMSO-d6) | MS(MH+) |
|---|---|---|---|
| 348 | 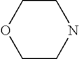 | 3.15(m, 2H), 3.47(m, 4H), 3.98(m, 2H), 4.70(d, 2H), 4.76(s, 2H), 7.11(s, 1H), 7.83(m, 3H), 8.16(s, 1H), 9.31(s, 1H) | 397 |
| 349 | 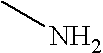 | 2.62(s, 3H), 4.44(bs, 2H), 4.76(s, 2H), 7.11(s, 1H), 7.75(s, 1H), 7.83(m, 2H), 8.11(s, 1H), 9.31(s, 1H) | 341 |
| 350 | 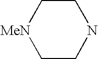 | 2.80(s, 3H), 3.05(m, 4H), 3.39(m, 2H), 3.90(bs, 2H), 4.76(s, 2H), 7.09(s, 1H), 7.47(s, 1H), 7.803(m, 23H), 7.95(s, 1H), 9.27(s, 1H) | 410 |
| 351 | 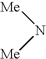 | 2.80(sm, 6H), 4.60(bs, 2H), 4.76(s, 2H), 7.11(s, 1H), 7.83(m, 3H), 8.18(s, 1H), 9.31(s, 1H) | 355 |
| 352 | 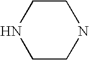 | 3.57(m, 8H), 4.68(s, 2H), 4.76(s, 2H), 7.10(s, 1H), 7.83(m, 3H), 8.15(s, 1H), 9.29(s, 1H) | 396 |
| 353 | 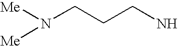 | 2.00(m, 2H), 2.78(s, 6H), 3.11(m, 2H), 3.13(m,2H),4.49(s, 2H), 4.76(s, 2H), 7.11(s, 1H), 7.76(s, 1H), 7.83(m, 2H), 8.13(s, 1H), 9.31(s, 1H), | 412 |

For the following compounds X=NH2:

| EX | R₁R₂N | ¹HNMR | MS(MH+) |
|---|---|---|---|
| 354 | 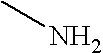 | 2.62(bs, 3H), 4.42(bs, 2H), 5.95(s, 1H), 7.76(m, 2H), 8.05(d, 1H), 8.11(s, 1H), 9.32(s, 1H) | 326 |
| 355 | 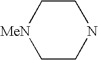 | 2.81(s, 3H), 357(m, 8H), 4.55(s, 2H), 5.96(s, 1H), 7.80(m, 2H), 8.05(d, 1H), 8.12(s, 1H), 9.31(s, 1H) | 395 |

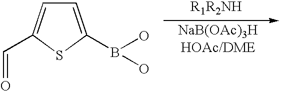

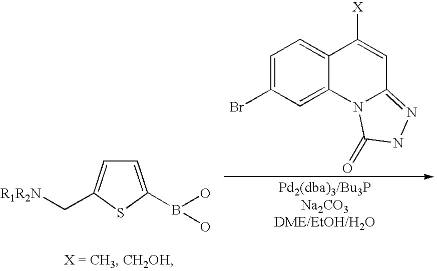

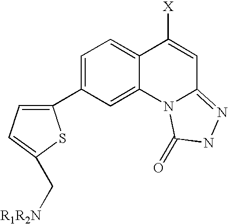

For the following compounds X=methyl

| EX | R₁R₂N | ¹HNMR(DMSO-d6) | MS(MH+) |
|---|---|---|---|
| 356 | 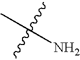 | 2.46(s, 3H), 2.61(bs, 3H), 4.42(bs, 2H), 7.06(s, 1H), 7.32(d, 1H), 7.64(d, 1H), 7.86(m, 2H), 9.28(s, 1H) | 294 |
| 357 | 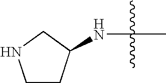 | 2.39(m, 2H), 2.54(s, 3H), 3.34(m, 1H), 3.61(m, 3H), 4.06(m, 1H), 4.60(s, 2H), 7.14(s, 1H), 7.51(d, 1H), 7.72(d, 1H), 7.93(m, 2H), 9.36(s, 1H) | 380 |
| 358 | 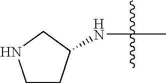 | 2.39(m, 2H), 2.55(s, 3H), 3.33(m, 1H), 3.61(m, 3H), 4.06(m, 1H), 4.61(s, 2H), 7.14(s, 1H), 7.54(d, lH), 7.72(d, 1H), 7.93(m, 2H), 9.36(s, 1H) | 380 |
| 359 | 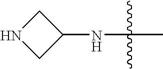 | 2.53(s, 3H), 3.63-4.50(m, 7H), 7.12(s, 1H), 7.39(d, 1H), 7.68(d, 1H), 7.92(m, 2H), 9.31(s, 1H) | 366 |

For the following compounds X=—CH₂OH

| EX | R₁R₂N | ¹HNMR(DMSO-d6) | MS(MH+) |
|---|---|---|---|
| 360 | 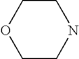 | 3.15-3.64(m, 6H), 3.95(m, 2H), 4.62(bs, 2H), 4.75(s, 2H), 7.12(s, 1H), 7.35(m, 1H), 7.65(m, 1H), 7.83(m, 2H), 9.30(s, 1H) | 397 |
| 361 | 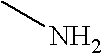 | 2.60(s, 3H), 4.42(s, 2H), 4.75(s, 2H), 7.11(s, 1H), 7.32(d, 1H), 7.62(d, 1H), 7.83(m, 2H), 9.29(s, 1H) | 341 |
| 362 | 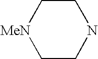 | 2.47(m, 2H), 2.80(s, 3H), 3.08(m, 4H), 3.39(m, 2H), 3.86(s, 2H), 4.74(s, 2H), 7.09(m, 2H), 7.583(d, 1H), 7.79(m, 2H), 9.23(s, 1H) | 410 |
| 363 | 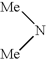 | 2.80(s, 6H), 4.57(s, 2H), 4.76(s, 2H), 7.12(s, 1H), 7.37(d, 1H), 7.68(d, 1H), 7.84(m, 2H), 9.30(s, 1H) | 355 |
| 364 | 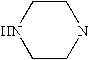 | 3.05(m, 2H), 3.62(m, 2H), 4.53(m, 6H), 4.75(s, 2H), 7.11(s, 1H), 7.48(d, 1H), 7.66(d, 1H), 7.83(m, 2H), 9.29(s, 1H) | 396 |

### Examples 365-373

8-Bromo-5-hydroxymethyl[1,2[4]triazolo[4,3-a]quinolin-1(2H)-one was coupled with the appropriate boronic acid under Suzuki conditions, or reacted with the appropriate stannane under Stille conditions to prepare the examples below:

| EX | R | ¹HNMR(DMSO-d6) | MS(MH+) |
|---|---|---|---|
| 365 | 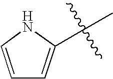 | 4.62(d, 2H), 5.54(t, 1H), 6.19(m, 1H), 6.57(m, 1H), 6.87(s, 1H), 6.96(s, 1H) 7.66(m, 2H), 9.11(s, 1H) 11.55(brs, 1H), 12.3(s, 1H) | 281 |
| 366 | 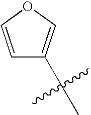 | 4.75(s, 2H), 5.58(br s, 1H), 6.95(s, 1H), 7.10(s, 1H), 7.72(m, 1H), 7.80(m, 2H) 8.29(s, 1H), 9.19(s, 1H) | 282 |
| 367 | 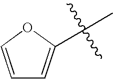 | 4.75(s, 2H), 5.56(br s, 1H), 6.54(br s, 1H), 7.03(s, 1H), 7.73(m, 2H), 8.11(br s 2H), 9.19(s, 1H) | 282 |
| 368 | 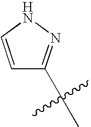 | 4.75(s, 2H), 5.56(brs, 1H), 6.54(brs, 1H), 7.03(s, 1H), 7.73(m, 2H), 8.11(br s, 2H), 9.19(s, 1H) | 282 |
| 369 | 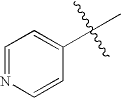 | 4.78(s, 2H), 7.20(s, 1H), 7.95(m, 4H), 8.82(d, 2H), 9.43(s, 1H) | 293 |
| 370 | 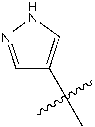 | 4.75(s, 2H), 5.56(brs, 1H), 6.54(br s, 1H), 7.03(s, 1H), 7.73(m, 2H), 8.11(br s, 2H), 9.19(s, 1H) | 282 |
| 371 | 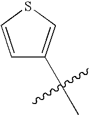 | 4.75(d, 2H), 5.6(t, 1H), 7.05(s, 1H), 7.6(d, 1H), 7.7(m, 1H), 7.8(s, 2H), 8.0(d, 1H), 9.3(s, 1H) | 298 |
| 372 | 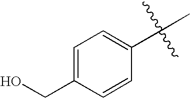 | 4.6(d, 2H), 4.7(d, 2H), 6.5(s, 1H), 7.1(s, 1H), 7.5(d, 2H), 7.7(d, 2H), 7.8(d, 1H), 7.9(d, 1H), 9.3(s, 1H) | 322 |

### Examples 373-397

The hydroxymethyl group on the triazolone scaffold was derivatized via successive bromination (as described previously) and amination followed by Suzuki coupling (under the conditions described above).

General Amination Procedure

To 8-bromo-5-bromomethyl[1,2[4]triazolo[4,3-a]quinolin-1(2H)-one (1 mmol, 1 eq) in THF (0.5-1 ml) was added a solution of the appropriate amine (1-2M in THF) and the mixture stirred at room temperature under nitrogen. On completion of the reaction (15 mins-1 hour) as determined by LC-MS, the THF was removed under reduced pressure and the resultant pasty solid triturated with a mixture of cold ether and hexanes (approximately 1:1). The crude product was dried under vacuum and used in the Suzuki coupling without further purification.

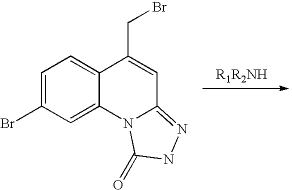

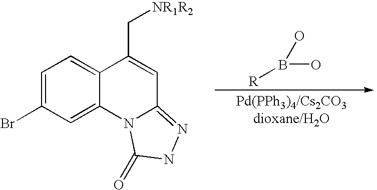

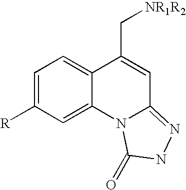

| EX | R | R¹R²N | ¹HNMR(DMSO-d6 unless otherwise stated) | MS(MH+) |
|---|---|---|---|---|
| 373 | 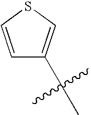 | 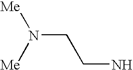 | 2.75(s, 6H), 3.10-3.80(m, 6H), 7.20(s, 1H), 7.52(d, 1H), 7.67(d, 1H), 7.68(d, 1H), 7.85(d, 1H), 7.92(s, 1H), 9.26(s, 1H) | 368 |
| 374 | 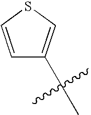 | 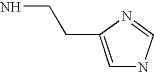 | 3.11(t, 2H), 3.41(t, 2H), 4.53(s, 2H), 7.31(s, 1H), 7.50(s, 1H), 7.57(d, 1H), 7.73(d, 1H), 7.89(d, 1H), 7.95(d, 1H), 8.04(s, 1H), 9.32(s, 1H) | 391 |
| 375 | 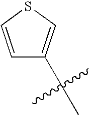 | 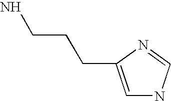 | 2.35(m, 2H), 3.19(t, 2H), 4.41(t, 2H), 4.57(s, 2H), 7.41(s, 1H), 7.67(d, 1H), 7.80(s, 1H), 7.84(d, 2H), 7.98(d, 1H), 8.02(d, 1H), 8.14(s, 1H), 9.42(s, 1H) | 405 |
| 376 | 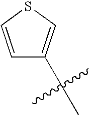 | 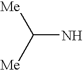 | 1.37(d, 6H), 3.55(m, 1H), 4.49(s, 2H), 7.33(s, 1H), 7.62(d, 1H), 7.76(d, 1H), 7.89(d, 1H), 7.96(d, 1H), 8.06(s, 1H), 9.34(s, 1H) | 339 |
| 377 | 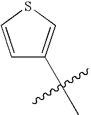 | 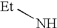 | 1.47(t, 3H), 3.34(m, 2H), 4.67(s, 2H), 7.51(s, 1H), 7.80(d, 1H), 7.93(d, 1H), 8.06(d, 1H), 8.15(d, 1H), 8.24(s, 1H), 9.52(s, 1H) | 325 |
| 378 | 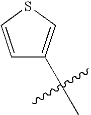 | 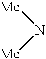 | 2.87(m, 6H), 4.62(m, 2H), 7.49(s, 1H), 7.62(m, 1H), 7.78(m, 1H), 7.89(m, 1H), 8.09(m, 1H), 8.12(m, 1H), 9.36(m, 1H), 10.17(s, 1H) | 325 |
| 379 | 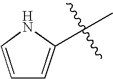 | NH₂ | 4.2(s, 2H), 6.09(m, 1H), 6.50(m, 1H), 6.84(m, 1H), 7.01(m, 1H), 7.69(m, 2H), 8.31(br s, 2H), 9.10(m, 1H), 11.5(br s, 1H) | 280 |
| 380 | 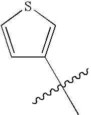 | 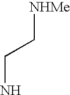 | 2.65(m, 3H), 3.70(m, 2H), 4.60(m, 2H), 7.43(m, 1H), 7.67(m, 1H), 7.78(m, 1H), 7.95(m, 1H), 8.04(m, 1H), 8.11(m, 1H), 9.10(br s, 1H), 9.38(s, 1H), 9.75(br s, 1H) | 354 |
| 381 | 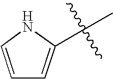 | 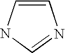 | 5.64(s, 2H), 6.16(m, 1H), 6.58(s, 1H), 6.81(s, 1H), 6.90(s, 1H), 7.68(m, 2H), 7.75(m, 2H), 9.15(s, 2H), 11.58(br s, 1H) | 331 |
| 382 | 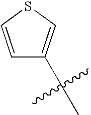 | 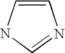 | 5.70(s, 2H), 6.98(s, 1H), 7.52(m, 1H), 7.68(m, 2H), 7.80(m, 3H), 7.97(s, 1H), 9.16(s, 1H), 9.28(s, 1H) | |
| 383 | 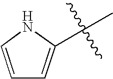 | 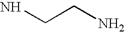 | (D2O) 3.40(m, 4H), 4.25(m, 2H), 6.27(m, 1H), 6.57(m, 1H), 6.90(m, 2H), 7.42(m, 2H), 8.35(s, 1H) | 324 |
| 384 | 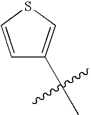 | 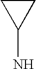 | 0.82(m, 2H), 0.95(m, 2H), 2.90(m, 1H), 4.60(m, 2H), 7.32(s, 1H), 7.60(m, 1H), 7.75(m, 1H), 7.89(m, 1H), 8.03(m, 2H), 9.27(br s, 2H), 9.36(s, 1H) | 337 |
| 385 | 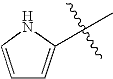 | 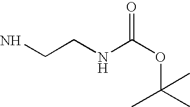 | 1.41(s, 9H), 3.25(m, 4H), 4.47(m, 2H), 6.20(m, 1H), 6.61(m, 1H), 6.99(m, 1H), 7.07(m, 1H), 7.23(s, 1H), 7.78(m, 1H), 7.91(m, 1H), 8.95(m, 2H), 9.22(m, 1H), 11.62(br s, 1H) | 423 |
| 386 | 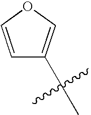 | 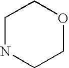 | 3.30(m, 2H), 3.78(m, 4H), 3.96(m, 2H), 4.66(br s, 2H), 6.98(s, 1H), 7.59(s, 1H), 7.78(m, 1H), 7.86(s, 1H), 8.19(m, 1H), 8.36(s, 1H), 9.21(s, 1H), 10.60(br s, 1H) | 351 |
| 387 | 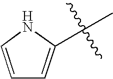 | 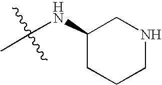 | (D₂O) 0.6(m, 1H), 1.01(m, 1H), 1.65(m, 3H), 2.05(m, 1H), 2.30(m, 1H), 2.86(m, 1H), 3.00(t, 1H), 3.35(m, 1H), 3.57(m, 2H), 3.69(d, 1H), 4.15(q, 2H), 6.18(m, 1H), 6.52(m, 1H), 6.80(s, 1H), 6.89(s, 1H), 7.31(s, 1H), 8.31(s, 1H) | 364 |
| 388 | 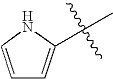 | 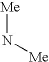 | (D₂O) 2.87(dd, 6H), 4.5(s, 2H), 6.25(m, 1H), 6.5(m, 1H), 6.9(m, 2H), 7.3(d, 1H), 7.4(d, 1H), 8.4(s, 1H) | 325 |
| 389 | 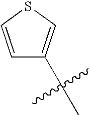 | 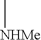 | 2.70(m, 3H), 4.42(m, 2H), 7.34(s, 1H), 7.60(m, 1H), 7.75(m, 1H), 7.89(m, 1H), 7.99(m, 1H), 8.03(m, 1H), 9.29(br s, 2H), 9.32(s, 1H) | 311 |
| 390 | Br— | 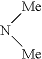 | 2.81(s, 6H), 4.60(m, 2H), 7.53(m, 1H), 7.71(m, 1H), 1H), 9.19(m, 1H), 10.01(br s, 1H) | 323 |
| 391 | 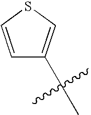 | 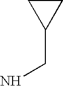 | 0.5(m, 2H), 0.75(m, 2H), 1.1(m, 1H), 3.0(m, 2H), 4.5 (m, 2H), 7.4(s, 1H), 7.6(d, 1H), 7.75(d, 1H), 7.85(dd, 1H), 8.0(s, 1H), 9.15(brs, 1H), 9.2(s, 1H) | 351 |
| 392 | 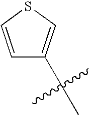 | 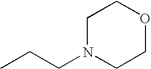 | 3.60(m, 4H), 4.00(m, 8H), 4.52(s, 2H), 7.41(m, 1H), 7.62(m, 1H), 7.77(m, 1H), 7.89(m, 1H), 7.98(m, 1H), 8.06(s, 1H), 9.36(s, 1H), 9.89(br s, 2H), 11.30(br s, 1H) | 411 |
| 393 | 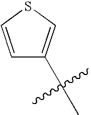 | 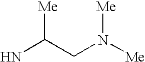 | 1.65(d, 3H), 2.96(s, 6H), 4.10(m, 2H), 4.68(m, 3H), 7.54(s, 1H), 7.68(d, 1H), 7.81(d, 1H), 7.94(d, 1H), 8.12(m, 2H), 9.40(s, 1H) | 382 |
| 394 | 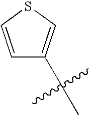 | 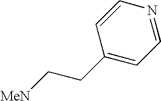 | 2.76(s, 3H), 3.45(t, 2H), 3.58(t, 2H), 4.61(s, 2H), 7.45-7.73(m, 6H), 7.91(s, 1H), 8.03-8.12(m, 2H), 8.56(d 1H), 9.20(s, 1H) | 416 |
| 395 | 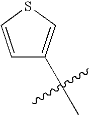 | 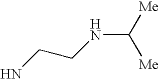 | 1.16(d, 6H), 2.72(m, 1H), 3.40(m, 4H), 4.45(bs, 2H), 7.31(s, 1H), 7.49(d, 1H), 7.62(d, 1H), 7.87(d, 1H), 7.93(m, 2H), 9.21(s, 1H) | 382 |
| 396 | 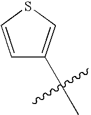 | NH₂ | 4.43(bs, 2H), 7.14(s, 1H), 7.54(d, 1H), 7.68(d, 1H), 7.82(m, 2H), 7.98(s, 1H), 9.27(s, 1H) | 297 |
| 397 | 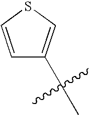 | 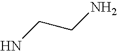 | 3.20-3.40(m, 4H), 4.50(s, 2H), 7.34(s, 1H), 7.55(d, 1H), 7.68(d, 1H), 7.82(d, 1H), 7.92(d, 1H), 7.99(s, 1H), 9.27(s, 1H) | 340 |

### Examples 398-410

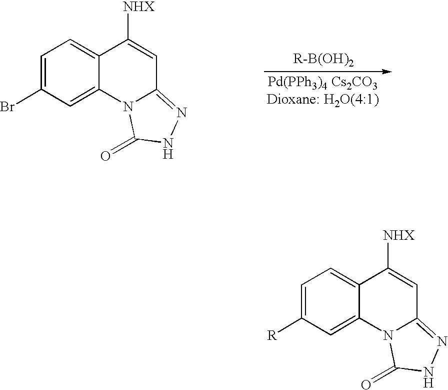

The starting material was synthesized from 6-bromoisatin as previously described and coupled with appropriate boronic acid under standard conditions. The amino group was modified in some cases via a standard HATU mediated coupling with the appropriate carboxylic acid to generate an amide prior to Suzuki coupling.

General Procedure for HATU Coupling

DIEA (9.74 mmols, 5 eq) was added to the carboxylic acid (1.95 mmols, 1 eq) in DMF (5 ml), followed by HATU (2.9 mmols, 1.5 eq). The resultant mixture was stirred at room temperature for 15 minutes and a solution of the required amine (2.9 mmols, 1.5 eq) in DMF (5 ml) added. The mixture was stirred at room temperature for an hour. Additional HATU (1.5 eq) and DIEA (5 eq) were added and the reaction was allowed to stir for an additional hour to complete the reaction (LC-MS). The reaction mixture was concentrated on under reduced pressure. The DMF solution containing crude product was used in subsequent reactions without extensive purification.

| EX | R | X | ¹HNMR(DMSO-d6) | MS(MH+) |
|---|---|---|---|---|
| 398 | 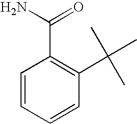 | H | 5.92(s, 1H), 6.32(s, 2H), 7.31(s, 1H), 7.41(m, 6H), 7.77(s, 1H), 7.96(d, 1H), 9.12(s, 1H), 11.79(s, 1H) | 320 |
| 399 | 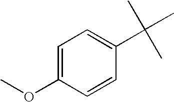 | H | 3.82(s, 3H), 5.93(s, 1H), 6.31(br s, 2H), 7.11(d, 2H), 7.70(m, 3H), 8.03(d, 1H), 9.30(s, 1H), 11.82(s, 1H) | 307 |
| 400 | 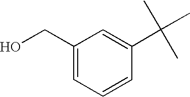 | H | 4.5(s, 1H), 5.9(s, 1H), 7.3(d, 1H), 7.4(m, 1H), 7.5(d, 1H), 7.6(s, 1H), 7.75(d, 1H), 9.2(s, 1H) | 307 |
| 401 | 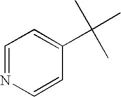 | H | 2.86(m, 6H), 3.32(m, 2H), 3.60(s, 3H), 3.72(m, 2H), 4.00(m, 10H), 7.46(m, 1H); 7.56(s, 1H), 7.71(m, 3H), 7.80(m, 3H), 7.98(m, 1H), 8.11(m, 1H), 9.01(m, 1H), 9.12(m, 1H), 9.32(m, 1H), 9.92(m, 1H), 10.04(brs, 1H) | 278 |
| 402 | 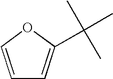 | H | 5.94(s, 1H), 6.68(m, 1H), 7.12(m, 1H), 7.79(d, 1H), 7.87(d, 1H), 8.04(d, 1H), 9.37(d, 1H), 11.87(br s, 1H) | 267 |
| 403 | 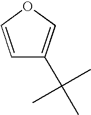 | H | 5.8(s, 1H), 6.3(brs, 2H), 6.9(s, 1H), 7.7(d, 1H), 7.8(s, 1H), 7.9(d, 1H), 8.4(s, 1H), 9.1(s, 1H), 11.8(s, 1H) | 267 |
| 404 | 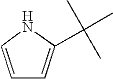 | H | 5.83(s, 1H), 6.19(m, 1H), 6.27(br s, 2H), 6.53(s, 1H), 6.90(s, 1H), 7.74(m, 1H), 7.91(m, 1H), 9.14(s, 1H), 11.48(s, 1H), 11.77(s, 1H) | 266 |
| 405 | 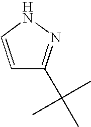 | H | 5.88(s, 1H), 6.31(s, 2H), 7.70(d, 1H), 7.98(d, 2H), 8.24(br s, 1H), 9.20(s, 1H), 11.78(s, 1H) | 267 |
| 406 | 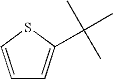 | H | 5.92(s, 1H), 6.37(s, 2H), 7.21(d, 1H), 7.68(m, 2H), 7.79(d, 1H), 8.04(d, 1H), 9.30(m, 1H), 11.86(s, 1H) | 283 |
| 407 | 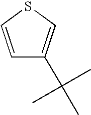 | H | 5.88(s, 1H), 6.28(br s, 2H), 7.51(d, 1H), 7.68(d, 1H), 7.75(d, 1H), 7.91(m, 2H), 9.24(s, 1H), 11.76(s, 1H) | 283 |
| 408 | 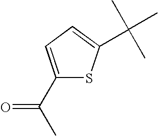 | H | 2.58(s, 3H), 5.99(s, 1H), 6.38(bs, 2H), 7.78(d, 1H), 7.90(d, 1H), 8.00(d, 1H), 8.08(d, 1H), 9.38(s, 1H), 11.76(s, 1H) | 325 |
| 409 | 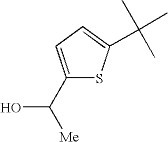 | H | 1.50(d, 3H), 4.98(q, 1H), 5.98(s, 1H), 6.92(d, 1H), 7.32(d, 1H), 7.62(d, 1H), 7.85(d, 2H), 9.24(s, 1H) | 327 |
| 410 | 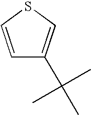 | 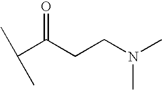 | 3.23(s, 3H), 3.42(s, 3H), 3.67(m, 4H), 6.03(s, 1H), 7.15(bs, 1H), 7.74(d, 1H), 7.89(d, 1H), 8.10(d, 1H), 8.19(s, 1H), 8.30(d, 1H), 9.31(s, 1H) | 382 |

### Examples 411-435

8-bromo-1-oxo-1,2-dihydro[1,2[4]triazolo[4,3-a]quinoline-5-carboxylic acid was coupled under the conditions described above prior to formation of the final products by Suzuki coupling under standard conditions.

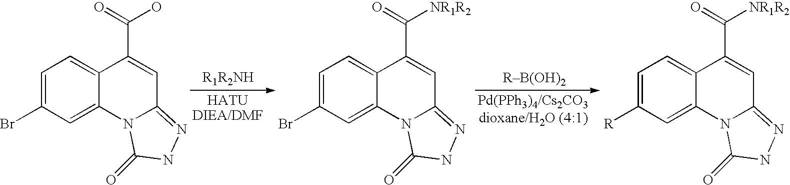

| EX | R | R₁R₂N | ¹HNMR(DMSO-d6 unless stated otherwise) | MS(MH+) |
|---|---|---|---|---|
| 411 | 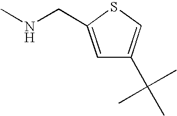 | 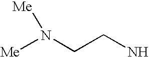 | 2.63(s, 3H), 2.89(s, 6H), 3.33(m, 2H), 3.67(m, 2H), 4.45(s, 2H), 7.48(s, 1H), 7.75(s, 1H), 7.83(d, 1H), 8.03(d, 1H), 8.12(s, 1H), 9.31(s, 1H) | 425 |
| 412 | 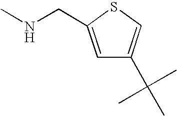 | 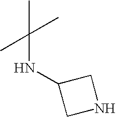 | 3.76(s, 3H), 4.30-4.50(m, 4H), 4.62(bs, 2H), 5.02(m, 1H), 7.64(s, 1H), 7.96(m, 2H), 8.20(d, 1H), 8.30(s, 1H), 9.50(s, 1H) | 409 |
| 413 | 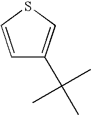 | 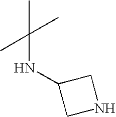 | 4.05(m, 4H), 4.9(m, 1H), 7.5(s, 1H), 7.65(d, 1H), 7.85(m, 2H), 8.0(m, 2H), 9.0(br s, 2H), 9.3(d, 1H), 9.6(m, 1H) | 366 |
| 414 | 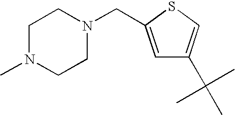 | 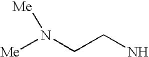 | 2.81(s, 2H), 2.85(m, 6H), 3.31(m, 2H), 3.59(s, 3H), 3.70(m, 2H), 4.25(m, 10H), 7.55(s, 1H), 7.70(m, 1H), 7.79(m, 1H), 8.02(m, 2H), 9.12(m, 1H), 9.30(s, 1H), 10.18(m, 1H) | 494 |
| 415 | 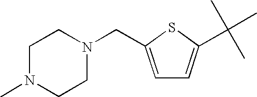 | 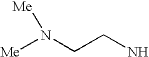 | 2.78(s, 3H), 2.91(d, 6H), 3.09(m, 2H), 3.55(m, 4H), 3.66(m, 2H), 3.87(s, 2H), 7.14(d, 1H), 7.50(s, 1H), 7.57(d, 1H), 7.83(d, 1H), 8.03(d, 1H), 9.02(t, 1H), 9.27(d, 1H) | 494 |
| 416 | 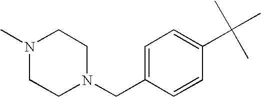 | 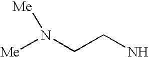 | 2.86(m, 6H), 3.32(m, 2H), 3.60(s, 3H), 3.72(m, 2H), 4.00(m, 10H), 7.46(m, 1H), 7.56(s, 1H), 7.71(m, 3H), 7.80(m, 3H), 7.98(m, 1H), 8.11(m, 1H), 9.01(m, 1H), 9.12(m, 1H), 9.32(m, 1H), 9.92(m, 1H), 10.04(br s, 1H) | 488 |
| 417 | 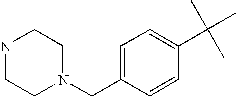 | 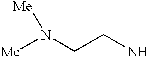 | 2.86(d, 6H), 3.30-3.70(m, 12H), 4.46(s, 2H), 7.60(m, 2H), 7.83(m, 4H), 8.09(d, 1H), 9.18(t, 1H), 9.34(s, 1H) | 474 |
| 418 | 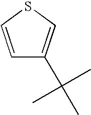 | 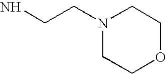 | 3.19(m, 2H), 3.38(m, 2H), 3.51(m, 2H), 3.71(m, 4H), 4.03(m, 2H), 7.38(s, 1H), 7.58(m, 1H), 7.75(m, 1H), 7.81(m, 1H), 8.00(m, 2H), 9.03(m, 1H), 9.32(s, 1H), 9.89(br s, 1H) | 424 |
| 419 | 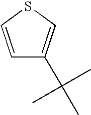 | 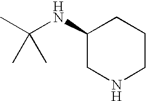 | (D2O) 1.00(m, 1H), 1.45(m, 1H), 1.60(m, 1H), 1.83(m, 2H), 2.40(m, 1H), 2.73(m, 2H), 3.18(m, 1H), 3.34(m, 1H), 3.96(m, 1H), 6.54(m, 1H), 6.97(m, 2H), 7.09(m, 1H), 7.14(m, 1H), 7.18(s, 1H), 8.00(s, 1H) | 394 |
| 420 | 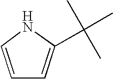 | 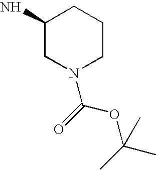 | 1.40(s, 9H), 1.47(m, 1H), 1.76(m, 1H), 1.91(m, 1H), 2.98(m, 2H), 3.67(m, 1H), 3.86(m, 2H), 6.18(m, 1H), 6.58(m, 1H), 6.91(m, 1H), 7.21(s, 1H), 7.72(m, 1H), 7.80(m, 1H), 8.67(d, 1H), 9.18(s, 1H), 11.57(s, 1H) | 477 |
| 421 | 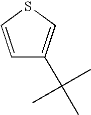 | 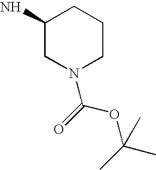 | 1.3(m, 1H), 1.4(s, 9H), 1.76(m, 1H), 1.91(m, 1H), 2.98(m, 2H), 3.67(m, 3H), 3.86(m, 2H), 7.2(s, 1H), 7.6(d, 1H), 7.75(m, 1H), 7.85(dd, 2H), 8.0(s, 1H), 8.8(d, 1H), 9.3(s, 1H) | 494 |
| 422 | 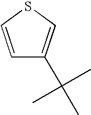 | 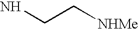 | 2.60(m, 2H), 3.13(m, 2H), 3.60(m, 3H), 7.50(s, 1H), 7.57(m, 1H), 7.76(m, 1H), 7.81(m, 1H), 8.01(m, 2H), 8.58(m, 2H), 8.96(m, 1H), 9.30(m, 1H) | 368 |
| 423 | 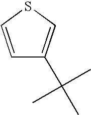 | 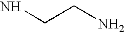 | (D₂O) 3.17(m, 2H), 3.60(m, 2H), 6.87(m, 1H), 7.11(m, 1H), 7.18(m, 1H), 7.32(m, 1H), 7.36(m, 1H), 7.42(s, 1H), 8.22(s, 1H) | 354 |
| 424 | 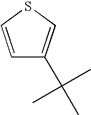 | 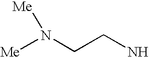 | (D₂O) 2.81(s, 6H), 3.24(m, 2H), 3.62(m, 2H), 6.91(s, 1H), 7.26(d, 1H), 7.31(m, 2H), 7.40(m, 1H), 7.52(s, 1H), 8.48(s, 1H) | 382 |
| 425 | 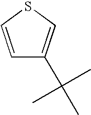 | 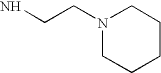 | 1.41(m, 1H), 1.70(m, 5H), 2.97(m, 2H), 3.27(m, 2H), 3.65(m, 4H), 7.47(s, 1H), 7.59(m, 1H), 7.56(m, 1H), 7.83(m, 1H), 8.01(m, 2H), 9.12(m, 1H), 9.37(s, 1H), 9.86(br s, 1H) | 422 |
| 426 | 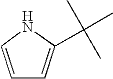 | 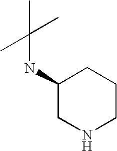 | (D2O) 1.65(m, 2H), 1.97(m, 2H), 2.89(m, 2H), 3.20(m, 1H), 3.43(m, 1H), 4.12(m, 1H), 6.12(m, 1H), 6.38(m, 1H), 6.70(m, 1H), 6.80(m, 1H), 7.07(m, 1H), 7.25(m, 1H), 8.23(m, 1H) | 377 |
| 427 | 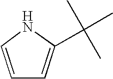 | 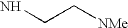 | 2.61(m, 3H), 3.15(m, 2H), 3.60(m, 2H), 6.21(m, 1H), 6.60(m, 1H), 6.98(m, 1H), 7.40(m, 1H), 7.73(m, 1H), 7.92(m, 1H), 8.60(m, 2H), 8.96(m, 1H), 9.20(m, 1H), 11.61(s, 1H) | 351 |
| 428 | 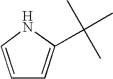 | 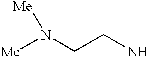 | (D2O) 2.97(s, 6H), 3.40(m, 2H), 3.78(m, 2H), 6.20(m, 1H), 6.48(d, 1H), 6.89(m, 2H), 7.18(d, 1H), 7.40(d, 1H), 8.26(s, 1H) | 365 |
| 429 | 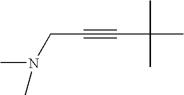 | 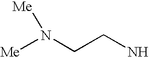 | 2.88(s, 6H), 2.92(s, 6H), 3.32(t, 2H), 3.65(m, 2H), 4.42(s, 2H), 7.57(s, 1H), 7.63(d, 13H), 8.04(d, 1H), 9.04(t, 1H), 9.12(s, 1H) | 381 |
| 430 | 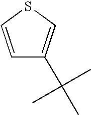 | 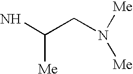 | 1.10(d, 3H), 2.69(s, 6H), 3.10(d, 2H), 4.34(m, 1H), 7.42(d, 1H), 7.46(s, 1H), 7.58(d, 13H), 7.65(m, 1H), 7.86(m, 2H), 9.15(s, 1H) | 396 |
| 431 | 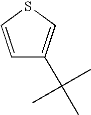 | 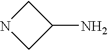 | 3.99(m, 3H), 4.32(m, 2H), 7.16(s, 1H), 7.50(d, 1H), 7.67(m, 1H), 7.77(m, 2H), 7.94(d, 1H), 9.24(s, 1H) | 366 |
| 432 | 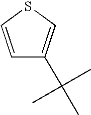 | 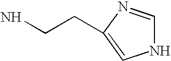 | 2.96(t, 2H), 3.62(t, 2H), 7.21(s, 1H), 7.55(m, 23H), 7.75-7.80(m, 3H), 8.00(s, 1H), 9.08(s, 1H), 9.30(s, 1H) | 405 |
| 433 | 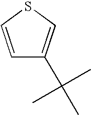 | 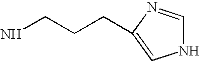 | 2.13(t, 2H), 3.32(m, 2H), 4.33(t, 2H), 7.35(s, 1H), 7.57(d, 1H), 7.73-8.00(m, 5H), 9.23(s, 1H), 9.32(s, 1H) | 419 |
| 434 | 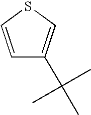 | 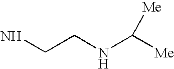 | 1.30(d, 6H), 3.17(t, 2H), 3.57-3.66(m, 3H), 7.51(s, 1H), 7.57(d, 1H), 7.76(d, 1H), 7.84(d, 1H), 8.00(m, 2H), 9.32(s, 1H) | 396 |
| 435 | 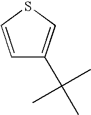 | 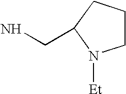 | 1.32(t, 3H), 1.93(m, 3H), 2.21(m, 1H), 3.13(m, 2H), 3.69(m, 4H), 3.87(m, 1H), 7.39(s, 1H), 7.58(d, 1H), 7.74(d, 1H), 7.80(d, 1H), 8.00(m, 2H), 9.32(s, 1H) | 422 |

### Examples 436-442

The following compounds were prepared using tie general scheme outlined below:

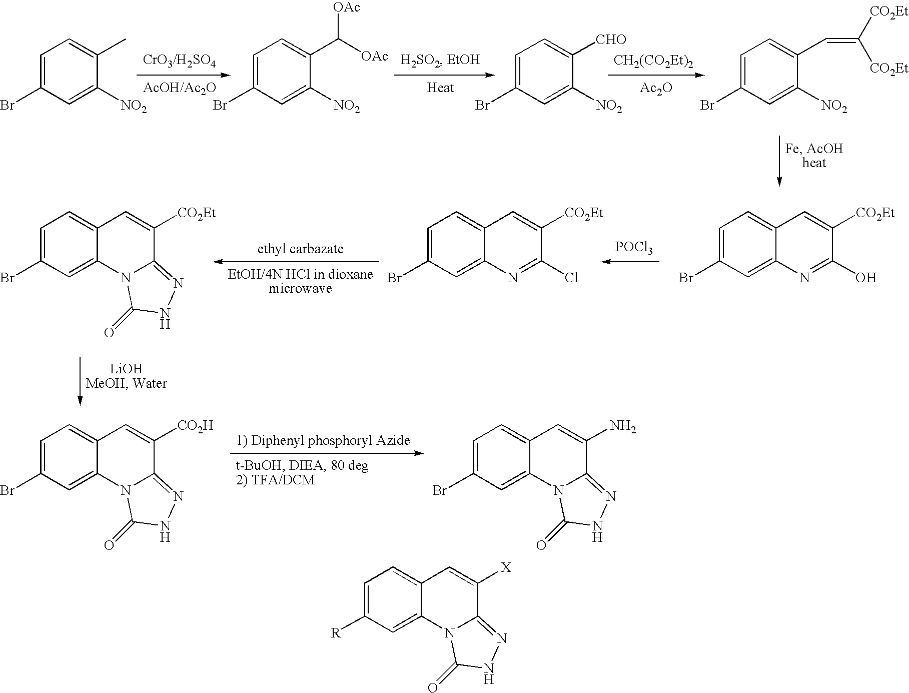

| EX | R | X | ¹HNMR(DMSO-d6 unless otherwise stated) | MS(MH+) |
|---|---|---|---|---|
| 436 | 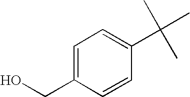 | NH₂ | 4.54(d, 2H), 6.45(s, 1H), 7.30-7.70(m, 6H), 9.12(s, 1H) | 307 |
| 437 | 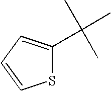 | NH₂ | 6.40(s, 1H), 7.16(d, 1H), 7.45-7.70(m, 4H), 9.10(s, 1H) | 283 |
| 438 | 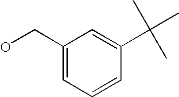 | NH₂ | 4.58(d, 2H), 6.45(s, 1H), 7.25-7.70(m, 6H), 9.12(s, 1H) | 307 |
| 439 | 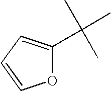 | NH₂ | 6.41(s, 1H), 6.61(s, 1H), 6.90(s, 1H), 7.48(d, 1H), 7.62(d, 1H), 7.77(s, 1H), 9.14(s, 1H) | 267 |
| 440 | 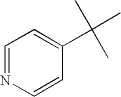 | CH₂OH | 4.62(s, 2H), 7.62(s, 1H), 7.95(m, 4H), 8.80(m, 2H), 9.35(s, 1H) | 293 |
| 441 | 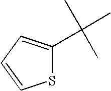 | CH₂OH | 4.60(s, 2H), 7.20(m, 1H), 7.52(s, 1H), 7.65(m, 2H), 7.78(d, 1H), 7.85(d, 1H), 9.20(s, 1H) | 298 |
| 442 | 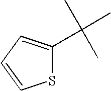 | 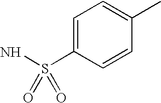 | 2.27(s, 3H), 6.60(s, 1H), 7.10(m, 1H), 7.16(s, 1H), 7.19(s, 1H), 7.25(d, 1H), 7.40(d, 1H), 7.46(m, 2H), 7.66(m, 2H), 9.062(s, 1H) | 437 |

### Example 443

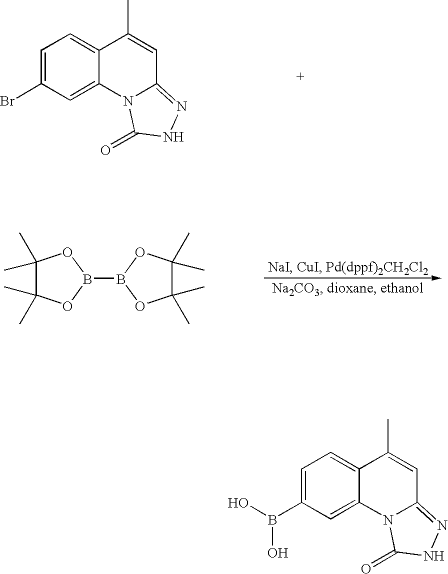

8-bromo-5-methyl[1,2,4]triazolo[4,3-a]quinolin-1(2H)-one was synthesized by the procedure described earlier in the text. The above compound (50 mg, 0.18 mmol), NaI (30 mg, 0.2 mmol), pinacolato diboron (68 mg, 0.268 mmol), CuI (catalytic amount), Pd(dppf)₂CH₂Cl₂ (13 mg, 10 mol %), Na₂CO₃ (57 mg, 0.54 mmol) were added to a reaction vial and dioxane:ethanol=1:1 (2 ml) was added and the mixture was heated at 90° C. overnight. After solvent evaporation the residue was dissolved the residue DMSO, was filtered and was subjected to reverse phase chromatography to afford the title compound. ¹H NMR (DMSO-d₆): 12.37 (s, 1H), 9.34 (s, 1H), 8.25 (s, 2H), 7.86 (d, 1H), 7.77 (d, 1H), 7.04 (s, 1H), 2.45 (s, 3H). MS (M+1): 244.

### Examples 444-446

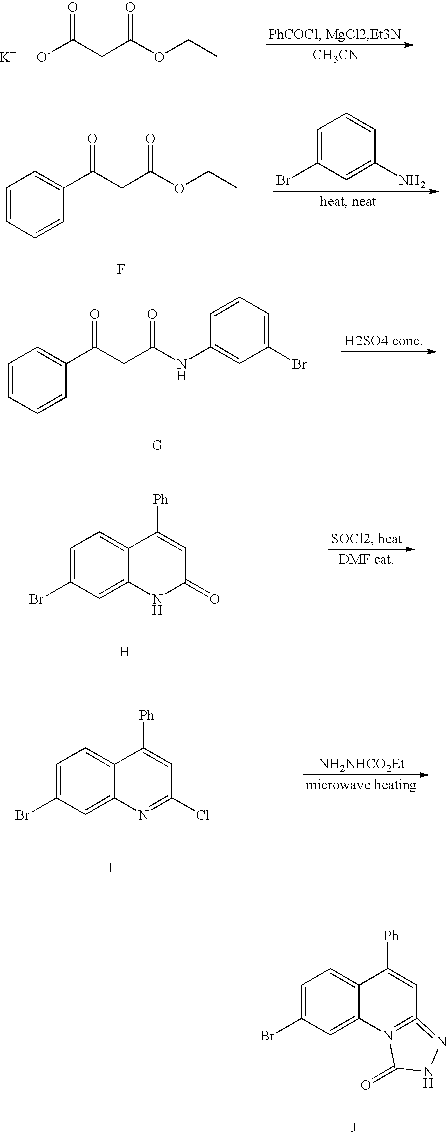

General procedure for preparation of 8-substituted-5-phenyl[1,2,4]triazolo[4,3-a]quinolin-1(2H)-one:

Potassium ethyl malonate (6.28 g, 37.0 mmol) was placed in flask under N₂, CH₃CN (55 ml) was added and the mixture was cooled to 10-15° C. Et₃N (3.68 g, 36.0 mmol) was added followed by addition of MgCl₂ (4.25 g, 45.0 mmol), and the mixture was stirred at room temperature for 2.5 hours. After cooling the reaction mixture to 0° C. benzoyl chloride (2.53 g, 18.0 mmol) was added slowly over 25 min followed by addition of more Et₃N (0.36 g, 4 mmol). The mixture was stirred at room temperature overnight. The solvent was removed under reduced pressure and 20 ml of toluene were added, folloed by evaporation under reduced pressure. 30 ml of toluene were added and the solution was cooled to 10-15° C. 25 ml 13% aqueous HCl were added while carefully keeping the temperature under 25° C. The aqueous layer was discarded and the organic layer was washed with 12% aqueous HCl (2×6.5 ml) and water (2×6 ml). After solvent removal under reduced pressure and Kugelrohr distillation intermediate (F) was obtained.

A mixture of 3-bromoaniline (1.98 g, 11.5 mmol) and ethyl 3-oxo-3-phenylpropanoate (2.85 g, 14.8 mmol) was stirred at 140-150° C. for 1 hour was cooled to room temperature and DCM/hexanes were used to induce precipitation. The solid was filtered off and washed with DCM to yield the intermediate (G).

A mixture of N-(3-bromophenyl)-3-oxo-3-phenylpropanamide (B) (9.6 mmol) and concentrated sulfuric acid (4 ml) were heated at 70-80° C. for 0.5 hours followed by heating at 100° C. for 1 hour. The mixture was cooled to room temperature and poured into crushed ice. The solid that precipitated was filtered off and recrystallized from ethanol to afford intermediate (H).

A mixture of 7-bromo-4-phenylquinolin-2(1H)-one (C) (45.0 mmol), DMF (3 ml), and thionyl chloride (150 ml) was heated at reflux for 3 h. The mixture was cooled to room temperature and the resultant solid was filtered off, was washed with acetone and was dried under vacuum to afford intermediate (I).

To a suspension of 7-bromo-2-chloro4-phenylquinoline (1.0 mmol) and ethyl carbazate (114 mg, 1.1 mmol) in 4 ml of ethanol 4 drops of HCl (4 N in 1,4-dioxane) were added. The reaction mixture was subject to irradiation with microwaves at 170° C. for 20 min. After cooling to room temperature the precipitated solid was filtered off, washed with methanol (3×10 ml) and dried under vacuum to give the desired intermediate (J).

To a 5 ml reaction vial 8-bromo-5-phenyl[1,2,4]triazolo[4,3-a]quinolin-1(2H)-one (75.8 mg, 0.223 mmol) and the appropriate boronic acids of general formula RB(OH)₂ (0.245 mmol), cesium carbonate (290 mg, 0.892 mmol), and tetrakis(trisphenylphosphine)palladium (0) (25.4 mg, 10 mol %) were added. A dioxane:water 4:1 (4 ml) mixture was added and the solution was degassed and back-filled with N₂. The reaction mixture was heated with stirring in a microwave synthesizer for 1200 seconds at 165° C. After cooling to ambient temperature, the solvent was evaporated under reduced pressure. The residual solid was dissolved in the minimum amount of DMSO followed by filtration. The crude product was purified by reverse phase chromatography to afford the title compounds.

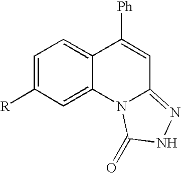

| Ex. | R | ¹H NMR(DMSO-d6) | MS(M + 1) |
|---|---|---|---|
| 444 | 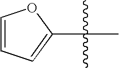 | 12.66(s, 1H), 9.42(s, 1H), 7.93(s, 1H), 7.78(d, 1H), 7.56(m, 6H), 7.11(d, 1H), 6.72(s, 1H), 6.54(s, 1H). | 328 |
| 445 | 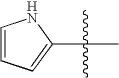 | 12.62(s, 1H), 11.60(s, 1H), 9.29(s, 1H), 7.65(m, 6H), 7.44(d, 1H), 6.98(d, 2H), 6.61(s, 1H), 6.24(s, 1H) | 327 |
| 446 | 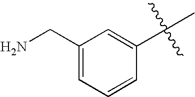 | 12.02(s, 1H), 8.32(s, 3H), 7.84(s, 1H), 7.71(m, 3H), 7.55(m, 8H), 6.43(s, 1H), 4.19(q, 2H). | 368 |

### Examples 447-484

General procedure of preparation of 8-substituted-5-[(alkylamino)methyl][1,2,4]triazolo[4,3-a]quinolin-1(2H)-one:

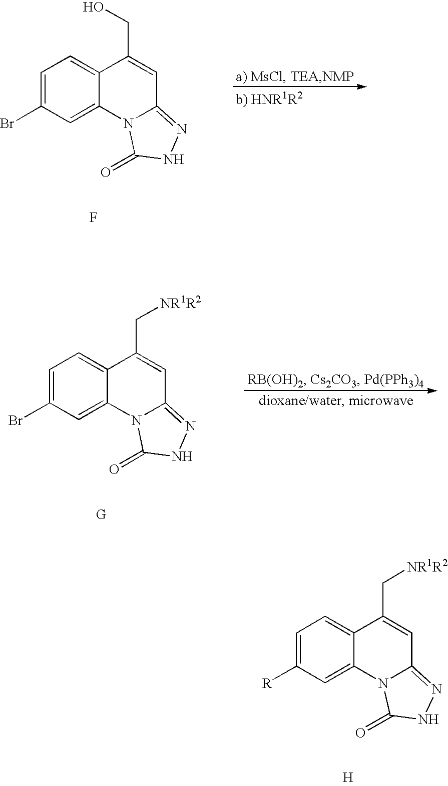

8-Bromo-5-(hydroxymethyl)[1,2,4]triazolo[4,3-a]quinolin-1(2H)-one (F) above was prepared from the ester precursor (396 mg., 1.16 mmols) which was dissolved in dry THF and followed by the very slow addition of LiAlH4 (1M in THF, 1.16 mmols, 1 eq.) while continuing to stir the reaction mixture at r.t. The reaction progress was monitored by LC-MS. The reaction was almost instantaneous but was stirred for 30-40 mins to ensure completion. At this time, any excess of the reducing agent was quenched by the addition of water until the evolution of hydrogen ceased. The resultant reaction mixture was acidified to pH4 and the precipitated product was filtered. The solvent was evaporated from the filtrate and the residue was washed well with water, methanol and hexanes before being vacuum dried. This intermediate (F) above (1.2 g, 4.08 mmol) was dissolved in 10 ml of dry NMP, triethylamine (906 mg, 8.98 mmol) was added to the solution and the reaction mixture was cooled to 0° C. Methanesulfonyl chloride was added slowly to the solution and the mixture was kept under stirring for 0.5 h. The mixture was allowed to reach room temperature and was kept under stirring for additional 0.5 h. The reaction mixture was divided into small aliquots each aliquot containing 50 mg (0.17 mmol) of intermediate. To each aliquot a particular amine NHR₁R₂ was added. The mixture was kept under stirring at room temperature for 10 mins to afford the intermediate (G). To each reaction mixture thus obtained boronic acid RB(OH)₂ (0.187 mmol), cesium carbonate (221 mg, 0.68 mmol), tetrakis(trisphenylphosphine)palladium (0) (19.6 mg, 10 mol %), triethylamine (0.3 ml) were added followed by addition of dioxane:water 4:1 (3 ml). The mixture was degassed and back-filled by N₂. The reaction mixture was heated with stirring in a microwave synthesizer for 1200 seconds at 165° C. After cooling to ambient temperature, the solvent was evaporated under reduced pressure. The residual solid was dissolved in the minimum amount of DMSO followed by filtration. In cases where the amines were Boc protected TFA: water 9:1 was added for Boc group removal. The crude product was purified by reverse phase chromatography to afford the title compounds.

| Ex. | NR₁R₂ | R | ¹H NMR(DMSO-d₆) | MS(M + 1) |
|---|---|---|---|---|
| 447 | 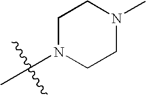 | 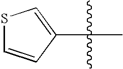 | 12.6(s, 1H), 9.50(br, 2H), 9.30(s, 1H), 8.01(m, 2H), 7.79(m, 2H), 7.58(m, 1H), 7.18(s, 1H), 3.80(s, 2H), 3.39(m, 4H), 3.09(m, 4H), 2.80(s, 3H) | 380 |
| 448 | 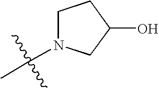 | 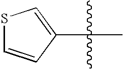 | 12.79(s, 1H), 10.38(s, 1H), 10.04(s, 1H), 9.34(s, 1H), 8.08(d, 2H), 7.90(d, 1H), 7.75(t, 1H), 7.62(d, 1H), 7.46(s, 1H), 4.69(d, 2H), 4.50(d, 1H), 3.30(t, 2H), 3.11(m, 2H), 1.88(m, 2H) | 367 |
| 449 | 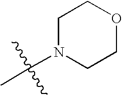 | 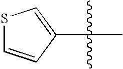 | 12.81(s, 1H), 11.07(s, 1H), 9.34(s, 1H), 8.23(d, 1H), 8.06(s, 1H), 7.88(d, 1H), 7.75(d, 1H), 7.62(m, 2H), 4.86(s, 2H), 3.87(m, 4H), 3.40(m, 4H). | 367 |
| 450 | 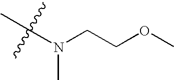 | 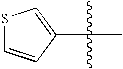 | 12.76(s, 1H), 10.51(s, 1H), 9.27(s, 1H), 8.07(d, 1H), 7.98(s, 1H), 7.82(d, 1H), 7.68(d, 1H), 7.55(d, 2H), 4.68(m, 2H), 4.53(m, 2H), 3.73(m, 2H), 3.27(s, 3H), 2.76(s, 3H) | 369 |
| 451 | 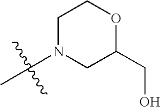 | 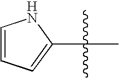 | 12.74(s, 1H), 11.81(s, 1H), 9.90(s, 1H), 9.21(s, 1H), 8.09(s, 1H), 7.79(s, 1H), 7.35(s, 1H), 8.98(s, 1H), 6.63(s, 1H), 6.21(s, 1H), 4.67(s, 2H), 4.00(m, 1H), 3.70(m, 4H), 3.12(m, 2H), 2.44(m, 2H) | 380 |
| 452 | 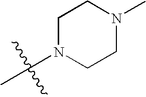 | 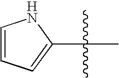 | 12.60(s, 1H), 11.64(s, 1H), 10.81(br, 1H), 9.69(s, 1H), 9.19(s, 1H), 7.99(d, 1H), 7.75(d, 1H), 7.23(s, 1H), 6.96(s, 1H), 6.60(s, 1H), 6.20(s, 1H), 4.04(m, 2H), 3.24(m, 8H), 2.77(s, 3H). | 363 |
| 453 | 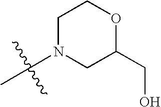 | 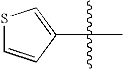 | 12.82(s, 1H), 11.30(br, 1H), 9.34(s, 1H), 8.27(d, 1H), 8.06(s, 1H), 7.86(d, 1H), 7.74(t, 1H), 7.69(s, 1H), 7.62(s, 1H), 7.28(m, 1H), 4.67(s, 2H), 4.29(d, 1H), 3.79(m, 4H), 3.15(m, 4H). | 397 |
| 454 | 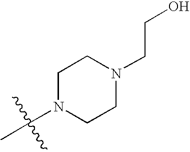 | 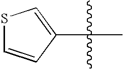 | 12.64(s, 1H), 10.31(br, 1H), 9.31(s, 1H), 8.06(d, 1H), 8.00(s, 1H), 7.83(d, 1H), 7.73(s, 1H), 7.44(d, 1H), 7.29(s, 1H), 3.97(s, 2H), 3.75(m, 4H), 3.16(m, 6H), 2.82(m, 2H). | 410 |
| 455 | 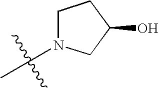 | 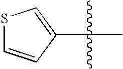 | 12.80(s, 1H), 10.80(br, 1H), 9.34(s, 1H), 8.10(d, 1H), 8.06(s, 1H), 7.89(d, 1H), 7.77(t, 1H), 7.62(d, 1H), 7.55(d, 1H), 4.75(d, 2H), 4.46(d, 1H), 3.41(m, 2H), 3.15(m, 2H), 1.99(m, 2H) | 367 |
| 456 | 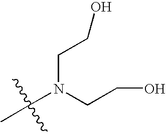 | 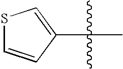 | 12.77(s, 1H), 9.34(s, 1H), 8.08(d, 1H), 8.04(s, 1H), 7.87(d, 1H), 7.75(d, 1H), 7.60(d, 1H), 7.47(s, 1H), 7.36(d, 1H), 7.19(s, 1H), 7.02(s, 1H), 4.63(s, 2H), 3.79(m, 4H), 3.26(m, 4H) | 385 |
| 457 | 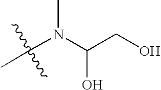 | 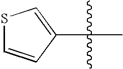 | 12.82(s, 1H), 9.55(br, 2H), 9.34(s, 1H), 8.12(d, 1H), 8.05(d, 1H), 7.88(d, 1H), 7.76(t, 1H), 7.61(d, 1H), 7.47(s, 1H), 4.79(d, 2H), 4.57(d, 2H), 3.98(d, 2H), 3.14(m, 1H), 2.87(s, 3H) | 385 |
| 458 | 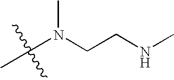 | 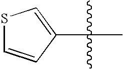 | 12.63(s, 1H), 9.31(s, 1H), 8.34(br, 2H), 8.01(d, 1H), 7.82(d, 1H), 7.74(t, 1H), 7.58(d, 1H), 7.31(s, 1H), 3.98(m, 2H), 3.15(m, 2H), 2.87(m, 2H), 2.56(s, 3H), 2.38(s, 3H) | 368 |
| 459 | 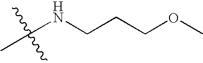 | 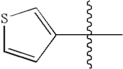 | 12.77(s, 1H), 9.33(s, 1H), 8.93(s, 2H), 8.05(s, 1H), 7.97(d, 1H), 7.90(d, 1H), 7.74(t, 1H), 7.59(d, 1H), 7.33(s, 1H), 4.50(s, 2H), 3.25(s, 3H), 3.16(m, 4H), 1.93(m, 2H) | 369 |
| 460 | 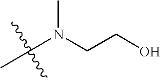 | 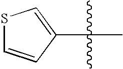 | 12.83(s, 1H), 10.17(s, 1H), 9.34(s, 1H), 8.12(d, 1H), 8.05(s, 1H), 7.86(d, 1H), 7.75(t, 1H), 7.59(d, 2H), 4.70(dd, 2H), 3.85(t, 2H), 3.32(m, 2H), 2.87(s, 3H) | 355 |
| 461 | 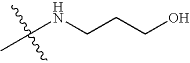 | 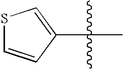 | 12.77(s, 1H), 9.33(s, 1H), 9.01(s, 2H), 8.05(s, 1H), 7.93(d, 1H), 7.90(d, 1H), 7.74(d, 1H), 7.60(d, 1H), 7.34(s, 1H), 4.50(s, 2H), 3.18(m, 4H), 1.84(m, 2H) | 355 |
| 462 | 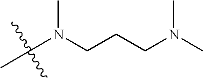 | 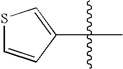 | 12.82(s, 1H), 10.81(s, 1H), 10.70(s, 1H), 9.36(s, 1H), 8.19(d, 1H), 8.05(s, 1H), 7.89(d, 1H), 7.75(t, 1H), 7.66(s, 1H), 7.62(d, 1H), 4.68(m, 2H), 3.33(m, 2H), 3.10(m, 2H), 2.83(s, 3H), 2.76(d, 6H), 2.25(m, 2H) | 396 |
| 463 | 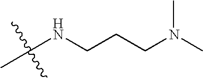 | 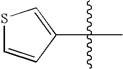 | 12.78(s, 1H), 10.26(s, 1H), 9.35(d, 2H), 8.07(s, 1H), 7.99(d, 1H), 7.91(d, 1H), 7.76(t, 1H), 7.60(s, 1H), 7.41(s, 1H), 4.51(s, 2H), 3.17(m, 4H), 2.79(s, 6H), 2.16(m, 2H) | 382 |
| 464 | 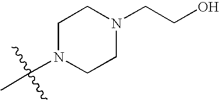 | 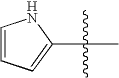 | 12.52(s, 1H), 11.56(s, 1H), 9.39(s, 1H), 9.17(s, 1H), 7.96(d, 1H), 7.70(d, 1H), 7.10(s, 1H), 6.95(s, 1H), 6.58(s, 1H), 6.20(s, 1H), 3.78(s, 2H), 3.69(m, 2H), 3.51(m, 4H), 3.18(s, 2H), 3.07(d, 4H). | 393 |
| 465 | 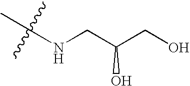 | 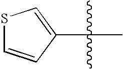 | 12.77(s, 1H), 9.35(s, 1H), 9.02(s, 2H), 8.06(s, 1H), 7.98(d, 1H), 7.91(d, 1H), 7.76(t, 1H), 7.62(d, 1H), 7.35(s, 1H), 5.54(m, 1H), 4.53(s, 2H), 3.90(m, 2H), 3.01(m, 2H) | 371 |
| 466 | 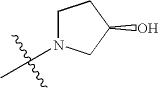 | 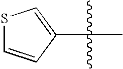 | 12.80(s, 1H), 10.32(s, 1H), 9.98(s, 1H), 9.35(s, 1H), 8.06(d, 2H), 7.91(d, 1H), 7.76(d, 1H), 7.62(d, 1H), 7.45(s, 1H), 5.47(d, 1H), 4.69(d, 2H), 4.42(d, 2H), 3.60(m, 2H), 2.00(m, 2H). | 367 |
| 467 | 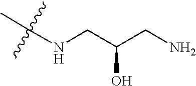 | 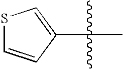 | 12.78(s, 1H), 9.35(s, 1H), 9.27(s, 1H), 8.06(s, 1H), 7.96(m, 7H), 7.76(d, 1H), 7.62(d, 1H), 7.35(s, 1H), 6.18(d, 2H), 4.52(s, 1H), 4.0(d, 2H), 2.82(m, 2H) | 370 |
| 468 | 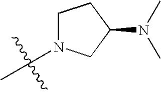 | 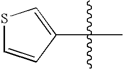 | 12.62(s, 1H), 9.72(s, 1H), 9.31(s, 1H), 8.05(d, 1H), 7.96(s, 1H), 7.52(d, 1H), 7.44(t, 1H), 7.58(d, 1H), 7.20(s, 1H), 4.51(s, 2H), 2.98(m, 2H), 2.73(s, 6H), 2.18(m, 2H), 2.25(m, 1H), 1.99(m, 2H). | 394 |
| 469 | 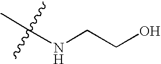 | 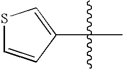 | 12.76(s, 1H), 9.34(s, 1H), 9.05(s, 2H), 8.06(d, 1H), 7.96(d, 1H), 7.91(d, 1H), 7.75(t, 1H), 7.60(d, 1H), 7.34(s, 1H), 5.31(br, 1H), 4.52(s, 2H), 3.75(t, 2H), 3.20(s, 2H) | 341 |
| 470 | 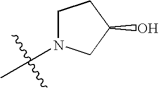 | 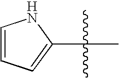 | 12.73(s, 1H), 11.64(s, 1H), 10.32(s, 1H), 9.95(s, 1H), 9.21(s, 1H), 8.08(d, 1H), 7.77(d, 1H), 7.36(s, 1H), 6.99(s, 1H), 6.63(s, 1H), 6.21(s, 1H), 5.48(m, 2H), 4.65(d, 2H), 4.41(d, 1H), 3.55(m, 2H), 1.98(d, 2H). | 350 |
| 471 | 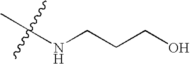 | 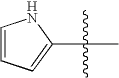 | 12.71(s, 1H), 11.65(s, 1H), 9.21(s, 1H), 8.89(s, 2H), 7.91(d, 1H), 7.77(d, 1H), 7.25(s, 1H), 6.98(s, 1H), 6.63(s, 1H), 6.21(t, 1H), 4.48(s, 2H), 3.52(m, 2H), 3.17(m, 2H), 1.84(m, 2H). | 338 |
| 472 | 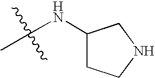 | 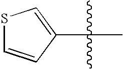 | 12.73(s, 1H), 9.34(s, 1H), 8.97(s, 2H), 8.05(s, 1H), 7.96(d, 1H), 7.90(d, 1H), 7.78(d, 1H), 7.60(d, 1H), 7.31(s, 1H), 4.42(d, 2H), 3.98(m, 2H), 2.18(m, 5H) | 366 |
| 473 | 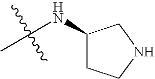 | 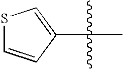 | 12.71(s, 1H), 9.33(s, 1H), 8.96(s, 2H), 8.04(s, 1H), 7.95(d, 1H), 7.86(d, 1H), 7.75(d, 1H), 7.61(d, 1H), 7.30(s, 1H), 4.43(d, 2H), 3.96(m, 2H), 2.15(m, 5H). | 366 |
| 474 | 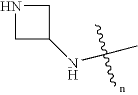 | 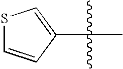 | 12.68(s, 1H), 9.33(s, 1H), 8.73(m, 2H), 8.02(s, 1H), 7.93(d, 1H), 7.88(d, 1H), 7.75(d, 1H), 7.60(t, 1H), 7.24(s, 1H), 4.20(s, 2H), 4.07(m, 5H) | 352 |
| 475 | 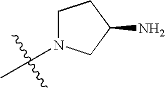 | 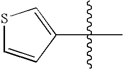 | 12.70(s, 1H), 9.33(s, 1H), 8.03(d, 5H), 7.86(d, 1H), 7.75(t, 1H), 7.60(d, 1H), 7.35(s, 1H), 4.28(m, 2H), 3.80(m, 2H), 3.24(m, 2H), 2.28(m, 2H), 1.88(m, 1H) | 366 |
| 476 | 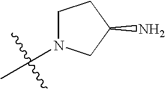 | 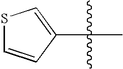 | 12.67(s, 1H), 9.33(s, 1H), 8.02(m, 5H), 7.82(d, 1H), 7.74(t, 1H) 7.00(d, 1H), 7.34(s, 1H), 4.27(m, 2H), 3.82(m, 2H), 3.22(m, 2H), 2.31(m, 2H), 1.86(m, 1H) | 366 |
| 477 | 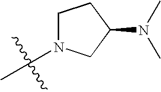 | 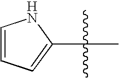 | 12.57(s, 1H), 11.58(s, 1H), 9.97(m, 2H), 9.18(s, 1H), 7.95(d, 1H), 7.71(d, 1H), 7.12(s, 1H), 6.96(s, 1H), 6.59(s, 1H), 6.20(s, 1H), 3.87(m, 2H), 3.06(m, 2H), 2.74(s, 6H), 2.22(m, 1H), 2.20(m, 2H), 1.98(m, 2H). | 377 |
| 478 | 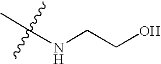 | 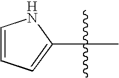 | 12.70(s, 1H), 11.64(s, 1H), 9.21(s, 1H), 9.00(s, 2H), 7.90(d, 1H), 7.79(d, 1H), 7.25(s, 1H), 6.98(s, 1H), 6.63(s, 1H), 6.21(s, 1H), 5.30(m, 1H), 4.48(s, 2H), 3.74(t, 2H), 3.18(t, 2H). | 324 |
| 479 | 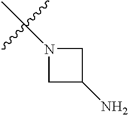 | 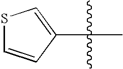 | 12.76(s, 1H), 9.33(d, 1H), 8.86(br, 3H), 8.05(s, 1H), 8.01(d, 1H), 7.90(t, 1H), 7.74(d, 1H), 7.60(d, 1H), 7.40(d, 1H), 4.95(m, 1H), 4.50(m, 2H), 4.08(m, 2H). | 352 |
| 480 | 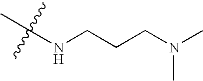 | 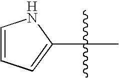 | 12.71(s, 1H), 11.66(s, 1H), 9.74(s, 1H), 9.22(s, 1H), 9.11(s, 2H), 7.90(d, 1H), 7.81(d, 1H), 7.24(s, 1H), 6.99(s, 1H), 6.64(s, 1H), 6.22(s, 1H), 4.48(s, 2H), 3.16(m, 4H), 2.80(s, 6H), 2.06(m, 2H). | 365 |
| 481 | 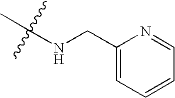 | 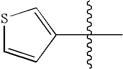 | 12.78(s, 1H), 9.69(s, 2H), 9.34(s, 1H), 8.68(d, 1H), 8.05(d, 2H), 7.92(t, 2H), 7.76(d, 1H), 7.62(d, 1H), 7.55(d, 1H), 7.47(t, 1H), 7.37(s, 1H), 4.60(s, 2H), 4.53(s, 2H). | 386 |
| 482 | 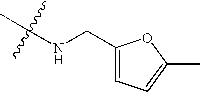 | 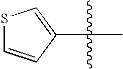 | 12.77(s, 1H), 9.55(s, 2H), 9.33(s, 1H), 8.06(d, 1H), 7.90(s, 2H), 7.75(t, 1H), 7.60(d, 1H), 7.30(s, 1H), 6.56(s, 1H), 6.17(s, 1H), 4.49(s, 2H), 4.37(s, 2H), 2.30(s, 3H). | 391 |
| 483 | 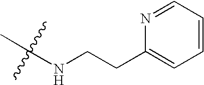 | 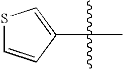 | 12.77(s, 1H), 9.34(s, 1H), 9.77(s, 2H), 8.53(d, 1H), 8.06(d, 1H), 8.03(s, 1H), 7.94(d, 1H), 7.82(t, 1H), 7.76(m, 1H), 7.63(d, 1H), 7.38(d, 2H), 7.33(d, 1H), 4.60(s, 2H), 3.30(m, 2H), 3.22(t, 2H) | 402 |
| 484 | 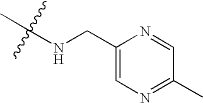 | 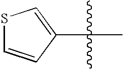 | 12.78(s, 1H), 9.66(s, 2H), 9.33(s, 1H), 8.87(d, 2H), 8.06(s, 1H), 7.99(d, 1H), 7.91(d, 1H), 7.77(t, 1H), 7.62(d, 1H), 7.36(s, 1H), 4.60(s, 2H), 4.53(s, 2H), 2.54(s, 3H). | 401 |

### Example 485

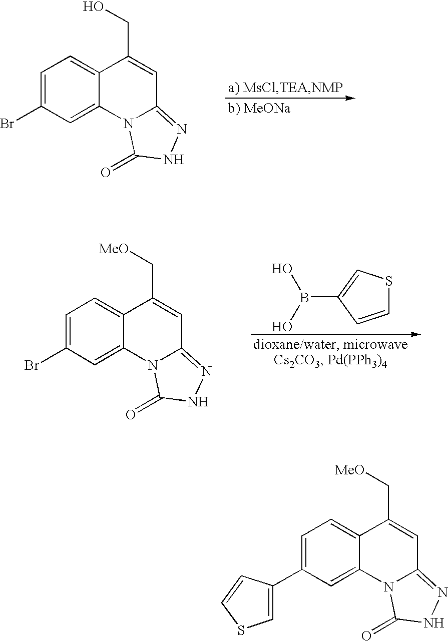

The general procedure of making 8-substituted-5-[(alkylamino)methyl][1,2,4]triazolo[4,3-a]quinolin-1(2H)-one was used with the exception of replacing the amines with sodium methoxide to afford the title compound. ¹H NMR (DMSO-d₆): 12.53 (s, 1H), 9.31 (s, 1H), 7.89 (d, 1H), 7.81 (s, 1H), 7.72 (d, 1H), 7.58 (d, 1H), 7.09 (s, 1H), 6.56 (s, 1H), 5.59 (s, 2H), 4.77 (s, 3H). MS (M+1): 312

### Examples 486-487

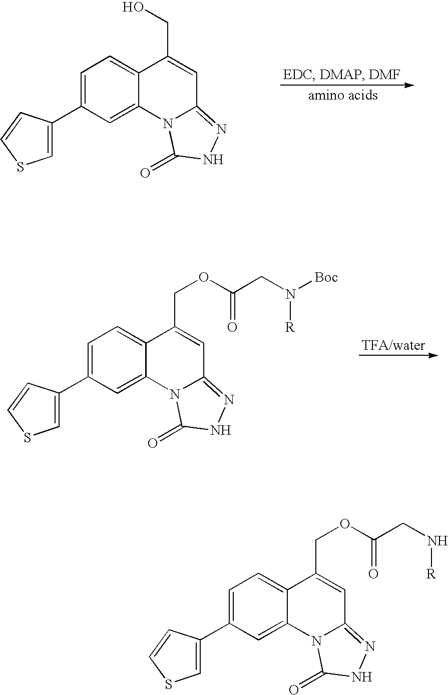

5-(hydroxymethyl)-8-thien-3-yl[1,2,4]triazolo[4,3,-a]quinolin-1(2H)-one was synthesized following the procedure described earlier in the text. The above compound (95 mg, 0.32 mmol) was dissolved in DME (3 ml),amino acids (0.64 mmol) were added followed by DMAP (9.8 mg, 0.078 mmol), and EDC (123 mg, 0.64 mmol). The mixture was kept under stirring at room temperature overnight. TFA:water 1:1 (20 ml) was added and stirred for 1 h. The solvents were evaporated under reduced pressure and the residue was dissolved in DMSO, followed by purification by reverse phase chromatography to afford the title compounds.

| | | | MS |
|---|---|---|---|
| Ex. | R | ¹H NMR (DMSO-d₆) | (H⁺) |
| 486 | H | 12.69(s, 1H), 9.32(s, 1H), 8.44(s, 3H), 8.02(s, | 355 |
| | | 1H), 7.85(s, 2H), 7.75(d, 1H), 7.59(d, 1H), | |
| | | 7.31(s, 1H), 5.55(s, 2H), 4.01(d, 2H). | |
| 487 | CH₃ | 12.57(s, 1H), 9.17(s, 1H), 8.95(br, 2H), 7.87(s, | 369 |
| | | 1H), 7.70(s, 2H), 7.61(t, 1H), 7.45(d, 1H), | |
| | | 7.15(s, 1H), 5.42(s, 2H), 4.04(s, 2H), 2.37(s, 3H). | |

### Examples 488-491

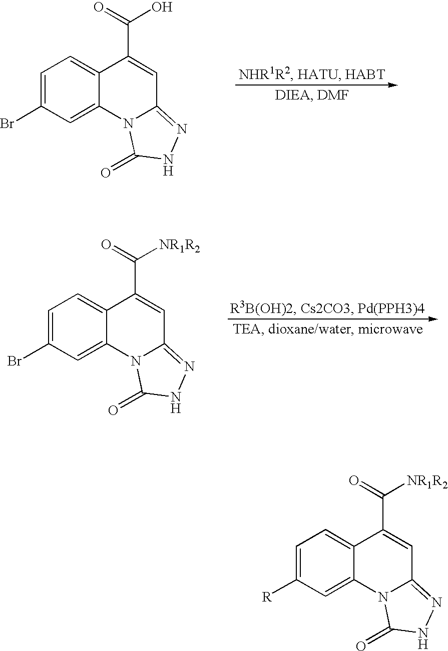

8-Bromo-1-oxo-1,2-dihydro[1,2,4]triazolo[4,3-a]quinoline-5-carboxylic acid (100 mg, 0.325 mmol), HATU (148.3 mg, 0.39 mmol), HABT (53.1 mg, 0.39 mmol) and DIEA (84.0 mg, 0.65 mmol) were added to a reaction vial. DMF was added (4 ml) and the mixture was stirred at room temperature for 0.5 h. Amine NHR¹R² (0.49 mmol) added to the reaction mixture and the solution was kept under stirring at r.t. overnight. The obtained solution was carried over to the next step without any evaporation and purification. The detailed procedure for the Suzuki coupling was described earlier in the text. All compounds were purified by reverse phase chromatography.

| Ex. | NR¹R² | R | ¹H NMR(DMSO-d6) | MS(H⁺) |
|---|---|---|---|---|
| 488 | H | 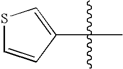 | 12.93(s, 1H), 9.33(s, 1H), 8.50(d, 1H), 7.99(s, 1H), 7.79(d, 1H), 7.73(d, 1H), 7.65(s, 1H), 7.58(d, 1H), 6.56(s, 1H). | 312 |
| 489 | 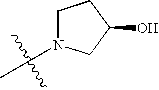 | 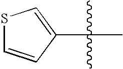 | 12.73(s, 1H), 9.30(s, 1H), 7.99(s, 1H), 7.82(d, 1H), 7.74(t, 1H), 7.57(d, 2H), 7.68(d, 1H), 6.55(s, 1H), 5.05(d, 1H), 4.30(d, 2H), 3.65(m, 2H), 2.00(m, 2H). | 381 |
| 490 | 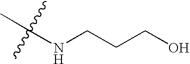 | 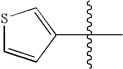 | 12.77(s, 1H), 9.31(s, 1H), 8.75(t, 1H), 7.99(s, 1H), 7.90(d, 1H), 7.84(d, 1H), 7.74(t, 1H), 7.58(d, 1H), 7.20(s, 1H), 3.51(t, 2H), 1.72(m, 4H). | 369 |
| 491 | 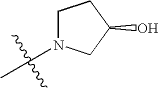 | 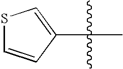 | 12.72(s, 1H), 9.30(s, 1H), 7.99(s, 1H), 7.80(d, 1H), 7.76(d, 1H), 7.57(d, 2H), 7.17(d, 1H), 5.01(m, 1H), 4.30(d, 2H), 3.57(m, 2H), 1.91(m, 2H). | 381 |

### Examples 492-498

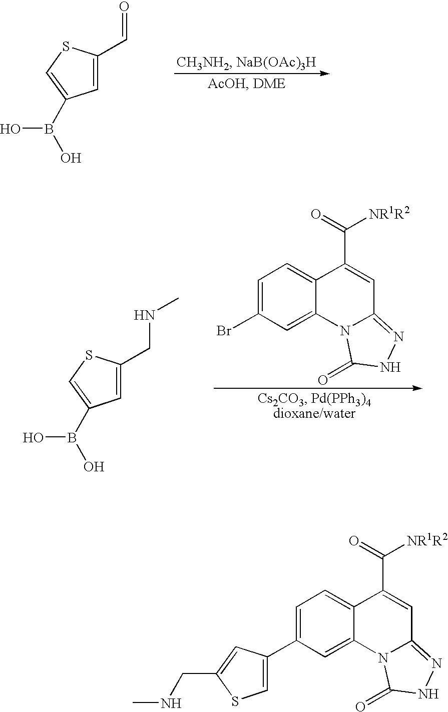

The general procedure to make the 5-[(substitutedamino)methyl]-8-{5-[(methylamino)methyl]thien-3-yl}[1,2,4]traizolo[4,3-a]quinoline-1(2H)-one was described earlier in the text.

| Ex. | R¹R²N | ¹H NMR(DMSO-d₆) | MS(H+) |
|---|---|---|---|
| 492 | 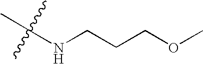 | 12.79(s, 1H), 9.33(s, 1H), 9.05(d, 3H), 8.18(s, 1H), 8.00(d, 1H), 7.89(d, 1H), 7.79(s, 1H), 7.39(s, 1H), 4.49(d, 2H), 3.30(s, 3H), 3.33(s, 3H), 3.07(m, 4H), 2.61(t, 2H), 1.94(m, 2H). | 412 |
| 493 | 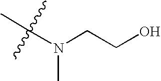 | 12.84(s, 1H), 9.95(br, 1H), 9.34(s, 1H), 9.13(d, 2H), 8.18(d, 1H), 8.13(s, 1H), 7.88(d, 1H), 7.81(s, 1H), 7.56(s, 1H), 4.79(d, 2H), 4.57(d, 2H), 4.42(t, 2H), 3.84(t, 2H), 3.17(s, 3H), 2.86(s, 3H). | 398 |
| 494 | 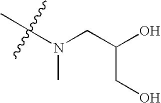 | 12.83(s, 1H), 9.34(s, 1H), 8.99(s, 2H), 8.18(s, 1H), 8.11(d, 1H), 7.86(d, 1H), 7.77(s, 1H), 7.48(s, 1H), 4.46(s, 4H), 3.98(m, 2H), 3.28(m, 2H), 3.17(m, 1H), 2.86(s, 3H), 2.63(s, 3H) | 428 |
| 495 | 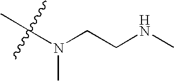 | 12.77(s, 1H), 9.35(s, 1H), 9.04(s, 2H), 8.39(s, 1H), 8.16(d, 1H), 7.90(d, 1H), 7.80(d, 1H), 7.78(s, 1H), 4.44(t, 2H), 3.70(m, 2H), 3.24(s, 3H), 2.91(s, 3H), 2.79(s, 3H), 2.60(m, 2H), 2.27(t, 2H). | 411 |
| 496 | 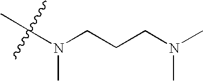 | 12.75(s, 1H), 9.35(s, 1H), 9.03(s, 2H), 8.18(d, 1H), 7.88(d, 1H), 7.80(s, 1H), 7.71(s, 1H), 7.61(s, 1H), 4.44(d, 2H), 3.70(m, 2H), 2.79(d, 2H), 2.77(s, 3H), 2.62(s, 3H), 2.27(s, 3H), 2.09(s, 3H), 1.28(d, 2H), 0.88(m, 2H). | 439 |
| 497 | 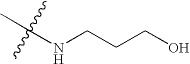 | 12.75(s, 1H), 9.33(s, 1H), 8.97(d, 2H), 8.19(s, 1H), 8.01(d, 1H), 7.89(d, 1H), 7.79(s, 1H), 7.38(s, 1H), 4.50(d, 1H), 2.89(d, 2H), 2.73(s, 3H), 2.58(m, 2H), 2.42(m, 2H), 1.84(m, 2H). | 398 |
| 498 | 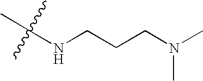 | 12.57(s, 1H), 9.62(s, 1H), 9.33(s, 1H), 9.15(s, 1H), 8.17(s, 1H), 8.02(d, 2H), 7.89(d, 1H), 7.81(s, 1H), 7.45(s, 1H), 7.15(t, 1H), 4.43(d, 2H), 3.68(m, 2H), 3.19(m, 2H), 3.05(m, 2H), 2.92(s, 3H), 3.76(s, 3H), 2.74(s, 3H), 2.62(t, 2H). | 425 |

### Example 499

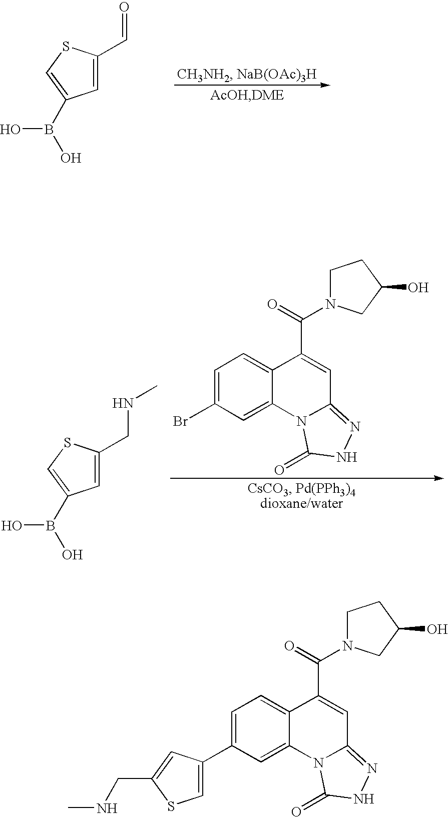

5-{[(3R)-3-hydroxypyrrolidin-1-yl]carbonyl}-8-{5-[methylamino)methyl]thien-3-yl}[1,2,4]tiazolo[4,3-a]quinolin-1(2H)-one was synthesized following the general procedures described earlier in the text. ¹H NMR (DMSO-d₆): 12.77 (s, 1H), 9.30 (s, 1H), 9.06 (br, 2H), 8.11 (s, 1H), 7.80 (d, 1H), 7.77 (s, 1H), 7.57 (t, 1H), 7.20 (s, 1H), 4.43 (d, 1H), 4.30 (d, 2H), 3.57 (m, 2H), 3.14 (d, 2H), 2.60 (s, 3H), 1.78 (m, 2H). MS (M+1): 393.

### Example 500

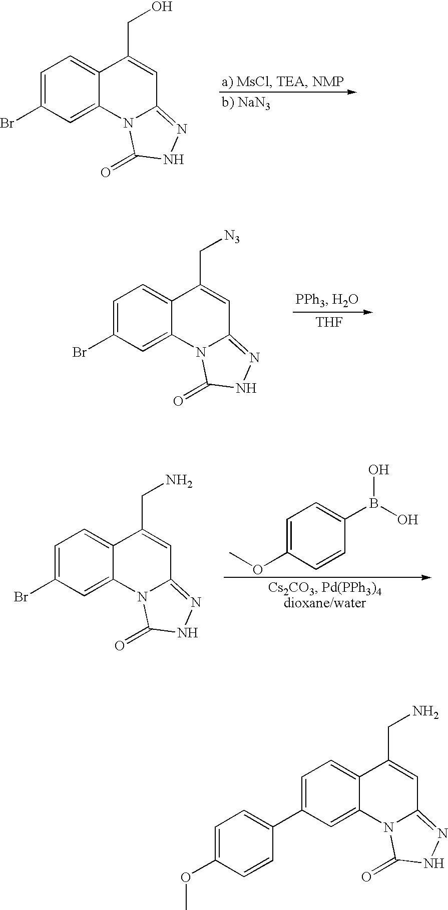

8-Bromo-5-(hydroxymethyl)[1,2,4]triazolo[4,3-a]quinolin-1(2H)-one (F) was prepared following the procedure described earlier in the text. This compound (100 mg, 0.34 mmol) was dissolved in NMP (2 ml), triethylamine (222 mg, 0.75 mmol) was added and the reaction mixture was cooled to 0° C. Methanesulfonyl chloride was added slowly and the solution was kept under stirring for 0.5 h. The mixture was allowed to reach room temperature and was stirred for additional 0.5 h. NaN₃ (88.4 mg, 1.36 mmol) was added and the reaction mixture was heated at 65° C. for 5 h. Ethyl acetate was added to the reaction mixture and was extracted twice with water and was washed with water and brine. After evaporation the intermediate 5-(azidomethyl)-8-bromo[1,2,4]triazolo[4,3-a]quinolin-1(2H)-one was obtained. This compound was carried over to the next step without further purification.

5-(azidomethyl)-8-bromo[1,2,4]triazolo[4,3-a]quinolin-1(2H)-one (0.34 mmol) was dissolved in THF (8 ml) and PPh₃ (129.4 mg, 0.68 mmol) and water (12.24 mg, 0.68 mmol) were added to the solution. The mixture was stirred at room temperature for 2 days, was evaporated and the residue was carried over to the next step without further purification. Example 458 was prepared by following the general Suzuki coupling procedure described earlier in the text. ¹H NMR (DMSO-₆): 12.75 (s, 1H), 9.35 (s, 1H), 8.48 (s, 2H), 8.02 (d, 1H), 7.96 (s, 1H), 7.81 (d, 1H), 7.77 (d, 2H), 7.52 (d, 2H), 7.28 (d, 1H), 7.23 (s, 1H), 4.50 (s, 2H), 4.41 (s, 3H), 3.28 (s, 2H). MS (H⁺): 335.

### Examples 501-502

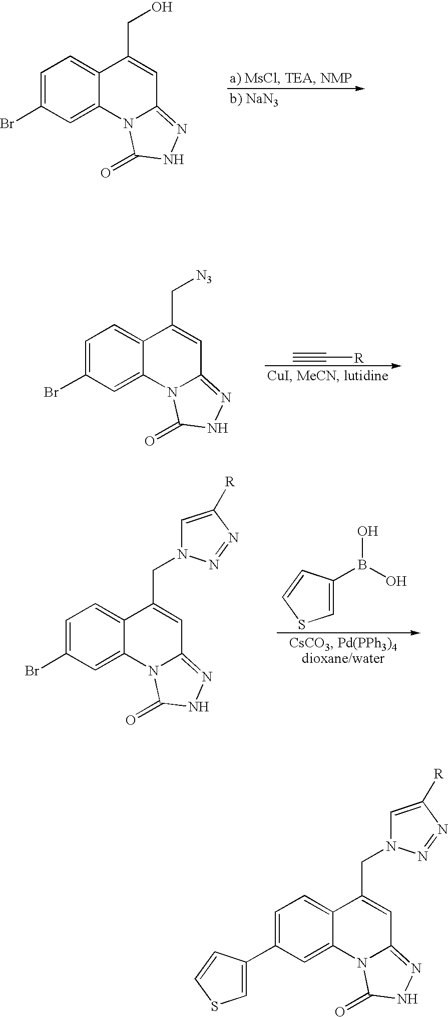

5-(azidomethyl)-8-bromo[1,2,4]triazolo[4,3-a]quinolin-1(2H)-one (50 mg, 0.17 mmol) (prepared following the procedure described above in the text) was dissolved in acetonitrile, 2,6-lutidine (20.3 mg, 0.188 mmol) and CuI (3 mg, 0.016 mmol) were added and the mixture was heated at 65° C. for 16 h to afford the intermediate which was used in the next without further purification. The general procedure for Suzuki coupling described earlier in this text afforded the examples listed below.

| | | | MS |
|---|---|---|---|
| Ex. | R | ¹H NMR (DMSO-d₆) | (M + 1) |
| 501 | CH₂OH | 12.68(s, 1H), 9.31(s, 1H), 8.11(s, 1H), | 379 |
| | | 7.98(d, 1H), 7.93(d, 1H), 7.86(d, 1H), | |
| | | 7.75(d, 1H), 7.58(d, 1H), 6.87(s, 1H), | |
| | | 5.92(s, 2H), 4.52(s, 2H). | |
| 502 | CH₂NHCH₃ | 12.65(s, 1H), 9.25(s, 1H), 8.78(br, 2H), | 392 |
| | | 8.28(s, 1H), 7.96(s, 1H), 7.88(d, 1H), | |
| | | 7.80(d, 1H), 7.68(d, 1H), 7.51(d, 1H), | |
| | | 6.79(s, 1H), 5.95(s, 2H), 4.19(s, 2H), | |
| | | 2.53(s, 3H). | |

### Example 503

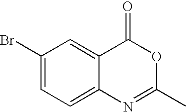

### 6-Bromo-2-methyl-4H-3,1-benzoxazin-4-one

5-Bromo-2-amino-benzoic acid (4.321 g, 20 mmol) was suspended in 50 mL acetic anhydride and heated at 150° C. for 3 h. The volatiles were evaporated under vacuum and the residue was dried in air to afford a cream-colored powder (4.601 g, 96%). ¹H NMR (400 MHz, CDCl₃) δ 8.29 (d, 1H), 7.86 (dd, 1H), 7.40 (d, 1H), 2.45 (s, 3H); M+1=241.

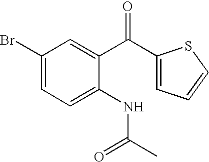

### N-[4-bromo-2-(thien-2-ylcarbonyl)phenyl]acetamide

6-Bromo-2-methyl-4H-3,1-benzoxazin-4-one (9.602 g, 40 mmol) was dissolved in dry THF under a N₂ atmosphere (100 mL) and cooled to 0° C. with an ice-water bath. A 1M solution in THF of 2-thienylmagnesium bromide (40 mL, 40 mmol) was added via syringe and the solution was allowed to reach room temperature overnight. Saturated aqueous ammonium chloride solution was added (50 mL) and the mixture was stirred for 1 h. The organic layer was separated and dried over MgSO₄. After filtration and solvent evaporation under vacuum a thick green oil was obtained. The oil was subjected to flash chromatography over silica gel with a gradient 5 to 35% ethyl acetate in hexanes over 55 min to afford a light brown solid (5.710 g, 44%). ¹H NMR (400 MHz, CDCl₃) δ 10.09 (brs, 1H), 8.46 (d, 11H), 7.90 (d, 1H), 7.80 (dd, 1H), 7.65 (dd, 1H), 7.60 (dd, 1H), 7.20 (dd, 1H), 2.18 (s, 3H); M+1=325.

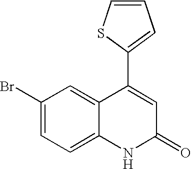

### 6-bromo-4-thien-2-ylquinolin-2(1H)-one

To dry dioxane (50 mL) under N₂ was added 1M potassium tert-butoxide in tert-butanol (34 mL, 34 mmol) and the solution was heated at 90° C. A solution of N-[4-bromo-2-(thien-2-ylcarbonyl)phenyl]acetamide (5.511 g, 17 mmol) in dry dioxane (60 mL) was added dropwise over 30 min. The mixture was heated at 90° C. with stirring for 2 h followed by cooling to room temperature. A 1N HCl aqueous solution was added (35 mL, 35 mmol) and all volatiles were removed under vacuum. The solid was taken in water (50 mL) and filtered through a fritted funnel and washed extensively with water (200 mL) followed by methanol (20 mL). The white solid was dried in the air to afford a white powder (4.443 g, 85%). ¹H NMR (400 MHz, DMSO-d₆) δ 12.04 (brs, 1H), 7.87 (d, 1H), 7.81 (dd, 1H), 7.72 (dd, 1H), 7.50 (dd, 1H), 7.35 (d, 1H), 7.28 (dd, 1H), 6.57 (s, 1H); M+1=307.

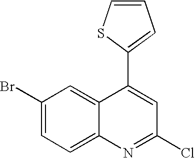

### 6-bromo-2-chloro4-thien-2-ylquinoline

6-Bromo-4-thien-2-ylquinolin-2(1H)-one was suspended in thionyl chloride (25 mL) and DMF was added (500 μL). The solution was stirred at 80° C. for 1.5 h and the solvent was evaporated under reduced pressure. The remaining solid was partitioned between ethyl acetate (300 mL) and saturated aqueous sodium bicarbonate solution (100 mL) and stirred for 30 min. The organic layer was separated, dried over MgSO4, filtered and solvent was evaporated to afford a flaky butter-colored solid (2.963 g, 91%). ¹H NMR (400 MHz, CDCl₃) δ 8.36 (d, 1H), 7.92 (d, 1H), 7.80 (dd, 1H), 7.56 (d, 1H), 7.44 (s, 1H), 7.37 (d, 1H), 7.23 (dd, 1H); M+1=325.

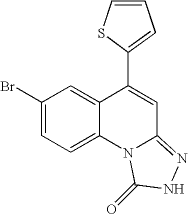

### 7-bromo-5-thien-2-yl[1,2,4]triazolo[4,3-a]quinolin-1(2H)-one

A 10 mL glass vial was loaded with 6-bromo-2-chloro4-thien-2-ylquinoline (324 mg, 1 mmol), ethyl carbazate (104 mg, 1 mmol), anhydrous ethanol (4 mL) and hydrogen bromide (10.9 μL, 0.2 mmol). The vial was sealed and subjected to microwave irradiation at 170° C. for 1 h. The cold vial was immersed in an ice-water bath and methanol was added (5 mL). The solid was filtered and washed with 2 mL DIEA and methanol (5×10 mL) on a frit funnel and dried in the air. A light pink powder was obtained (202 mg, 58%). ¹H NMR (400 MHz, DMSO-d₆) δ 12.72 (s, 1H), 8.97 (d, 1H), 7.89 (d, 1H), 7.87 (dd, 1H), 7.80 (dd, 1H), 7.45 (dd, 1H), 7.29 (dd, 1H), 7.25 (s, 1H); M+1=347.

### Example 504

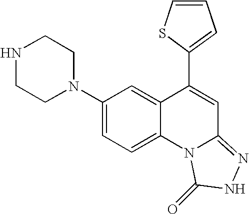

### 7-piperazin-1-yl-5-thien-2-yl[1,2,4]triazolo[4,3-a]quinolin-1(2H)-one

7-Bromo-5-thien-2-yl[1,2,4]triazolo[4,3-a]quinolin-1(2H)-one (173 mg, 0.5 mmol), 1-(diphenylmethyl)piperazine (151.4 mg, 0.5 mmol), Pd2(dba)3 (4.6 mg) and 2′-(dicyclohexylphosphino)-N,-dimethylbiphenyl-2-amine (4.2 mg) were loaded onto a dry flask and a 1M LiHMDS in THF solution was added under N₂ (1.1 mL). The dark solution was stirred at 65° C. for 16 h. The solvent was evaporated and the solid was triturated with methanol and the solid was collected on a fritted funnel. The solid was taken in triethylsilane (3 mL) and trifluorocetic acid was added and the solution was heated at reflux for 3 h. The solvent was evaporated and the residue was purified by reverse phase chromatography to afford a white solid (65 mg TFA salt). ¹H NMR (400 MHz, DMSO-d₆) δ 12.58 (brs, 1H), 8.95 (d, 1H), 8.76 (brs, 2H), 7.74 (dd, 1H), 7.44 (dd, 1H), 7.39-7.33 (m, 2H), 7.21 (dd, 1H), 7.12 (s, 1H), 3.32-3.26 (m, 8H); M+1=352.

### Example 505

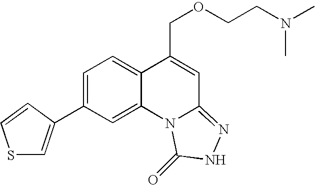

### 5-{[2-(dimethylamino)ethoxy]methyl}-8-thien-3-yl[1,2,4]triazolo[4,3-a]quinolin-1(2H)-one

5-(Hydroxymethyl)-8-(3-thienyl)[1,2,4]triazolo[4,3-a]quinolin-1(2H)-one (74 mg, 0.25 mmol) was suspended in 2 mL dry NMP under N₂ and triethylamine (77 μL, 0.55 mmol) was added. To the stirred solution methylsulfonyl chloride (43 μL, 0.55 mmol) was added and the mixture was stirred at room temperature for 2 h. 2-Dimethylaminoethanol (59 μL, 0.60 mmol) was dissolved in dry 1,4-dioxane under N₂ and cooled to 0° C. under stirring. N-BuLi 1.6 M in hexanes was added (375 μL, 0.60 mmol) and stirring was continued for 20 min. The solution was transferred to the previously prepared mesylate via a canula. After 5 h stirring at room temperature the solvents were evaporated under high vacuum and the residue was treated with 1N NaOH aqueous solution (2 mL) and stirred for 20 min. TFA was added (3 mL) and the solvents were evaporated. The remaining solid was subjected to reverse phase chromatography to afford a white solid (29 mg, 31%). ¹H NMR (400 MHz, DMSO-d₆) δ 12.87 (s, 1H), 9.36 (d, 1H), 8.30 (d, 1H), 8.06 (dd, 1H), 7.85 (dd, 1H), 7.74 (dd, 1H), 7.60 (dd, 1H), 7.55 (s, 1H), 4.87 (s, 2H), 3.93 (brs, 2H), 3.58 (brs, 2H), 3.12 (s, 6H); M+1=369.

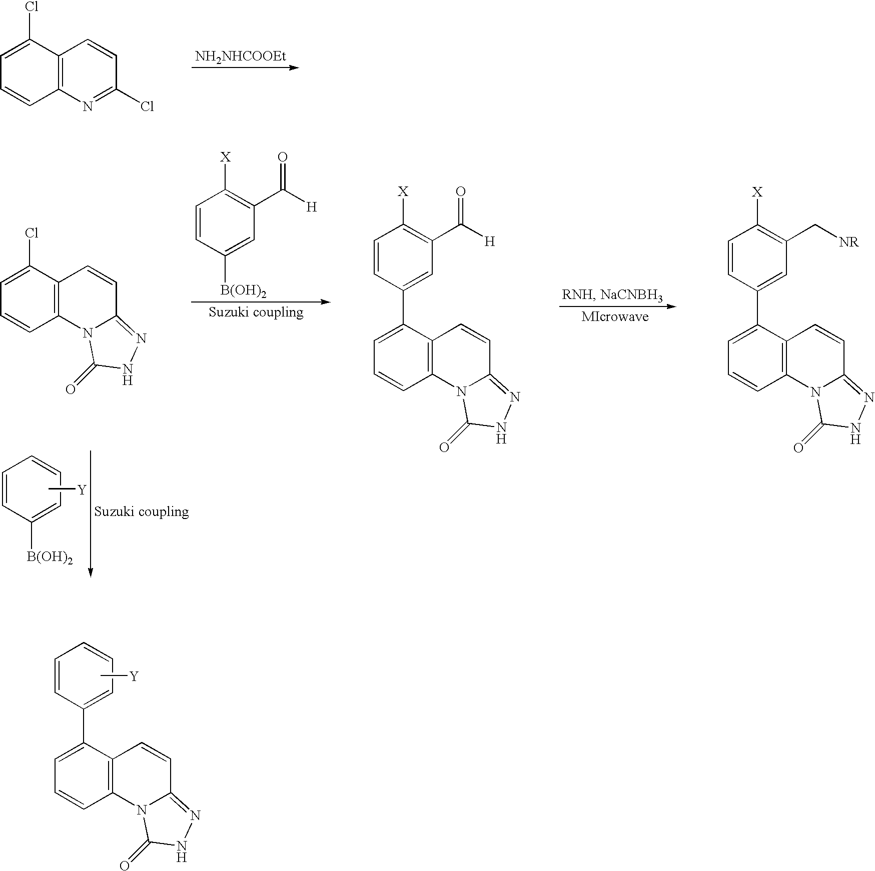

### 6-chloro-2H-[1,2,4]triazolo[4,3-α]quinolin-1-one

To a suspension of 2,4-dichloroquinoline (297 mg, 1.5 mmol) and ethyl carbazate (173 mg 1.66 mmol) in 3.3 ml of ethanol was added 6 drops of HCl (4N in dioxane). The reaction mixture was subject to microwave irradiation with microwave at 170° C. for 20 min. After cooling to room temperature the yellow precipitate was filtered off, rinsed with methanol (3×10 ml), and dried under vacuum to yield the desired compound as abrown solid (66%). m/z: 220

### Example 506-516

### 2-substituted-5-(1-oxo-1,2-dihydro-[1,2,4]triazolo[4,3-α]quinolin-6-yl)benzaldehyde

To a 5 ml vial, 6-chloro-2H-[1,2,4]triazolo[4,3-α]quinolin-1-one (110 mg, 0.5 mmol), boronic acid (0.6 mmol), cesium carbonate (651 mg, 2.0 mmol), and tetrakis(trisphenylphosphine)palladium (40 mg, 7 mol %) were added in 3.7 ml of dioxane:water (4:1). The reaction was subjected to microwave irradiation at 165° C. for 20 min. After cooling down, the lower layer was removed, the solid was filtered from the upper layer and rinsed with hot ethanol, and the filtrate concentrated to 10 ml. The solid that precipitated was filtered off and rinsed with methanol. The isolated solids were combined and used in subsequent steps without further purification.

The following examples were prepared by the following procedure using the appropriate amine.

To a suspension of the appropriate benzaldehyde (0.5 mmol) in 4 ml of DMF, amine (1 mmol) was added. The mixture was stirred overnight at room temperature. Then NaCNBH₃ (63 mg, 1 mmol) and 2 drops of AcOH were added to the mixture. The reaction was subjected to microwave irradiation at 150° C. for 5 min. 1 ml of water was added, the crude product separated purified by HPLC.

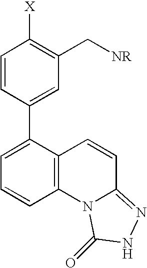

| Ex. | RN— | X | ¹H NMR(400 MHz, DMSO-D6) | m/z |
|---|---|---|---|---|
| 506 | 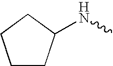 | H | 1.55-1.70n(m, 6H), 3.17(m, 1H), 4.26(s, 2H), 7.13-7.96(m, 8H), 8.89(s, br, 1H), 9.08(d, J=8.4Hz, 1H), 12.63(s, 1H) | 358 |
| 507 | 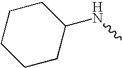 | H | 1.05-1.30(m, 5H), 1.62(m, 1H), 1.80(m, 2H), 2.15(m, 2H), 3.10(m, 1H), 4.28(s, 2H), 7.13-7.78(m, 8H), 8.79(s, br, 1H), 9.07(d, J=8.4Hz, 1H), 12.63(s, 1H). | 372 |
| 508 | 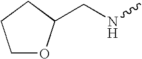 | H | 1.55(m, 1H), 1.85(m, 2H), 1.98(m, 1H), 2.95(m, 1H), 3.12(m, 1H), 3.85(m, 2H), 4.15(m, 1H), 4.27(s, 2H), 7.12-7.79(m, 8H), 9.04(s, br, 1H), 9.07(d, J=8.4Hz, 1H), 12.62(s, 1H). | 374 |
| 509 | 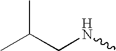 | H | 0.95(d, 6H), 2.01(m, 1H), 2.80(m, 2H), 4.26(s, 2H), 7.12-7.78(m, 8H), 8.78(s, br, 1H), 9.07(d, J=8.4Hz, 1H), 12.62(s, 1H) | 346 |
| 510 | 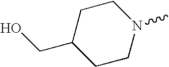 | H | 1.42(m, 2H), 1.61(m, 1H), 1.76-1.87(m, 3H), 2.95(m, 2H), 3.17(m, 1H), 3.27(m, 2H), 4.38(d, 2H), 7.13-7.72(m, 8H), 9.08(d, J=8.0Hz, 1H), 9.32(s, br, 1H), 12.62(s, 1H) | 388 |
| 511 | 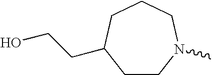 | H | 2.15(m, 2H), 2.55(m, 4H), 3.25(m, 4H), 3.73(m, 4H), 4.39(s, 2H), 5.40(s, br, 1H), 7.13-7.96(m, 8H), 9.08(d, J=8.0Hz, 1H), 12.63(s, 1H) | 417 |
| 512 | 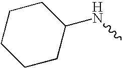 | OMe | 1.00-2.10(m, 10H), 3.07(m, 1H), 3.94(s, 3H), 4.21(s, 2H), 5.81(s, br, 1H), 7.12-7.72(m, 7H), 8.53(s, br, 1H), 9.04(d, J=8.4Hz, 1H), 12.61(s, 1H). | 402 |
| 513 | 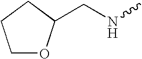 | OMe | 1.55(m, 1H), 1.90(m, 2H), 2.03(m, 1H), 3.02(m, 1H), 3.12(m, 1H), 3.75(m, 1H), 3.85(s, 1H), 3.89(m, 4H), 4.15(s, 2H), 7.10-7.75(m, 7H), 8.84(s, br, 1H), 9.04(d, J=8.4Hz, 1H), 12.60(s, 1H). | 404 |
| 514 | 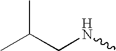 | OMe | 0.96(d, 6H), 2.00(m, 1H), 2.80(m, 2H), 3.94(s, 3H), 4.20(s, 2H), 7.12-7.54(m, 7H), 8.50(s, br, 1H), 9.05(d, 1H), 12.61(s, 1H) | 376 |
| 515 | 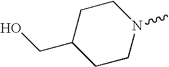 | OMe | 1.15(m, 2H), 1.33(m, 1H), 1.62(m, 2H), 1.97(m, 2H), 2.88(m, 2H), 3.21(m, 2H), 3.51(s, 2H), 3.85(s, 3H), 4.37(t, J=5.2Hz, 1H), 7.08-7.68(m, 7H), 9.01(d, J=8.4Hz, 1H), 12.50(s, 1H) | 418 |
| 516 | 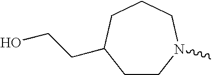 | OMe | 2.17(m, 2H), 2.50(m, 4H), 3.25(m, 4H), 3.73(m, 4H), 3.99(s, 3H), 4.40(s, 2H), 5.50(s, br, 1H), 7.12-7.95(m, 7H), 9.05(d, J=8.4Hz, 1H), 12.62(s, 1H) | 447 |

### Examples 517-522

The following examples were prepared by the following procedure using appropriate amine.

The appropriate methoxy compound (0.2 mmol) in 5 ml BBr₃ (1M in CH₂Cl₂) was stirred overnight at room temperature. Crushed ice was added and the solvent was removed under reduced pressure. The residue was dissolved in the minimum amount of DMSO and purified by HPLC.

| Ex. | RN— | ¹H NMR(400 MHz, DMSO-d6) | m/z |
|---|---|---|---|
| 517 | 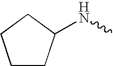 | 1.69-1.71(m, 6H), 2.00(m, 2H), 3.52(m, 1H), 4.15(s, 2H), 7.07-7.68(m, 7H), 8.60(s, br, 1H), 9.03(d, J=8.4Hz, 1H), 10.53(s, 1H), 12.60(s, 1H). | 374 |
| 518 | 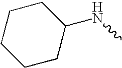 | 1.05-2.10(m, 10H), 3.05(m, 1H), 4.18(s, 2H), 7.07-7.70(m, 7H), 8.50(s, br, 1H), 9.02(d, J=8.4Hz, 1H), 10.53(s, 1H), 12.60(s, 1H). | 388 |
| 519 | 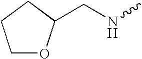 | 1.45-2.05(m, 4H), 2.80-3.10(m, 3H), 3.75-3.80(m, 2H), 4.20(s, 2H), 7.09-7.70(m, 7H), 8.70(m, 1H), 9.02(d, J=8.4Hz, 1H), 10.55(s, 1H), 12.59(s, 1H). | 390 |
| 520 | 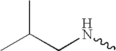 | 0.95(d, 6H), 2.00(m, 1H), 2.85(m, 2H), 4.18(s, 2H), 7.08-7.70(m, 7H), 8.57(s, br, 1H), 9.02(d, J=8.4Hz, 1H), 10.60(s, 1H), 12.59(s, 1H). | 362 |
| 521 | 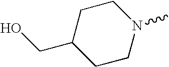 | 1.30-1.80(m, 6H), 3.00(m, 2H), 3.20-3.40(m, 3H), 4.29(s, 2H), 7.09-7.70(m, 7H), 9.03(d, J=8.4Hz, 1H), 10.55(s, 1H), 12.59(s, 1H). | 404 |
| 522 | 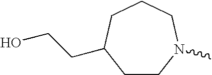 | 2.18(m, 2H), 2.59(m, 4H), 3.25(m, 4H), 3.73(m, 4H), 4.37(s, 2H), 5.42(s, br, 1H), 7.11-7.70(m, 7H), 9.03(d, J=8.4Hz, 1H), 9.92(s, br, 1H), 12.60(s, 1H). | 433 |

### Example 523

### 6-(3-hydroxymethyl-phenyl-2H-[1,2,4]triazolo[4,3-α]quinolin-1-one

To a 5 ml vial, 6-chloro-2H-[1,2,4]triazolo[4,3-α]quinolin-1-one (50 mg, 0.228 mmol), 3-aminophenylboronic acid (41.5 mg, 0.274 mmol), cesium carbonate (148.6 mg, 0.456 mmol), and tetrakis(trisphenylphosphine)palladium (18 mg, 7 mol %) were added in 3 ml of dioxane:water (4:1). The reaction was subject to microwave irradiation at 165° C. for 20 min. After cooling down, the upper layer was separated and concentrated. The residue was dissolved in the minimum amount of DMSO, filtered through a 2 μl cartridge, and the filtrate purified by HPLC to yield the title compound as a white solid (75.5%).

¹H NMR (400 MHz, DMSO-d6): 4.60 (s, 2H), 5.28 (s, br, 1H), 7.10-7.78 (m, 8H), 9.04 (d, J=8.4 Hz, 1H), 12.58 (s, 1H). m/z: 291

### Examples 524-525

The following examples were prepared by the following procedure using appropriate boronic acid

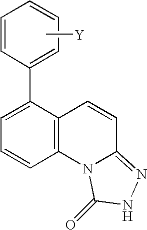

| Exp. | Y— | ¹H NMR(400 MHz, DMSO-d6) | m/z |
|---|---|---|---|
| 524 | 4-hydroxy- | 4.60(s, 2H), 5.30(s, br, 1H), | 291 |
| | methyl | 7.10-7.78(m, 8H), 9.03(d, J=8.4Hz, 1H), | |
| | | 12.57(s, 1H). | |
| 525 | 3-amino | 6.86-7.78(m, 8H), 9.03(d, J=8.4Hz, 1H), | |
| | | 12.56(s, 1H). | 276 |

The compounds of the present invention have utility for the treatment of neoplastic disease by acting upon checkpoint kinase. Methods of treatment target checkpoint kinase activity. Thus, inhibitors of checkpoint kinase have been shown to allow cells to progress inappropriately to the metaphase of mitosis leading to apoptosis of effected cells, and to therefore have anti-proliferative effects. Thus checkpoint kinase inhibitors act as modulators of cell division and are expected to be active against neoplastic disease such as carcinoma of the breast, ovary, lung, colon, prostate or other tissues, as well as leukemias and lymphomas, tumors of the central and peripheral nervous system, and other tumor types such as melanoma, fibrosarcoma and osteosarcoma. Checkpoint kinase inhibitors are also expected to be useful for the treatment other proliferative diseases including but not limited to autoimmune, inflammatory, neurological, and cardiovascular diseases.

Generally, the compounds of the present invention have been identified in one or both assays described below as having an IC50 value of 25 micromolar or less.

Checkpoint Kinase 1 Assay: This in vitro assay measures the inhibition of CHK1 kinase by compounds. The kinase domain is expressed in baculovirus and purified by the GST tag. Purified protein and biotinylated peptide substrate (Cdc25C) is then used in a 384 well automated Scintillation Proximity Assay (SPA). Specifically, peptide, enzyme and reaction buffer are mixed and aliquoted into a 384 well plate containing dilution series of compounds and controls. Cold and hot ATP are then added to initiate the reaction. After 2 hours, a SPA bead slurry, CsCl2 and EDTA are added to stop the reaction and capture the biotinylated peptide. Plates are then counted on a Topcount. Data is analyzed and IC50s determined for individual compounds.

Abrogation Assay: This cellular assay measures the ability of CHK1 inhibitors to abrogate the DNA-damage induced G2/M checkpoint. Compounds active against the enzyme (<2 uM) are tested in the cellular assay. Briefly HT29 cells (colon cancer cell line, p 53 null) are plated in 96 well plates on day 1. The following day, cells are treated with camptothecin for 2 hours to induce DNA damage. After 2 hours, camptothecin is removed and cells are treated for an additional 18 hours with test compound and nocodazole, a spindle poison that traps in cells in mitosis that abrogate the checkpoint. Cells are then fixed with formaldehyde, stained for the presence of phosphohistone H3, a specific marker for mitosis and labeled with Hoechst dye so that cell number can be measured. Plates are scanned using the Mitotic Index protocol on the Array Scan (Cellomics). As a positive control for abrogation, 4 mM caffeine is used. Compounds are tested in a 12-point dose response in triplicate. Data is analyzed and EC50s determined for individual compounds.

1. A compound having formula (I): 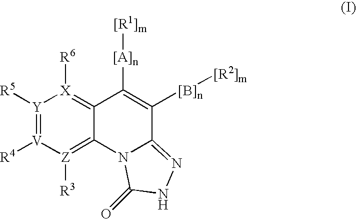
wherein:
m is independently selected at each occurrence from 0,1 or 2;
n is independently selected at each occurrence from 0 or 1;
A is optionally substituted phenyl, optionally substituted phenol, optionally substituted heterocyclic;
B is optionally substituted phenyl, optionally substituted phenol, optionally substituted heterocyclic;
R¹ is H, OH, F, Cl, Br, I, NH₂, —C(═O)R^{c}—C(═O)NHR^{c}, C(═O)CH₂R^{c}—C(═O)(CH₂)₂R^{c}, C(═O)(CH₂)₃R^{c}, —C(═O)NH(CH₂)NH₂, —C(═O)NH(CH₂)₂NH₂, —C(═O)NH(CH₂)₃NH₂, —C(═O)NH(CH₂)N(CH₃)₂, —C(═O)NH(CH₂)₂N(CH₃)₂, —C(═O)NH(CH₂)₃N(CH₃)₂, —C(═O)NH(CH₂)₂NHCH₃, —C(═O)NH(CH₂)₃OH, —C(═O)NHNH₂, —C(═O)NHCH(CH₃)CH₂N(CH₃)₂, —C(═O)NH(CH₂)₂NHC(CH₃)₂, (CH₂)₁₋₃OH, —C(═O)OR^{a}, —C(═O)NHNH₂, —NH(CH₂)₁₋₃R^{a}, —CH₂NH(CH₂)₁₋₃R^{a}, —NHC(═O)OR, —(C₆H₄)NH-cycloalkyl, —(C₆H₄)NH-optionally substituted heterocycle, —(C₆H₄)CH₂NH-alkyl-OH, —(C₆H₄)N(CH₃)₂, —O-alkyl-NH₂, optionally substituted alkyl, optionally substituted N-alkyl, optionally substituted alkenyl, optionally substituted alkynyl, optionally substituted cycloalkyl, optionally substituted cycloalkenyl, optionally substituted cycloalkynyl, optionally substituted aryl, optionally substituted alkoxy, optionally substituted heterocycle, or optionally substituted fused heterocycle;
R² is H, OH, F, Cl, Br, I, NH₂, (CH₂)₁₋₃OH, —C(═O)OR^{a}, —C(═O)NHNH₂, —NH(CH₂)₁₋₃R^{a}, —CH₂NH(CH₂)₁₋₃R^{a}, —NHC(═O)OR, optionally substituted alkyl, optionally substituted N-alkyl, optionally substituted alkenyl, optionally substituted alkynyl, optionally substituted cycloalkyl, optionally substituted cycloalkenyl, optionally substituted cycloalkynyl, optionally substituted aryl, optionally substituted alkoxy, optionally substituted heterocycle, or optionally substituted fused heterocycle.
R³ is is H, OH, F, Cl, Br, I, NH₂, CH₃;
R⁴ is H, OH, F, Cl, Br, I, NH₂, R^{a}, OCH3, —C(═O)OR^{a}, —C(═O)NHNH₂, —NH(CH₂)₁₋₃R^{a}, —CH₂NH(CH₂)₁₋₃R^{a}, —NHC(═O)OR^{a}, —(C₆H₄)CH₂NH(CH₂)₁₋₃R^{a}, —(C₆H₄)CH₂N(CH₃)(CH₂)₁₋₃R^{a}, —(C₆H₄)(CH₂)₀₋₃R^{a}, —(C₆H₄)(R^{b})CH₂R^{a}, —(C₆H₄)CH₂ NHR^{a}, —(C₆H₄)C(═O)R^{a}—(C₆H₄)NHC(═O)R^{a}, —(C₆H₄)CH₂NH(CH₂)₁₋₃R^{a}R^{b}, —(C₆H₄)NHSO₂CH₃, optionally substituted alkyl, optionally substituted N-alkyl, optionally substituted alkenyl, optionally substituted alkynyl, optionally substituted cycloalkyl, optionally substituted cycloalkenyl, optionally substituted cycloalkynyl, optionally substituted aryl, optionally substituted alkoxy, optionally substituted heterocycle, or optionally substituted fused heterocycle;
R⁵ is H, OH, F, Cl, Br, I, NH₂, OCH₃, —C(═O)OR^{a}, —C(═O)NHNH₂, —NH(CH₂)₁₋₃R^{a}, —CH₂NH(CH₂)₁₋₃R^{a}, —NHC(═O)OR^{a}, optionally substituted alkyl, optionally substituted N-alkyl, optionally substituted alkenyl, optionally substituted alkynyl, optionally substituted cycloalkyl, optionally substituted cycloalkenyl, optionally substituted cycloalkynyl, optionally substituted aryl, optionally substituted alkoxy, optionally substituted heterocycle, or optionally substituted fused heterocycle;
R⁶ is H, OH, F, Cl, Br, I, NH₂, NHC₁₋₆alkyl, N(C₁₋₆alkyl)₂, —(C₆H₄)CH₂R^{a}, —(C₆H₄)CH₂NR^{a}R^{b}, optionally substituted aryl;
R^{a} is H, OH, OCH₃, C₁₋₆alkyl, C₁₋₆alkoxy, NH₂, NHCH₃, N(CH₃)₂, CH₂C(CH₃)₂, optionally substituted phenyl, optionally substituted cycloalkyl, optionally substituted 5 or 6 or 7 membered heterocycle having 1 or 2 oxygen or 1 or 2 nitrogen or 1 nitrogen and 1 oxygen or 1 nitrogen and 1 sulfur or 1 oxygen and 1 sulfur ring atoms;
R^{b} is H, OH, OCH₃, C₁₋₆alkyl, C₁₋₆alkoxy;
R^{c} is optionally substituted C₄₋₇ heterocycle;
X is CH, substituted C, N, O, or any combination thereof;
Y is CH, substituted C, N, O, or any combination thereof;
Z is CH, substituted C, N, O, or any combination thereof;
V is CH, substituted C, N, O, or any combination thereof;
or a pharmaceutically aceptable salt thereof.

2. A compound of formula (I) as claimed in claim 1 wherein m is 0.

3. A compound of formula (I) as claimed in claim 1 wherein n is 0.

4. A compound of formula (I) as claimed in claim 1 wherein R¹—C(═O)R^{c}—C(═O)NHR^{c}, C(═O)CH₂R^{c}—C(═O)(CH₂)₂R^{c}, C(═O)(CH₂)₃R^{c}, —C(═O)NH(CH₂)NH₂, —C(═O)NH(CH₂)₂NH₂, —C(═O)NH(CH₂)₃NH₂, —C(═O)NH(CH₂)N(CH₃)₂, —C(═O)NH(CH₂)₂N(CH₃)₂, —C(═O)NH(CH₂)₃N(CH₃)₂, —C(═O)NH(CH₂)₂NHCH₃, —C(═O)NH(CH₂)₃OH, —C(═O)NHNH₂, —C(═O)NHCH(CH₃)CH₂N(CH₃)₂, —C(═O)NH(CH₂)₂NHC(CH₃)₂.

5. A compound of formula (I) as claimed in claim 1 wherein R¹ is NH₂, CH₃, or (CH₂)₁₋₃OH, —(C₆H₄)NHcycloalkyl, O(CH₂)₁₋₃NH₂, —(C₆H₄)NH-cycloalkyl, —(C₆H₄)NH-optionally substituted heterocycle, —(C₆H₄)CH₂NH-alkyl-OH, —(C₆H₄)N(CH₃)₂, —O-alkyl-NH₂.

6. A compound of formula (I) as claimed in claim 1 wherein R² is H or (CH₂)₁₋₃OH.

7. A compound of formula (I) as claimed in claim 1 wherein R³ is H.

8. A compound of formula (I) as claimed in claim 1 wherein R⁴ is H, OCH₃, —(C₆H₄)CH₂NH(CH₂)₁₋₃R^{a}, —(C₆H₄)CH₂N(CH₃)(CH₂)₁₋₃R^{a}, —(C₆H₄)CH₂R^{a}, —(C₆H₄)(R^{b})CH₂R^{a}, —(C₆H₄)CH₂ NHR^{a}, —(C₆H₄)C(═O)R^{a}, —(C₆H₄)NHC(═O)R^{a}, —(C₆H₄)CH₂NH(CH₂)₁₋₃R^{a}R^{b}, —(C₆H₄)NHSO₂CH₃, optionally substituted aryl, or optionally substituted heterocycle.

9. A compound of formula (I) as claimed in claim 1 wherein R⁴ is halogen, or an optionally substituted 5-membered heterocycle wherein said substitution is selected from —N(CH₃)₂, —NCH₂NCH₃, —CH₂NCH₃, CH₂-piperazine, or CH₂-methylpiperazine.

10. A compound of formula (I) as claimed in claim 1 wherein R⁴ is halogen or an optionally substituted furan, optionally substituted pyridine, or optionally substituted thiophene.

11. A compound of formula (I) as claimed in claim 1 wherein R⁴ is optionally substituted furan, optionally substituted pyridine, or optionally substituted thiophene wherein said substitution is selected from —N(CH₃)₂, —NCH₂NCH₃, —CH₂NCH₃, CH₂-piperazine, CH₂-methylpiperazine.

12. A compound of formula (I) as claimed in claim 1 wherein R⁵ is H, OH, or OCH₃.

13. A compound of formula (I) as claimed in claim 1 wherein R⁶is H, —(C₆H₄)CH₂R^{a}, —(C₆H₄)CH₂NR^{a}R^{b}.

14. A compound of formula (I) as claimed in claim 1 wherein X is CH or N.

15. A compound of formula (I) as claimed in claim 1 wherein Y is CH or N.

16. A compound of formula (I) as claimed in claim 1 wherein Z is CH or N.

17. A compound of formula (I) as claimed in claim 1 wherein V is an optionally substituted carbon.

18. A compound of formula (I) as claimed in claim 1 wherein:
m is 0 or 1;
n is 0;
R¹ is NH₂, CH₃, or (CH₂)₁₋₃OH; —(C₆H₄)NHcycloalkyl, O(CH₂)₁₋₃NH₂, —(C₆H₄)NH-cycloalkyl, —(C₆H₄)NH-optionally substituted heterocycle, —(C₆H₄)CH₂NH-alkyl-OH, —(C₆H₄)N(CH₃)₂, —O-alkyl-NH₂;
R²is H or (CH₂)₁₋₃OH;
R³is H;
R⁴ is OCH₃, —(C₆H₄)CH₂NH(CH₂)₁₋₃R^{a}, —(C₆H₄)CH₂N(CH₃)(CH₂)₁₋₃R^{a}, —(C₆H₄)CH₂R^{a}, —(C₆H₄)(R^{b})CH₂R^{a}, —(C₆H₄)CH₂ NHR^{a}—(C₆H₄)C(═O)R^{a}—(C₆H₄)NHC(═O)R^{a}, —(C₆H₄)CH₂NH(CH₂)₁₋₃R^{a}R^{b}, —(C₆H₄)NHSO₂CH₃, optionally substituted aryl, or optionally substituted heterocycle;
R⁵ is H, OH, or OCH₃;
R⁶ is H; —(C₆H₄)CH₂R^{a}, —(C₆H₄)CH₂NR^{a}R^{b};
R^{a} is OH, OCH₃, C₁₋₆alkyl, NH₂, NHCH₃, N(CH₃)₂, CH₂C(CH₃)₂, optionally substituted cycloalkyl, optionally substituted 5 or 6 or 7 membered heterocycle having 1 or 2 oxygen, or 1 or 2 nitrogen, or 1 nitrogen and 1 oxygen, or 1 nitrogen and 1 sulfur, or 1 oxygen and 1 sulfur ring atoms;
R^{b} is OH, OCH₃, C₁₋₆alkyl;
X, Y, Z and V are CH.

19. A compound of formula (I) as claimed in claim 1 wherein:
m is 1;
n is 0;
R¹ is —C(═O)R^{c}—C(═O)NHR^{c}, C(═O)CH₂R^{c}—C(═O)(CH₂)₂R^{c}, C(═O)(CH₂)₃R^{c}, —C(═O)NH(CH₂)NH₂, —C(═O)NH(CH₂)₂NH₂, —C(═O)NH(CH₂)₃NH₂, —C(═O)NH(CH₂)N(CH₃)₂, —C(═O)NH(CH₂)₂N(CH₃)₂, —C(═O)NH(CH₂)₃N(CH₃)₂, —C(═O)NH(CH₂)₂NHCH₃, —C(═O)NH(CH₂)₃OH, —C(═O)NHNH₂, —C(═O)NHCH(CH₃)CH₂N(CH₃)₂, —C(═O)NH(CH₂)₂NHC(CH₃)₂;
R² is H;
R³ is H;
R⁴ is halogen, or an optionally substituted 5-membered heterocycle;
R⁵ is H;
R⁶ is H;
X, Y, Z and V are CH.

20. A compound of formula (I) as claimed in claim 1 wherein:
m is 1;
n is 0;
R¹ is —C(═O)R^{c}—C(═O)NHR^{c}, C(═O)CH₂R^{c}—C(═O)(CH₂)₂R^{c}, C(═O)(CH₂)₃R^{c}, —C(═O)NH(CH₂)NH₂, —C(═O)NH(CH₂)₂NH₂, —C(═O)NH(CH₂)₃NH₂, —C(═O)NH(CH₂)N(CH₃)₂, —C(═O)NH(CH₂)₂N(CH₃)₂, —C(═O)NH(CH₂)₃N(CH₃)₂, —C(═O)NH(CH₂)₂NHCH₃, —C(═O)NH(CH₂)₃OH, —C(═O)NHNH₂, —C(═O)NHCH(CH₃)CH₂N(CH₃)₂, —C(═O)NH(CH₂)₂NHC(CH₃)₂;
R² is H;
R³ is H;
R⁴ is halogen, or an optionally substituted 5-membered heterocycle wherein said substitution is selected from —N(CH₃)₂, —NCH₂NCH₃, —CH₂NCH₃, —CH₂-piperazine or —CH₂-methylpiperazine.
R⁵ is H;
R⁶ is H;
X, Y, Z and V are CH.

21. A compound selected from:
5-methyl[1,2,4]triazolo[4,3-a]quinolin-1(2H)-one;
5,9-dimethyl[1,2,4]triazolo[4,3-a]quinolin-1(2H)-one;
8-methoxy-5-methyl[1,2,4]triazolo[4,3-a]quinolin-1(2H)-one;
8-fluoro-5-methyl[1,2,4]triazolo[4,3-a]quinolin-1(2H)-one;
5-bromo-5-(hydroxymethyl)[1,2,4]triazolo[4,3-a]quinolin-1(2H)-one [1,2,4]triazolo[4,3-a]quinolin-1(2H)-one;
ethyl-7-bromo-1-oxo-1,2-dihydro[1,2,4]triazolo[4,3-a]quinoline-5-carboxylate;
Ethyl-7-methyl-1-oxo-1,2-dihydro[1,2,4]triazolo[4,3-a]quinoline-5-carboxylate;
7-methyl-1-oxo-1,2-dihydro[1,2,4]triazolo[4,3-a]quinoline-5-carbohydrazide;
5-amino[1,2,4]triazolo[4,3-a]quinolin-1(2H)-one;
5-amino-7-bromo[1,2,4]triazolo[4,3-a]quinolin-1(2H)-one;
7-methoxy-5-methyl[1,2,4]triazolo[4,3-a]quinolin-1(2H)-one;
7-hydroxy-5-methyl[1,2,4]triazolo[4,3-a]quinolin-1(2H)-one;
8-hydroxy-5-methyl[1,2,4]triazolo[4,3-a]quinolin-1(2H)-one;
5,7-dimethyl[1,2,4]triazolo[4,3-a]quinolin-1(2H)-one;
5,8-dimethyl[1,2,4]triazolo[4,3-a]quinolin-1(2H)-one;
9-hydroxy[1,2,4]triazolo[4,3-a]quinolin-1(2H)-one;
T-butyl-7-bromo-1-oxo-1,2-dihydro[1,2,4]triazolo[4,3-a]quinolin-5-ylcarbamate;
7,8-dihydroxy-5-methyl[1,2,4]triazolo[4,3-a]quinolin-1(2H)-one;
7,8-methoxy-5-methyl[1,2,4]triazolo[4,3-a]quinolin-1(2H)-one;
7,8-methoxy[1,2,4]triazolo[4,3-a]quinolin-1(2H)-one;
7,8-dihydroxy[1,2,4]triazolo[4,3-a]quinolin-1(2H)-one;
5-chloro[1,2,4]triazolo[4,3-a]quinolin-1(2H)-one;
1-oxo-1,2-dihydro[1,2,4]triazolo[4,3-a]quinoline-5-carbohydrazide;
7-bromo-5-methyl[1,2,4]triazolo[4,3-a]quinolin-1(2H)-one;
7-iodo-5-methyl[1,2,4]triazolo[4,3-a]quinolin-1(2H)-one;
7-(3-aminophenyl)-5-methyl[1,2,4]triazolo[4,3-a]quinolin-1(2H)-one;
7-(3-hydroxyphenyl)-5-methyl[1,2,4]triazolo[4,3-a]quinolin-1(2H)-one;
8-(3-hydroxyphenyl)-5-methyl[1,2,4]triazolo[4,3-a]quinolin-1(2H)-one;
8-[3-(hydroxymethyl)phenyl-5-methyl[1,2,4]triazolo[4,3-a]quinolin-1(2H)-one;
8-[4-(hydroxymethyl)phenyl-5-methyl[1,2,4]triazolo[4,3-a]quinolin-1(2H)-one;
8-(3-aminophenyl)-5-methyl[1,2,4]triazolo[4,3-a]quinolin-1(2H)-one;
5-(3-aminophenyl)[1,2,4]triazolo[4,3-a]quinolin-1(2H)-one;
5-[4-(hydroxymethyl)phenyl[1,2,4]triazolo[4,3-a]quinolin-1(2H)-one;
Ethyl 7-methyl-1-oxo-1,2-dihydro[1,2,4]triazolo[4,3-a]quinoline-5-carboxylate;
5-amino-7-(3-aminophenyl)[1,2,4]triazolo[4,3-a]quinolin-1(2H)-one;
7-(2-hydroxyphenyl)-5-methyl[1,2,4]triazolo[4,3-a]quinolin-1(2H)-one;
4-amino[1,2,4]triazolo[4,3-a]quinolin-1(2H)-one;
5-amino-7-(3-hydroxyphenyl)[1,2,4]triazolo[4,3-a]quinolin-1(2H)-one;
5-amino-7-[3-(hydroxymethyl)phenyl][1,2,4]triazolo[4,3-a]quinolin-1(2H)-one;
5-(1-benzothien-2-yl)[1,2,4]triazolo[4,3-a]quinolin-1(2H)-one;
5-[3-(hydroxymethyl)phenyl][1,2,4]triazolo[4,3-a]quinolin-1(2H)-one;
5-[(E)-2-(4-chlorophenyl)vinyl][1,2,4]triazolo[4,3-a]quinolin-1(2H)-one;
5-(2,4-dihydroxyphenyl)[1,2,4]triazolo[4,3-a]quinolin-1(2H)-one;
7-(2-hydroxyphenyl)[1,2,4]triazolo[4,3-a]quinolin-1(2H)-one;
5-(2-furyl)[1,2,4]triazolo[4,3-a]quinolin-1(2H)-one;
7-(2,4-dihydroxyphenyl)-5-methyl[1,2,4]triazolo[4,3-a]quinolin-1(2H)-one;
5-phenyl[1,2,4]triazolo[4,3-a]quinolin-1(2H)-one;
5-{[2-(3,4-dimethoxyphenyl)ethyl]amino}[1,2,4]triazolo[4,3-a]quinolin-1(2H)-one;
5-[2,6-difluorobenzyl)amino][1,2,4]triazolo[4,3-a]quinolin-1(2H)-one;
Ethyl 1-oxo-1,2-dihydro[1,2,4]triazolo[4,3-a]quinoline-5-carboxylate;
5-{4-{[(2-pyridin-2-ylethyl)amino]methyl}phenyl}[1,2,4]triazolo[4,3-a]quinolin-1(2H)-one;
5-{4-{[(2-hydroxyethyl)amino]methyl}phenyl}[1,2,4]triazolo[4,3-a]quinolin-1(2H)-one 8-bromo-1-oxo-1,2-dihydro[1,2,4]triazolo[4,3-a]quinoline-5-carboxylic acid;
7-[(4-hydroxymethyl)phenyl]-5-methyl[1,2,4]triazolo[4,3-a]quinolin-1(2H)-one;
5-{4-[(4-methylpiperazin-1-yl)methyl]phenyl}[1,2,4]triazolo[4,3-a]quinolin-1(2H)-one;
5-(benzylamino)-7-bromo[1,2,4]triazolo[4,3-a]quinolin-1(2H)-one;
Ethyl 7-methoxy-1-oxo-1,2-dihydro[1,2,4]triazolo[4,3-a]quinoline-5-carboxylate;
5-[4-{{[3-(dimethylamino)propyl]amino}methyl}phenyl][1,2,4]triazolo[4,3-a]quinolin-1(2H)-one;
5-{4-{[(3-morpholin-4-ylpropyl)amino]methyl}phenyl}][1,2,4]triazolo[4,3-a]quinolin-1(2H)-one;
5-amino-7-methyl[1,2,4]triazolo[4,3-a]quinolin-1(2H)-one;
T-butyl 7-methoxy-1-oxo-1,2-dihydro[1,2,4]triazolo[4,3-a]quinolin-5-yl carbamate;
5-amino-7-methoxy[1,2,4]triazolo[4,3-a]quinolin-1(2H)-one;
8-dimethylamino-5-methyl[1,2,4]triazolo[4,3-a]quinolin-1(2H)-one;
5-amino-8-[4-(hydroxymethyl)phenyl][1,2,4]triazolo[4,3-a]quinolin-1(2H)-one;
5-(4-{[(2S)-2-(hydroxymethyl)pyrrolidin-1-yl]methyl}phenyl)[1,2,4]triazolo[4,3-a]quinolin-1(2H)-one;
5-[4-({[1-(hydroxymethyl)-2-methylpropyl]amino}methyl)phenyl][1,2,4]triazolo[4,3-a]quinolin-1(2H)-one;
5-(4-{[4-(3-methylphenyl)piperazin-1-yl]methyl}phenyl)[1,2,4]triazolo[4,3-a]quinolin-1(2H)-one;
7-[4-({[3-(dimethylamino)propyl]amino}methyl)phenyl]-5-methyl[1,2,4]triazolo[4,3-a]quinolin-1(2H)-one;
5-methyl-7-(4-{[(3-morpholin-4-ylpropyl)amino]methyl}phenyl)[1,2,4]triazolo[4,3-a]quinolin-1(2H)-one;
5-methyl-7-(4-{[(2-pyridin-2-ylethyl)amino]methyl}phenyl)[1,2,4]triazolo[4,3-a]quinolin-1(2H)-one;
7-(4-{[(2-hydroxyethyl)amino]methyl}phenyl)-5-methyl[1,2,4]triazolo[4,3-a]quinolin-1(2H)-one;
5-methyl-7-{4-[(4-methylpiperazin-1-yl)methyl]phenyl}[1,2,4]triazolo[4,3-a]quinolin-1(2H)-one;
7-(4-{[(2S)-2-(hydroxymethyl)pyrrolidin-1-yl]methyl}phenyl)-5-methyl[1,2,4]triazolo[4,3-a]quinolin-1(2H)-one;
7-[4-({[1-(hydroxymethyl)-2-methylpropyl]amino}methyl)phenyl]-5-methyl[1,2,4]triazolo[4,3-a]quinolin-1(2H)-one;
7-(4-{[ethyl(pyridin-4-ylmethyl)amino]methyl}phenyl)-5-methyl[1,2,4]triazolo[4,3-a]quinolin-1(2H)-one;
5-methyl-7-[4-({[3-(2-oxopyrrolidin-1-yl)propyl]amino}methyl)phenyl][1,2,4]triazolo[4,3-a]quinolin-1(2H)-one;
5-methyl-7-[4-({4-[3-(trifluoromethyl)phenyl]piperazin-1-yl}methyl)phenyl][1,2,4]triazolo[4,3-a]quinolin-1(2H)-one;
7-{4-[(isobutylamino)methyl]phenyl}-5-methyl[1,2,4]triazolo[4,3-a]quinolin-1(2H)-one;
5-[3-({[3-(dimethylamino)propyl]amino}methyl)-4-methoxyphenyl][1,2,4]triazolo[4,3-a]quinolin-1(2H)-one;
5-amino-8-[3-(hydroxymethyl)phenyl][1,2,4]triazolo[4,3-a]quinolin-1(2H)-one;
5-{3-[(dimethylamino)methyl]phenyl}[1,2,4]triazolo[4,3-a]quinolin-1(2H)-one;
5-{4-[(dimethylamino)methyl]phenyl}[1,2,4]triazolo[4,3-a]quinolin-1(2H)-one;
8-[3-(dimethylamino)phenyl]-5-methyl[1,2,4]triazolo[4,3-a]quinolin-1(2H)-one;
5-methyl-7-[4-({[2-(1H-pyrrol-1-yl)phenyl]amino}methyl)phenyl][1,2,4]triazolo[4,3-a]quinolin-1(2H)-one;
3-hydroxy-2-(1-oxo-1,2-dihydro[1,2,4]triazolo[4,3-a]quinolin-5-yl)benzaldehyde;
7-[4-({[3-(1H-imidazol-1-yl)propyl]amino}methyl)phenyl]-5-methyl[1,2,4]triazolo[4,3-a]quinolin-1(2H)-one;
5-(4-methoxy-3-{[(2-pyridin-2-ylethyl)amino]methyl}phenyl)[1,2,4]triazolo[4,3-a]quinolin-1(2H)-one;
5-[3-({[1-(hydroxymethyl)-2-methylpropyl]amino}methyl)-4-methoxyphenyl][1,2,4]triazolo[4,3-a]quinolin-1(2H)-one;
5-{4-methoxy-3-[(4-methylpiperazin-1-yl)methyl]phenyl}[1,2,4]triazolo[4,3-a]quinolin-1(2H)-one;
5-(3-{[(2R)-2-(hydroxymethyl)pyrrolidin-1-yl]methyl}-4-methoxyphenyl)[1,2,4]triazolo[4,3-a]quinolin-1(2H)-one;
5-[2-({[3-(dimethylamino)propyl]amino}methyl)-6-methoxyphenyl][1,2,4]triazolo[4,3-a]quinolin-1(2H)-one;
5-(2-{[(2S)-2-(hydroxymethyl)pyrrolidin-1-yl]methyl}-6-methoxyphenyl)[1,2,4]triazolo[4,3-a]quinolin-1(2H)-one;
5-(2-methoxy-6-{[(2-pyridin-2-ylethyl)amino]methyl}phenyl)[1,2,4]triazolo[4,3-a]quinolin-1(2H)-one;
5-{2-methoxy-6-[(4-methylpiperazin-1-yl)methyl]phenyl}[1,2,4]triazolo[4,3-a]quinolin-1(2H)-one;
5-[4-({[3-(1H-imidazol-1-yl)propyl]amino}methyl)phenyl][1,2,4]triazolo[4,3-a]quinolin-1(2H)-one;
5-(4-{[ethyl(pyridin-4-ylmethyl)amino]methyl}phenyl)[1,2,4]triazolo[4,3-a]quinolin-1(2H)-one;
5-[4-({[3-(2-oxopyrrolidin-1-yl)propyl]amino}methyl)phenyl][1,2,4]triazolo[4,3-a]quinolin-1(2H)-one;
5-[4-({[2-(1H-pyrrol-1-yl)phenyl]amino}methyl)phenyl][1,2,4]triazolo[4,3-a]quinolin-1(2H)-one;
5-(4-hydroxy-3-{[(2R)-2-(hydroxymethyl)pyrrolidin-1-yl]methyl}phenyl)[1,2,4]triazolo[4,3-a]quinolin-1(2H)-one;
5-[4-hydroxy-3-({[1-(hydroxymethyl)-2-methylpropyl]amino}methyl)phenyl][1,2,4]triazolo[4,3-a]quinolin-1(2H)-one;
5-(2-hydroxy-6-{[(2S)-2-(hydroxymethyl)pyrrolidin-1-yl]methyl}phenyl)[1,2,4]triazolo[4,3-a]quinolin-1(2H)-one;
5-{2-hydroxy-6-[(4-methylpiperazin-1-yl)methyl]phenyl}[1,2,4]triazolo[4,3-a]quinolin-1(2H)-one;
5-[4-({[4-(4-methylpiperazin-1-yl)phenyl]amino}methyl)phenyl][1,2,4]triazolo[4,3-a]quinolin-1(2H)-one;
5-(4-{[methyl(2-pyridin-2-ylethyl)amino]methyl}phenyl)[1,2,4]triazolo[4,3-a]quinolin-1(2H)-one;
5-(4-{[(2-furylmethyl)amino]methyl}phenyl)[1,2,4]triazolo[4,3-a]quinolin-1(2H)-one;
5-(4-{[(3-furylmethyl)amino]methyl}phenyl)[1,2,4]triazolo[4,3-a]quinolin-1(2H)-one;
5-{4-[({2-[({5-[(dimethylamino)methyl]-2-furyl}methyl)thio]ethyl}amino)methyl]phenyl}[1,2,4]triazolo[4,3-a]quinolin-1(2H)-one;
5-(4-{[(2,3-dihydro-1-benzofuran-3-ylmethyl)amino]methyl}phenyl)[1,2,4]triazolo[4,3-a]quinolin-1(2H)-one;
5-[4-({[(1-methyl-1H-pyrrol-2-yl)methyl]amino}methyl)phenyl][1,2,4]triazolo[4,3-a]quinolin-1(2H)-one;
5-[4-({[2-(4-benzylpiperazin-1-yl)ethyl]amino}methyl)phenyl][1,2,4]triazolo[4,3-a]quinolin-1(2H)-one;
5-(4-{[(pyridin-4-ylmethyl)amino]methyl}phenyl)[1,2,4]triazolo[4,3-a]quinolin-1(2H)-one;
5-(4-{[(4-morpholin-4-ylphenyl)amino]methyl}phenyl)[1,2,4]triazolo[4,3-a]quinolin-1(2H)-one;
5-amino-8-bromo[1,2,4]triazolo[4,3-a]quinolin-1(2H)-one;
7-(4-{[4-(hydroxymethyl)piperidin-1-yl]methyl}phenyl)-5-methyl[1,2,4]triazolo[4,3-a]quinolin-1(2H)-one;
5-(4-{[4-(hydroxymethyl)piperidin-1-yl]methyl}phenyl)[1,2,4]triazolo[4,3-a]quinolin-1(2H)-one;
7-(4-{[(2-furylmethyl)amino]methyl}phenyl)-5-methyl[1,2,4]triazolo[4,3-a]quinolin-1(2H)-one;
5-(4-{[4-(2-hydroxyethyl)piperidin-1-yl]methyl}phenyl)[1,2,4]triazolo[4,3-a]quinolin-1(2H)-one;
5-{4-[(4-pyridin-2-ylpiperazin-1-yl)methyl]phenyl}[1,2,4]triazolo[4,3-a]quinolin-1(2H)-one;
5-[4-({4-[4-(trifluoromethyl)pyrimidin-2-yl]-1,4-diazepan-1-yl}methyl)phenyl][1,2,4]triazolo[4,3-a]quinolin-1(2H)-one;
4-[4-(1-oxo-1,2-dihydro[1,2,4]triazolo[4,3-a]quinolin-5-yl)benzyl]piperazine-1-carbaldehyde;
4-[4-(1-oxo-1,2-dihydro[1,2,4]triazolo[4,3-a]quinolin-5-yl)benzyl]piperazine-1-carboxamide;
5-(4-{[(piperidin-4-ylmethyl)amino]methyl}phenyl)[1,2,4]triazolo[4,3-a]quinolin-1(2H)-one;
5-(4-{[4-(2-hydroxyethyl)-1,4-diazepan-1-yl]methyl}phenyl)[1,2,4]triazolo[4,3-a]quinolin-1(2H)-one;
5-[4-({4-[3-chloro-5-(trifluoromethyl)pyridin-2-yl]-1,4-diazepan-1-yl}methyl)phenyl][1,2,4]triazolo[4,3-a]quinolin-1(2H)-one;
5-(4-{[4-(3-nitropyridin-2-yl)-1,4-diazepan-1-yl]methyl}phenyl)[1,2,4]triazolo[4,3-a]quinolin-1(2H)-one;
7-(4-methoxy-3-{[(3-morpholin-4-ylpropyl)amino]methyl}phenyl)-5-methyl[1,2,4]triazolo[4,3-a]quinolin-1(2H)-one;
7-(3-{[(2-hydroxyethyl)amino]methyl}-4-methoxyphenyl)-5-methyl[1,2,4]triazolo[4,3-a]quinolin-1(2H)-one;
7-(4-methoxy-3-{[(2-pyridin-2-ylethyl)amino]methyl}phenyl)-5-methyl[1,2,4]triazolo[4,3-a]quinolin-1(2H)-one;
7-(3-{[4-(2-hydroxyethyl)piperidin-1-yl]methyl}-4-methoxyphenyl)-5-methyl[1,2,4]triazolo[4,3-a]quinolin-1(2H)-one;
7-{3-[({2-[({5-[(dimethylamino)methyl]-2-furyl}methyl)thio]ethyl}amino)methyl]-4-methoxyphenyl}-5-methyl[1,2,4]triazolo[4,3-a]quinolin-1(2H)-one;
5-{2-[({2-[({5-[(dimethylamino)methyl]-2-furyl}methyl)thio]ethyl}amino)methyl]-6-methoxyphenyl}[1,2,4]triazolo[4,3-a]quinolin-1(2H)-one;
5-{2-methoxy-6-[(4-pyridin-2-ylpiperazin-1-yl)methyl]phenyl}[1,2,4]triazolo[4,3-a]quinolin-1(2H)-one;
5-(3-{[(3-furylmethyl)amino]methyl}-4-methoxyphenyl)[1,2,4]triazolo[4,3-a]quinolin-1(2H)-one;
5-{3-[({2-[({5-[(dimethylamino)methyl]-2-furyl}methyl)thio]ethyl}amino)methyl]-4-methoxyphenyl}[1,2,4]triazolo[4,3-a]quinolin-1(2H)-one;
5-[3-({[2-(4-benzylpiperazin-1-yl)ethyl]amino}methyl)-4-methoxyphenyl][1,2,4]triazolo[4,3-a]quinolin-1(2H)-one;
5-{4-methoxy-3-[(4-pyridin-2-ylpiperazin-1-yl)methyl]phenyl}[1,2,4]triazolo[4,3-a]quinolin-1(2H)-one;
8-chloro-7-methoxy-5-methyl[1,2,4]triazolo[4,3-a]quinolin-1(2H)-one;
7-methoxy-5-methyl-8-pyridin-4-yl[1,2,4]triazolo[4,3-a]quinolin-1(2H)-one;
8-[3-(benzyloxy)phenyl]-7-methoxy-5-methyl[1,2,4]triazolo[4,3-a]quinolin-1(2H)-one;
7-methoxy-8-[4-(methoxymethyl)phenyl]-5-methyl[1,2,4]triazolo[4,3-a]quinolin-1(2H)-one;
Tert-butyl 3-(7-methoxy-5-methyl-1-oxo-1,2-dihydro[1,2,4]triazolo[4,3-a]quinolin-8-yl)benzylcarbamate;
8-[4-(aminomethyl)phenyl]-7-methoxy-5-methyl[1,2,4]triazolo[4,3-a]quinolin-1(2H)-one 4-methoxy-3-(7-methoxy-5-methyl-1-oxo-1,2-dihydro[1,2,4]triazolo[4,3-a]quinolin-8-yl)benzaldehyde;
8-(3,4-dimethoxyphenyl)-7-methoxy-5-methyl[1,2,4]triazolo[4,3-a]quinolin-1(2H)-one;
8-(3-chloro-4-fluorophenyl)-7-methoxy-5-methyl[1,2,4]triazolo[4,3-a]quinolin-1(2H)-one;
8-[4-(dimethylamino)phenyl]-7-methoxy-5-methyl[1,2,4]triazolo[4,3-a]quinolin-1(2H)-one;
8-(3-aminophenyl)-7-methoxy-5-methyl[1,2,4]triazolo[4,3-a]quinolin-1(2H)-one;
8-(2,6-dimethoxyphenyl)-7-methoxy-5-methyl[1,2,4]triazolo[4,3-a]quinolin-1(2H)-one;
7-methoxy-8-(3-methoxyphenyl)-5-methyl[1,2,4]triazolo[4,3-a]quinolin-1(2H)-one;
8-(4-chlorophenyl)-7-methoxy-5-methyl[1,2,4]triazolo[4,3-a]quinolin-1(2H)-one;
7-methoxy-8-[3-(methoxymethyl)phenyl]-5-methyl[1,2,4]triazolo[4,3-a]quinolin-1(2H)-one;
8-[3-(hydroxymethyl)phenyl]-7-methoxy-5-methyl[1,2,4]triazolo[4,3-a]quinolin-1(2H)-one;
8-[4-(hydroxymethyl)phenyl]-7-methoxy-5-methyl[1,2,4]triazolo[4,3-a]quinolin-1(2H)-one;
8-[3-(aminomethyl)phenyl]-7-methoxy-5-methyl[1,2,4]triazolo[4,3-a]quinolin-1(2H)-one;
8-(3-{[(2-hydroxyethyl)amino]methyl}phenyl)-7-methoxy-5-methyl[1,2,4]triazolo[4,3-a]quinolin-1(2H)-one;
7-methoxy-5-methyl-8-[3-({[(1-methyl-1H-pyrrol-2-yl)methyl]amino}methyl)phenyl][1,2,4]triazolo[4,3-a]quinolin-1(2H)-one;
8-(3-{[4-(2-hydroxyethyl)piperidin-1-yl]methyl}phenyl)-7-methoxy-5-methyl[1,2,4]triazolo[4,3-a]quinolin-1(2H)-one;
7-methoxy-5-methyl-8-(3-{[(pyridin-4-ylmethyl)amino]methyl}phenyl)[1,2,4]triazolo[4,3-a]quinolin-1(2H)-one;
8-{3-[(isobutylamino)methyl]phenyl}-7-methoxy-5-methyl[1,2,4]triazolo[4,3-a]quinolin-1(2H)-one;
7-methoxy-5-methyl-8-(3-{[(3-morpholin-4-ylpropyl)amino]methyl}phenyl)[1,2,4]triazolo[4,3-a]quinolin-1(2H)-one;
8-(3-{[4-(hydroxymethyl)piperidin-1-yl]methyl}phenyl)-7-methoxy-5-methyl[1,2,4]triazolo[4,3-a]quinolin-1(2H)-one;
8-[3-({[3-(1H-imidazol-1-yl)propyl]amino}methyl)phenyl]-7-methoxy-5-methyl[1,2,4]triazolo[4,3-a]quinolin-1(2H)-one;
8-(3-{[(3-chlorobenzyl)amino]methyl}phenyl)-7-methoxy-5-methyl[1,2,4]triazolo[4,3-a]quinolin-1(2H)-one;
7-methoxy-5-methyl-8-[3-({methyl[2-(methylamino)ethyl]amino}methyl)phenyl][1,2,4]triazolo[4,3-a]quinolin-1(2H)-one;
8-(4-methoxyphenyl)-5-methyl[1,2,4]triazolo[4,3-a]quinolin-1(2H)-one;
5-hydroxy-8-[4-(hydroxymethyl)phenyl][1,2,4]triazolo[4,3-a]quinolin-1(2H)-one;
5-methyl-8-pyridin-4-yl[1,2,4]triazolo[4,3-a]quinolin-1(2H)-one;
7-hydroxy-8-[2-(hydroxymethyl)phenyl]-5-methyl[1,2,4]triazolo[4,3-a]quinolin-1(2H)-one;
8-[3-(aminomethyl)phenyl]-7-hydroxy-5-methyl[1,2,4]triazolo[4,3-a]quinolin-1(2H)-one;
8-[3-({[3-(dimethylamino)propyl]amino}methyl)phenyl]-7-methoxy-5-methyl[1,2,4]triazolo[4,3-a]quinolin-1(2H)-one;
8-[3-({[3-(dimethylamino)propyl]amino}methyl)-4-methoxyphenyl]-7-methoxy-5-methyl[1,2,4]triazolo[4,3-a]quinolin-1(2H)-one;
7-methoxy-8-{4-methoxy-3-[(4-methylpiperazin-1-yl)methyl]phenyl}-5-methyl[1,2,4]triazolo[4,3-a]quinolin-1(2H)-one;
8-(3-{[(2-hydroxyethyl)amino]methyl}-4-methoxyphenyl)-7-methoxy-5-methyl[1,2,4]triazolo[4,3-a]quinolin-1(2H)-one;
7-methoxy-5-methyl-8-(1H-pyrrol-2-yl)[1,2,4]triazolo[4,3-a]quinolin-1(2H)-one;
N-[4-(5-methyl-1-oxo-1,2-dihydro[1,2,4]triazolo[4,3-a]quinolin-8-yl)phenyl]acetamide;
5-methyl-8-thien-2-yl[1,2,4]triazolo[4,3-a]quinolin-1(2H)-one;
5-amino-8-thien-2-yl[1,2,4]triazolo[4,3-a]quinolin-1(2H)-one;
5-amino-8-(2-furyl)[1,2,4]triazolo[4,3-a]quinolin-1(2H)-one;
5-methyl-8-(1H-pyrrol-2-yl)[1,2,4]triazolo[4,3-a]quinolin-1(2H)-one;
8-{2-furyl)-5-methyl[1,2,4]triazolo[4,3-a]quinolin-1(2H)-one;
5-amino-8-thien-3-yl[1,2,4]triazolo[4,3-a]quinolin-1(2H)-one;
5-methyl-8-thien-3-yl[1,2,4]triazolo[4,3-a]quinolin-1(2H)-one.
N,N-dimethyl-3-(5-methyl-1-oxo-1,2-dihydro[1,2,4]triazolo[4,3-a]quinolin-8-yl)benzamide;
5-{4-[(cyclopentylamino)methyl]phenyl}[1,2,4]triazolo[4,3-a]quinolin-1(2H)-one;
5-(4-{[(tetrahydrofuran-2-ylmethyl)amino]methyl}phenyl)[1,2,4]triazolo[4,3-a]quinolin-1(2H)-one;
5-(4-{[(2-hydroxypropyl)amino]methyl}phenyl)[1,2,4]triazolo[4,3-a]quinolin-1(2H)-one T-butyl 4-{[4-(1-oxo-1,2-dihydro[1,2,4]triazolo[4,3-a]quinolin-5-yl)benzyl]amino}piperidine-1-carboxylate;
7-hydroxy-8-(2-{[(2-hydroxypropyl)amino]methyl}phenyl)-5-methyl[1,2,4]triazolo[4,3-a]quinolin-1(2H)-one;
7-hydroxy-5-methyl-8-phenyl[1,2,4]triazolo[4,3-a]quinolin-1(2H)-one;
8-(4-chlorophenyl)-7-hydroxy-5-methyl[1,2,4]triazolo[4,3-a]quinolin-1(2H)-one;
8-(3-chloro-4-fluorophenyl)-7-hydroxy-5-methyl[1,2,4]triazolo[4,3-a]quinolin-1(2H)-one;
8-[4-(dimethylamino)phenyl]-7-hydroxy-5-methyl[1,2,4]triazolo[4,3-a]quinolin-1(2H)-one;
8-(3-aminophenyl)-7-hydroxy-5-methyl[1,2,4]triazolo[4,3-a]quinolin-1(2H)-one;
7-hydroxy-8-(2-methoxypyridin-4-yl)-5-methyl[1,2,4]triazolo[4,3-a]quinolin-1(2H)-one;
5-(3-aminopropoxy)[1,2,4]triazolo[4,3-a]quinolin-1(2H)-one;
8-(2-aminophenyl)-7-hydroxy-5-methyl[1,2,4]triazolo[4,3-a]quinolin-1(2H)-one;
8-{2-[(cyclopentylamino)methyl]phenyl}-7-hydroxy-5-methyl[1,2,4]triazolo[4,3-a]quinolin-1(2H)-one;
7-hydroxy-8-(3-hydroxyphenyl)-5-methyl[1,2,4]triazolo[4,3-a]quinolin-1(2H)-one;
7-hydroxy-5-methyl-8-[3-({methyl[2-(methylamino)ethyl]amino}methyl)phenyl][1,2,4]triazolo[4,3-a]quinolin-1(2H)-one;
7-hydroxy-8-[3-({[3-(1H-imidazol-1-yl)propyl]amino}methyl)phenyl]-5-methyl[1,2,4]triazolo[4,3-a]quinolin-1(2H)-one;
8-[3-({[3-(dimethylamino)propyl]amino}methyl)phenyl]-7-hydroxy-5-methyl[1,2,4]triazolo[4,3-a]quinolin-1(2H)-one;
7-hydroxy-5-methyl-8-(3-{[(pyridin-4-ylmethyl)amino]methyl}phenyl)[1,2,4]triazolo[4,3-a]quinolin-1(2H)-one;
4-amino-8-[4-(hydroxymethyl)phenyl][1,2,4]triazolo[4,3-a]quinolin-1(2H)-one;
7-hydroxy-5-methyl-8-(3-{[(3-morpholin-4-ylpropyl)amino]methyl}phenyl)[1,2,4]triazolo[4,3-a]quinolin-1(2H)-one;
8-(3-{[(3-chlorobenzyl)amino]methyl}phenyl)-7-hydroxy-5-methyl[1,2,4]triazolo[4,3-a]quinolin-1(2H)-one;
5-[3-({[2-(4-benzylpiperazin-1-yl)ethyl]amino}methyl)-4-hydroxyphenyl][1,2,4]triazolo[4,3-a]quinolin-1(2H)-one;
5-{2-hydroxy-6-[(4-pyridin-2-ylpiperazin-1-yl)methyl]phenyl}[1,2,4]triazolo[4,3-a]quinolin-1(2H)-one;
5-[2-({[2-(4-benzylpiperazin-1-yl)ethyl]amino}methyl)-6-hydroxyphenyl][1,2,4]triazolo[4,3-a]quinolin-1(2H)-one;
4-amino-8-thien-2-yl[1,2,4]triazolo[4,3-a]quinolin-1(2H)-one;
4-amino-8-[3-(hydroxymethyl)phenyl][1,2,4]triazolo[4,3-a]quinolin-1(2H)-one;
6-[3-(aminomethyl)phenyl][1,2,4]triazolo[4,3-a]quinolin-1(2H)-one;
5-(hydroxymethyl)-8-[3-(hydroxymethyl)phenyl]-7-methoxy[1,2,4]triazolo[4,3-a]quinolin-1(2H)-one;
8-(3-aminophenyl)-5-(hydroxymethyl)-7-methoxy[1,2,4]triazolo[4,3-a]quinolin-1(2H)-one;
7-hydroxy-5-(hydroxymethyl)-8-[2-(hydroxymethyl)phenyl][1,2,4]triazolo[4,3-a]quinolin-1(2H)-one;
7-hydroxy-5-(hydroxymethyl)-8-[3-(hydroxymethyl)phenyl][1,2,4]triazolo[4,3-a]quinolin-1(2H)-one;
8-(3-aminophenyl)-7-hydroxy-5-(hydroxymethyl)[1,2,4]triazolo[4,3-a]quinolin-1(2H)-one;
6-{3-[(cyclohexylamino)methyl]phenyl}[1,2,4]triazolo[4,3-a]quinolin-1(2H)-one;
6-{3-[(cyclopentylamino)methyl]phenyl}[1,2,4]triazolo[4,3-a]quinolin-1(2H)-one;
6-(3-{[(tetrahydrofuran-2-ylmethyl)amino]methyl}phenyl)[1,2,4]triazolo[4,3-a]quinolin-1(2H)-one;
6-(3-{[4-(hydroxymethyl)piperidin-1-yl]methyl}phenyl)[1,2,4]triazolo[4,3-a]quinolin-1(2H)-one;
4-(hydroxymethyl)-8-pyridin-4-yl[1,2,4]triazolo[4,3-a]quinolin-1(2H)-one;
8-(3-furyl)-5-methyl[1,2,4]triazolo[4,3-a]quinolin-1(2H)-one;
6-{3-[(isobutylamino)methyl]phenyl}[1,2,4]triazolo[4,3-a]quinolin-1(2H)-one;
6-(3-{[4-(2-hydroxyethyl)-1,4-diazepan-1-yl]methyl}phenyl)[1,2,4]triazolo[4,3-a]quinolin-1(2H)-one;
8-(2-aminophenyl)-7-hydroxy-5-(hydroxymethyl)[1,2,4]triazolo[4,3-a]quinolin-1(2H)-one;
6-(4-hydroxy-3-{[(tetrahydrofuran-2-ylmethyl)amino]methyl}phenyl)[1,2,4]triazolo[4,3-a]quinolin-1(2H)-one;
6-{4-hydroxy-3-[(isobutylamino)methyl]phenyl}[1,2,4]triazolo[4,3-a]quinolin-1(2H)-one;
4-(hydroxymethyl)-8-thien-2-yl[1,2,4]triazolo[4,3-a]quinolin-1(2H)-one;
5-(hydroxymethyl)-8-thien-2-yl[1,2,4]triazolo[4,3-a]quinolin-1(2H)-one;
5-(hydroxymethyl)-8-thien-3-yl[1,2,4]triazolo[4,3-a]quinolin-1(2H)-one;
7-methoxy-5-methyl-8-thien-3-yl[1,2,4]triazolo[4,3-a]quinolin-1(2H)-one;
7-hydroxy-5-methyl-8-thien-3-yl[1,2,4]triazolo[4,3-a]quinolin-1(2H)-one;
5-amino-7-hydroxy-8-thien-3-yl[1,2,4]triazolo[4,3-a]quinolin-1(2H)-one;
N-[3-(5-methyl-1-oxo-1,2-dihydro[1,2,4]triazolo[4,3-a]quinolin-8-yl)phenyl]methanesulfonamide;
5-amino-8-(1H-pyrrol-2-yl)[1,2,4]triazolo[4,3-a]quinolin-1(2H)-one;
5-(hydroxymethyl)-8-(1H-pyrrol-2-yl)[1,2,4]triazolo[4,3-a]quinolin-1(2H)-one;
5-methyl-8-(1H-pyrazol-4-yl)[1,2,4]triazolo[4,3-a]quinolin-1(2H)-one;
5-amino-8-(1H-pyrazol-4-yl)[1,2,4]triazolo[4,3-a]quinolin-1(2H)-one;
5-amino-8-(3-furyl)[1,2,4]triazolo[4,3-a]quinolin-1(2H)-one;
5-methyl-8-(4-methylthien-2-yl)[1,2,4]triazolo[4,3-a]quinolin-1(2H)-one;
8-[5-(hydroxymethyl)thien-2-yl]-5-methyl[1,2,4]triazolo[4,3-a]quinolin-1(2H)-one;
8-[5-(1-hydroxyethyl)thien-2-yl]-5-methyl[1,2,4]triazolo[4,3-a]quinolin-1(2H)-one;
Tert-butyl 4-{[(1-oxo-8-thien-3-yl-1,2-dihydro[1,2,4]triazolo[4,3-a]quinolin-5-yl)methyl}piperidine-1-carboxylate;
8-bromo-5-(hydroxymethyl)[1,2,4]triazolo[4,3-a]quinolin-1(2H)-one;
7-methoxy-8-[4-methoxy-3-({[(1-methyl-1H-pyrrol-2-yl)methyl]amino}methyl)phenyl]-5-methyl[1,2,4]triazolo[4,3-a]quinolin-1(2H)-one;
7-methoxy-8-(4-methoxy-3-{[(pyridin-4-ylmethyl)amino]methyl}phenyl)-5-methyl[1,2,4]triazolo[4,3-a]quinolin-1(2H)-one;
8-(3-{[4-(2-hydroxyethyl)piperidin-1-yl]methyl}-4-methoxyphenyl)-7-methoxy-5-methyl[1,2,4]triazolo[4,3-a]quinolin-1(2H)-one;
7-methoxy-8-(4-methoxy-3-{[(2-pyridin-2-ylethyl)amino]methylphenyl)-5-methyl[1,2,4]triazolo[4,3-a]quinolin-1(2H)-one;
8-(3-{[4-(hydroxymethyl)piperidin-1-yl]methyl}-4-methoxyphenyl)-7-methoxy-5-methyl[1,2,4]triazolo[4,3-a]quinolin-1(2H)-one;
7-methoxy-8-(4-methoxy-3-{[(2-methoxyethyl)amino]methyl}phenyl)-5-methyl[1,2,4]triazolo[4,3-a]quinolin-1(2H)-one;
7-methoxy-8-(3-{[(2-methoxyethyl)amino]methyl}phenyl)-5-methyl[1,2,4]triazolo[4,3-a]quinolin-1(2H)-one;
8-{3-[(cyclopentylamino)methyl]-4-methoxyphenyl}-7-methoxy-5-methyl[1,2,4]triazolo[4,3-a]quinolin-1(2H)-one;
8-(3-{[(4-fluorobenzyl)amino]methyl}-4-methoxyphenyl)-7-methoxy-5-methyl[1,2,4]triazolo[4,3-a]quinolin-1(2H)-one;
8-{3-[(cyclobutylamino)methyl]-4-methoxyphenyl}-7-methoxy-5-methyl[1,2,4]triazolo[4,3-a]quinolin-1(2H)-one;
8-{3-[(cyclohexylamino)methyl]-4-methoxyphenyl}-7-methoxy-5-methyl[1,2,4]triazolo[4,3-a]quinolin-1(2H)-one;
8-{3-[(cyclopentylamino)methyl]phenyl}-7-methoxy-5-methyl[1,2,4]triazolo[4,3-a]quinolin-1(2H)-one;
8-{3-[(cyclobutylamino)methyl]phenyl}-7-methoxy-5-methyl[1,2,4]triazolo[4,3-a]quinolin-1(2H)-one;
8-{3-[(cyclohexylamino)methyl]phenyl}-7-methoxy-5-methyl[1,2,4]triazolo[4,3-a]quinolin-1(2H)-one;
8-(3-{[(2-hydroxypropyl)amino]methyl}phenyl)-7-methoxy-5-methyl[1,2,4]triazolo[4,3-a]quinolin-1(2H)-one;
8-(3-{[4-(2-hydroxyethyl)-1,4-diazepan-1-yl]methyl}phenyl)-7-methoxy-5-methyl[1,2,4]triazolo[4,3-a]quinolin-1(2H)-one;
8-{3-[(cyclopropylamino)methyl]phenyl}-7-methoxy-5-methyl[1,2,4]triazolo[4,3-a]quinolin-1(2H)-one;
7-methoxy-5-methyl-8-(3-{[(tetrahydrofuran-2-ylmethyl)amino]methyl}phenyl)[1,2,4]triazolo[4,3-a]quinolin-1(2H)-one;
7-methoxy-5-methyl-8-(3-{[(2-phenoxyethyl)amino]methyl}phenyl)[1,2,4]triazolo[4,3-a]quinolin-1(2H)-one;
7-methoxy-5-methyl-8-[3-({[2-(2-thienyl)ethyl]amino}methyl)phenyl][1,2,4]triazolo[4,3-a]quinolin-1(2H)-one;
8-{3-[(cyclopropylamino)methyl]-4-methoxyphenyl}-7-methoxy-5-methyl[1,2,4]triazolo[4,3-a]quinolin-1(2H)-one;
5-methyl-8-pyridin-3-yl[1,2,4]triazolo[4,3-a]quinolin-1(2H)-one;
7-hydroxy-8-{3-[(isobutylamino)methyl]phenyl}-5-methyl[1,2,4]triazolo[4,3-a]quinolin-1(2H)-one;
7-hydroxy-8-(3-{[4-(2-hydroxyethyl)piperidin-1-yl]methyl}phenyl)-5-methyl[1,2,4]triazolo[4,3-a]quinolin-1(2H)-one;
7-hydroxy-5-methyl-8-{3-[(4-methylpiperazin-1-yl)methyl]phenyl}[1,2,4]triazolo[4,3-a]quinolin-1(2H)-one;
7-hydroxy-8-(4-hydroxy-3-{[(pyridin-4-ylmethyl)amino]methyl}phenyl)-5-methyl[1,2,4]triazolo[4,3-a]quinolin-1(2H)-one;
8-{3-[(cyclopentylamino)methyl]phenyl}-7-hydroxy-5-methyl[1,2,4]triazolo[4,3-a]quinolin-1(2H)-one;
8-(3-{[(4-fluorobenzyl)amino]methyl}phenyl)-7-hydroxy-5-methyl[1,2,4]triazolo[4,3-a]quinolin-1(2H)-one;
8-[2-(hydroxymethyl)phenyl]-7-methoxy-5-methyl[1,2,4]triazolo[4,3-a]quinolin-1(2H)-one;
7-methoxy-5-methyl-8-[4-(morpholin-4-ylcarbonyl)phenyl][1,2,4]triazolo[4,3-a]quinolin-1(2H)-one;
7-methoxy-5-methyl-8-[4-(pyrrolidin-1-ylcarbonyl)phenyl][1,2,4]triazolo[4,3-a]quinolin-1(2H)-one;
7-methoxy-5-methyl-8-[4-(piperidin-1-ylcarbonyl)phenyl][1,2,4]triazolo[4,3-a]quinolin-1(2H)-one;
8-chloro-7-hydroxy-5-methyl[1,2,4]triazolo[4,3-a]quinolin-1(2H)-one;
8-{3-[(cyclobutylamino)methyl]phenyl}-7-hydroxy-5-methyl[1,2,4]triazolo[4,3-a]quinolin-1(2H)-one;
7-hydroxy-5-methyl-8-(3-{[(tetrahydrofuran-2-ylmethyl)amino]methyl}phenyl)[1,2,4]triazolo[4,3-a]quinolin-1(2H)-one;
7-hydroxy-8-(3-{[4-(2-hydroxyethyl)-1,4-diazepan-1-yl]methyl}phenyl)-5-methyl[1,2,4]triazolo[4,3-a]quinolin-1(2H)-one;
8-{3-[(cyclopropylamino)methyl]phenyl}-7-hydroxy-5-methyl[1,2,4]triazolo[4,3-a]quinolin-1(2H)-one;
8-{3-[(cyclopropylamino)methyl]phenyl}-7-hydroxy-5-methyl[1,2,4]triazolo[4,3-a]quinolin-1(2H)-one;
8-{3-[(cyclohexylamino)methyl]phenyl}-7-hydroxy-5-methyl[1,2,4]triazolo[4,3-a]quinolin-1(2H)-one;
N-cyclohexyl-4-(7-methoxy-5-methyl-1-oxo-1,2-dihydro[1,2,4]triazolo[4,3-a]quinolin-8-yl)benzamide,
8-(2-{[(4-fluorobenzyl)amino]methyl}phenyl)-7-methoxy-5-methyl[1,2,4]triazolo[4,3-a]quinolin-1(2H)-one;
8-(2-{[(2-hydroxyethyl)amino]methyl}phenyl)-7-methoxy-5-methyl[1,2,4]triazolo[4,3-a]quinolin-1(2H)-one;
8-(2-{[4-(hydroxymethyl)piperidin-1-yl]methyl}phenyl)-7-methoxy-5-methyl[1,2,4]triazolo[4,3-a]quinolin-1(2H)-one;
8-(2-{[4-(hydroxymethyl)piperidin-1-yl]methyl}phenyl)-7-methoxy-5-methyl[1,2,4]triazolo[4,3-a]quinolin-1(2H)-one;
7-bromo-N-(4-methoxybenzyl)-1-oxo-1,2-dihydro[1,2,4]triazolo[4,3-a]quinoline-5-carboxamide;
8-(benzylamino)-5-methyl[1,2,4]triazolo[4,3-a]quinolin-1(2H)-one; N,N-dimethyl-4-(5-methyl-1-oxo-1,2-dihydro[1,2,4]triazolo[4,3-a]quinolin-8-yl)benzamide;
5-methyl-8-[4-(pyrrolidin-1-ylcarbonyl)phenyl][1,2,4]traizolo[4,3-a]quinolin-1(2H)-one;
5-methyl-8-[4-(piperidin-1-ylcarbonyl)phenyl][1,2,4]triazolo[4,3-a]quinolin-1(2H)-one; [4-(5-methyl-1-oxo-1,2-dihydro[1,2,4]triazolo[4,3-a]quinolin-8-yl)phenyl]acetonitrile;
8-[3-(hydroxymethyl)phenyl]-1-oxo-1,2-dihydro[1,2,4]triazolo[4,3-a]quinoline-5-carboxylic acid;
3-(5-methyl-1-oxo-1,2-dihydro[1,2,4]triazolo[4,3-a]quinolin-8-yl)benzonitrile;
5-methyl-8-[4-(morpholin-4-ylcarbonyl)phenyl][1,2,4]triazolo[4,3-a]quinolin-1(2H)-one;
8-[2-(hydroxymethyl)phenyl]-1-oxo-N-piperidin-4-yl-1,2-dihydro[1,2,4]triazolo[4,3-a]quinoline-5-carboxamide;
7-[3-(hydroxymethyl)phenyl]-1-oxo-N-piperidin-4-yl-1,2-dihydro[1,2,4]triazolo[4,3-a]quinoline-5-carboxamide;
[3-(7-methoxy-5-methyl-1-oxo-1,2-dihydro[1,2,4]triazolo[4,3-a]quinolin-8-yl)phenyl]acetonitrile;
N-(2-cyanoethyl)-3-(7-methoxy-5-methyl-1-oxo-1,2-dihydro[1,2,4]triazolo[4,3-a]quinolin-8-yl)benzamide;
6-chloro[1,2,4]triazolo[4,3-a]quinolin-1(2H)-one;
7-hydroxy-5-methyl-8-[4-(piperidin-1-ylcarbonyl)phenyl][1,2,4]triazolo[4,3-a]quinolin-1(2H)-one;
N-cyclohexyl-4-(7-hydroxy-5-methyl-1-oxo-1,2-dihydro[1,2,4]triazolo[4,3-a]quinolin-8-yl)benzamide;
6-[4-(hydroxymethyl)phenyl][1,2,4]triazolo[4,3-a]quinolin-1(2H)-one;
6-[3-(hydroxymethyl)phenyl][1,2,4]triazolo[4,3-a]quinolin-1(2H)-one;
6-(3-aminophenyl)[1,2,4]triazolo[4,3-a]quinolin-1(2H)-one;
7-methoxy-4-{[(pyridin-4-ylmethyl)amino]methyl}[1,2,4]triazolo[4,3-a]quinolin-1(2H)-one;
5-amino-8-pyridin-4-yl[1,2,4]triazolo[4,3-a]quinolin-1(2H)-one;
3-(5-methyl-1-oxo-1,2-dihydro[1,2,4]triazolo[4,3-a]quinolin-8-yl)benzamide;
2-(5-methyl-1-oxo-1,2-dihydro[1,2,4]triazolo[4,3-a]quinolin-8-yl)benzamide;
8-chloro-7-(3-chloropropoxy)-5-methyl[1,2,4]triazolo[4,3-a]quinolin-1(2H)-one;
8-chloro-7-(2-methoxyethoxy)-5-methyl[1,2,4]triazolo[4,3-a]quinolin-1(2H)-one;
8-[3-(hydroxymethyl)phenyl]-7-(2-methoxyethoxy)-5-methyl[1,2,4]triazolo[4,3-a]quinolin-1(2H)-one;
7-(3-aminopropoxy)-8-[3-(hydroxymethyl)phenyl]-5-methyl[1,2,4]triazolo[4,3-a]quinolin-1(2H)-one;
7-(3-aminopropoxy)-8-[2-(hydroxymethyl)phenyl]-5-methyl[1,2,4]triazolo[4,3-a]quinolin-1(2H)-one;
7-hydroxy-8-(2-hydroxyphenyl)-5-methyl[1,2,4]triazolo[4,3-a]quinolin-1(2H)-one;
8-bromo-4-(hydroxymethyl)[1,2,4]triazolo[4,3-a]quinolin-1(2H)-one;
5-methyl-8-(2-thienyl)[1,2,4]triazolo[4,3-a]quinolin-1(2H)-one;
N,N-dimethyl-3-(5-methyl-1-oxo-1,2-dihydro[1,2,4]triazolo[4,3-a]quinolin-8-yl)benzamide
5-{4-[(cyclopentylamino)methyl]phenyl}[1,2,4]triazolo[4,3-a]quinolin-1(2H)-one;
5-(4-{[(tetrahydrofuran-2-ylmethyl)amino]methyl}phenyl)[1,2,4]triazolo[4,3-a]quinolin-1(2H)-one;
5-(4-{[(2-hydroxypropyl)amino]methyl}phenyl)[1,2,4]triazolo[4,3-a]quinolin-1(2H)-one;
tert-butyl 4-{[4-(1-oxo-1,2-dihydro[1,2,4]triazolo[4,3-a]quinolin-5-yl)benzyl]amino}piperidine-1-carboxylate;
7-hydroxy-8-(2-{[(2-hydroxypropyl)amino]methyl}phenyl)-5-methyl[1,2,4]triazolo[4,3-a]quinolin-1(2H)-one;
8-(2-{[(4-fluorobenzyl)amino]methyl}phenyl)-7-hydroxy-5-methyl[1,2,4]triazolo[4,3-a]quinolin-1(2H)-one;
7-hydroxy-8-(2-{[4-(hydroxymethyl)piperidin-1-yl]methyl}phenyl)-5-methyl[1,2,4]triazolo[4,3-a]quinolin-1(2H)-one;
4-(hydroxymethyl)-8-methoxy[1,2,4]triazolo[4,3-a]quinolin-1(2H)-one;
8-{2-[(cyclopentylamino)methyl]phenyl}-7-methoxy-5-methyl[1,2,4]triazolo[4,3-a]quinolin-1(2H)-one;
4-[(cyclobutylamino)methyl]-7-hydroxy[1,2,4]triazolo[4,3-a]quinolin-1(2H)-one
7-hydroxy-5-methyl-8-phenyl[1,2,4]triazolo[4,3-a]quinolin-1(2H)-one;
8-(4-chlorophenyl)-7-hydroxy-5-methyl[1,2,4]triazolo[4,3-a]quinolin-1(2H)-one;
4-{[(4-fluorobenzyl)amino]methyl}-7-hydroxy[1,2,4]triazolo[4,3-a]quinolin-1(2H)-one;
8-[4-(dimethylamino)phenyl]-7-hydroxy-5-methyl[1,2,4]triazolo[4,3-a]quinolin-1(2H)-one;
8-(3-aminophenyl)-7-hydroxy-5-methyl[1,2,4]triazolo[4,3-a]quinolin-1(2H)-one;
8-(2-aminophenyl)-7-methoxy-5-methyl[1,2,4]triazolo[4,3-a]quinolin-1(2H)-one;
7-hydroxy-8-(2-methoxypyridin-4-yl)-5-methyl[1,2,4]triazolo[4,3-a]quinolin-1(2H)-one;
5-(3-aminopropoxy)[1,2,4]triazolo[4,3-a]quinolin-1(2H)-one;
8-[2-(hydroxymethyl)-4-methoxyphenyl]-5-methyl[1,2,4]triazolo[4,3-a]quinolin-1(2H)-one;
8-(2-aminophenyl)-7-hydroxy-5-methyl[1,2,4]triazolo[4,3-a]quinolin-1(2H)-one;
8-{2-[(cyclopentylamino)methyl]phenyl}-7-hydroxy-5-methyl[1,2,4]triazolo[4,3-a]quinolin-1(2H)-one;
7-hydroxy-8-(3-hydroxyphenyl)-5-methyl[1,2,4]triazolo[4,3-a]quinolin-1(2H)-one;
7-hydroxy-5-methyl-8-[3-({methyl[2-(methylamino)ethyl]amino}methyl)phenyl][1,2,4]triazolo[4,3-a]quinolin-1(2H)-one;
7-hydroxy-8-[3-({[3-(1H-imidazol-1-yl)propyl]amino}methyl)phenyl]-5-methyl[1,2,4]triazolo[4,3-a]quinolin-1(2H)-one;
7-hydroxy-5-methyl-8-(3-{[(pyridin-4-ylmethyl)amino]methyl}phenyl)[1,2,4]triazolo[4,3-a]quinolin-1(2H)-one;
4-amino-8-[4-(hydroxymethyl)phenyl][1,2,4]triazolo[4,3-a]quinolin-1(2H)-one;
8-hydroxy-4-(hydroxymethyl)[1,2,4]triazolo[4,3-a]quinolin-1(2H)-one;
2-(5-amino-1-oxo-1,2-dihydro[1,2,4]triazolo[4,3-a]quinolin-8-yl)benzamide;
7-hydroxy-5-methyl-8-(3-{[(3-morpholin-4-ylpropyl)amino]methyl}phenyl)[1,2,4]triazolo[4,3-a]quinolin-1(2H)-one;
8-(3-{[(3-chlorobenzyl)amino]methyl}phenyl)-7-hydroxy-5-methyl[1,2,4]triazolo[4,3-a]quinolin-1(2H)-one;
5-[3-({[2-(4-benzylpiperazin-1-yl)ethyl]amino}methyl)-4-hydroxyphenyl][1,2,4]triazolo[4,3-a]quinolin-1(2H)-one;
5-{2-hydroxy-6-[(4-pyridin-2-ylpiperazin-1-yl)methyl]phenyl}[1,2,4]triazolo[4,3-a]quinolin-1(2H)-one;
ethyl 8-chloro-7-methoxy-1-oxo-1,2-dihydro[1,2,4]triazolo[4,3-a]quinoline-5-carboxylate;
2-(5-hydroxy-1-oxo-1,2-dihydro[1,2,4]triazolo[4,3-a]quinolin-8-yl)benzamide;
5-[2-({[2-(4-benzylpiperazin-1-yl)ethyl]amino}methyl)-6-hydroxyphenyl][1,2,4]triazolo[4,3-a]quinolin-1(2H)-one;
4-amino-8-(2-thienyl)[1,2,4]triazolo[4,3-a]quinolin-1(2H)-one;
8-chloro-7-hydroxy-5-(hydroxymethyl)[1,2,4]triazolo[4,3-a]quinolin-1(2H)-one;
5-(hydroxymethyl)-8-[2-(hydroxymethyl)phenyl]-7-methoxy[1,2,4]triazolo[4,3-a]quinolin-1(2H)-one;
5-amino-8-(4-methoxyphenyl)[1,2,4]triazolo[4,3-a]quinolin-1(2H)-one;
5-methyl-8-(1H-pyrrol-2-yl)[1,2,4]triazolo[4,3-a]quinolin-1(2H)-one;
6-[4-(aminomethyl)phenyl][1,2,4]triazolo[4,3-a]quinolin-1(2H)-one;
N-[2-(1-oxo-1,2-dihydro[1,2,4]triazolo[4,3-a]quinolin-6-yl)phenyl]acetamide;
6-[2-(hydroxymethyl)phenyl][1,2,4]triazolo[4,3-a]quinolin-1(2H)-one;
4-amino-8-[3-(hydroxymethyl)phenyl][1,2,4]triazolo[4,3-a]quinolin-1(2H)-one;
4-amino-8-(2-furyl)[1,2,4]triazolo[4,3-a]quinolin-1(2H)-one;
6-[3-(aminomethyl)phenyl][1,2,4]triazolo[4,3-a]quinolin-1(2H)-one;
5-(hydroxymethyl)-8-[3-(hydroxymethyl)phenyl]-7-methoxy[1,2,4]triazolo[4,3-a]quinolin-1(2H)-one;
8-(3-aminophenyl)-5-(hydroxymethyl)-7-methoxy[1,2,4]triazolo[4,3-a]quinolin-1(2H)-one;
7-hydroxy-5-(hydroxymethyl)-8-[2-(hydroxymethyl)phenyl][1,2,4]triazolo[4,3-a]quinolin-1(2H)-one;
7-hydroxy-5-(hydroxymethyl)-8-[3-(hydroxymethyl)phenyl][1,2,4]triazolo[4,3-a]quinolin-1(2H)-one;
8-(3-aminophenyl)-7-hydroxy-5-(hydroxymethyl)[1,2,4]triazolo[4,3-a]quinolin-1(2H)-one;
6-{3-[(cyclohexylamino)methyl]-4-methoxyphenyl}[1,2,4]triazolo[4,3-a]quinolin-1(2H)-one;
6-(4-methoxy-3-{[(tetrahydrofuran-2-ylmethyl)amino]methyl}phenyl)[1,2,4]triazolo[4,3-a]quinolin-1(2H)-one;
6-{3-[(cyclohexylamino)methyl]phenyl}[1,2,4]triazolo[4,3-a]quinolin-1(2H)-one;
6-{3-[(cyclopentylamino)methyl]phenyl}[1,2,4]triazolo[4,3-a]quinolin-1(2H)-one;
6-(3-{[(tetrahydrofuran-2-ylmethyl)amino]methyl}phenyl)[1,2,4]triazolo[4,3-a]quinolin-1(2H)-one;
6-(3-{[4-(hydroxymethyl)piperidin-1-yl]methyl}-4-methoxyphenyl)[1,2,4]triazolo[4,3-a]quinolin-1(2H)-one;
6-(3-{[4-(hydroxymethyl)piperidin-1-yl]methyl}phenyl)[1,2,4]triazolo[4,3-a]quinolin-1(2H)-one;
6-(3-hydroxyphenyl)[1,2,4]triazolo[4,3-a]quinolin-1(2H)-one;
4-(hydroxymethyl)-8-pyridin-4-yl[1,2,4]triazolo[4,3-a]quinolin-1(2H)-one;
4-methyl-N-[1-oxo-8-(2-thienyl)-1,2-dihydro[1,2,4]triazolo[4,3-a]quinolin-4-yl]benzenesulfonamide;
6-{3-[(isobutylamino)methyl]-4-methoxyphenyl}[1,2,4]triazolo[4,3-a]quinolin-1(2H)-one;
6-(3-{[4-(2-hydroxyethyl)-1,4-diazepan-1-yl]methyl}-4-methoxyphenyl)[1,2,4]triazolo[4,3-a]quinolin-1(2H)-one;
6-{3-[(isobutylamino)methyl]phenyl}[1,2,4]triazolo[4,3-a]quinolin-1(2H)-one;
6-(3-{[4-(2-hydroxyethyl)-1,4-diazepan-1-yl]methyl}phenyl)[1,2,4]triazolo[4,3-a]quinolin-1(2H)-one;
8-(2-aminophenyl)-7-hydroxy-5-(hydroxymethyl)[1,2,4]triazolo[4,3-a]quinolin-1(2H)-one;
6-(4-hydroxy-3-{[(tetrahydrofuran-2-ylmethyl)amino]methyl}phenyl)[1,2,4]triazolo[4,3-a]quinolin-1(2H)-one;
6-{4-hydroxy-3-[(isobutylamino)methyl]phenyl}[1,2,4]triazolo[4,3-a]quinolin-1(2H)-one;
6-(4-hydroxy-3-{[4-(hydroxymethyl)piperidin-1-yl]methyl}phenyl)[1,2,4]triazolo[4,3-a]quinolin-1(2H)-one;
4-(hydroxymethyl)-8-(2-thienyl)[1,2,4]triazolo[4,3-a]quinolin-1(2H)-one;
6-(4-hydroxy-3-{[4-(2-hydroxyethyl)-1,4-diazepan-1-yl]methyl}phenyl)[1,2,4]traizolo[4,3-a]quinolin-1(2H)-one;
5-amino-8-(3-thienyl)[1,2,4]triazolo[4,3-a]quinolin-1(2H)-one;
5-amino-8-chloro-7-hydroxy[1,2,4]triazolo[4,3-a]quinolin-1(2H)-one;
7-methoxy-5-methyl-8-(3-thienyl)[1,2,4]triazolo[4,3-a]quinolin-1(2H)-one;
7-hydroxy-5-methyl-8-(3-thienyl)[1,2,4]triazolo[4,3-a]quinolin-1(2H)-one;
5-amino-7-hydroxy-8-(3-thienyl)[1,2,4]triazolo[4,3-a]quinolin-1(2H)-one;
N-[2-(5-methyl-1-oxo-1,2-dihydro[1,2,4]triazolo[4,3-a]quinolin-8-yl)phenyl]methanesulfonamide;
8-(1H-indol-3-yl)-5-methyl[1,2,4]triazolo[4,3-a]quinolin-1(2H)-one;
N-[3-(5-methyl-1-oxo-1,2-dihydro[1,2,4]triazolo[4,3-a]quinolin-8-yl)phenyl]methanesulfonamide;
5-amino-8-(1H-pyrrol-2-yl)[1,2,4]triazolo[4,3-a]quinolin-1(2H)-one;
5-(hydroxymethyl)-8-(1H-pyrrol-2-yl)[1,2,4]triazolo[4,3-a]quinolin-1(2H)-one;
5-methyl-8-(1H-pyrazol-4-yl)[1,2,4]triazolo[4,3-a]quinolin-1(2H)-one;
5-amino-8-(1H-pyrazol-4-yl)[1,2,4]triazolo[4,3-a]quinolin-1(2H)-one;
5-amino-8-(3-furyl)[1,2,4]triazolo[4,3-a]quinolin-1(2H)-one;
5-methyl-8-(4-methyl-2-thienyl)[1,2,4]triazolo[4,3-a]quinolin-1(2H)-one;
8-(3-furyl)-5-(hydroxymethyl)[1,2,4]triazolo[4,3-a]quinolin-1(2H)-one;
8-[5-(hydroxymethyl)-2-thienyl]-5-methyl[1,2,4]triazolo[4,3-a]quinolin-1(2H)-one;
8-[5-(1-hydroxyethyl)-2-thienyl]-5-methyl[1,2,4]triazolo[4,3-a]quinolin-1(2H)-one;
5-(5-methyl-1-oxo-1,2-dihydro[1,2,4]triazolo[4,3-a]quinolin-8-yl)thiophene-2-carboxylic acid;
tert-butyl 4-({[1-oxo-8-(3-thienyl)-1,2-dihydro[1,2,4]triazolo[4,3-a]quinolin-5-yl]amino}methyl)piperidine-1-carboxylate;
5-amino-8-[5-(1-hydroxyethyl)-2-thienyl][1,2,4]triazolo[4,3-a]quinolin-1(2H)-one;
8-(1H-imidazol-4-yl)-5-methyl[1,2,4]triazolo[4,3-a]quinolin-1(2H)-one;
5-(hydroxymethyl)-8-(1H-pyrazol-4-yl)[1,2,4]triazolo[4,3-a]quinolin-1(2H)-one;
8-bromo-5-[(dimethylamino)methyl][1,2,4]triazolo[4,3-a]quinolin-1(2H)-one;
8-(2-furyl)-5-(hydroxymethyl)[1,2,4]triazolo[4,3-a]quinolin-1(2H)-one;
5-methyl-8-(1,3-thiazol-2-yl)[1,2,4]triazolo[4,3-a]quinolin-1(2H)-one;
5-[(dimethylamino)methyl]-8-(1H-pyrrol-2-yl)[1,2,4]triazolo[4,3-a]quinolin-1(2H)-one;
5-methyl-8-pyrazin-2-yl[1,2,4]triazolo[4,3-a]quinolin-1(2H)-one;
5-(hydroxymethyl)-8-pyridin-4-yl[1,2,4]triazolo[4,3-a]quinolin-1(2H)-one;
(5-methyl-1-oxo-1,2-dihydro[1,2,4]triazolo[4,3-a]quinolin-8-yl)boronic acid;
8-(2-furyl)-5-phenyl[1,2,4]triazolo[4,3a]quinolin-1(2H)-one;
5-phenyl-8-(1H-pyrrol-2-yl)[1,2,4]triazolo[4,3-a]quinolin-1(2H)-one;
8-(3-furyl)-5-(morpholin-4-ylmethyl)[1,2,4]triazolo[4,3-a]quinolin-1(2H)-one;
tert-butyl [2-({[1-oxo-8-(1H-pyrrol-2-yl)-1,2-dihydro[1,2,4]triazolo[4,3-a]quinolin-5-yl]methyl}amino)ethyl]carbamate;
5-{[(2-aminoethyl)amino]methyl}-8-(1H-pyrrol-2-yl)[1,2,4]triazolo[4,3-a]quinolin-1(2H)-one;
N-(2-aminoethyl)-8-bromo-1-oxo-1,2-dihydro[1,2,4]triazolo[4,3-a]quinoline-5-carboxamide;
8-(3-furyl)-1-oxo-1,2-dihydro[1,2,4]triazolo[4,3-a]quinoline-5-carboxylic acid;
8-[3-(aminomethyl)phenyl]-5-phenyl[1,2,4]triazolo[4,3-a]quinolin-1(2H)-one;
N-[2-(dimethylamino)ethyl]-8-(3-furyl)-1-oxo-1,2-dihydro[1,2,4]triazolo[4,3-a]quinoline-5-carboxamide;
5-methyl-8-[4-(2-morpholin-4-ylethoxy)phenyl][1,2,4]triazolo[4,3-a]quinolin-1(2H)-one;
8-{4-[2-(diethylamino)ethoxy]phenyl}-5-methyl[1,2,4]triazolo[4,3-a]quinolin-1(2H)-one;
8-[3-(dimethylamino)prop-1-yn-1-yl]-5-methyl[1,2,4]triazolo[4,3-a]quinolin-1(2H)-one;
7-piperazin-1-yl-5-(2-thienyl)[1,2,4]triazolo[4,3-a]quinolin-1(2H)-one;
5-methyl-8-[3-(methylamino)prop-1-yn-1-yl][1,2,4]triazolo[4,3-a]quinolin-1(2H)-one;
5-methyl-8-[4-(morpholin-4-ylmethyl)phenyl][1,2,4]triazolo[4,3-a]quinolin-1(2H)-one;
N-[2-(dimethylamino)ethyl]-1-oxo-8-(1H-pyrrol-2-yl)-1,2-dihydro[1,2,4]triazolo[4,3-a]quinoline-5-carboxamide;
N-[2-(dimethylamino)ethyl]-1-oxo-8-(3-thienyl)-1,2-dihydro[1,2,4]triazolo[4,3-a]quinoline-5-carboxamide;
5-{[(3R)-piperidin-3-ylamino]methyl}-8-(1H-pyrrol-2-yl)[1,2,4]triazolo[4,3-a]quinolin-1(2H)-one;
5-methyl-8-{4-[(4-methylpiperazin-1-yl)methyl]phenyl}[1,2,4]triazolo[4,3-a]quinolin-1(2H)-one;
tert-butyl (3S)-3-({[1-oxo-8-(3-thienyl)-1,2-dihydro[1,2,4]triazolo[4,3-a]quinolin-5-yl]carbonyl}amino)piperidine-1-carboxylate;
5-methyl-8-{4-[(methylamino)methyl]phenyl}[1,2,4]triazolo[4,3-a]quinolin-1(2H)-one;
8-(3,3-diethoxyprop-1-yn-1-yl)-5-methyl[1,2,4]triazolo[4,3-a]quinolin-1(2H)-one;
5-methyl-8-[5-(morpholin-4-ylmethyl)-3-thienyl][1,2,4]triazolo[4,3-a]quinolin-1(2H)-one;
tert-butyl 4-[4-(5-methyl-1-oxo-1,2-dihydro[1,2,4]triazolo[4,3-a]quinolin-8-yl)benzyl]piperazine-1-carboxylate;
5-methyl-8-{5-[(methylamino)methyl]-3-thienyl}[1,2,4]triazolo[4,3-a]quinolin-1(2H)-one;
5-methyl-8-{5-[(4-methylpiperazin-1-yl)methyl]-3-thienyl}[1,2,4]triazolo[4,3-a]quinolin-1(2H)-one;
tert-butyl 4-{[4-(5-methyl-1-oxo-1,2-dihydro[1,2,4]triazolo[4,3-a]quinolin-8-yl)-2-thienyl]methyl}piperazine-1-carboxylate;
5-methyl-8-[4-(piperazin-1-ylmethyl)phenyl][1,2,4]triazolo[4,3-a]quinolin-1(2H)-one;
1-oxo-N-[(3S)-piperidin-3-yl]-8-(3-thienyl)-1,2-dihydro[1,2,4]triazolo[4,3-a]quinoline-5-carboxamide;
tert-butyl (3S)-3-({[1-oxo-8-(1H-pyrrol-2-yl)-1,2-dihydro[1,2,4]triazolo[4,3-a]quinolin-5-yl]carbonyl}amino)piperidine-1-carboxylate;
5-methyl-8-[5-(piperazin-1-ylmethyl)-3-thienyl][1,2,4]triazolo[4,3-a]quinolin-1(2H)-one;
N-(2-aminoethyl)-1-oxo-8-(3-thienyl)-1,2-dihydro[1,2,4]triazolo[4,3-a]quinoline-5-carboxamide;
1-oxo-N-[(3S)-piperidin-3-yl]-8-(1H-pyrrol-2-yl)-1,2-dihydro[1,2,4]triazolo[4,3-a]quinoline-5-carboxamide;
5-methyl-8-(1H-pyrrol-3-yl)[1,2,4]triazolo[4,3-a]quinolin-1(2H)-one;
5-methyl-8-(3-thienylethynyl)[1,2,4]triazolo[4,3-a]quinolin-1(2H)-one;
8-[5-({[3-(dimethylamino)propyl]amino}methyl)-3-thienyl]-5-methyl[1,2,4]triazolo[4,3-a]quinolin-1(2H)-one;
5-methyl-8-{5-[(methylamino)methyl]-2-thienyl}[1,2,4]triazolo[4,3-a]quinolin-1(2H)-one;
5-(hydroxymethyl)-8-[5-(morpholin-4-ylmethyl)-3-thienyl][1,2,4]triazolo[4,3-a]quinolin-1(2H)-one;
5-(hydroxymethyl)-8-{5-[(methylamino)methyl]-3-thienyl}[1,2,4]triazolo[4,3-a]quinolin-1(2H)-one;
5-(hydroxymethyl)-8-{5-[(4-methylpiperazin-1-yl)methyl]-3-thienyl}[1,2,4]triazolo[4,3-a]quinolin-1(2H)-one;
8-{5-[(dimethylamino)methyl]-3-thienyl}-5-(hydroxymethyl)[1,2,4]triazolo[4,3-a]quinolin-1(2H)-one;
5-(hydroxymethyl)-8-[5-(piperazin-1-ylmethyl)-3-thienyl][1,2,4]triazolo[4,3-a]quinolin-1(2H)-one;
N-[2-(methylamino)ethyl]-1-oxo-8-(3-thienyl)-1,2-dihydro[1,2,4]triazolo[4,3-a]quinoline-5-carboxamide;
N-[2-(methylamino)ethyl]-1-oxo-8-(1H-pyrrol-2-yl)-1,2-dihydro[1,2,4]triazolo[4,3-a]quinoline-5-carboxamide;
8-bromo-5-{[(2-methoxyethyl)(methyl)amino]methyl}[1,2,4]triazolo[4,3-a]quinolin-1(2H)-one;
N-[2-(dimethylamino)ethyl]-8-{5-[(4-methylpiperazin-1-yl)methyl]-3-thienyl}-1-oxo-1,2-dihydro[1,2,4]triazolo[4,3-a]quinoline-5-carboxamide;
8-bromo-5-{[2-(dimethylamino)ethoxy]methyl}[1,2,4]triazolo[4,3-a]quinolin-1(2H)-one;
N-(2-morpholin-4-ylethyl)-1-oxo-8-(3-thienyl)-1,2-dihydro[1,2,4]triazolo[4,3-a]quinoline-5-carboxamide;
5-[(4-methylpiperazin-1-yl)methyl]-8-(3-thienyl)[1,2,4]triazolo[4,3-a]quinolin-1(2H)-one;
8-[5-({[3-(dimethylamino)propyl]amino}methyl)-3-thienyl]-5-(hydroxymethyl)[1,2,4]triazolo[4,3-a]quinolin-1(2H)-one;
5-amino-8-{5-[(methylamino)methyl]-3-thienyl}[1,2,4]triazolo[4,3-a]quinolin-1(2H)-one;
5-(hydroxymethyl)-8-[5-(morpholin-4-ylmethyl)-2-thienyl][1,2,4]triazolo[4,3-a]quinolin-1(2H)-one;
5-(hydroxymethyl)-8-{5-[(methylamino)methyl]-2-thienyl}[1,2,4]triazolo[4,3-a]quinolin-1(2H)-one;
5-(hydroxymethyl)-8-{5-[(4-methylpiperazin-1-yl)methyl]-2-thienyl}[1,2,4]triazolo[4,3-a]quinolin-1(2H)-one;
8-{5-[(dimethylamino)methyl]-2-thienyl}-5-(hydroxymethyl)[1,2,4]triazolo[4,3-a]quinolin-1(2H)-one;
5-[(3-hydroxypyrrolidin-1-yl)methyl]-8-(3-thienyl)[1,2,4]triazolo[4,3-a]quinolin-1(2H)-one;
5-amino-8-{5-[(4-methylpiperazin-1-yl)methyl]-3-thienyl}[1,2,4]triazolo[4,3-a]quinolin-1(2H)-one;
5-(hydroxymethyl)-8-[5-(piperazin-1-ylmethyl)-2-thienyl][1,2,4]triazolo[4,3-a]quinolin-1(2H)-one;
5-(morpholin-4-ylmethyl)-8-(3-thienyl)[1,2,4]triazolo[4,3-a]quinolin-1(2H)-one;
5-{[(2-methoxyethyl)(methyl)amino]methyl}-8-(3-thienyl)[1,2,4]triazolo[4,3-a]quinolin-1(2H)-one;
1-oxo-N-(2-piperidin-1-ylethyl)-8-(3-thienyl)-1,2-dihydro[1,2,4]triazolo[4,3-a]quinoline-5-carboxamide;
5-{[(2-morpholin-4-ylethyl)amino]methyl}-8-(3-thienyl)[1,2,4]triazolo[4,3-a]quinolin-1(2H)-one;
5-[(dimethylamino)methyl]-8-(3-thienyl)[1,2,4]triazolo[4,3-a]quinolin-1(2H)-one;
N-[2-(dimethylamino)ethyl]-8-{4-[(4-methylpiperazin-1-yl)methyl]phenyl}-1-oxo-1,2-dihydro[1,2,4]triazolo[4,3-a]quinoline-5-carboxamide;
[1-oxo-8-(3-thienyl)-1,2-dihydro[1,2,4]triazolo[4,3-a]quinolin-5-yl]methyl glycinate;
5-{[2-(hydroxymethyl)morpholin-4-yl]methyl}-8-(1H-pyrrol-2-yl)[1,2,4]triazolo[4,3-a]quinolin-1(2H)-one;
5-[(4-methylpiperazin-1-yl)methyl]-8-(1H-pyrrol-2-yl)[1,2,4]triazolo[4,3-a]quinolin-1(2H)-one;
5-{[2-(hydroxymethyl)morpholin-4-yl]methyl}-8-(3-thienyl)[1,2,4]triazolo[4,3-a]quinolin-1(2H)-one;
5-{[4-(2-hydroxyethyl)piperazin-1-yl]methyl}-8-(3-thienyl)[1,2,4]triazolo[4,3-a]quinolin-1(2H)-one;
N³,N³-dimethyl-N¹-[1-oxo-8-(3-thienyl)-1,2-dihydro[1,2,4]triazolo[4,3-a]quinolin-5-yl]-beta-alaninamide;
5-{[(3R)-3-hydroxypyrrolidin-1-yl]methyl}-8-(3-thienyl)[1,2,4]triazolo[4,3-a]quinolin-1(2H)-one;
5-{[(3R)-3-hydroxypyrrolidin-1-yl]methyl}-8-(3-thienyl)[1,2,4]triazolo[4,3-a]quinolin-1(2H)-one;
5-{[(2,3-dihydroxypropyl)(methyl)amino]methyl}-8-(3-thienyl)[1,2,4]triazolo[4,3-a]quinolin-1(2H)-one;
5-({methyl[2-(methylamino)ethyl]amino}methyl)-8-(3-thienyl)[1,2,4]triazolo[4,3-a]quinolin-1(2H)-one;
5-{[(3-methoxypropyl)amino]methyl}-8-(3-thienyl)[1,2,4]triazolo[4,3-a]quinolin-1(2H)-one;
5-{[(2-hydroxyethyl)(methyl)amino]methyl}-8-(3-thienyl)[1,2,4]triazolo[4,3-a]quinolin-1(2H)-one;
5-{[(3-hydroxypropyl)amino]methyl}-8-(3-thienyl)[1,2,4]triazolo[4,3-a]quinolin-1(2H)-one;
N-[2-(dimethylamino)ethyl]-8-{5-[(methylamino)methyl]-3-thienyl}-1-oxo-1,2-dihydro[1,2,4]triazolo[4,3-a]quinoline-5-carboxamide;
N-[2-(dimethylamino)ethyl]-8-[3-(dimethylamino)prop-1-yn-1-yl]-1-oxo-1,2-dihydro[1,2,4]triazolo[4,3-a]quinoline-5-carboxamide;
5-{[[3-(dimethylamino)propyl](methyl)amino]methyl}-8-(3-thienyl)[1,2,4]triazolo[4,3-a]quinolin-1(2H)-one;
5-({[3-(dimethylamino)propyl]amino}methyl)-8-(3-thienyl)[1,2,4]triazolo[4,3-a]quinolin-1(2H)-one;
N-[2-(dimethylamino)ethyl]-1-oxo-8-[4-(piperazin-1-ylmethyl)phenyl]-1,2-dihydro[1,2,4]triazolo[4,3-a]quinoline-5-carboxamide;
5-(aminomethyl)-8-(1H-pyrrol-2-yl)[1,2,4]triazolo[4,3-a]quinolin-1(2H)-one;
[1-oxo-8-(3-thienyl)-1,2-dihydro[1,2,4]triazolo[4,3-a]quinolin-5-yl]methyl N-methylglycinate;
5-{[(3-methoxypropyl)amino]methyl}-8-{5-[(methylamino)methyl]-3-thienyl}[1,2,4]triazolo[4,3-a]quinolin-1(2H)-one;
5-{[(2-hydroxyethyl)(methyl)amino]methyl}-8-{5-[(methylamino)methyl]-3-thienyl}[1,2,4]triazolo[4,3-a]quinolin-1(2H)-one;
5-{[4-(2-hydroxyethyl)piperazin-1-yl]methyl}-8-(1H-pyrrol-2-yl)[1,2,4]triazolo[4,3-a]quinolin-1(2H)-one;
5-{[(2,3-dihydroxypropyl)(methyl)amino]methyl}-8-{5-[(methylamino)methyl]-3-thienyl}[1,2,4]triazolo[4,3-a]quinolin-1(2H)-one;
5-{[(3R)-3-hydroxypyrrolidin-1-yl]carbonyl}-8-(3-thienyl)[1,2,4]triazolo[4,3-a]quinolin-1(2H)-one;
1-oxo-8-(3-thienyl)-1,2-dihydro[1,2,4]triazolo[4,3-a]quinoline-5-carboxylic acid;
8-{5-[(methylamino)methyl]-3-thienyl}-5-({methyl[2-(methylamino)ethyl]amino}methyl)[1,2,4]triazolo[4,3-a]quinolin-1(2H)-one;
5-{[[3-(dimethylamino)propyl](methyl)amino]methyl}-8-{5-[(methylamino)methyl]-3-thienyl}[1,2,4]triazolo[4,3-a]quinolin-1(2H)-one;
5-{[(3-hydroxypropyl)amino]methyl}-8-{5-[(methylamino)methyl]-3-thienyl}[1,2,4]triazolo[4,3-a]quinolin-1(2H)-one;
5-({[3-(dimethylamino)propyl]amino}methyl)-8-{5-[(methylamino)methyl]-3-thienyl}[1,2,4]triazolo[4,3-a]quinolin-1(2H)-one;
5-methyl-8-[3-(piperazin-1-ylmethyl)phenyl][1,2,4]triazolo[4,3-a]quinolin-1(2H)-one;
N-[2-(dimethylamino)ethyl]-8-{5-[(4-methylpiperazin-1-yl)methyl]-2-thienyl}-1-oxo-1,2-dihydro[1,2,4]triazolo[4,3-a]quinoline-5-carboxamide;
5-{[(3R)-3-hydroxypyrrolidin-1-yl]carbonyl}-8-{5-[(methylamino)methyl]-3-thienyl}[1,2,4]triazolo[4,3-a]quinolin-1(2H)-one;
5-[(methylamino)methyl]-8-(3-thienyl)[1,2,4]triazolo[4,3-a]quinolin-1(2H)-one;
5-({[2-(methylamino)ethyl]amino}methyl)-8-(3-thienyl)[1,2,4]triazolo[4,3-a]quinolin-1(2H)-one;
5-methyl-8-(5-{[(3S)-pyrrolidin-3-ylamino]methyl}-2-thienyl)[1,2,4]triazolo[4,3-a]quinolin-1(2H)-one;
5-methyl-8-(5-{[(3R)-pyrrolidin-3-ylamino]methyl}-2-thienyl)[1,2,4]triazolo[4,3-a]quinolin-1(2H)-one;
N-azetidin-3-yl-1-oxo-8-(3-thienyl)-1,2-dihydro[1,2,4]triazolo[4,3-a]quinoline-5-carboxamide
8-{5-[(azetidin-3-ylamino)methyl]-2-thienyl}-5-methyl[1,2,4]triazolo[4,3-a]quinolin-1(2H)-one;
5-{[2-(dimethylamino)ethoxy]methyl}-8-(3-thienyl)[1,2,4]triazolo[4,3-a]quinolin-1(2H)-one;
5-({[(2S)-2,3-dihydroxypropyl]amino}methyl)-8-(3-thienyl)[1,2,4]triazolo[4,3-a]quinolin-1(2H)-one;
5-{[(3S)-3-hydroxypyrrolidin-1-yl]methyl}-8-(3-thienyl)[1,2,4]triazolo[4,3-a]quinolin-1(2H)-one;
5-{[(3-amino-2-hydroxypropyl)amino]methyl}-8-(3-thienyl)[1,2,4]triazolo[4,3-a]quinolin-1(2H)-one;
5-{[(3R)-3-(dimethylamino)pyrrolidin-1-yl]methyl}-8-(3-thienyl)[1,2,4]triazolo[4,3-a]quinolin-1(2H)-one;
5-{[(2-hydroxyethyl)amino]methyl}-8-(3-thienyl)[1,2,4]triazolo[4,3-a]quinolin-1(2H)-one;
5-(aminomethyl)-8-[4-(methoxymethyl)phenyl][1,2,4]triazolo[4,3-a]quinolin-1(2H)-one;
N-(3-hydroxypropyl)-1-oxo-8-(3-thienyl)-1,2-dihydro[1,2,4]triazolo[4,3-a]quinoline-5-carboxamide;
5-{[(3S)-3-hydroxypyrrolidin-1-yl]methyl}-8-(1H-pyrrol-2-yl)[1,2,4]triazolo[4,3-a]quinolin-1(2H)-one;
5-{[(3S)-3-hydroxypyrrolidin-1-yl]carbonyl}-8-(3-thienyl)[1,2,4]triazolo[4,3-a]quinolin-1(2H)-one;
5-({[2-(dimethylamino)ethyl]amino}methyl)-8-(1H-pyrrol-2-yl)[1,2,4]triazolo[4,3-a]quinolin-1(2H)-one;
8-{5-[(ethylamino)methyl]-3-thienyl}-5-methyl[1,2,4]triazolo[4,3-a]quinolin-1(2H)-one;
8-{5-[(isopropylamino)methyl]-3-thienyl}-5-methyl[1,2,4]triazolo[4,3-a]quinolin-1(2H)-one;
N-azetidin-3-yl-8-{5-[(methylamino)methyl]-3-thienyl}-1-oxo-1,2-dihydro[1,2,4]triazolo[4,3-a]quinoline-5-carboxamide;
5-(1H-imidazol-1-ylmethyl)-8-(1H-pyrrol-2-yl)[1,2,4]triazolo[4,3-a]quinolin-1(2H)-one;
5-(1H-imidazol-1-ylmethyl)-8-(3-thienyl)[1,2,4]triazolo[4,3-a]quinolin-1(2H)-one;
5-{[(3-hydroxypropyl)amino]methyl}-8-(1H-pyrrol-2-yl)[1,2,4]triazolo[4,3-a]quinolin-1(2H)-one;
5-[(pyrrolidin-3-ylamino)methyl]-8-(3-thienyl)[1,2,4]triazolo[4,3-a]quinolin-1(2H)-one;
5-{[(3R)-pyrrolidin-3-ylamino]methyl}-8-(3-thienyl)[1,2,4]triazolo[4,3-a]quinolin-1(2H)-one;
5-[(azetidin-3-ylamino)methyl]-8-(3-thienyl)[1,2,4]triazolo[4,3-a]quinolin-1(2H)-one;
5-{[(3S)-3-aminopyrrolidin-1-yl]methyl}-8-(3-thienyl)[1,2,4]triazolo[4,3-a]quinolin-1(2H)-one;
5-{[(3R)-3-aminopyrrolidin-1-yl]methyl}-8-(3-thienyl)[1,2,4]triazolo[4,3-a]quinolin-1(2H)-one;
5-{[(3R)-3-(dimethylamino)pyrrolidin-1-yl]methyl}-8-(1H-pyrrol-2-yl)[1,2,4]triazolo[4,3-a]quinolin-1(2H)-one;
5-{[(2-hydroxyethyl)amino]methyl}-8-(1H-pyrrol-2-yl)[1,2,4]triazolo[4,3-a]quinolin-1(2H)-one;
5-[(3-aminoazetidin-1-yl)methyl]-8-(3-thienyl)[1,2,4]triazolo[4,3-a]quinolin-1(2H)-one;
5-({[2-(dimethylamino)ethyl]amino}methyl)-8-(3-thienyl)[1,2,4]triazolo[4,3-a]quinolin-1(2H)-one;
5-({[2-(1H-imidazol-4-yl)ethyl]amino}methyl)-8-(3-thienyl)[1,2,4]triazolo[4,3-a]quinolin-1(2H)-one;
5-({[3-(1H-imidazol-4-yl)propyl]amino}methyl)-8-(3-thienyl)[1,2,4]triazolo[4,3-a]quinolin-1(2H)-one;
5-[(isopropylamino)methyl]-8-(3-thienyl)[1,2,4]triazolo[4,3-a]quinolin-1(2H)-one;
5-[(ethylamino)methyl]-8-(3-thienyl)[1,2,4]triazolo[4,3-a]quinolin-1(2H)-one;
5-[(cyclopropylamino)methyl]-8-(3-thienyl)[1,2,4]triazolo[4,3-a]quinolin-1(2H)-one;
5-{[(cyclopropylmethyl)amino]methyl}-8-(3-thienyl)[1,2,4]triazolo[4,3-a]quinolin-1(2H)-one;
5-({[2-(dimethylamino)-1-methylethyl]amino}methyl)-8-(3-thienyl)[1,2,4]triazolo[4,3-a]quinolin-1(2H)-one;
N-[2-(dimethylamino)-1-methylethyl]-1-oxo-8-(3-thienyl)-1,2-dihydro[1,2,4]triazolo[4,3-a]quinoline-5-carboxamide;
5-{[methyl(2-pyridin-4-ylethyl)amino]methyl}-8-(3-thienyl)[1,2,4]triazolo[4,3-a]quinolin-1(2H)-one;
5-[(3-aminoazetidin-1-yl)carbonyl]-8-(3-thienyl)[1,2,4]triazolo[4,3-a]quinolin-1(2H)-one;
N-[2-(1H-imidazol-4-yl)ethyl]-1-oxo-8-(3-thienyl)-1,2-dihydro[1,2,4]triazolo[4,3-a]quinoline-5-carboxamide;
N-[3-(1H-imidazol-4-yl)propyl]-1-oxo-8-(3-thienyl)-1,2-dihydro[1,2,4]triazolo[4,3-a]quinoline-5-carboxamide;
5-({[2-(isopropylamino)ethyl]amino}methyl)-8-(3-thienyl)[1,2,4]triazolo[4,3-a]quinolin-1(2H)-one;
N-[2-(isopropylamino)ethyl]-1-oxo-8-(3-thienyl)-1,2-dihydro[1,2,4]triazolo[4,3-a]quinoline-5-carboxamide;
N-[(1-ethylpyrrolidin-2-yl)methyl]-1-oxo-8-(3-thienyl)-1,2-dihydro[1,2,4]triazolo[4,3-a]quinoline-5-carboxamide;
5-({[3-(dimethylamino)propyl]amino}methyl)-8-(1H-pyrrol-2-yl)[1,2,4]triazolo[4,3-a]quinolin-1(2H)-one;
5-{[4-(hydroxymethyl)-1H-1,2,3-triazol-1-yl]methyl}-8-(3-thienyl)[1,2,4]triazolo[4,3-a]quinolin-1(2H)-one;
5-{[(pyridin-2-ylmethyl)amino]methyl}-8-(3-thienyl)[1,2,4]triazolo[4,3-a]quinolin-1(2H)-one;
5-({[(5-methyl-2-furyl)methyl]amino}methyl)-8-(3-thienyl)[1,2,4]triazolo[4,3-a]quinolin-1(2H)-one;
5-{[(2-pyridin-2-ylethyl)amino]methyl}-8-(3-thienyl)[1,2,4]triazolo[4,3-a]quinolin-1(2H)-one;
5-(methoxymethyl)-8-(3-thienyl)[1,2,4]triazolo[4,3-a]quinolin-1(2H)-one;
5-({[(5-methylpyrazin-2-yl)methyl]amino}methyl)-8-(3-thienyl)[1,2,4]triazolo[4,3-a]quinolin-1(2H)-one;
5-({4-[(methylamino)methyl]-1H-1,2,3-triazol-1-yl}methyl)-8-(3-thienyl)[1,2,4]triazolo[4,3-a]quinolin-1(2H)-one;
5-(aminomethyl)-8-(3-thienyl)[1,2,4]triazolo[4,3-a]quinolin-1(2H)-one;
5-{[(2-aminoethyl)amino]methyl}-8-(3-thienyl)[1,2,4]triazolo[4,3-a]quinolin-1(2H)-one;
5-chloro-2H-[1,2,4]triazolo[4,3-α]quinolin-1-one;
4-(1-oxo-1,2-dihydro-[1,2,4]triazolo[4,3-α]quinolin-5-yl)-benzaldehyde;
3-methoxy-2-(1-oxo-1,2-dihydro[1,2,4]triazolo[4,3-a]quinolin-5-yl)benzaldehyde;
8-bromo-1-oxo-1,2-dihydro-[1,2,4]-triazolo[4,3-a]quinoline-5-caroxylic acid;
8-bromo-1-oxo-1,2-dihydro-[1,2,4]-triazolo[4,3-a]quinoline-5-caroxylic acid ethyl ester;
7-chloro-5-methyl-2H-[1,2,4]triazolo[4,3-α]quinolin-1-one;
4-(5-methyl-1-oxo-1,2-dihydro-[1,2,4]triazolo[4,3-α]quinolin-7-yl)-benzaldehyde;
2-methoxy-5-(5-methyl-1-oxo-1,2-dihydro[1,2,4]triazolo[4,3-a]quinolin-7-yl)benzaldehyde;
3-(7-methoxy-5-methyl-1-oxo-1,2-dihydro-[1,2,4]triazolo[4,3-α]quinolin-8-yl)-benzaldehyde
2-methoxy-5-(7-methoxy-5-methyl-1-oxo-1,2-dihydro[1,2,4]triazolo[4,3-a]quinolin-8-yl)benzaldehyde;
2-(7-methoxy-5-methyl-1-oxo-1,2-dihydro[1,2,4]triazolo[4,3-a]quinolin-8-yl)benzaldehyde;
8-bromo-5-bromomethyl[1,2[4]triazolo[4,3-a]quinolin-1(2H)-one;
8-bromo-5-hydroxymethyl[1,2[4]triazolo[4,3-a]quinolin-1(2H)-one;
8-bromo-1-oxo-1,2-dihydro[1,2,4]triazolo[4,3-a]quinoline-4-carboxylic acid;
4-amino-8-bromo[1,2,4]triazolo[4,3-a]quinolin-1(2H)-one;
8-bromo-5-phenyl[1,2,4]triazolo[4,3-a]quinolin-1(2H)-one;
8-bromo-5-methoxymethy[1,2,4]triazolo[4,3-a]quinolin-1(2H)-one;
8-Bromo-5-(aminomethyl)[1,2,4]triazolo[4,3-a]quinolin-1(2H)-one;
5-(azidomethyl)-8-bromo[1,2,4]triazolo[4,3-a]quinolin-1(2H)-one;
7-bromo-5-thien-2-yl[1,2,4]triazolo[4,3-a]quinolin-1(2H)-one ethyl 8-chloro-7-methoxy-1-oxo-1,2-dihydro[1,2,4]triazolo[4,3-a]quinoline-5-carboxylate;
8-chloro-7-methoxy-1-oxo-1,2-dihydro[1,2,4]triazolo[4,3-a]quinoline-5-carboxylic acid;
5-amino-8-chloro-7-methoxy[1,2,4]triazolo[4,3-a]quinolin-1(2H)-one;
8-chloro-5-(hydroxymethyl)-7-methoxy[1,2,4]triazolo[4,3-a]quinolin-1(2H)-one;
2-{3-[(1-oxo-1,2-dihydro[1,2,4]triazolo[4,3-a]quinolin-5-yl)oxy]propyl}-1H-isoindole-1,3(2H)-dione;
2-{3-[(8-chloro-5-methyl-1-oxo-1,2-dihydro[1,2,4]triazolo[4,3-a]quinolin-7-yl)oxy]propyl}-1H-isoindole-1,3(2H)-dione;
7-(3-aminopropoxy)-8-chloro-5-methyl[1,2,4]triazolo[4,3-a]quinolin-1(2H)-one.

22-25. (canceled)

26. A method of treatment of a human or animal by limiting cell replication comprising administering to said human or animal an effective amount of a compound according to claim 1 or a pharmaceutically acceptable salt of said compound.

27. A method of treatment of a human or animal suffering from cancer comprising administering to said human or animal an effective amount of a compound according to claim 1 or a pharmaceutically acceptable salt of said compound.

28. A method of treatment of a human or animal suffering from a neoplastic disease comprising administering to said human or animal an effective amount of a compound according to claim 1 or a pharmaceutically acceptable salt of said compound.

29. A method of treatment of a human or animal suffering from proliferative diseases comprising administering to said human or animal an effective amount of a compound according to claim 1 or a pharmaceutically acceptable salt of said compound.

30-33. (canceled)
